(19) United States
(12) Patent Application Publication
(10) Pub. No.: US 2025/0122173 A1
(43) Pub. Date: Apr. 17, 2025

(54) MODULATORS OF RHO-ASSOCIATED PROTEIN KINASE

(71) Applicant: Redx Pharma Limited, Macclesfield (GB)

(72) Inventors: Nicolas E.S. Guisot, Macclesfield (GB); Clifford D. Jones, Macclesfield (GB)

(21) Appl. No.: 18/726,176

(22) PCT Filed: Jan. 19, 2023
(86) PCT No.: PCT/GB23/50109

§ 371 Date: Jul. 2, 2024

(30) Foreign Application Priority Data
Jan. 20, 2022 (GB) 2200735.5

(51) Int. Cl. C07D 403/14; A61K 31/4196; A61K 31/4439; A61K 31/444; A61K 31/454; C07D 401/14; C07D 471/04

(52) U.S. Cl. CPC C07D 403/14; A61K 31/4196; A61K 31/4439; A61K 31/444; A61K 31/454; C07D 401/14; C07D 471/04

(57) ABSTRACT

This invention relates to novel compounds and pharmaceutical compositions comprising the novel compounds. More specifically, the invention relates to compounds useful as modulators of Rho-associated protein kinase (ROCK), for example ROCK1 and/or ROCK2 inhibitors. This invention also relates to processes for preparing the compounds, uses of the compounds and methods of treatment employing the compounds. The compounds of the invention may therefore be used in treating ROCK-mediated diseases.

This invention relates to novel compounds and pharmaceutical compositions comprising the novel compounds. More specifically, the invention relates to compounds useful as modulators of Rho-associated protein kinase (ROCK), for example ROCK1 and/or ROCK2 inhibitors. This invention also relates to processes for preparing the compounds, uses of the compounds and methods of treatment employing the compounds. The compounds of the invention may therefore be used in treating ROCK-mediated diseases.

### BACKGROUND

Rho-associated coiled-coil Kinase (ROCK) is a member of the AGC family of serine/threonine kinases that are involved in many aspects of cellular signalling [Lock et at 2012]. Highly conserved in mammalian species, two isoforms of ROCK are known to exist ROCK1 and ROCK2. They share 65% homology in their amino acid sequence and 92% homology in their kinase domain. In addition, they contain a long coiled-coil domain, a PH domain and a Rho binding domain, suggesting they are activated by Rho GTPases such as RhoA that link several classes of cell surface receptor to internal kinase signalling. ROCK1 and ROCK2 have been shown to bind to and phosphorylate a large number of substrate proteins involved in processes such as migration, pro-fibrotic cytokine production, cell adhesion and proliferation, but the most studied are those associated with actin cytoskeleton rearrangements [Surma et at 2014]. The best characterized ROCK substrate is the Myosin Phosphatase Target Subunit 1 (MYPT1), a regulatory subunit of myosin phosphatase. MYPT1 counteracts the activity of Myosin Light Chain (MLC) kinase and thus decreases the contraction of smooth muscle cells. ROCK phosphorylates and deactivates MYPT1 and that in turn leads to mLC kinase phosphorylation, activation and actomyosin contraction [Feng et al 1999; Velasco et al 2002]. When administered systemically pan-ROCK inhibitors induce vasodilation, hyperaemia and hypotension, limiting their use as therapeutic agents in the context of fibrosis and most other diseases [Kast et al 2007]. ROCK is also known to phosphorylate LIM-kinases (LIMK1 and LIMK2) which in turn phosphorylate cofilin leading to increased cellular actin filaments [Maekawa et al 1999]. Many additional substrates have been identified, some of which have been suggested to be more specifically phosphorylated by either ROCK1 or ROCK2. [Hartmann et al 2015]. ROCK isoforms are differentially expressed in different tissues with ROCK1 predominant in the kidney, liver, lungs, and testis, while ROCK2 is predominantly expressed in the brain and muscle tissues [Nakagawa et a 1996]. Isoform expression can also alter during disease, with ROCK2 often increasing in expression in comparison to ROCK1; taken together all these data strongly suggest that ROCK1 and ROCK2 have both overlapping and distinct functions in homeostasis and disease.

Both ROCK1 and ROCK2 are involved in numerous pro-fibrotic processes with pan-ROCK inhibitors able to strongly suppress TGFβ stimulated myofibroblast activation, chemokine driven fibroblast migration and EMT in response to profibrotic mediators such as Lysophosphatidic acid (LPA) and endothelin [Sakai et al 2016]. The specific role of each ROCK isoform in disease is largely unknown at present although both independent and redundant roles have been suggested. Studies using haplozygous knockout mice (ROCK2+/−) where one allele of ROCK2 has been removed (reducing the expression of ROCK2) have shown that multiple aspects of disease can be attenuated. Knipe et al (Knipe R Pharmacol Rev. 2015; 67(1):103-17; Knipe R Am J Respir Cell Mol Biol. 2018 April; 58(4):471-481) demonstrated that both ROCK1+/− mice and ROCK2+/− mice were protected in the bleomycin induced lung fibrosis model of IPF. A more recent study looked at both ROCK2+/− mice and ROCK2 haplozygous mice, with one allele of ROCK2 mutated to a kinase activity dead configuration, thereby reducing signalling but not expression of ROCK2 [Wei et al 2020]. In both models the knockout mice showed remarkable protection from weight gain when fed a high fat diet, with some resistance to obesity induced type 2 diabetes and liver steatosis. The results mirrored work carried out by Soliman et al 2016 that also showed that ROCK2+/− mice were protected from type 2 diabetes insulin resistance when fed a high fat diet. An earlier study also looking at ROCK2 in the context of poor nutrition demonstrated a role for ROCK2 in atherosclerosis. Mice deficient in Low Density Lipoprotein receptor (LDLr−/−) develop atherosclerosis when fed a high cholesterol diet. Bone marrow specific deletion of ROCK2 in these mice led to dramatic reduction in atherosclerotic lesions and reduced inflammatory cytokines in macrophages, suggesting ROCK2 may play a significant role in macrophage derived fatty foam cell formation that is a key pathogenic driver in atherosclerosis [Zhou et al 2012]. ROCK2 may also play a role in the often-fatal thrombosis observed in atherosclerosis. LDLr−/− mice with platelet specific knockout of ROCK2 showed a significant reduction in circulating blood clots when fed a high cholesterol diet [Sladojevic et al 2017]. Further tissue specific knockouts of ROCK2 have also shown protection in models of human disease. Deletion of ROCK2 in cardiac specific fibroblasts reduced both cardiac hypertrophy and fibrosis and improved heart function in an angiotensin II infusion model of heart failure [Shimizu et al 2017]. Similar protection was also observed in the same model when ROCK2 was specifically deleted in cardiomyocytes [Okamoto et al 2013]. Taken together the knockout data shows that ROCK2 is not only involved in fibrosis but also in a number of the deleterious processes that lead to fibrosis and tissue remodelling making ROCK2 a nodal point for disease progression.

ROCK also plays a significant role in pathologies of the central nervous system (CNS). For example, ROCK-signalling has been demonstrated to be elevated in the serum, spleen, brain and spinal cord of Multiple Sclerosis (MS) patients compared to healthy individuals. Dysregulation of autophagy contributes to the development of misfolded tau aggregates in early Alzheimer's disease (AD). When inhibited, ROCK2, which is expressed in excitatory neurons, can induce autophagy pathways and inhibition of ROCK2 could be a therapeutic approach for AD.

Furthermore, it is an aim of certain embodiments of this invention to provide new treatments, for example treatments for diabetes, inflammation, Alzheimer's, muscular dystrophies, hypertension, fibrosis, cancer, pathologies of the central nervous system and other conditions associated with ROCK1 and/or ROCK2. In particular, it is an aim of certain embodiments of this invention to provide compounds which have comparable activity to existing ROCK therapies.

It is an aim of certain embodiments of this invention to provide compounds which exhibit reduced cytotoxicity or increased solubility relative to prior art compounds and existing therapies.

Another aim of certain embodiments of this invention is to provide compounds having a convenient pharmacokinetic profile and a suitable duration of action following dosing. A further aim of certain embodiments of this invention is to provide compounds in which the metabolised fragment or fragments of the drug after absorption are GRAS (Generally Regarded As Safe).

Certain embodiments of the present invention satisfy some or all of the above aims.

### BRIEF SUMMARY OF THE DISCLOSURE

The present invention provides a compound of formula (I) and pharmaceutically acceptable salts thereof:

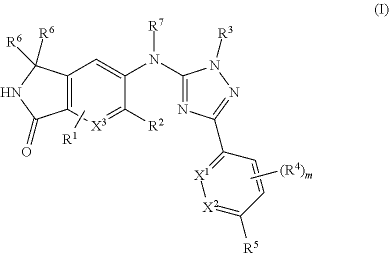

X¹ and X² are each independently selected from carbon and nitrogen; wherein at least one of X¹ and X² is carbon;
X³ is selected from carbon and nitrogen;
R¹ and R² are each independently selected from H, halo, nitro, cyano, NR⁸R⁸, OR¹⁰, SR⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl, C₁-C₄-alkyl substituted with NR⁸R⁹, C₁-C₄-alkyl substituted with OR¹⁰, and cyclopropyl;
R³ is independently selected from C₁-C₄-alkyl, C₁-C₄-haloalkyl, C₀-C₃-alkylene-R^{3a}, and C₂-C₄-alkylene-R^{3b}; wherein R^{3a} is independently at each occurrence selected from cyclopropyl and azetidinyl, said cyclopropyl or azetidinyl groups being optionally substituted with from 1 to 4 R¹¹ groups; wherein R^{3b} is independently at each occurrence selected from NR⁸R⁸, OR¹⁰ and SR⁸;
R⁴ and R¹² are each independently at each occurrence selected from halo, nitro, cyano, NR⁸R⁹, OR¹⁰, SRI, SO₂R⁸, SO₂NR⁸R⁸, CO₂R⁸, C(O)R⁸, CONR⁸R⁸, CR⁸R⁸NR⁸R⁹, CR⁸R⁸OR⁸, C₁-C₄-alkyl, C₂-C₄-alkenyl, C₂-C₄-alkynyl, C₁-C₄-haloalkyl and cyclopropyl;
R⁵ is independently selected from CONR⁸R⁸, unsubstituted phenyl, phenyl substituted with from 1 to 4 R¹¹ groups, and 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is optionally substituted with a single R¹³ group and/or from 1 to 3 R¹² groups;
R⁶ is independently at each occurrence selected from H, halo, C₁-C₄-alkyl, C₁-C₄-alkyl substituted with NR⁵R⁸, C₁-C₄-alkyl substituted with OR¹⁰, and cyclopropyl, or the two R⁶ groups and the carbon atom to which they are attached may together form a C₃-C₅ cycloalkyl ring;
R⁷ and R⁸ are each independently selected from H, C₁-C₄-alkyl and C₁-C₄-haloalkyl;
R⁹ is independently at each occurrence selected from H, C₁-C₄-alkyl, C(O)—C₁-C₄-alkyl and S(O)₂—C₁-C₄-alkyl; or R⁵ and R⁹, together with the nitrogen atom to which they are attached together form a C₅-C₈-heterocycloalkyl group optionally substituted with from 0 to 4 R¹¹ groups;
R¹⁰ is independently at each occurrence selected from H, C₁-C₄-alkyl, C(O)—C₁-C₄-alkyl and C₁-C₄-haloalkyl;
R¹¹ is independently at each occurrence selected from ═O, halo, nitro, cyano, NR⁸R⁸, OR¹⁴, SR⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl and cyclopropyl;
R¹³ is independently selected from C₃-C₆-cycloalkyl, 3- to 6-membered-heterocycloalkyl, CR⁸R⁸NR⁸R⁹ and CR⁸R⁸OR⁸; wherein where R¹³ is cycloalkyl or heterocycloalkyl, R¹³ is optionally substituted with from 1 to 4 R¹¹ groups;
R¹⁴ is independently at each occurrence selected from H, C₁-C₄-alkyl; C₁-C₄-haloalkyl and C(O)—C₁-C₄-alkyl; and
m is an integer selected from 0, 1, 2, 3 and 4;
wherein any of the aforementioned alkyl or cycloalkyl (e.g. cyclopropyl) groups is optionally substituted, where chemically possible, by 1 to 5 substituents which are each independently at each occurrence selected from the group consisting of: halo, oxo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In an embodiment, the compound of formula (I) is a compound of formula (II):

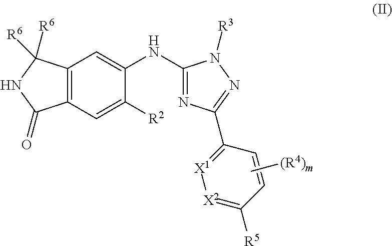

wherein X¹, X², R², R³, R⁴, R⁵, R⁶ and m are as described above for compounds of formula (I).

In an embodiment, the compound of formula (I) is a compound of formula (III):

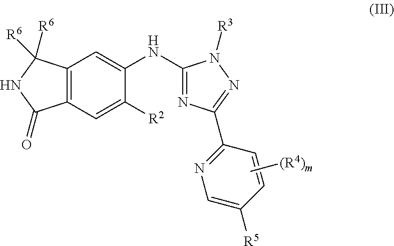

wherein R², R³, R⁴, R⁵, R⁶ and m are as described above for compounds of formula (I).

In an embodiment, the compound of formula (I) is a compound of formula (IV):

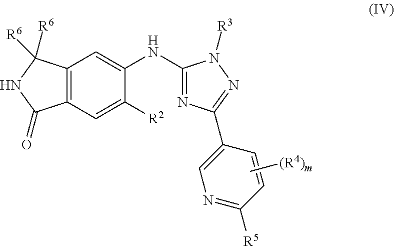

wherein R², R³, R⁴, R⁵, R⁶ and m are as described above for compounds of formula (I).

In an embodiment, the compound of formula (I) is a compound of formula (V):

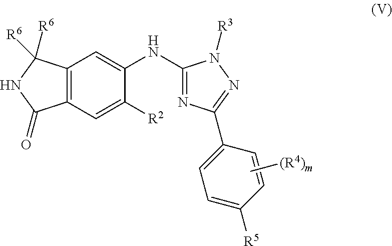

wherein R², R³, R⁴, R⁵, R⁶ and m are as described above for compounds of formula (I).

In an embodiment, the compound of formula (I) is a compound of formula (VI):

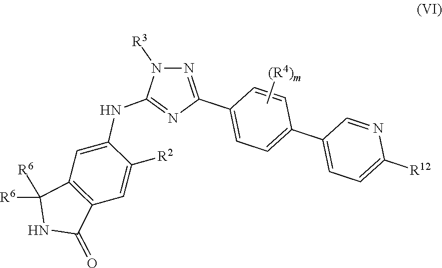

wherein R², R³, R⁴, R⁶, R¹² and m are as described above for compounds of formula (I).

In an embodiment, the compound of formula (I) is a compound of formula (VII):

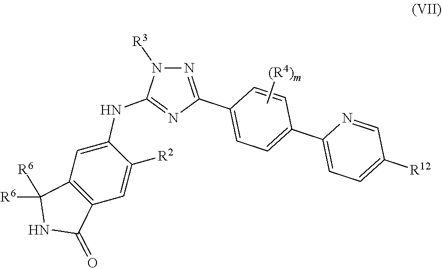

wherein R², R³, R⁴, R⁶, R¹² and m are as described above for compounds of formula (I).

The following embodiments apply to compounds of any of formulae (I)-(VII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

It may be that X¹ is carbon and X² is nitrogen. It may be that X¹ is nitrogen and X² is carbon. It may be that X¹ is CR^{4a} and X² is nitrogen; wherein R^{4a} is independently selected from halo, nitro, cyano, OR¹⁰, C₁-C₄-alkyl and C₁-C₄-haloalkyl and cyclopropyl. It may be that X¹ is nitrogen and X² is CR^{4a}; wherein R^{4a} is independently selected from halo, nitro, cyano, OR¹⁰, C₁-C₄-alkyl and C₁-C₄-haloalkyl and cyclopropyl.

It may be that X³ is carbon. It may be that X³ is nitrogen.

It may be that R¹, if present, is attached to X³. For the absence of doubt, this embodiment applies where X³ is carbon.

It may be that R¹ and R² are each independently selected from H, halo, nitro, cyano, OR¹⁰, C₁-C₄-alkyl, C₁-C₄-haloalkyl and cyclopropyl.

It may be that R¹ is independently selected from H, halo, nitro, cyano, OR¹⁰, C₁-C₄-alkyl and C₁-C₄-haloalkyl and cyclopropyl. It may be that R¹ is independently selected from H, fluoro, C₁-C₃-alkyl, e.g. methyl, C₁-C₃-fluoroalkyl and cyclopropyl. It may be that R¹ is H.

It may be that R² is independently selected from H, halo, nitro, cyano, OR¹⁰, C₁-C₄-alkyl, C₁-C₄-haloalkyl and cyclopropyl. It may be that R² is independently selected from H, fluoro, C₁-C₃-alkyl, C₁-C₃-fluoroalkyl and cyclopropyl. It may be that R² is H. It may be that R² is independently selected from fluoro, C₁-C₃-alkyl, C₁-C₃-fluoroalkyl and cyclopropyl. It may be that R² is independently halo, e.g. fluoro. It may be that R² is independently C₁-C₃-alkyl, e.g. methyl.

R³ may be independently selected from C₁-C₄-alkyl and cyclopropyl. R³ may be C₁-C₄-alkyl, e.g. methyl or ethyl.

m may be 0. m may be selected from 1 and 2. m may be 1.

R⁴ may be independently at each occurrence selected from halo, nitro, cyano, OR¹⁰, C₁-C₄-alkyl, C₁-C₄-haloalkyl, CR⁸R⁸NR⁸R⁹, CR⁸R⁸OR⁸, and cyclopropyl. R⁴ may be independently at each occurrence selected from halo, nitro, cyano, OR¹⁰, C₁-C₄-alkyl, C₁-C₄-haloalkyl, CR⁸R⁸NR⁸R⁹, CR⁸R⁸OR⁸, and cyclopropyl. It may be that R⁴ is independently at each occurrence selected from fluoro, C₁-C₃-alkyl, C₁-C₃-fluoroalkyl and cyclopropyl. It may be that R⁴ is independently at each occurrence selected from fluoro, C₁-C₃-alkyl, C₁-C₃-fluoroalkyl and cyclopropyl. It may be that R⁴ is independently at each occurrence halo, e.g. fluoro. It may be that R⁴ is independently at each occurrence C₁-C₃-alkyl, e.g. methyl.

It may be that m is 1 and R⁴ is C₁-C₃-alkyl, e.g. methyl. The single R⁴ group may be ortho to the R⁵ group. Alternatively, the single R⁴ group may be meta to the R⁵ group.

R⁵ may be independently selected from CONR⁸R⁸, unsubstituted phenyl, phenyl substituted with from 1 to 4 R¹¹ groups, and 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is substituted with a single R¹³ group and/or from 1 to 3 R¹² groups.

R⁵ may be independently 5- or 6-membered monocyclic heterocyclyl, wherein said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is substituted with a single R¹³ group and/or from 1 to 3 R¹² groups. R⁵ may be independently 5- or 6-membered monocyclic heterocyclyl, wherein said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is optionally substituted with a single R¹³ group and/or from 1 to 3 R¹² groups. R⁵ may be independently 5- or 6-membered monocyclic heteroaryl, optionally substituted with a single R¹³ group and/or from 1 to 3 R¹² groups. R⁵ may be independently 5-membered monocyclic heteroaryl, optionally substituted with a single R¹³ group and/or from 1 to 3 R¹² groups. R⁵ may be independently 6-membered monocyclic heteroaryl, optionally substituted with a single R¹³ group and/or from 1 to 3 R¹² groups. R⁵ may be independently 5-membered monocyclic heteroaryl, optionally substituted with a single R¹³ group and/or from 1 to 3 R¹² groups. The R⁵ group may comprise a nitrogen in the ring system. R⁵ may selected from imidazole and pyridine, optionally substituted with a single R¹³ group and/or from 1 to 3 R¹² groups. R⁵ may be imidazole, optionally substituted with a single R¹³ group and/or from 1 to 3 R¹² groups. R⁵ may be pyridine, optionally substituted with a single R¹³ group and/or from 1 to 3 R¹² groups.

R⁵ may be independently selected from 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is substituted with a single R¹³ group and/or from 1 to 3 R¹² groups. R⁵ is independently selected from 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is optionally substituted with a single R¹³ group and/or from 1 to 3 R¹² groups.

R⁵ may be independently selected from 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with from 1 to 4 R¹¹ groups and wherein any unsaturated ring is substituted with from 1 to 3 R¹² groups. R⁵ may be independently selected from 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with from 1 to 4 R¹¹ groups and wherein any unsaturated ring is optionally substituted with from 1 to 3 R¹² groups.

R⁵ may be independently 5- or 6-membered monocyclic heterocyclyl, wherein said heterocyclyl group may be saturated, unsaturated or partially unsaturated; wherein any saturated ring or partially unsaturated ring is optionally substituted with from 1 to 4 R¹¹ groups and wherein any unsaturated ring is substituted with from 1 to 3 R¹² groups. R⁵ may be independently 5- or 6-membered monocyclic heterocyclyl, wherein said heterocyclyl group may be saturated, unsaturated or partially unsaturated; wherein any saturated ring or partially unsaturated ring is optionally substituted with from 1 to 4 R¹¹ groups and wherein any unsaturated ring is optionally substituted with from 1 to 3 R¹² groups. R⁵ may be independently 5- or 6-membered monocyclic heteroaryl, optionally substituted with from 1 to 3 R¹² groups. R⁵ may be independently 5-membered monocyclic heteroaryl, optionally substituted with from 1 to 3 R¹² groups. R⁵ may be independently 6-membered monocyclic heteroaryl, optionally substituted with from 1 to 3 R¹² groups. R⁵ may be independently 5-membered monocyclic heteroaryl, optionally substituted with from 1 to 3 R¹² groups. The R⁵ group may comprise a nitrogen in the ring system. R⁵ may selected from imidazole and pyridine, optionally substituted with from 1 to 3 R¹² groups. R⁵ may be imidazole, optionally substituted with from 1 to 3 R¹² groups. R⁵ may be pyridine, optionally substituted with from 1 to 3 R¹² groups.

R⁵ may be unsubstituted phenyl. R⁵ may be phenyl substituted with from 1 to 4 R¹¹ groups. R⁵ may be phenyl substituted with R¹¹ group. R⁵ may be phenyl substituted with cyclopropyl.

R⁵ may have the structure:

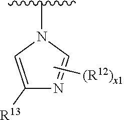

wherein x1 is an integer independently selected from 0, 1 and 2. x1 may be 0.

R⁵ may have the structure:

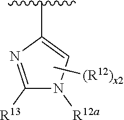

wherein x2 is an integer independently selected from 0 and 1; and R^{12a} is independently selected from: H and C₁-C₄-alkyl. x2 may be 0. R^{12a} may be C₁-C₄-alkyl, e.g. methyl.

R⁵ may have the structure:

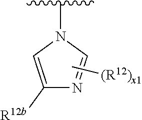

wherein R^{12b} is independently selected from halo, C₁-C₄-alkyl, C₂-C₄-alkenyl, C₂-C₄-alkynyl, C₁-C₄-haloalkyl and cyclopropyl; and x1 is an integer independently selected from 0, 1 and 2. x1 may be 0. R^{12b} may be selected from C₁-C₄-cycloalkyl and cyclopropyl.

R⁵ may have the structure:

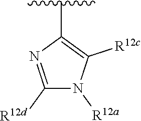

wherein R^{12d} and R^{12c} are each independently selected from H, halo, C₁-C₄-alkyl, C₂-C₄-alkenyl, C₂-C₄-alkynyl, C₁-C₄-haloalkyl, CR⁸R⁸NR⁸R⁹, CR⁸R⁸OR⁸ and cyclopropyl; and R^{12a} is independently selected from: H, C₁-C₄-alkyl and cyclopropyl. R^{12a} may be C₁-C₄-alkyl, e.g. methyl. R^{12d} may be H. R^{12d} may be selected from C₁-C₄-cycloalkyl and cyclopropyl. R^{12c} may be H. R^{12c} may be selected from C₁-C₄-cycloalkyl and cyclopropyl.

R⁵ may have the structure:

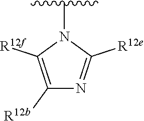

R^{12b} wherein R^{12b} is independently selected from halo, C₁-C₄-alkyl, C₂-C₄-alkenyl, C₂-C₄-alkynyl, C₁-C₄-haloalkyl, CR⁸R⁸NR⁸R⁹, CR⁸R⁸OR⁸ and cyclopropyl; and wherein R^{12f} and R^{12e} are each independently selected from H, halo, C₁-C₄-alkyl, C₂-C₄-alkenyl, C₂-C₄-alkynyl, C₁-C₄-haloalkyl and cyclopropyl. R¹ may be H. R^{12e} may be H. R^{12e} may be selected from C₁-C₄-cycloalkyl and cyclopropyl. R^{12b} may be selected from C₁-C₄-cycloalkyl and cyclopropyl.

R⁵ may have the structure:

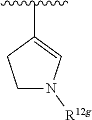

wherein R^{12g} is independently selected from H, C₁-C₄-alkyl, C₁-C₄-haloalkyl and cyclopropyl. R^{12g} may be C₁-C₄-haloalkyl e.g. trifluoroethyl.

R⁵ may have the structure:

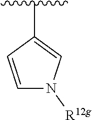

wherein R^{12g} is independently selected from H, C₁-C₄-alkyl, C₁-C₄-haloalkyl and cyclopropyl. R^{12g} may be C₁-C₄-haloalkyl e.g. trifluoroethyl.

R⁵ may have the structure:

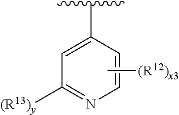

wherein y is an integer independently selected from 0 and 1; and x3 is an integer independently selected from 0, 1, 2 and 3. x3 may be 0. x3 may be 1. y may be 0. y may be 1.

R⁵ may have the structure:

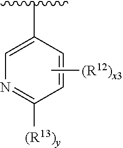

wherein y is an integer independently selected from 0 and 1; and x3 is an integer independently selected from 0, 1, 2 and 3. x3 may be 0. x3 may be 1. y may be 0. y may be 1.

R⁵ may have the structure:

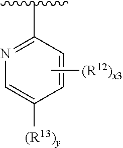

wherein y is an integer independently selected from 0 and 1; and x3 is an integer independently selected from 0, 1, 2 and 3. x3 may be 0. x3 may be 1. y may be 0. y may be 1.

R⁵ may have the structure:

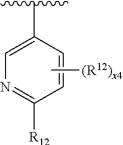

wherein x4 is an integer independently selected from 0, 1 and 2. x4 may be 0. x4 may be 1.

R⁵ may have the structure:

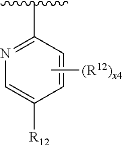

wherein x4 is an integer independently selected from 0, 1 and 2. x4 may be 0. x4 may be 1.

R⁵ may have the structure:

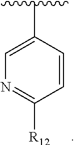

R⁵ may have the structure:

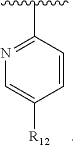

R⁵ may have the structure:

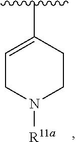

wherein R^{11a} is selected from H, C₁-C₄-alkyl, C₁-C₄-haloalkyl and cyclopropyl. R^{11a} may be C₁-C₄-alkyl, e.g. methyl. R^{11a} may be C₁-C₄-haloalkyl.

R⁵ may have the structure:

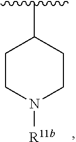

wherein R^{11b} is selected from H, C₁-C₄-alkyl, C₁-C₄-haloalkyl and cyclopropyl. R^{11b} may be C₁-C₄-haloalkyl, e.g. trifluoroethyl or trifluoropropyl.

R⁵ may have the structure:

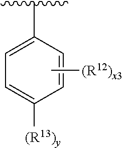

wherein y is an integer independently selected from 0 and 1; and x3 is an integer independently selected from 0, 1, 2 and 3. x3 may be 0. x3 may be 1. y may be 0. y may be 1. R³ may be cyclopropyl.

R⁵ may have the structure:

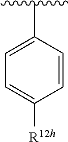

wherein R^{12h} is selected from H, C₁-C₄-alkyl, C₁-C₄-haloalkyl and cyclopropyl. R^{12h} may be cyclopropyl.

R¹² may be independently at each occurrence selected from halo, nitro, cyano, OR¹⁰, C₁-C₄-alkyl and C₁-C₄-haloalkyl and cyclopropyl. It may be that R¹² is independently at each occurrence selected from fluoro, C₁-C₃-alkyl, C₁-C₃-fluoroalkyl and cyclopropyl. It may be that R¹² is independently at each occurrence selected from fluoro, C₁-C₃-alkyl, C₁-C₃-fluoroalkyl and cyclopropyl. It may be that R¹² is independently at each occurrence selected from C₁-C₃-alkyl, e.g. methyl, and cyclopropyl. It may be that R¹² is independently at each occurrence halo, e.g. fluoro. It may be that R¹² is independently at each occurrence C₁-C₃-alkyl, e.g. methyl.

R¹³ may be C₃-C₆-cycloalkyl. R¹³ may be CR⁸R⁸NR⁸R⁹. R¹³ may be CR⁸R⁸OR⁸. R¹³ may be 3- to 6-membered-heterocycloalkyl. R¹³ may be C₃-C₄-cycloalkyl. R¹³ may be 4-membered-heterocycloalkyl. R¹³ may be selected from oxetane, azetidine, cyclopropyl and cyclobutyl. R¹³ may be cyclopropyl. R¹³ may be C₁-C₄-alkyl, e.g. methyl.

Illustrative R⁵ groups include:

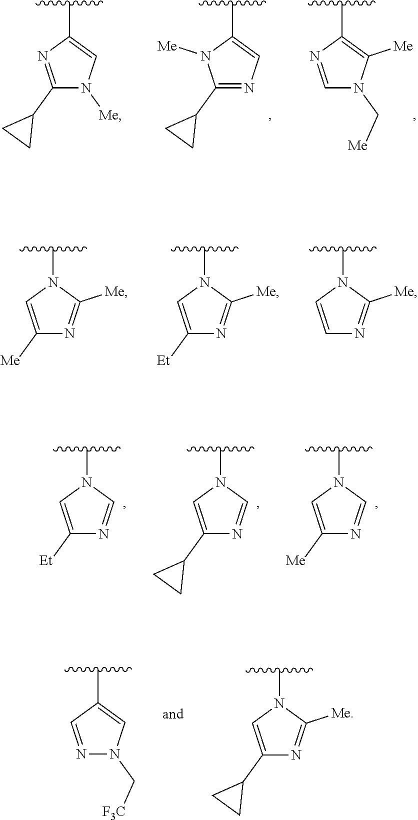

Further illustrative R⁵ groups include:

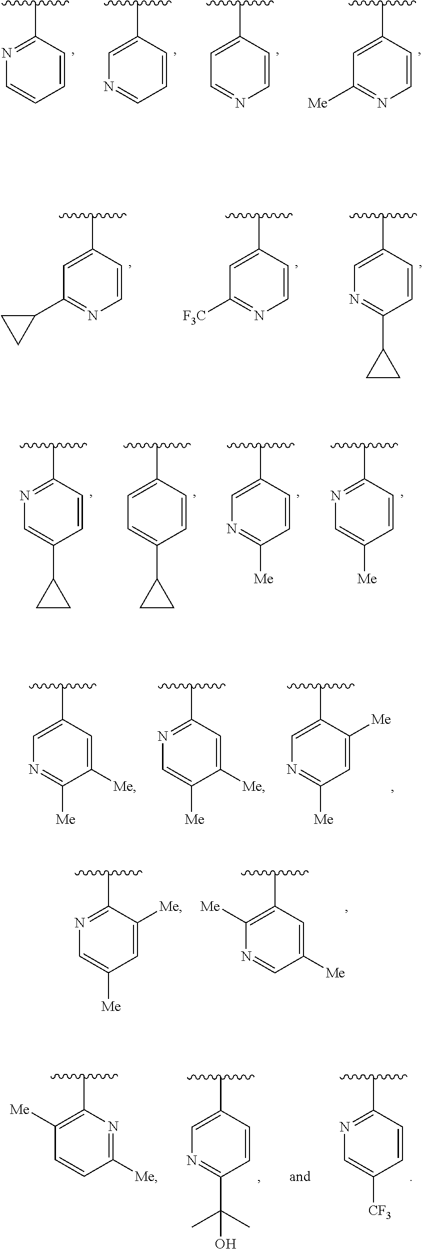

Further illustrative R⁵ groups include:

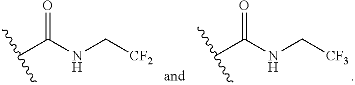

Further illustrative R⁵ groups include:

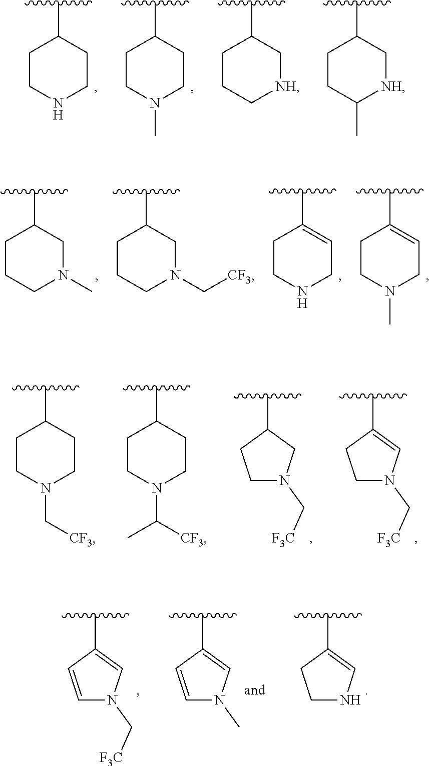

It may be that the two R⁶ groups and the carbon atom to which they are attached together form a C₃-C₆ cycloalkyl ring. It may be that the two R⁶ groups and the carbon atom to which they are attached together form a cyclopropyl ring.

R⁶ may be independently at each occurrence selected from H, fluoro, C₁-C₄-alkyl and cyclopropyl. R⁵ may be independently at each occurrence selected from H and C₁-C₄-alkyl. R⁶ may at each occurrence be H. R⁶ may at each occurrence be C₁-C₄-alkyl, e.g. Me. It may be that a single R⁶ group is H and a single R⁶ group is C₁-C₄-alkyl, e.g. Me.

R⁷ may be H. R⁷ may be C₁-C₄-alkyl, e.g. methyl.

The compound of formula (I) may be selected from:

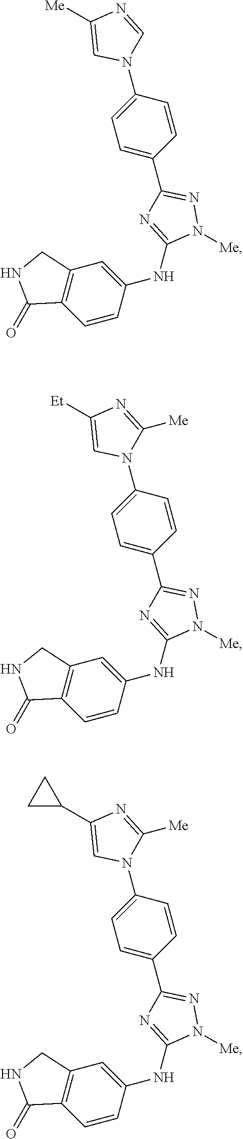 [SMILES: Cc1cn(-c2ccc(-c3nc(Nc4ccc5c(c4)CNC5=O)n(C)n3)cc2)cn1 ; CCc1cn(-c2ccc(-c3nc(Nc4ccc5c(c4)CNC5=O)n(C)n3)cc2)c(C)n1 ; Cc1nc(C2CC2)cn1-c1ccc(-c2nc(Nc3ccc4c(c3)CNC4=O)n(C)n2)cc1]

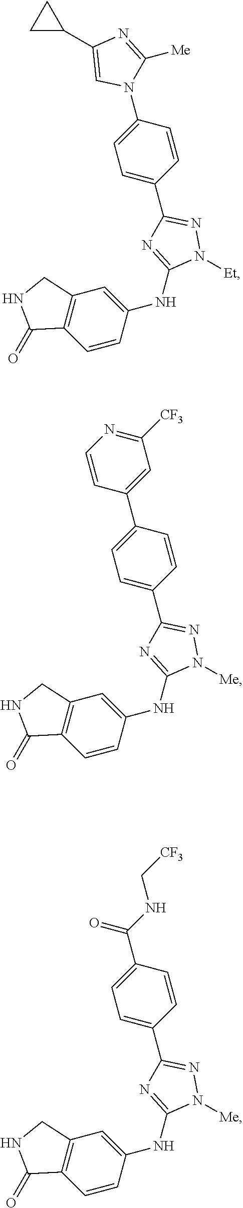 [SMILES: CCn1nc(-c2ccc(-n3cc(C4CC4)nc3C)cc2)nc1Nc1ccc2c(c1)CNC2=O ; Cn1nc(-c2ccc(-c3ccnc(C(F)(F)F)c3)cc2)nc1Nc1ccc2c(c1)CNC2=O ; Cn1nc(-c2ccc(C(=O)NCC(F)(F)F)cc2)nc1Nc1ccc2c(c1)CNC2=O]

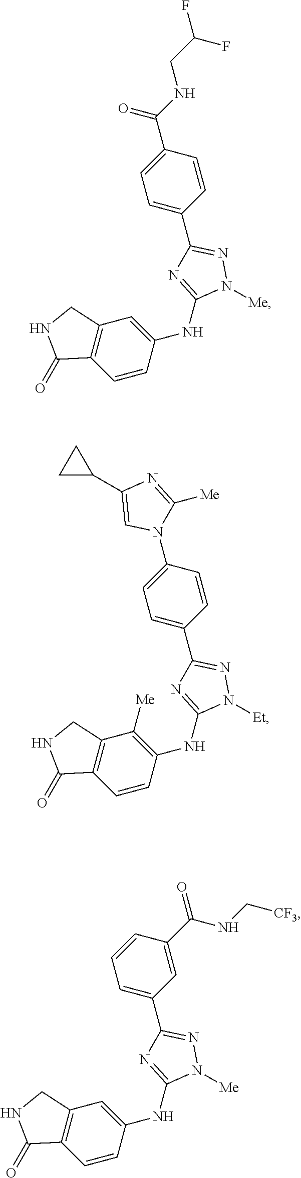 [SMILES: Cn1nc(-c2ccc(C(=O)NCC(F)F)cc2)nc1Nc1ccc2c(c1)CNC2=O ; CCn1nc(-c2ccc(-n3cc(C4CC4)nc3C)cc2)nc1Nc1ccc2c(c1C)CNC2=O ; Cn1nc(-c2cccc(C(=O)NCC(F)(F)F)c2)nc1Nc1ccc2c(c1)CNC2=O]

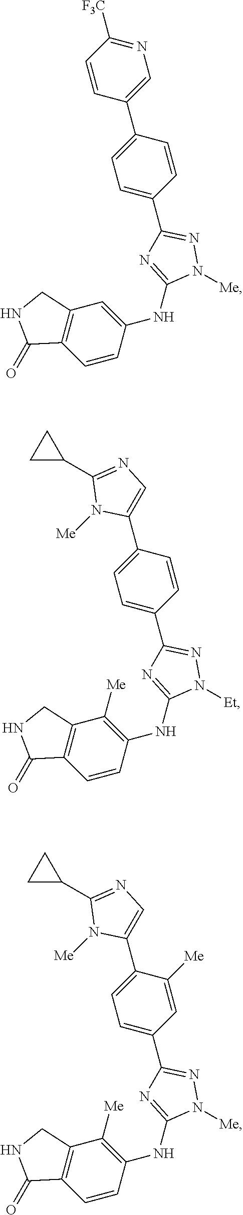 [SMILES: Cn1nc(-c2ccc(-c3ccc(C(F)(F)F)nc3)cc2)nc1Nc1ccc2c(c1)CNC2=O ; CCn1nc(-c2ccc(-c3cnc(C4CC4)n3C)cc2)nc1Nc1ccc2c(c1C)CNC2=O ; Cc1cc(-c2nc(Nc3ccc4c(c3C)CNC4=O)n(C)n2)ccc1-c1cnc(C2CC2)n1C]

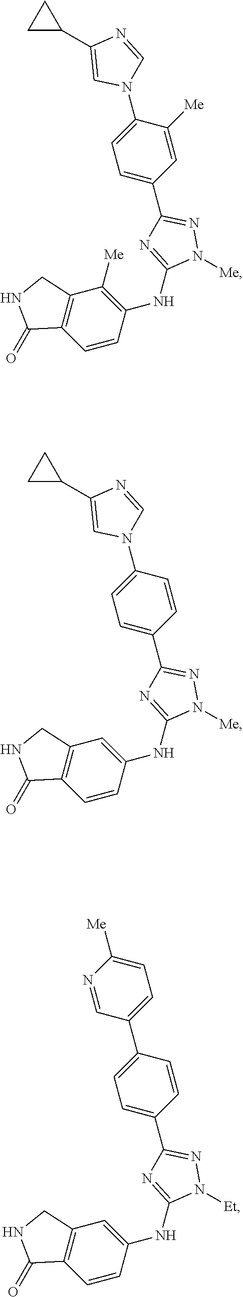 [SMILES: Cc1cc(-c2nc(Nc3ccc4c(c3C)CNC4=O)n(C)n2)ccc1-n1cnc(C2CC2)c1 ; Cn1nc(-c2ccc(-n3cnc(C4CC4)c3)cc2)nc1Nc1ccc2c(c1)CNC2=O ; CCn1nc(-c2ccc(-c3ccc(C)nc3)cc2)nc1Nc1ccc2c(c1)CNC2=O]

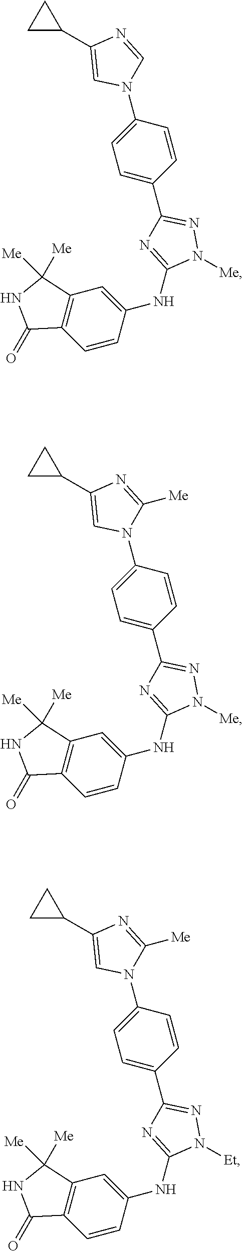 [SMILES: Cn1nc(-c2ccc(-n3cnc(C4CC4)c3)cc2)nc1Nc1ccc2c(c1)C(C)(C)NC2=O ; Cc1nc(C2CC2)cn1-c1ccc(-c2nc(Nc3ccc4c(c3)C(C)(C)NC4=O)n(C)n2)cc1 ; CCn1nc(-c2ccc(-n3cc(C4CC4)nc3C)cc2)nc1Nc1ccc2c(c1)C(C)(C)NC2=O]

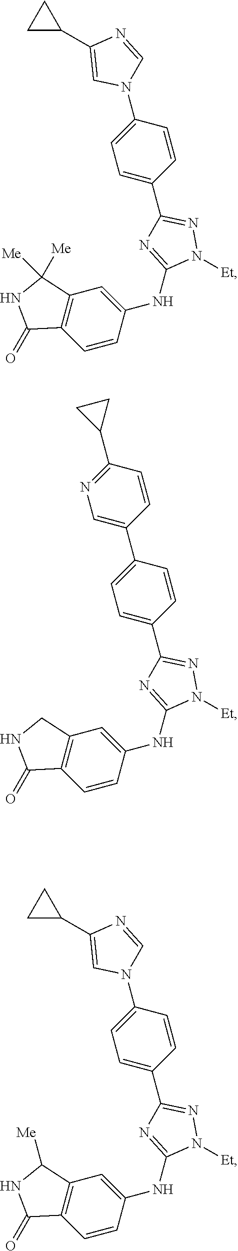 [SMILES: CCn1nc(-c2ccc(-n3cnc(C4CC4)c3)cc2)nc1Nc1ccc2c(c1)C(C)(C)NC2=O ; CCn1nc(-c2ccc(-c3ccc(C4CC4)nc3)cc2)nc1Nc1ccc2c(c1)CNC2=O ; CCn1nc(-c2ccc(-n3cnc(C4CC4)c3)cc2)nc1Nc1ccc2c(c1)C(C)NC2=O]

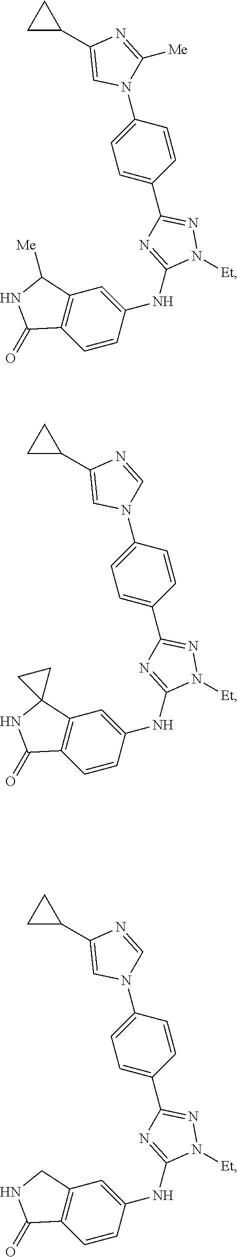 [SMILES: CCn1nc(-c2ccc(-n3cc(C4CC4)nc3C)cc2)nc1Nc1ccc2c(c1)C(C)NC2=O ; CCn1nc(-c2ccc(-n3cnc(C4CC4)c3)cc2)nc1Nc1ccc2c(c1)C1(CC1)NC2=O ; CCn1nc(-c2ccc(-n3cnc(C4CC4)c3)cc2)nc1Nc1ccc2c(c1)CNC2=O]

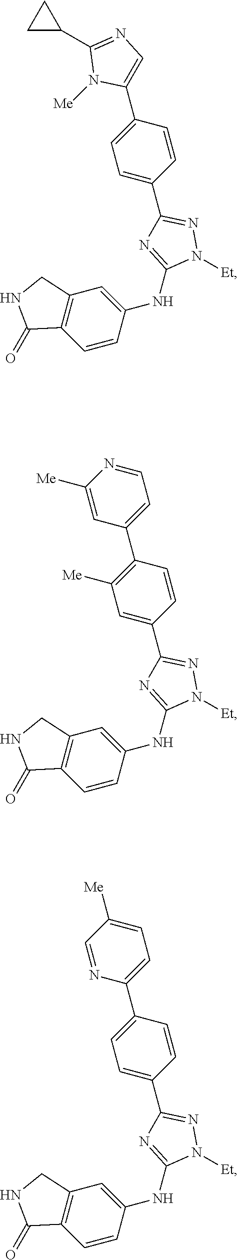 [SMILES: CCn1nc(-c2ccc(-c3cnc(C4CC4)n3C)cc2)nc1Nc1ccc2c(c1)CNC2=O ; CCn1nc(-c2ccc(-c3ccnc(C)c3)c(C)c2)nc1Nc1ccc2c(c1)CNC2=O ; CCn1nc(-c2ccc(-c3ccc(C)cn3)cc2)nc1Nc1ccc2c(c1)CNC2=O]

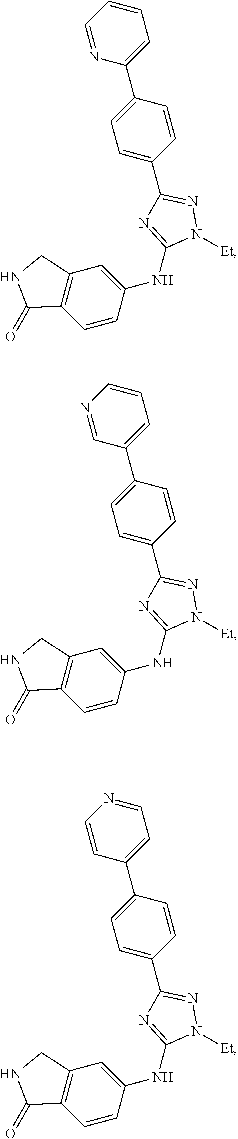 [SMILES: CCn1nc(-c2ccc(-c3ccccn3)cc2)nc1Nc1ccc2c(c1)CNC2=O ; CCn1nc(-c2ccc(-c3cccnc3)cc2)nc1Nc1ccc2c(c1)CNC2=O ; CCn1nc(-c2ccc(-c3ccncc3)cc2)nc1Nc1ccc2c(c1)CNC2=O]

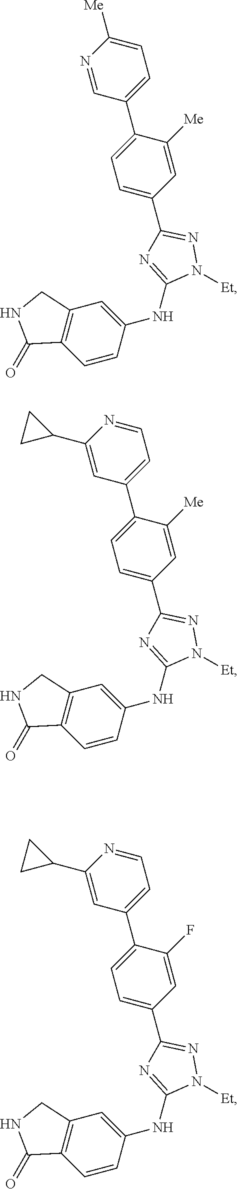

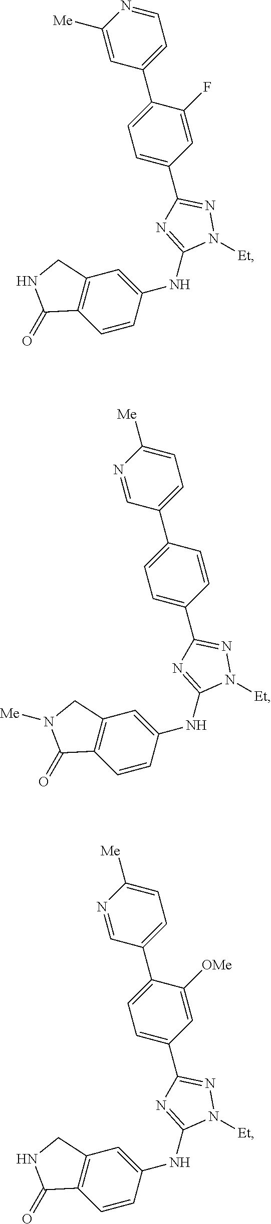 [SMILES: CCn1nc(-c2ccc(-c3ccnc(C)c3)c(F)c2)nc1Nc1ccc2c(c1)CNC2=O ; CCn1nc(-c2ccc(-c3ccc(C)nc3)cc2)nc1Nc1ccc2c(c1)CN(C)C2=O ; CCn1nc(-c2ccc(-c3ccc(C)nc3)c(OC)c2)nc1Nc1ccc2c(c1)CNC2=O]

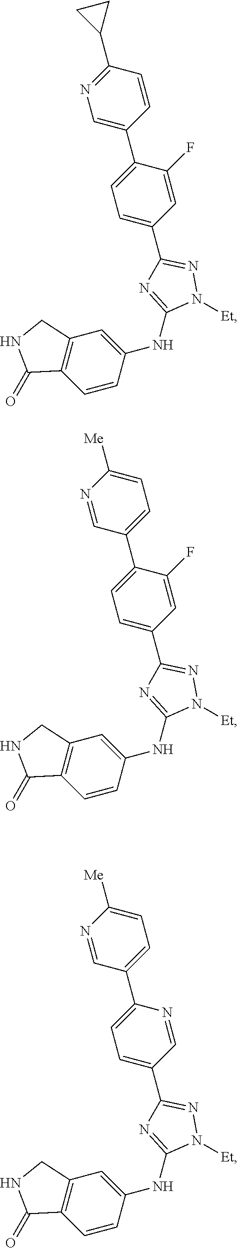 [SMILES: CCn1nc(-c2ccc(-c3ccc(C4CC4)nc3)c(F)c2)nc1Nc1ccc2c(c1)CNC2=O ; CCn1nc(-c2ccc(-c3ccc(C)nc3)c(F)c2)nc1Nc1ccc2c(c1)CNC2=O ; CCn1nc(-c2ccc(-c3ccc(C)nc3)nc2)nc1Nc1ccc2c(c1)CNC2=O]

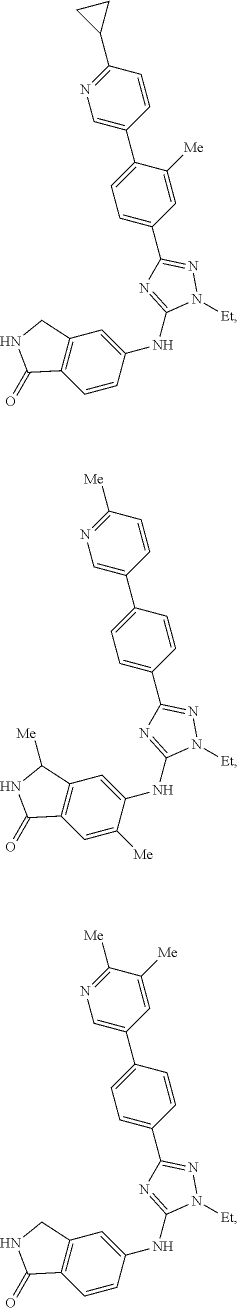 [SMILES: CCn1nc(-c2ccc(-c3ccc(C4CC4)nc3)c(C)c2)nc1Nc1ccc2c(c1)CNC2=O ; CCn1nc(-c2ccc(-c3ccc(C)nc3)cc2)nc1Nc1cc2c(cc1C)C(=O)NC2C ; CCn1nc(-c2ccc(-c3cnc(C)c(C)c3)cc2)nc1Nc1ccc2c(c1)CNC2=O]

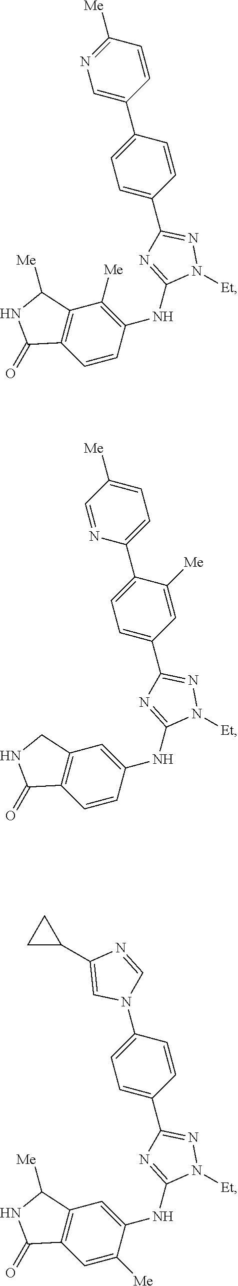 [SMILES: CCn1nc(-c2ccc(-c3ccc(C)nc3)cc2)nc1Nc1ccc2c(c1C)C(C)NC2=O ; CCn1nc(-c2ccc(-c3ccc(C)cn3)c(C)c2)nc1Nc1ccc2c(c1)CNC2=O ; CCn1nc(-c2ccc(-n3cnc(C4CC4)c3)cc2)nc1Nc1cc2c(cc1C)C(=O)NC2C]

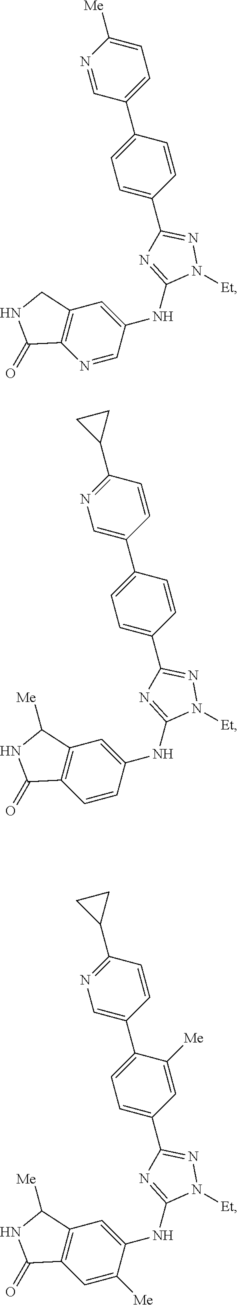 [SMILES: CCn1nc(-c2ccc(-c3ccc(C)nc3)cc2)nc1Nc1cnc2c(c1)CNC2=O ; CCn1nc(-c2ccc(-c3ccc(C4CC4)nc3)cc2)nc1Nc1ccc2c(c1)C(C)NC2=O ; CCn1nc(-c2ccc(-c3ccc(C4CC4)nc3)c(C)c2)nc1Nc1cc2c(cc1C)C(=O)NC2C]

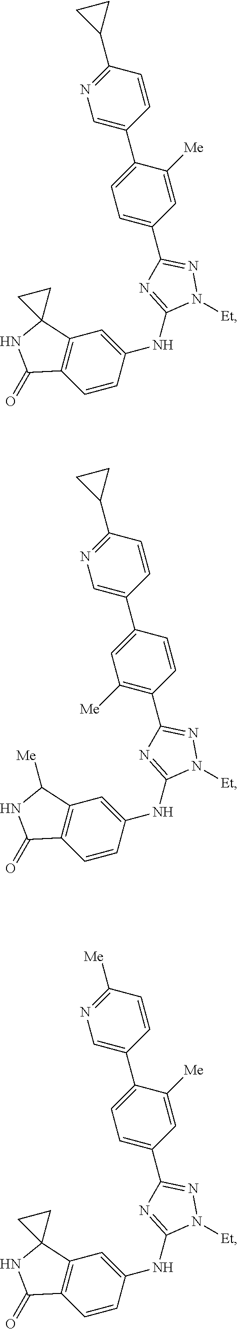 [SMILES: CCn1nc(-c2ccc(-c3ccc(C4CC4)nc3)c(C)c2)nc1Nc1ccc2c(c1)C1(CC1)NC2=O ; CCn1nc(-c2ccc(-c3ccc(C4CC4)nc3)cc2C)nc1Nc1ccc2c(c1)C(C)NC2=O ; CCn1nc(-c2ccc(-c3ccc(C)nc3)c(C)c2)nc1Nc1ccc2c(c1)C1(CC1)NC2=O]

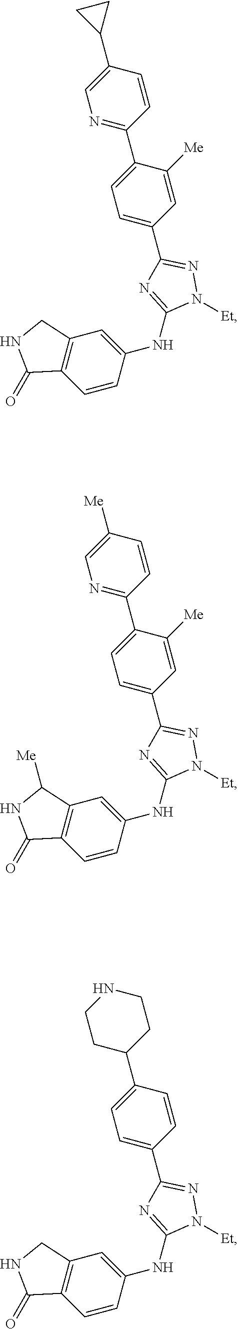 [SMILES: CCn1nc(-c2ccc(-c3ccc(C4CC4)cn3)c(C)c2)nc1Nc1ccc2c(c1)CNC2=O ; CCn1nc(-c2ccc(-c3ccc(C)cn3)c(C)c2)nc1Nc1ccc2c(c1)C(C)NC2=O ; CCn1nc(-c2ccc(C3CCNCC3)cc2)nc1Nc1ccc2c(c1)CNC2=O]

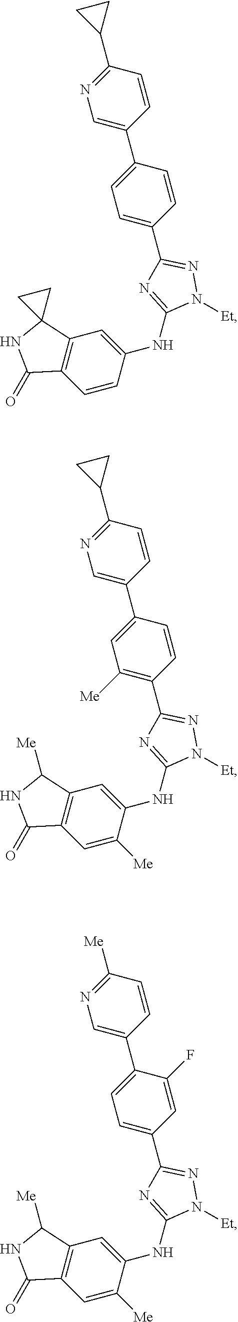 [SMILES: CCn1nc(-c2ccc(-c3ccc(C4CC4)nc3)cc2)nc1Nc1ccc2c(c1)C1(CC1)NC2=O ; CCn1nc(-c2ccc(-c3ccc(C4CC4)nc3)cc2C)nc1Nc1cc2c(cc1C)C(=O)NC2C ; CCn1nc(-c2ccc(-c3ccc(C)nc3)c(F)c2)nc1Nc1cc2c(cc1C)C(=O)NC2C]

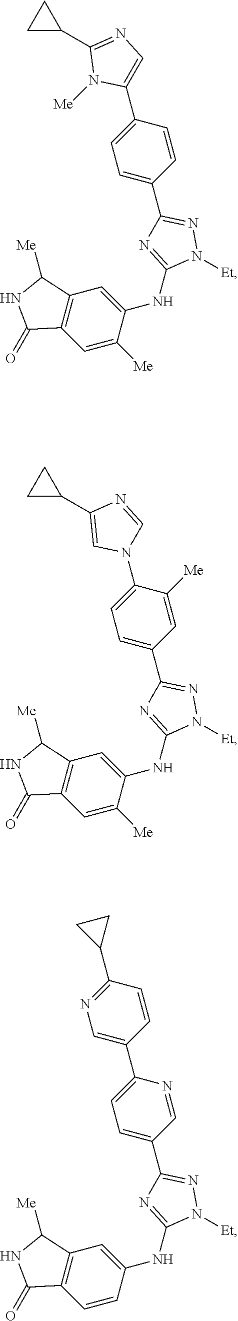 [SMILES: CCn1nc(-c2ccc(-c3cnc(C4CC4)n3C)cc2)nc1Nc1cc2c(cc1C)C(=O)NC2C ; CCn1nc(-c2ccc(-n3cnc(C4CC4)c3)c(C)c2)nc1Nc1cc2c(cc1C)C(=O)NC2C ; CCn1nc(-c2ccc(-c3ccc(C4CC4)nc3)nc2)nc1Nc1ccc2c(c1)C(C)NC2=O]

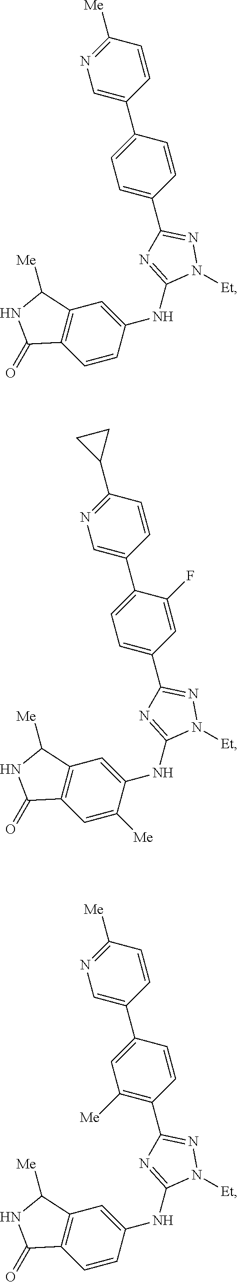 [SMILES: CCn1nc(-c2ccc(-c3ccc(C)nc3)cc2)nc1Nc1ccc2c(c1)C(C)NC2=O ; CCn1nc(-c2ccc(-c3ccc(C4CC4)nc3)c(F)c2)nc1Nc1cc2c(cc1C)C(=O)NC2C ; CCn1nc(-c2ccc(-c3ccc(C)nc3)cc2C)nc1Nc1ccc2c(c1)C(C)NC2=O]

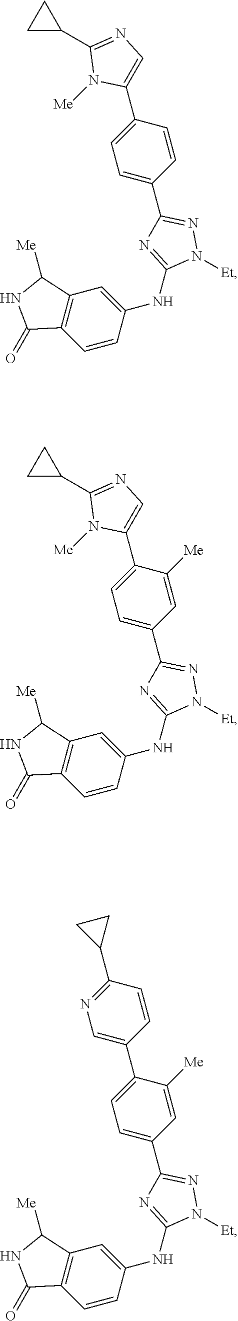 [SMILES: CCn1nc(-c2ccc(-c3cnc(C4CC4)n3C)cc2)nc1Nc1ccc2c(c1)C(C)NC2=O ; CCn1nc(-c2ccc(-c3cnc(C4CC4)n3C)c(C)c2)nc1Nc1ccc2c(c1)C(C)NC2=O ; CCn1nc(-c2ccc(-c3ccc(C4CC4)nc3)c(C)c2)nc1Nc1ccc2c(c1)C(C)NC2=O]

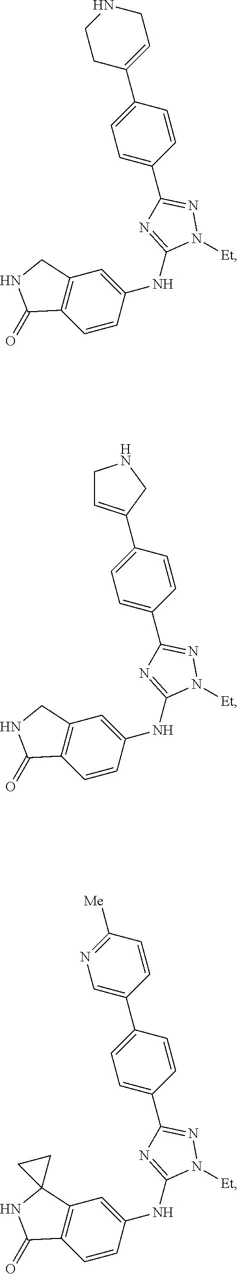 [SMILES: CCn1nc(-c2ccc(C3=CCNCC3)cc2)nc1Nc1ccc2c(c1)CNC2=O ; CCn1nc(-c2ccc(C3=CCNC3)cc2)nc1Nc1ccc2c(c1)CNC2=O ; CCn1nc(-c2ccc(-c3ccc(C)nc3)cc2)nc1Nc1ccc2c(c1)C1(CC1)NC2=O]

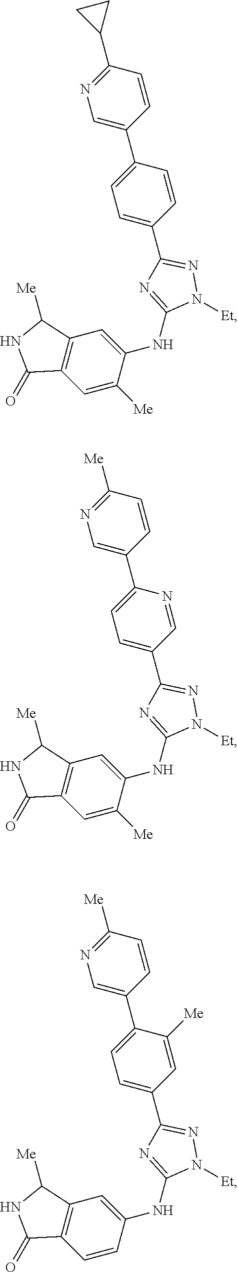 [SMILES: CCn1nc(-c2ccc(-c3ccc(C4CC4)nc3)cc2)nc1Nc1cc2c(cc1C)C(=O)NC2C ; CCn1nc(-c2ccc(-c3ccc(C)nc3)nc2)nc1Nc1cc2c(cc1C)C(=O)NC2C ; CCn1nc(-c2ccc(-c3ccc(C)nc3)c(C)c2)nc1Nc1ccc2c(c1)C(C)NC2=O]

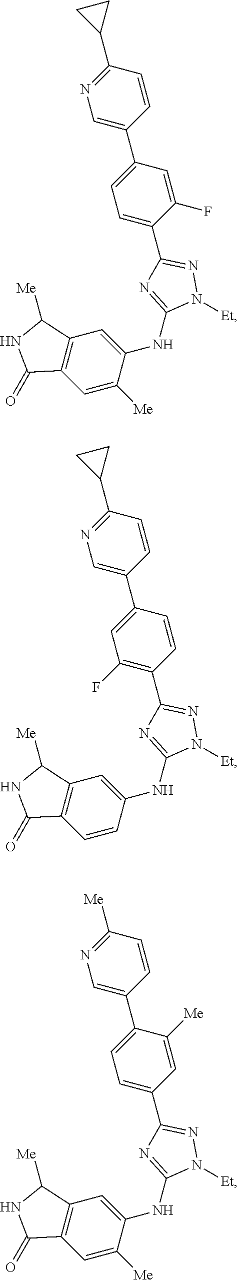 [SMILES: CCn1nc(-c2ccc(-c3ccc(C4CC4)nc3)cc2F)nc1Nc1cc2c(cc1C)C(=O)NC2C ; CCn1nc(-c2ccc(-c3ccc(C4CC4)nc3)cc2F)nc1Nc1ccc2c(c1)C(C)NC2=O ; CCn1nc(-c2ccc(-c3ccc(C)nc3)c(C)c2)nc1Nc1cc2c(cc1C)C(=O)NC2C]

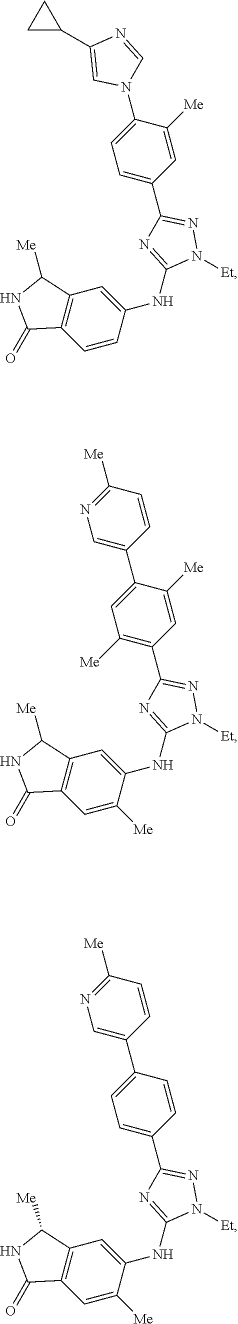 [SMILES: CCn1nc(-c2ccc(-n3cnc(C4CC4)c3)c(C)c2)nc1Nc1ccc2c(c1)C(C)NC2=O ; CCn1nc(-c2cc(C)c(-c3ccc(C)nc3)cc2C)nc1Nc1cc2c(cc1C)C(=O)NC2C ; CCn1nc(-c2ccc(-c3ccc(C)nc3)cc2)nc1Nc1cc2c(cc1C)C(=O)N[C@@H]2C]

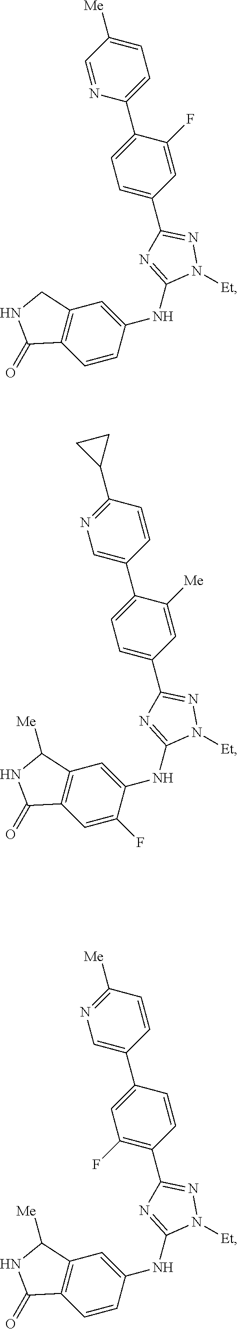 [SMILES: CCn1nc(-c2ccc(-c3ccc(C)cn3)c(F)c2)nc1Nc1ccc2c(c1)CNC2=O ; CCn1nc(-c2ccc(-c3ccc(C4CC4)nc3)c(C)c2)nc1Nc1cc2c(cc1F)C(=O)NC2C ; CCn1nc(-c2ccc(-c3ccc(C)nc3)cc2F)nc1Nc1ccc2c(c1)C(C)NC2=O]

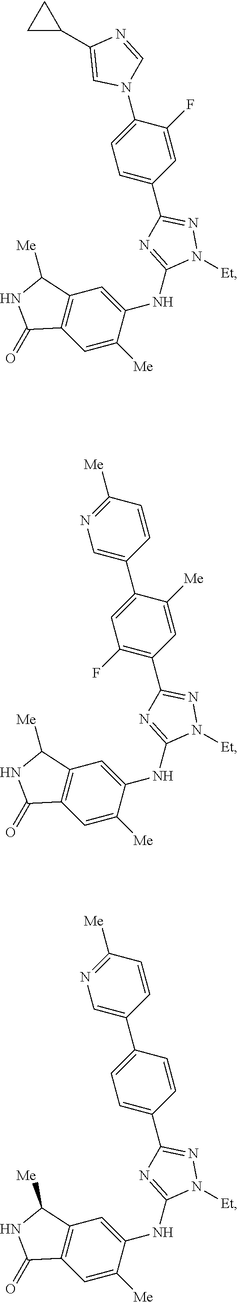 [SMILES: CCn1nc(-c2ccc(-n3cnc(C4CC4)c3)c(F)c2)nc1Nc1cc2c(cc1C)C(=O)NC2C ; CCn1nc(-c2cc(C)c(-c3ccc(C)nc3)cc2F)nc1Nc1cc2c(cc1C)C(=O)NC2C ; CCn1nc(-c2ccc(-c3ccc(C)nc3)cc2)nc1Nc1cc2c(cc1C)C(=O)N[C@H]2C]

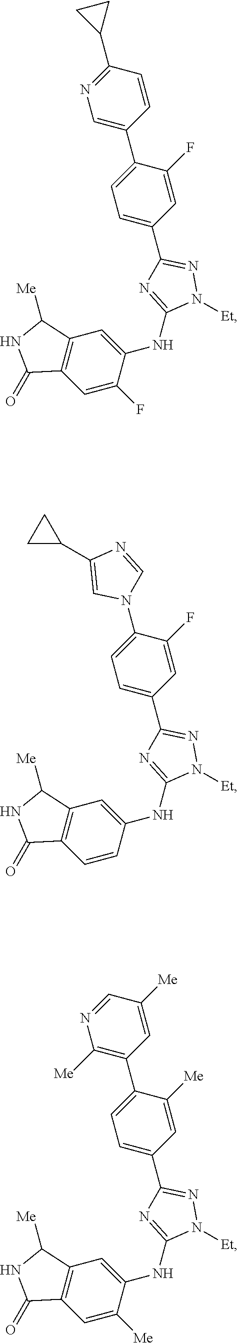 [SMILES: CCn1nc(-c2ccc(-c3ccc(C4CC4)nc3)c(F)c2)nc1Nc1cc2c(cc1F)C(=O)NC2C ; CCn1nc(-c2ccc(-n3cnc(C4CC4)c3)c(F)c2)nc1Nc1ccc2c(c1)C(C)NC2=O ; CCn1nc(-c2ccc(-c3cc(C)cnc3C)c(C)c2)nc1Nc1cc2c(cc1C)C(=O)NC2C]

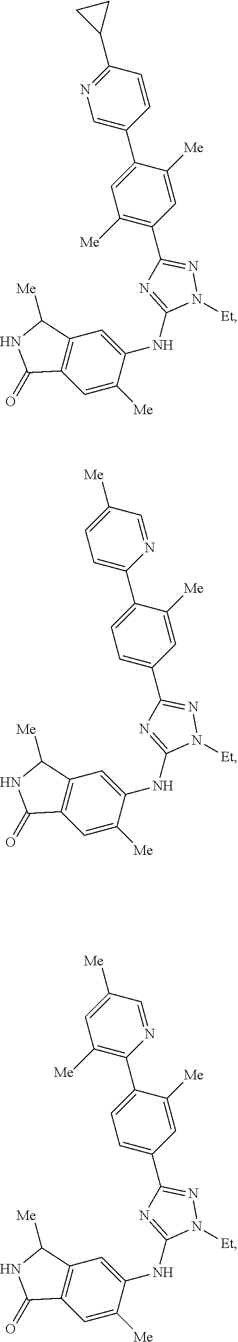 [SMILES: CCn1nc(-c2cc(C)c(-c3ccc(C4CC4)nc3)cc2C)nc1Nc1cc2c(cc1C)C(=O)NC2C ; CCn1nc(-c2ccc(-c3ccc(C)cn3)c(C)c2)nc1Nc1cc2c(cc1C)C(=O)NC2C ; CCn1nc(-c2ccc(-c3ncc(C)cc3C)c(C)c2)nc1Nc1cc2c(cc1C)C(=O)NC2C]

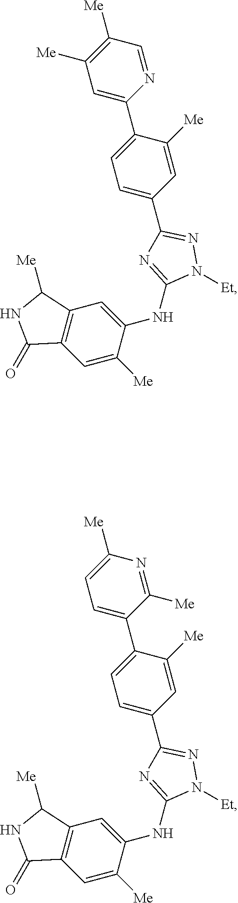 [SMILES: CCn1nc(-c2ccc(-c3cc(C)c(C)cn3)c(C)c2)nc1Nc1cc2c(cc1C)C(=O)NC2C ; CCn1nc(-c2ccc(-c3ccc(C)nc3C)c(C)c2)nc1Nc1cc2c(cc1C)C(=O)NC2C]

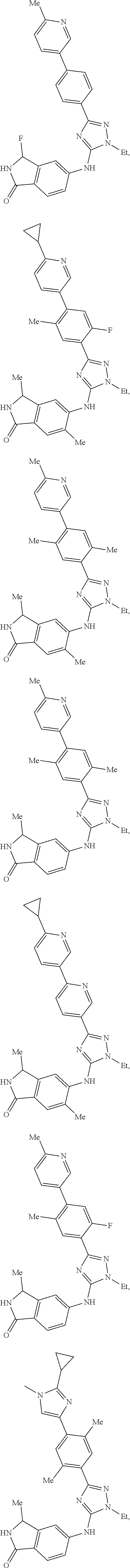

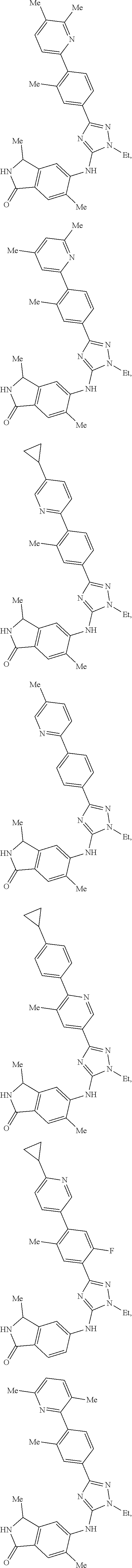

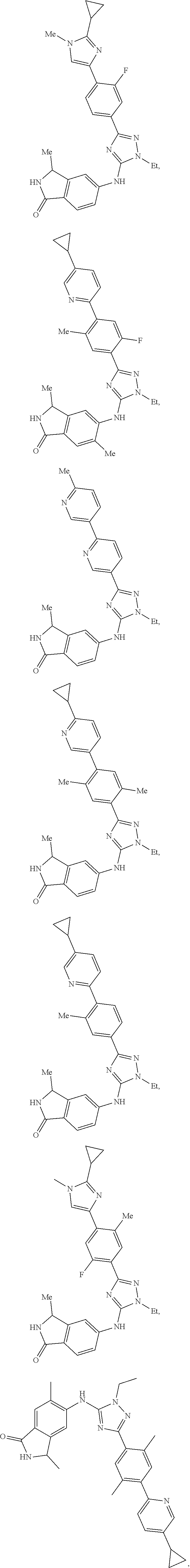

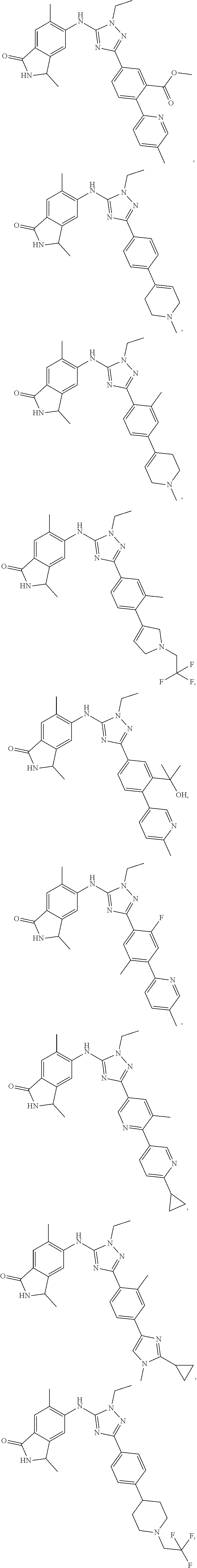

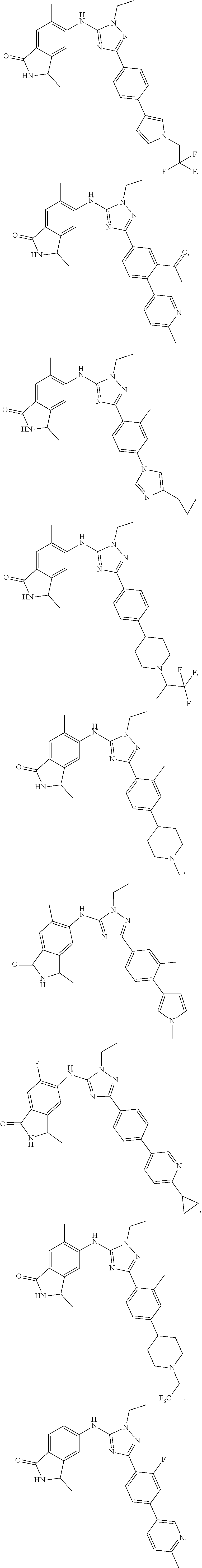

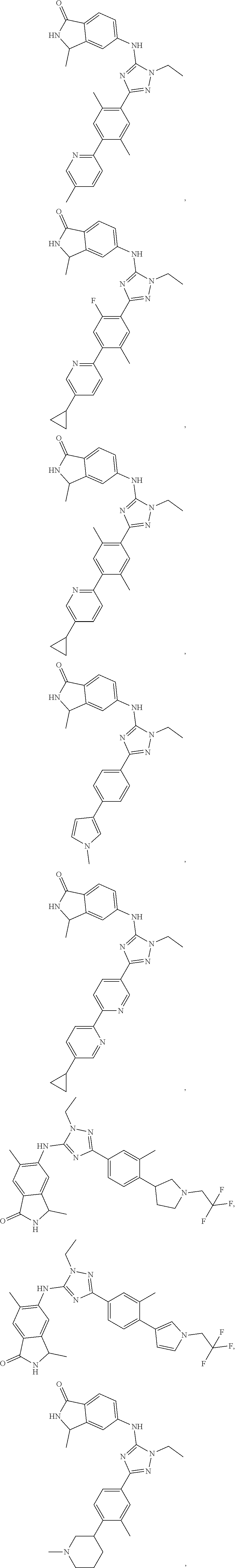

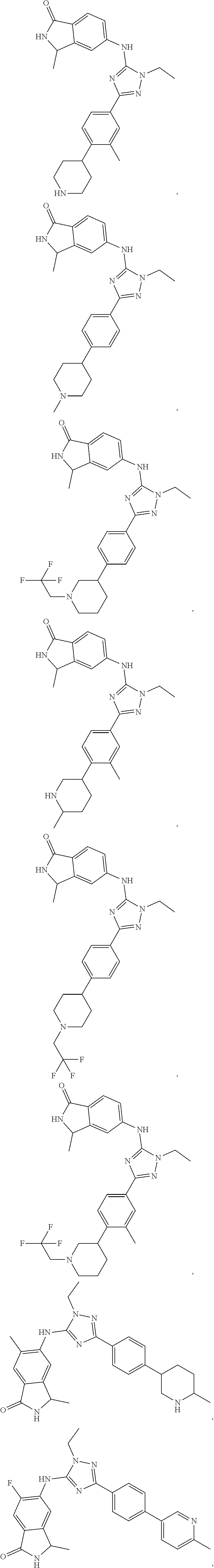

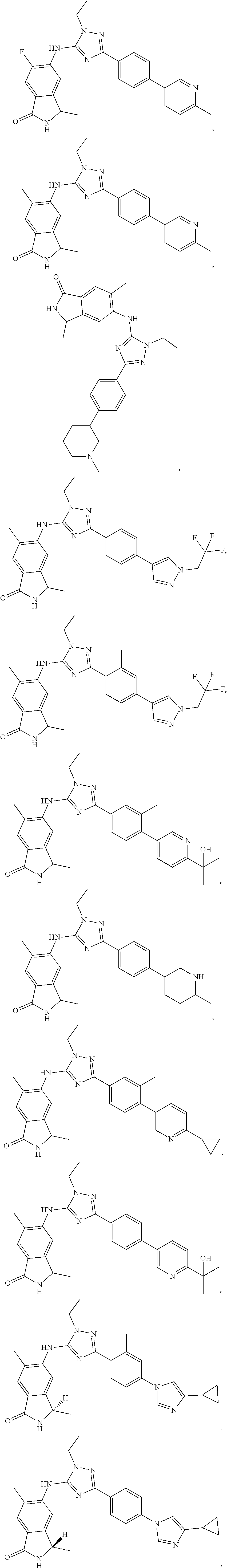

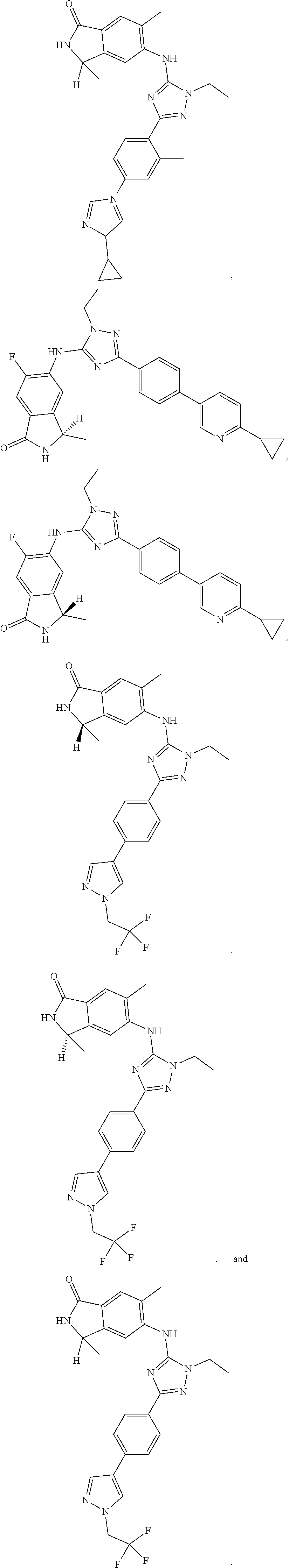

In an embodiment there is provided compounds of the present invention having a ROCK2 binding affinity within category +++ or ++++ as defined elsewhere herein. In an embodiment there is provided compounds of the present invention having a ROCK2 binding affinity IC50 value of <3 μM. In an embodiment there is provided compounds of the present invention having a ROCK2 binding affinity IC50 value of <0.3 μM. Optionally, the binding activity is determined using the assay for ROCK2 inhibition defined in the examples.

In an aspect of the invention there is provided the compounds of the present invention for use as a medicament.

In accordance with another aspect, the present invention provides a pharmaceutical formulation comprising a compound of the present invention and a pharmaceutically acceptable excipient.

In an embodiment, the pharmaceutical composition may be a combination product comprising an additional pharmaceutically active agent. The additional pharmaceutically active agent may be, for example anti-inflammatory agents, anti-fibrotic agents, chemotherapeutics, anti-cancer agents, immunosuppressants, anti-tumour vaccines, cytokine therapy, or tyrosine kinase inhibitors.

In accordance with another aspect, there is provided a compound of the present invention for use in the treatment of a condition which is modulated by ROCK1 and/or ROCK2. Usually conditions that are modulated by ROCKs (please note that ROCKs refer to either or both of ROCK1 and ROCK2) are conditions that would be treated by the inhibition of ROCKs using a compound of the present invention. A compound of any formula disclosed herein may be for use in the treatment of a condition treatable by the inhibition of ROCKS. As discussed above, ROCK signalling is instrumental in a number of conditions.

In another aspect of the invention, there is provided a compound of the present invention for use in the treatment of a disease or disorder selected from: fibrotic diseases, auto-immune diseases, muscular dystrophy, inflammatory conditions, central nervous system disorders, or cancer.

In an aspect of the invention there is provided a method of treating a disease or disorder which is modulated by ROCK1 and/or ROCK2 wherein the method comprises administering a therapeutic amount of a compound of the invention, to a patient in need thereof.

The method of treatment may be a method of treating a condition treatable by the inhibition of ROCK1 and/or ROCK2.

The invention also provides a method of treating a disease or disorder selected from: fibrotic diseases, auto-immune, inflammatory-fibrotic conditions, inflammatory conditions, central nervous system disorders, or cancer, wherein the method comprises administering a therapeutic amount of a compound of any formula disclosed herein, to a patient in need thereof.

The disease or disorder may be selected from: Sarcoidosis, sclerosis, primary biliary sclerosis, sclerosing cholangitis, dermatitis, atopic dermatitis, Still's disease, chronic obstructive pulmonary disease, Guillain-Barre disease, Graves' disease, Addison's disease, Raynaud's phenomenon, or autoimmune hepatitis. Arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include Achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, cerebral cavernous malformations, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, endometriosis, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, yersinial arthritis and conditions involving vascularization and/or inflammation, include atherosclerosis, rheumatoid arthritis (RA), hemangiomas, angiofibromas, and psoriasis. Other non-limiting examples of angiogenic disease are retinopathy of prematurity (retrolental fibroplastic), corneal graft rejection, corneal neovascularization related to complications of refractive surgery, corneal neovascularization related to contact lens complications, corneal neovascularization related to pterygium and recurrent pterygium, corneal ulcer disease, and non-specific ocular surface disease, insulin-dependent diabetes mellitus, multiple sclerosis, myasthenia gravis, Chrorfs disease, autoimmune nephritis, primary biliary cirrhosis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osier-Weber syndrome, restinosis, and fungal, parasitic and viral infections, including cytomegalo viral infections.

Any of the conditions disclosed above as being treatable by ROCK1 and/or ROCK2 inhibition may be treated by a compound of the invention, or may be treated in a method comprising administering a compound of the invention, or may be treated by a medicament manufactured through the use of a compound of the present invention.

In embodiments, the disease or disorder may be selected from: Idiopathic Pulmonary Fibrosis (IPF); systemic sclerosis (SSC); interstitial lung disease (ILD); type 1 and type 2 diabetes; diabetic nephropathy; Nonalcoholic Steatohepatitis (NASH); Nonalcoholic fatty liver disease (NAFLD); hypertension, atherosclerosis, restenosis, stroke, heart failure, coronary vasospasm, cerebral vasospasm, peripheral circulatory disorder, peripheral artery occlusive disease, ischemia/reperfusion injury, pulmonary hypertension and angina, erectile dysfunction, fibroid lung, fibroid liver and fibroid kidney. glaucoma, ocular hypertension, retinopathy, rheumatoid arthritis, psoriasis, psoriatic arthritis, Sjogren's syndrome, asthma, adult respiratory distress syndrome, chronic obstructive pulmonary disease (COPD), SLE, cGVHD, inflammatory bowel disease, stenosis of the bowel, disorders involving neuronal degeneration or physical injury to neural tissue, Huntington's disease, Parkinson's Disease, Alzheimer's, Amyotrophic lateral sclerosis (ALS), multiple sclerosis, liver cancer, bladder cancer, hepatoma, squamous carcinoma of the lung, non-small cell lung cancer, adenocarcinoma of the lung, small-cell lung cancer, various types of head and neck cancer, breast cancer, colon cancer, colorectal cancer, cancer of the peritoneum, hepatocellular cancer, gastrointestinal cancer, esophageal cancer, endometrial or uterine carcinoma, salivary gland carcinoma, squamous cell cancer, pituitary cancer, astrocytoma, soft tissue sarcoma, pancreatic cancer, glioblastoma, cervical cancer, ovarian cancer, kidney cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer and melanoma, wherein the method comprises administering a therapeutic amount of a compound of any formula disclosed herein, to a patient in need thereof.

In certain embodiments, the disease or disorder is selected from: Idiopathic Pulmonary Fibrosis (IPF); systemic sclerosis (SSC); interstitial lung disease (ILD); type 1 and type 2 diabetes; diabetic nephropathy; Nonalcoholic Steatohepatitis (NASH); Nonalcoholic fatty liver disease (NAFLD); hypertension, atherosclerosis, restenosis, stroke, heart failure, coronary vasospasm, cerebral vasospasm, peripheral circulatory disorder, peripheral artery occlusive disease, ischemia/reperfusion injury, pulmonary hypertension and angina, and erectile dysfunction, fibroid lung, fibroid liver and fibroid kidney.

In certain embodiments the disease or disorder is selected from: glaucoma, ocular hypertension, retinopathy, rheumatoid arthritis, psoriasis, psoriatic arthritis, Sjogren's syndrome, asthma, adult respiratory distress syndrome, chronic obstructive pulmonary disease (COPD), SLE and cGVHD, inflammatory bowel disease and stenosis of the bowel.

In certain embodiments, compounds of the invention are for use in the treatment of or are used in a method of treatment of central nervous system disorders. Such disorders may involve neuronal degeneration or physical injury to neural tissue, including without limitation, Huntington's disease, Parkinson's Disease, Alzheimer's, Amyotrophic lateral sclerosis (ALS), or multiple sclerosis.

In certain embodiments compounds of the invention are for use in the treatment of or are used in a method of treatment of cancer. Examples include but are not limited to: liver cancer, bladder cancer, hepatoma, squamous carcinoma of the lung, non-small cell lung cancer, adenocarcinoma of the lung, small-cell lung cancer, various types of head and neck cancer, breast cancer, colon cancer, colorectal cancer, cancer of the peritoneum, hepatocellular cancer, gastrointestinal cancer, esophageal cancer, endometrial or uterine carcinoma, salivary gland carcinoma, squamous cell cancer, pituitary cancer, astrocytoma, soft tissue sarcoma, pancreatic cancer, glioblastoma, cervical cancer, ovarian cancer, kidney cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer and melanoma.

In another aspect of the invention there is provided a pharmaceutical composition, wherein the composition comprises a compound of the invention and pharmaceutically acceptable excipients.

In an embodiment the pharmaceutical composition may be a combination product comprising an additional pharmaceutically active agent. The additional pharmaceutically active agent may be one disclosed elsewhere herein.

In an aspect of the present invention there is provided the use of a compound of the invention in the manufacture of a medicament for use in the treatment of any condition disclosed herein.

### DETAILED DESCRIPTION

Given below are definitions of terms used in this application. Any term not defined herein takes the normal meaning as the skilled person would understand the term.

The term “halo” refers to one of the halogens, group 17 of the periodic table. In particular, the term refers to fluorine, chlorine, bromine and iodine. Preferably, the term refers to chlorine or fluorine.

The term “alkyl” refers to a linear or branched hydrocarbon chain. For example, the term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “Alkylene” groups may likewise be linear or branched and is divalent, i.e. it attached at two positions to other portions of the molecule. Furthermore, an alkylene group may, for example, correspond to one of those alkyl groups listed in this paragraph. The alkyl and alkylene groups may be unsubstituted or substituted by one or more substituents.

The term “haloalkyl” refers to a hydrocarbon chain substituted with at least one halogen atom independently chosen at each occurrence, for example fluorine, chlorine, bromine and iodine.

For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. The term “fluoroalkyl” refers to a hydrocarbon chain substituted with at least one fluorine atom.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₅ alkynyl” may be ethynyl, propynyl, butynyl, pentynyl and hexynyl.

The term “heteroalkyl” refers to a branched or linear hydrocarbon chain containing at least one heteroatom selected from N, O and S positioned between any carbon in the chain or at an end of the chain. For example, the term “C₁₋₆ heteroalkyl” refers to a branched or linear hydrocarbon chain containing 1, 2, 3, 4, 5, or 6 carbon atoms and at least one heteroatom selected from N, O and S positioned between any carbon in the chain or at an end of the chain. For example, the hydrocarbon chain may contain one or two heteroatoms. The C₁₋₆ heteroalkyl may be bonded to the rest of the molecule through a carbon or a heteroatom. For example, the “C₁₋₆ heteroalkyl” may be C₁₋₆ N-alkyl, C₁₋₆ N,N-alkyl, or C₁₋₆ O-alkyl.

The term “heterocycle” refers to a saturated, unsaturated or aromatic ring system containing at least one heteroatom selected from N, O or S. A “heterocyclic” system may contain 1, 2, 3 or 4 heteroatoms, for example 1 or 2. A “heterocyclic” system may be monocyclic or a fused polycyclic ring system, for example, bicyclic or tricyclic. A “heterocyclic” moiety may contain from 3 to 14 carbon atoms, for example, 3 to 8 carbon atoms in a monocyclic system and 7 to 14 carbon atoms in a polycyclic system. “Heterocyclic” encompasses heterocycloalkyl moieties, heterocycloalkenyl moieties and heteroaryl moieties. For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran. Heterocyclyl includes groups such as pyridones and N-alkyl-pyridones.

The term “C₃₋₈ cycloalkyl” refers to a saturated hydrocarbon ring system containing 3, 4, 5, 6, 7 or 8 carbon atoms. For example, the “C₃₋₈ cycloalkyl” may be cyclopropyl, cyclobutyl, cyclopentyl, cyclohexyl, cycloheptyl and cyclooctyl.

The term “C₃₋₈ cycloalkenyl” refers to an unsaturated hydrocarbon ring system containing 3, 4, 5, 6, 7 or 8 carbon atoms that is not aromatic. The ring may contain more than one double bond provided that the ring system is not aromatic. For example, the “C₃₋₈ cycloalkyl” may be cyclopropenyl, cyclobutenyl, cyclopentenyl, cyclopentadienyl, cyclohexenyl, cyclohexadienly, cycloheptenyl, cycloheptadiene, cyclooctenyl and cycloatadienyl.

The term “heterocycloalkyl” refers to a saturated hydrocarbon ring system containing carbon atoms and at least one heteroatom within the ring selected from N, O and S. For example, there may be 1, 2 or 3 heteroatoms, optionally 1 or 2. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be a “C₃₋₈ heterocycloalkyl”. The term “C₃₋₈ heterocycloalkyl” refers to a saturated hydrocarbon ring system containing 3, 4, 5, 6, 7 or 8 atoms at least one of the atoms being a heteroatom within the ring selected from N, O and S. The “heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “aromatic” when applied to a substituent as a whole means a single ring or polycyclic ring system with 4n+2 electrons in a conjugated π system within the ring or ring system where all atoms contributing to the conjugated r system are in the same plane.

The term “aryl” refers to an aromatic hydrocarbon ring system. The ring system has 4n+2 electrons in a conjugated π system within a ring where all atoms contributing to the conjugated T system are in the same plane. For example, the “aryl” may be phenyl and naphthyl. The aryl system itself may be substituted with other groups.

The term “heteroaryl” refers to an aromatic hydrocarbon ring system with at least one heteroatom within a single ring or within a fused ring system, selected from O, N and S. The ring or ring system has 4n+2 electrons in a conjugated n system where all atoms contributing to the conjugated T system are in the same plane. For example, the “heteroaryl” may be imidazole, oxazole, isoxazole, thiazole, isothiazole, thiene, furan, thianthrene, pyrrole, benzimidazole, pyrazole, pyrazine, pyridine, pyrimidine and indole.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

A bond drawn as a solid line and a dotted line represents a bond which can be either a single bond or a double bond, where chemically possible. For example, the bond drawn below could be a single bond or a double bond.

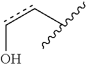

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without inappropriate effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

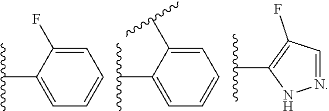

“Meta” substitution is a substitution pattern where two substituents are on carbons one carbon removed from each other, i.e with a single carbon atom between the substituted carbons. In other words there is a substituent on the second atom away from the atom with another substituent. For example the groups below are meta substituted.

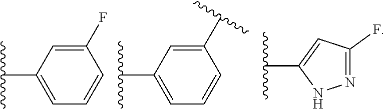

“Para” substitution is a substitution pattern where two substituents are on carbons two carbons removed from each other, i.e with two carbon atoms between the substituted carbons. In other words there is a substituent on the third atom away from the atom with another substituent. For example the groups below are para substituted.

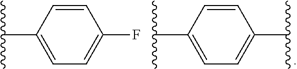

Throughout the description the disclosure of a compound also encompasses pharmaceutically acceptable salts, solvates and stereoisomers thereof. Where a compound has a stereocentre, both (R) and (S) stereoisomers are contemplated by the invention, equally mixtures of stereoisomers or a racemic mixture are completed by the present application. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diastereomeric mixtures. Where the mixture is a mixture of enantiomers the enantiomeric excess may be any of those disclosed above. Where the compound is a single stereoisomer the compounds may still contain other diastereoisomers or enantiomers as impurities. Hence a single stereoisomer does not necessarily have an enantiomeric excess (e.e.) or diastereomeric excess (d.e.) of 100% but could have an e.e. or d.e. of about at least 85%, at least 60% or less. For example, the e.e. or d.e. may be 90% or more, 90% or more, 80% or more, 70% or more, 60% or more, 50% or more, 40% or more, 30% or more, 20% or more, or 10% or more.

The invention contemplates pharmaceutically acceptable salts of the compounds of the invention. These may include the acid addition and base salts of the compounds. These may be acid addition and base salts of the compounds. In addition the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the invention include compounds of a number of formula as herein defined, including all polymorphs and crystal habits thereof, prodrugs and isomers thereof (including optical, geometric and tautomeric isomers) as hereinafter defined and isotopically-labelled compounds of the invention.

The present invention also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of the invention, it may be necessary to protect potential reactive functions that are not wished to react, and to cleave said protecting groups in consequence. In such a case, any compatible protecting radical can be used. In particular methods of protection and deprotection such as those described by T. W. GREENE (Protective Groups in Organic Synthesis, A. Wiley—Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography.

One or more compounds of the invention may be combined with one or more pharmaceutical agents, for example anti-inflammatory agents, anti-fibrotic agents, chemotherapeutics, anti cancer agents, immunosuppressants, anti-tumour vaccines, cytokine therapy, or tyrosine kinase inhibitors, for the treatment of conditions modulated by the inhibition of ROCK, for example fibrotic diseases, auto-immune, inflammatory-fibrotic conditions, inflammatory conditions, central nervous system disorders, or cancer.

The method of treatment or the compound for use in the treatment of fibrotic diseases, auto-immune, inflammatory-fibrotic conditions, inflammatory conditions, central nervous system disorders, or cancer as defined hereinbefore may be applied as a sole therapy or be a combination therapy with an additional active agent.

The method of treatment or the compound for use in the treatment of fibrotic diseases, auto-immune, inflammatory-fibrotic conditions, inflammatory conditions, central nervous system disorders diseases may involve, in addition to the compound of the invention, additional active agents. The additional active agents may be one or more active agents used to treat the condition being treated by the compound of the invention and additional active agent. The additional active agents may include one or more of the following active agents:—
(i) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(ii) TNF inhibitors for example etanercept; monoclonal antibodies (e.g. infliximab (Remicade), adalimumab (Humira), certolizumab pegol (Cimzia), golimumab (Simponi)); fusion proteins (e.g. etanercept (Enbrel)); and 5-HT_{2A} agonists (e.g. 2,5-dimethoxy-4-iodoamphetamine, TCB-2, lysergic acid diethylamide (LSD), lysergic acid dimethylazetidide);
(iii) anti-inflammatory drugs, for example non-steroidal anti-inflammatory drugs;
(iv) dihydrofolate reductase inhibitors/antifolates, for example methotrexate, trimethoprim, brodimoprim, tetroxoprim, iclaprim, pemetrexed, ralitrexed and pralatrexate; and
(v) immunosuppressants for example cyclosporins, tacrolimus, sirolimus pimecrolimus, angiotensin II inhibitors (e.g. Valsartan, Telmisartan, Losartan, Irbesatan, Azilsartan, Olmesartan, Candesartan, Eprosartan) and ACE inhibitors e.g. sulfhydryl-containing agents (e.g. Captopril, Zofenopril), dicarboxylate-containing agents (e.g. Enalapril, Ramipril, Quinapril, Perindopril, Lisinopril, Benazepril, Imidapril, Zofenopril, Trandolapril), phosphate-containing agents (e.g. Fosinopril), casokinins, lactokinins and lactotripeptides.
(vi) Anti-fibrotic agents for example: Pirfenidone, Nintedanib, Anti-CTGF monoclonal antibodies (e.g. Pamrevlumab), anti-avβ6 monoclonal antibodies (e.g. PLN-74809), Anti-IL-13 monoclonal antibodies (e.g. Tralokinumab, QAX576, Lebrikizumab), Simtuzumab, lysophosphatidic acid receptor antagonists (e.g. BMS-986020, AM966), LOXL2 inhibitors, BET bromodomain inhibitors (e.g. JQ1), HDAC inhibitors (e.g. Vorinostat), thrombin inhibitors (e.g. Dabigatran), FactorXa inhibitors (e.g. Apixban, Rivaroxaban) 15PGDH inhibitors, PAR1 inhibitors, Nox4 inhibitors and PAI-1 inhibitors.
(vii) CNS therapies, for example: Levodopa, Dopamine agonists, Apomorphine, Glutamate antagonist, Anticholinergics, COMT inhibitors, MAO-B inhibitors, riluzole (Rilutek), Tetrabenazine (Xenazine), haloperidol (Haldol), chlorpromazine, risperidone (Risperdal), quetiapine (Seroquel), amantadine, levetiracetam (Keppra), clonazepam (Klonopin), Donepezil (Aricept), Galantamine (Razadyne), Rivastigmine (Exelon), Memantine (Ebixa, Axura), Aducanumab, Ocrelizumab, interferon beta-1a (Avonex, Rebif), peginterferon beta-1a (Plegridy), teriflunomide (Aubagio), fingolimod (Gilenya), mitoxantrone (Novantrone), dimethyl fumarate (Tecfidera), natalizumab (Tysabri)

The method of treatment or the compound for use in the treatment of cancer, sarcoma, melanoma, skin cancer, haematological tumors, lymphoma, carcinoma, leukemia, and central nervous system disorders may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-a), etoposide, and teniposide;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-I0, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 modulator;
(v) antiangiogenic agents such as those which inhibit the effects of vascular endothelial growth factor, for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine; PD-1, PD-L1, PD-L2 and CTL4-A modulators, antibodies and vaccines; other IDO inhibitors (such as indoximod); anti-PD-1 monoclonal antibodies (such as MK-3475 and nivolumab); anti-PD-L1 monoclonal antibodies (such as MEDI-4736 and RG-7446); anti-PD-L2 monoclonal antibodies; and anti-CTLA-4 antibodies (such as ipilimumab);
(xii) chimeric antigen receptors, anticancer vaccines and arginase inhibitors.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The compounds of the invention may be prepared according to or analogously to the General Schemes 1 and 2 and Examples 1 to 132.

### Examples and Synthesis

As used herein the following terms have the meanings given: “Ac” refers to acetyl; “ADP” refers to adenosine diphosphate; “ATP” refers to adenosine triphosphate; “Boc” refers to tert-butoxycarbonyl; “dba” refers to dibenzylideneacetone; “d” refers to doublet; “DCE” refers to 1,2-dichloroethane; “DCM” refers to dichloromethane; “DIPEA” refers to N,N-Diisopropylethylamine; “DMAP” refers to 4-(dimethylamino)pyridine; “DMF” refers to N,N-dimethylformamide; “DMSO” refers to dimethylsulfoxide; “dppf” refers to 1,1′-bis(diphenylphosphino)ferrocene; “DTT” refers to dithiothreitol; “EDC” refers to N-(3-dimethylaminopropyl)-N′-ethylcarbodiimide; “EDTA” refers to ethylenediamine tetra-acetic acid; “ES-API” refers to atmospheric pressure ionization electrospray; “ESI” refers to electrospray ionization; “Et” refers to ethyl; “EtOAc” refers to ethyl acetate; “HATU” refers to 1-[bis(dimethylamino)methylene]-1H-1,2,3-triazolo[4,5-b]pyridinium 3-oxide hexafluorophosphate; “HEPES” refers to (4-(2-hydroxyethyl)-1-piperazineethanesulfonic acid); “HOBt” refers to 1-hydroxybenzotriazole hydrate; “HPLC” refers to high performance liquid chromatography; “IPA” refers to 2-propanol; “J” refers to coupling constant; “LCMS” or “LC-MS” refers to liquid chromatography/mass spectrometry; LiHMDS” refers to lithium bis(trimethylsilyl)amide; “m” refers to multiplet; “Me” refers to methyl; “MIM” refers to monoisotopic mass; “min” refers to min; “MS” refers to mass spectrometry; “NMR” refers to nuclear magnetic resonance; “Pet. Ether” refers to Pet. Ether; “PG” refers to protecting group; “PTSA” refers to p-toluenesulfonic acid monohydrate; “q” refers to quartet; “quint” refers to quintet; “Rf” refers to Retention factor; “RT” refers to retention time; “r.t.” refers to room temperature; “s” refers to singlet; “SCX” refers to strong cation exchange; “SEM” refers to 2-(trimethylsilyl)ethoxymethyl; “t” refers to triplet; “TBME” refers to tert-butyl methyl ether; “TEA” refers to triethylamine; “TFA” refers to trifluoroacetic acid; “TFE” refers to trifluoroethanol; “THF” refers to tetrahydrofuran; “THP” refers to tetrahydropyran; “TLC” refers to thin layer chromatography; “TMS” refers to tetramethylsilane; “UPLC” refers to ultra-performance liquid chromatography; “UV” refers to ultraviolet; “XantPhos” refers to 4,5-bis(diphenylphosphino)-9,9-dimethylxanthene; and “XPhos” refers to 2-dicyclohexylphosphino-2′,4′,6′-triisopropylbiphenyl.

Solvents, reagents and starting materials were purchased from commercial vendors and used as received unless otherwise described. All reactions were performed at r.t. unless otherwise stated. Compound identity and purity confirmations were performed by LCMS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in the below. Retention times RT are reported in min.

| | | |
|---|---|---|
| Time (min) | % A | % B |

Method A: Short Acidic

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method B: Long Acidic

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Compound identity confirmations were also performed by LCMS UV using the following:

### Method C

LC: Agilent Technologies 1290 series
Binary Pump
Diode Array Detector
Column: Agilent Eclipse Plus RRHD C18, 1.8 μm, 3.0×50 mm
Column Temperature: 40° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.8 mL/min
Gradient:

| T (min) | A (%) | B (%) |
|---|---|---|
| 0.00 | 60 | 40 |
| 2.65 | 0 | 100 |
| 3.00 | 0 | 100 |

MS: G6120A, Quadrupole LC/MS
Ion Source: ESI
Signal: positive
TIC: 70˜1000 m/z
Fragmentor: 60
Threshold: 5
Gain: 1
Drying gas flow: 10 L/min
Nebulizer pressure: 35 psi

### Method D

LC: Agilent Technologies 1290 series
Binary Pump
Diode Array Detector
Column: Agilent Eclipse Plus RRHD C18, 1.8 μm, 3.0×50 mm
Column Temperature: 40° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.8 mL/min
Gradient:

| T (min) | A(%) | B(%) |
|---|---|---|
| 0.00 | 80 | 20 |
| 2.65 | 20 | 80 |
| 3.00 | 20 | 80 |

MS: G6120A, Quadrupole LC/MS
Ion Source: ESI
Signal: positive
TIC: 70˜1000 m/z
Fragmentor: 60
Threshold: 5
Gain: 1
Drying gas flow: 10 L/min
Nebulizer pressure: 35 psi

### Method E

LC: Agilent Technologies 1290 series
Binary Pump
Diode Array Detector.
Column: Agilent Eclipse Plus RRHD C18, 1.8 μm, 3.0×50 mm
Column Temperature: 40° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.8 mL/min
Gradient:

| T (min) | A (%) | B (%) |
|---|---|---|
| 0.00 | 92 | 8 |
| 2.50 | 40 | 60 |
| 3.00 | 20 | 80 |

MS: G6120A, Quadrupole LC/MS
Ion Source: ESI
Signal: positive
TIC: 70˜1000 m/z
Fragmentor: 60
Threshold: 5
Gain: 1
Drying gas flow: 10 L/min
Nebulizer pressure: 35 psi

### Method F

LC: Shimadzu 2020 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in ACN (v/v)
Run time: 5.0 min
Flow rate: 1.0 mL/min
Gradient:

| T (min) | A (%) | B (%) |
|---|---|---|
| 0.00 | 50 | 50 |
| 0.50 | 50 | 50 |
| 4.00 | 0 | 100 |
| 4.50 | 0 | 100 |
| 4.51 | 50 | 50 |
| 5.00 | 50 | 50 |

MS: Ion Source: ESI
Signal: positive and negative
TIC: 100˜900 m/z
Fragmentor: 60
Threshold: 5
Gain: 1
Drying gas: 20 L/min
Nebulizing Gas: 1.5 L/min

### Method G

LC: Shimadzu 2020 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in ACN (v/v)
Run time: 5.0 min
Flow rate: 1.0 mL/min
Gradient:

| T (min) | A (%) | B (%) |
|---|---|---|
| 0.01 | 85 | 15 |
| 1.0 | 85 | 15 |
| 4.0 | 0 | 100 |
| 4.5 | 0 | 100 |
| 4.51 | 85 | 15 |
| 5.0 | 85 | 15 |

MS: Ion Source: ESI
Signal: positive and negative
TIC: 100˜900 m/z
Fragmentor: 60
Threshold: 5
Gain: 1
Drying gas: 20 L/min
Nebulizing Gas: 1.5 L/min

### Method H

LC: Shimadzu LC-20XR series
Binary Pump
Diode Array Detector
Column: Waters ACQUITY UPLC HSS C18, 1.8 μm, 3.0×50 mm
Column Temperature: 25° C.
Acquisition wavelength: 214 nm, 254 nm
Mobile phase: A: 0.05% Formic acid in water (v/v), B: 0.05% Formic acid in ACN (v/v)
Run time: 3.0 min
Flow Rate: 0.5 mL/min
Gradient:

| T (min) | A (%) | B (%) |
|---|---|---|
| 0.01 | 70 | 30 |
| 2.00 | 5 | 95 |
| 2.50 | 5 | 95 |
| 2.51 | 70 | 30 |
| 3.00 | 70 | 30 |

MS: 2020, Quadrupole LC/MS, Ion Source: API-ESI
TIC: 100˜900 m/z
Drying gas flow: 15 L/min
Nebulizer pressure: 1.5 L/min
Drying gas temperature: 250° C.
Vcap: 4500V.

### Method 1

LC: Shimadzu 2020 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in ACN (v/v)
Run time: 5.0 min
Flow rate: 1.0 mL/min
Gradient:

| T (min) | A (%) | B (%) |
|---|---|---|
| 0.01 | 97 | 3 |
| 1.0 | 97 | 3 |
| 4.0 | 30 | 70 |
| 4.01 | 0 | 100 |
| 4.5 | 0 | 100 |
| 4.51 | 97 | 3 |
| 5.0 | 97 | 3 |

MS: Ion Source: ESI
Signal: positive and negative
TIC: 100˜900 m/z
Fragmentor: 60
Threshold: 5
Gain: 1
Drying gas: 20 L/min
Nebulizing Gas: 1.5 L/min

### Method J

LC: Shimadzu LC-20XR series
Binary Pump
Diode Array Detector
Column: Waters ACQUITY UPLC HSS C18, 1.8 μm, 3.0×50 mm
Column temperature: 25° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.6 mL/min
Gradient:

| T (min) | A (%) | B (%) |
|---|---|---|
| 0.01 | 50 | 50 |
| 2.00 | 0 | 100 |
| 2.50 | 0 | 100 |
| 2.51 | 50 | 50 |
| 3.00 | 50 | 50 |

| | |
|---|---|
| 3.01 | STOP |

MS: 2020, Quadrupole LC/MS
Ion Source: API-ESI
TIC: 100˜900 m/z
Drying gas flow: 15 L/min
Nebulizer pressure: 1.5 L/min
Drying gas temperature: 250° C.
Vcap: 4500V

### Method K

LC: Shimadzu LC-20XR series
Binary Pump
Diode Array Detector
Column: Waters ACQUITY UPLC HSS C18, 1.8 μm, 3.0×50 mm column
Column temperature: 25° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.6 mL/min
Gradient:

| T (min) | A (%) | B (%) |
|---|---|---|
| 0.01 | 80 | 20 |
| 2.00 | 15 | 85 |
| 2.50 | 15 | 85 |
| 2.51 | 80 | 20 |
| 3.00 | 80 | 20 |

| | |
|---|---|
| 3.01 | STOP |

MS: 2020, Quadrupole LC/MS
Ion Source: API-ESI
TIC: 100˜900 m/z
Drying gas flow: 15 L/min
Nebulizer pressure: 1.5 L/min
Drying gas temperature: 250° C.
Vcap: 4500V

### Method L

LC: Agilent Technologies 1290 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in MeCN (v/v)
Run time: 5.0 min
Flow rate: 1.0 mL/min
Gradient:

| T (min) | A(%) | B(%) |
|---|---|---|
| 0.0 | 90 | 10 |
| 0.5 | 90 | 10 |
| 4.0 | 10 | 90 |
| 4.5 | 0 | 100 |
| 4.51 | 90 | 10 |
| 5.0 | 90 | 10 |

MS: G6120A, Quadrupole LC/MS
Ion Source: ESI
Signal: positive and negative
TIC: 70˜1000 m/z
Fragmentor: 60
Threshold: 5
Gain: 1
Drying gas flow: 10 L/min
Nebulizer pressure: 35 psi

### Method M

LC: Agilent Technologies 1290 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in MeCN (v/v)
Run time: 5.0 min
Flow rate: 1.0 mL/min
Gradient:

| T (min) | A(%) | B(%) |
|---|---|---|
| 0.0 | 97 | 3 |
| 0.5 | 97 | 3 |
| 4.0 | 30 | 70 |
| 4.5 | 0 | 100 |
| 4.51 | 97 | 3 |
| 5.0 | 97 | 3 |

MS: G6120A, Quadrupole LC/MS
Ion Source: ESI
Signal: positive and negative
TIC: 70˜1000 m/z
Fragmentor: 60
Threshold: 5
Gain: 1
Drying gas flow: 10 L/min
Nebulizer pressure: 35 psi

### Method N

LC: Agilent Technologies 1290 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formate in water (v/v), B: 0.05% Formate in ACN (v/v)
Run time: 5.0 min
Flow rate: 1.0 mL/min
Gradient:

| T (min) | A (%) | B (%) |
|---|---|---|
| 0.0 | 90 | 10 |
| 0.5 | 90 | 10 |
| 4.0 | 10 | 90 |
| 4.5 | 0 | 100 |
| 4.51 | 90 | 10 |
| 5.0 | 90 | 10 |

MS: G6120A, Quadrupole LC/MS
Ion Source: ESI
Signal: positive
TIC: 70˜1000 m/z
Fragmentor: 60
Threshold: 5
Gain: 1
Drying gas flow: 10 L/min
Nebulizer pressure: 35 psi

### Method O

LC: Shimadzu LC-20XR series
Binary Pump
Diode Array Detector
Column: Waters ACQUITY UPLC HSS C18, 1.8 μm, 3.0×50 mm column
Column temperature: 25° C.
Acquisition wavelength: 214 nm, 254 nm
Mobile phase: A: 0.05% Formic acid in water (v/v), B: 0.05% Formic acid in MeCN (v/v)
Run time: 3.0 min
Flow Rate: 0.6 mL/min
Gradient:

| T (min) | A (%) | B (%) |
|---|---|---|
| 0.01 | 95 | 5 |
| 1.50 | 50 | 50 |
| 2.00 | 0 | 100 |
| 2.50 | 0 | 100 |
| 2.51 | 95 | 5 |
| 3.00 | 95 | 5 |

| | |
|---|---|
| 3.01 | STOP |

MS: 2020, Quadrupole LC/MS
Ion Source: API-ESI
TIC: 100˜900 m/z
Drying gas flow: 15 L/min
Nebulizer pressure: 1.5 L/min
Drying gas temperature: 250° C.
Vcap: 4500V.

### Method P

LC: Shimadzu LC-20AD series
Binary Pump
Diode Array Detector
Column: Waters Sunfire, 3.5 μm, 4.6×50 mm column
Column temperature: 25° C.
Acquisition wavelength: 214 nm, 254 nm
Mobile phase: A: 0.05% Formic acid in water (v/v), B: 0.05% Formic acid in MeCN (v/v)
Run time: 5.0 min
Flow Rate: 1 mL/min
Gradient:

| T (min) | A(%) | B(%) |
|---|---|---|
| 0.00 | 85 | 15 |
| 0.50 | 85 | 15 |
| 4.00 | 0 | 100 |
| 4.50 | 0 | 100 |
| 4.51 | 85 | 15 |
| 5.00 | 85 | 15 |

MS: 2020, Quadrupole LC/MS
Ion Source: API-ESI
TIC: 100˜900 m/
Drying gas flow: 15 L/min,
Nebulizer pressure: 1.5 L/min
Drying gas temperature: 250° C.
Vcap: 4500V.

### Method Q

LC: Shimadzu LC-20AD series
Binary Pump
Diode Array Detector
Column: Waters Sunfire, 3.5 μm, 4.6×50 mm column
Column temperature: 25° C.
Acquisition wavelength: 214 nm, 254 nm
Mobile phase: A: 0.05% Formic acid in water (v/v), B: 0.05% Formic acid in MeCN (v/v)
Run time: 5.0 min.
Flow Rate: 1 mL/min
Gradient:

| T (min) | A (%) | B (%) |
|---|---|---|
| 0.00 | 85 | 15 |
| 0.50 | 85 | 15 |
| 4.00 | 0 | 100 |
| 4.50 | 0 | 100 |
| 4.51 | 85 | 15 |
| 5.00 | 85 | 15 |

MS: 2020, Quadrupole LC/MS
Ion Source: API-ESI
TIC: 100˜900 m/z
Drying gas flow: 15 L/min
Nebulizer pressure: 1.5 L/min
Drying gas temperature: 250° C.
Vcap: 4500V.

### Method R

LC: Agilent Technologies 1290 series
Binary Pump
Diode Array Detector
Column: Agilent EclipsePlus RRHD C18, 1.8 μm, 3.0×50 mm
Column temperature: 25° C.
Acquisition wavelength: 214 nm, 254 nm
Mobile phase: 0.05% Formic acid in water (v/v), B: 0.05% Formic acid in ACN (v/v)
Flow Rate: 0.8 mL/min
Gradient:

| T (min) | A (%) | B (%) |
|---|---|---|
| 0.00 | 30 | 70 |
| 2.65 | 0 | 100 |
| 3.00 | 0 | 100 |

MS: G6120A, Quadrupole LC/MS, Ion Source: API-ES
TIC: 70˜1000 m/z
Fragmentor: 70
Drying gas flow: 12 L/min
Nebulizer pressure: 36 psi
Drying gas temperature: 350° C.
Vcap: 3000V.

### Method S

LC: Shimadzu 2020 series
Binary Pump
Diode Array Detector
Column: Agilent Poroshell 120 EC—C18, 2.7 μm, 4.6×50 mm
Column Temperature: 35° C.
Acquisition wavelength: 214 nm, 254 nm
Mobile Phase: A: 0.05% Formic acid in water (v/v), B: 0.05% Formic acid in ACN (v/v)
Run time: 5.0 min
Flow rate: 1.0 mL/min
Gradient:

| T (min) | A (%) | B (%) |
|---|---|---|
| 0.01 | 85 | 15 |
| 1.0 | 85 | 15 |
| 4.0 | 0 | 100 |
| 4.5 | 0 | 100 |
| 4.51 | 85 | 15 |
| 5.0 | 85 | 15 |

### MS: Ion Source: ESI

Signal: positive/negative
TIC: 100˜900 m/z
Fragmentor: 60
Threshold: 5
Gain: 1
Drying gas: 20 L/min
Nebulizing Gas: 1.5 L/min

NMR was also used to characterise final compounds. ¹H NMR spectra were obtained at r.t., unless otherwise stated, on a Bruker AVI 500 with either a 5 mm Dual or 5 mm QNP probe with Z gradients, a Bruker DRX500 with a 5 mm QNP probe with Z gradients or a Bruker AVIII 400 Nanobay with 5 mm BBFO probe. Chemical shifts are reported in ppm and referenced to either TMS (0.00 ppm), DMSO-d₆ (2.50 ppm), CDCl₃ (7.26 ppm) or MeOD-d₄ (3.31 ppm). NH or OH signals that exchange with deuterated solvent are not reported.

Optionally, compound Rf values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in the table below.

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

Compound identity confirmations were also performed by analytical Supercritical Fluid Chromatography (SFC) using a Waters UPC2 analytical SFC (SFC-H). A 10 μL aliquot was injected onto an HPLC column (Cellulose-2, 150×4.6 mm I.D., 3 μm) at RT which was controlled at 35° C. The samples were eluted at a flow rate of 2.0 mL/min with a mobile phase system composed of A for CO₂ and B for methanol (0.05% DEA, V/V) eluting under isocratic elution (50% phase B).

Compound purification was also performed by preparative Supercritical Fluid Chromatography (SFC). SFC purification was performed using a MG II preparative SFC(SFC-13). Samples were eluted at a flow rate of 80 mL/min on a Cellulose-2, 250×30 mm I.D., 10 μm particle size with a mobile phase system composed of A for CO₂ and B for Methanol (0.1% NH₃H₂O) under isocratic elution (50% phase B).

| | Analytical separation | | Preparative separation | |
|---|---|---|---|---|
| Method | method | | method | |
| Instrument: | Waters UPC2 analytical SFC | | MG II preparative SFC(SFC- | |
| | (SFC-H) | | 13) | |
| Column: | Cellulose-2, 150 × 4.6 mm | | Cellulose-2, 250 × 30 mm | |
| | I.D., 3 μm | | I.D., 10 μm | |
| Mobile phase | A for CO2 and B for Methanol | | A for CO2 and B for Methanol | |
| | (0.05% DEA) | | (0.1% NH₃•H₂O) | |
| Gradient: | B 50% | | B 50% | |
| Flow rate | 2.0 | mL/min | 80 | mL/min |
| Back pressure | 100 | bar | 100 | bar |
| Column temperature | 35° | C. | 38° | C. |
| Wavelength | 254 | nm | 220 | nm |
| Cycle time | | | ~15 | min |

HPLC Purity method (Agilent 17 min)

| Instrument | Agilent 1260 | |
|---|---|---|
| Column | CHIRALPAK ID 250*4.6 mm, 5 μm | |
| Mobile Phase | IPA/EtOH = 8:2 (v/v) | |
| Column Temperature | 35° | C. |
| Detector Wavelength | 300 | nm |
| Flow Rate | 0.7 | mL/min |
| Run Time | 17 | min |
| Injection volume | 5 | μL |
| Dilution | EtOH | |
| Sample Conc(mg/ml) | 0.5 | mg/mL |

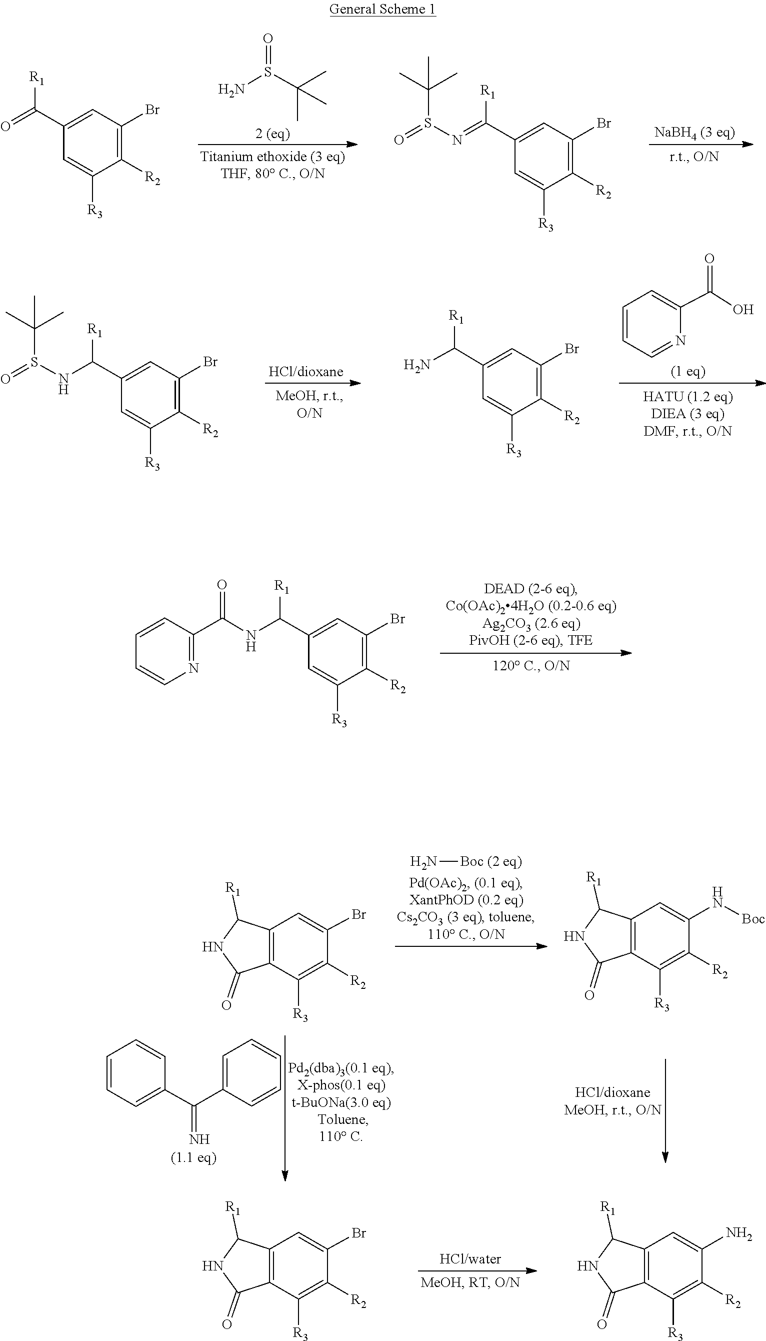

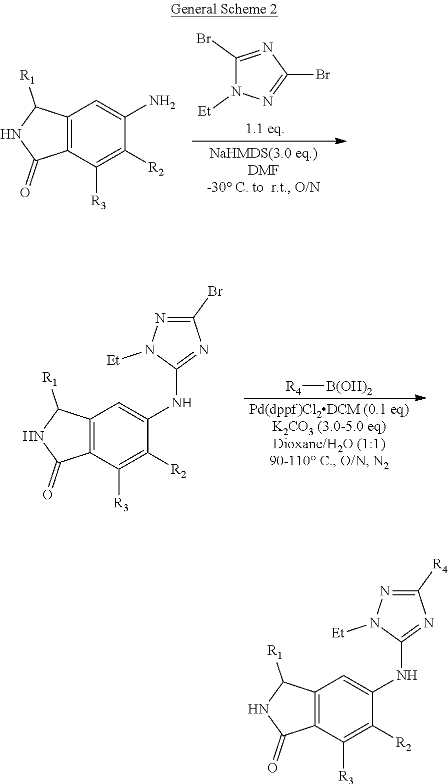

### General Method for the Synthesis of Intermediates 1 and 2

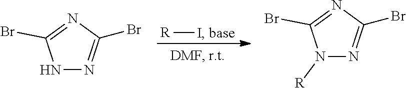

### Intermediate 1: 3,5-dibromo-1-methyl-1,2,4-triazole

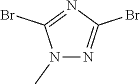

To a solution of 3,5-dibromo-1H-1,2,4-triazole (10.0 g, 44.1 mmol) in DMF (75 mL) and potassium carbonate (12.2 g, 88.2 mmol, 2.0 eq.) was added iodomethane (3.02 mL, 48.5 mmol, 1.1 eq.) in one portion. This gave rise to a strong exotherm from 17° C. to 38° C. after one minute. The reaction mixture was stirred overnight, diluted with 150 mL of EtOAc and then filtered to remove most of the inorganics. The solvent was removed under reduced pressure and the resultant yellow oily solid was partitioned between EtOAc (250 mL) and water (100 mL) and the aqueous washed with EtOAc (150 mL). The combined organics were washed with washed with brine (50 mL), dried over magnesium sulfate, filtered and the solvent removed in vacuo to give 3,5-dibromo-1-methyl-1,2,4-triazole (6.2 g, 25.8 mmol, 58% yield) as a yellow solid.

UPLC-MS (ES⁺, Method A): 1.79 min, m/z 241.7 [M+H]⁺. ¹H NMR (400 MHz, DMSO-d₆) δ 3.83 (3H).

### Intermediate 2: 3,5-dibromo-1-ethyl-1,2,4-triazole

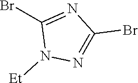

Following the procedure described for intermediate 1, a solution of 3,5-dibromo-1H-1,2,4-triazole (60 g, 266.88 mmol, 1.0 eq.), iodoethane (45.36 g, 293.52 mmol, 1.1 eq.) and K₂CO₃ (73.2 g, 533.64 mmol, 2.0 eq.) in DMF (216 mL) gave after work-up 3,5-dibromo-1-ethyl-1,2,4-triazole (59 g, 231.45 mmol, 88%).

LC-MS (ES-API, Method N): 1.58 min, m/z, 254.1 [M]⁺. ¹H NMR (400 MHz, DMSO-d₆) δ 4.18 (q, J=7.2 Hz, 2H), 1.36 (t, J=7.2 Hz, 3H).

### Intermediate 3: 5-amino-3,3-dimethyl-isoindolin-1-one

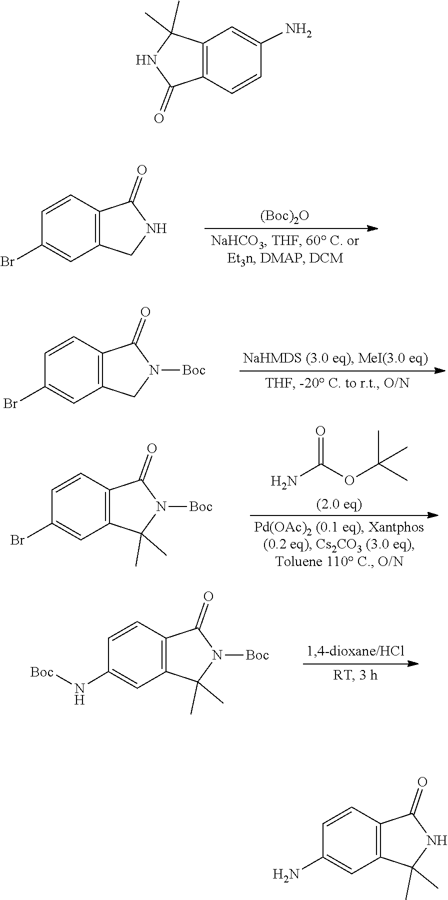

Step 1: tert-butyl 5-bromo-1-oxo-isoindoline-2-carboxylate

A mixture of 5-bromo-2,3-dihydroisoindol-1-one (1 g, 4.72 mmol), di-tert-butyl dicarbonate (1.23 g, 5.66 mmol, 1.2 eq.), triethylamine (0.79 mL, 5.67 mmol, 1.2 eq.) and 4-dimethylamino pyridine DMAP (28.8 mg, 0.24 mmol, 0.05 eq.) in DCM (47 mL) was stirred at 25° C. overnight. The mixture was concentrated and the residue purified by flash chromatography eluting in 0-40% EtOAc in Pet. ether, concentrated under reduced pressure to afford tert-butyl 5-bromo-1-oxo-isoindoline-2-carboxylate (1.40 g, 4.49 mmol, 95% yield) as a white solid.

UPLC-MS (ES⁺, Method A): 1.79 min, m/z 255.9 [M-tBu]⁺

¹H NMR (400 MHz, CDCl₃) δ 7.79-7.75 (1H, m), 7.66-7.61 (2H, m), 4.73 (2H, s), 1.60 (9H, s).

Step 2: tert-butyl 6-bromo-1,1-dimethyl-3-oxo-isoindoline-2-carboxylate To a solution of tert-butyl 5-bromo-1-oxo-isoindoline-2-carboxylate (1000 mg, 3.2 mmol) in anhydrous THF (11 mL) at −20° C. under N₂ was slowly added sodium bis(trimethylsilyl)amide (2.0M in THF, 4.8 mL, 9.6 mmol, 3.0 eq.) keeping the reaction mixture below −20° C. After 30 minute stirring, iodomethane (606.42 μL, 9.74 mmol, 3.0 eq.) was then added and the mixture was allowed to warm slowly to room temperature and stirred overnight. Methanol (1 mL) was carefully added and the mixture was concentrated under reduced pressure. The crude material was purified by flash column chromatography eluting with 0-30% EtOAc in Pet. Ether to afford tert-butyl 6-bromo-1,1-dimethyl-3-oxo-isoindoline-2-carboxylate (770 mg, 2.26 mmol, 71% yield) as a pale yellow solid.

UPLC-MS (ES⁺, Method A): 1.94 min, m/z 284.0 [M-tBu]⁺

### Step 3: tert-butyl 6-(tert-butoxycarbonylamino)-1,1-dimethyl-3-oxo-isoindoline-2-carboxylate

A mixture of tert-butyl 6-bromo-1,1-dimethyl-3-oxoisoindoline-2-carboxylate (757 mg, 2.23 mmol, 1.0 eq), tert-butyl carbamate (521 mg, 4.45 mmol, 2.0 eq), Cs₂CO₃ (2.17 g, 6.675 mmol, 3.0 eq), XantPhos (257 mg, 0.445 mmol, 0.2 eq) and Pd(OAc)₂ (50 mg, 0.2225 mmol, 0.1 eq) in toluene (10 mL) was stirred at 110° C. under N₂ overnight, cooled and concentrated in vacuum. Further purification by flash column chromatography eluting with 25% EtOAc in Pet. Ether gave tert-butyl 6-(tert-butoxycarbonylamino)-1,1-dimethyl-3-oxo-isoindoline-2-carboxylate (704 mg, 1.87 mmol, 84% yield).

LC-MS (ES-API, Method D): 2.736 min, m/z 321.1 [M+H−tBu]⁺.

### Step 4: 5-amino-3,3-dimethyl-isoindolin-1-one

A solution of tert-butyl 6-((tert-butoxycarbonyl)amino)-1,1-dimethyl-3-oxoisoindoline-2-carboxylate (704 mg, 1.87 mmol, 1.0 eq.) in a 4 M hydrogen chloride solution in 1,4-dioxane, (7 mL) was stirred at room temperature overnight. The mixture was diluted with NaHCO₃ aq. (15 mL) and extracted with EtOAc (8 mL×3). The combined organic layers were washed with brine (30 mL), dried over Na₂SO₄ and concentrated in vacuum to give 5-amino-3,3-dimethyl-isoindolin-1-one (220 mg, 1.25 mmol, 67% yield) as yellow solid.

LCMS (ES-API, Method D): 0.17 min, m/z 177.1 [M+H]⁺

¹H NMR (400 MHz, DMSO-d₆) δ 8.00 (s, 1H), 7.23 (d, J=8.9 Hz, 1H), 6.58-6.53 (m, 2H), 5.72 (s, 2H), 1.36 (s, 6H)

### Intermediate 4: 5′-aminospiro[cyclopropane-1,3′-isoindoline]-1′-one

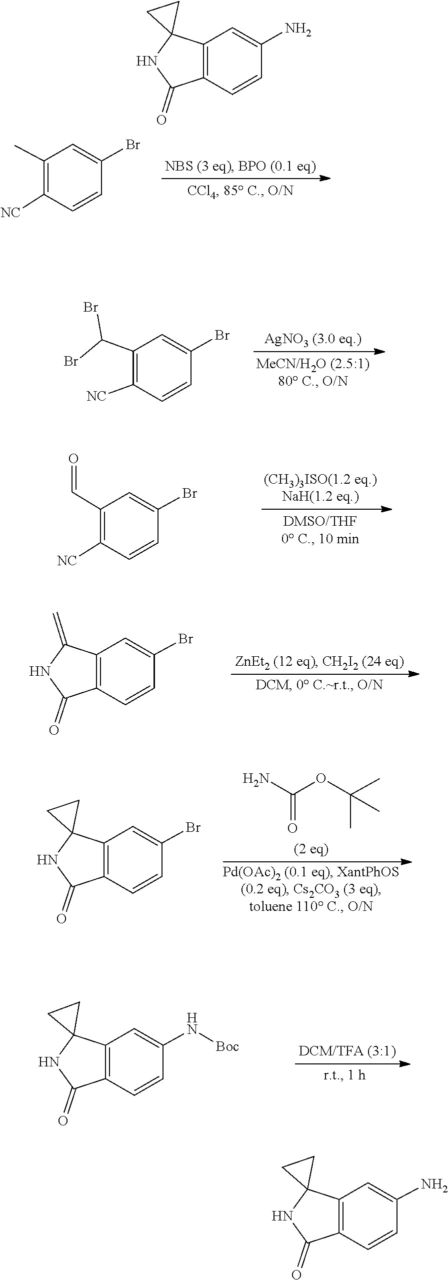

### Step 1: 4-bromo-2-(dibromomethyl)benzonitrile

A solution of 4-bromo-2-methylbenzonitrile (100 g, 510 mmol, 1.0 eq.) and NBS (261 g, 1530 mmol, 2.0 eq.) and BPO (12.36 g, 51 mmol, 0.1 eq.) in CCl₄ (1500 mL) was stirred at 85° C. overnight under N₂ atmosphere. The reaction mixture was filtered and concentrated under reduced pressure. The crude material was purified by flash column chromatography eluting with EtOAc/Pet. Ether (100:1) to give 4-bromo-2-(dibromomethyl)benzonitrile (133.5 g, 339 mmol, 66% yield).

¹H NMR (400 MHz, DMSO-d₆) δ 8.09-8.06 (m, 1H), 7.98-7.93 (m, 1H), 7.89-7.84 (m, 1H), 7.42 (s, 1H).

### Step 2: 4-bromo-2-formyl-benzonitrile

A solution of 4-bromo-2-(dibromomethyl)benzonitrile (124.3 g, 354 mmol, 1.0 eq.) and AgNO₃ (150 g, 888 mmol, 3.0 eq.) in MeCN/H₂O (200 mL/80 mL) was stirred at 80° C. overnight, filtrated and concentrated. The crude material was purified by flash column chromatography (eluting with 0-5% EtOAc in Pet. Ether to give 4-bromo-2-formyl-benzonitrile (46 g, 219 mmol, 62% yield).

¹H NMR (400 MHz, DMSO-d₆) δ 10.09 (s, 1H), 8.37-8.35 (d, J=2.1 Hz, 1H), 8.18-8.14 (dd, J=8.1, 2.1 Hz, 1H), 8.07-8.02 (d, J=8.4 Hz, 1H).

### Step 3: 5-bromo-3-methylene-isoindolin-1-one

To a solution of iodo-trimethyl-oxo-A{6}-sulfane (10.66 g, 48.45 mmol, 1.2 eq.) in dry DMSO (50 mL) and dry THF (30 mL) was added NaH (1.93 g, 48.45 mmol, 1.2 eq.) at 0° C. and stirred for 30 min and then 4-bromo-2-formylbenzonitrile (8.48 g, 40.38 mmol, 1.0 eq.) in dry DMSO (50 mL) and dry THF (30 mL) was added at 0° C. and stirred for 10 min. The mixture was quenched with sat. NH₄Cl and extracted with EtOAc (150 mL×3). The combined organic layers were washed with brine, dried over Na₂SO₄ and concentrated to give crude 5-bromo-3-methylene-isoindolin-1-one (7.35 g, 32.81 mmol, 81% yield) which was used without any further purification in the next step.

LCMS (ES-API, Method D): 1.64 min, m/z 224/226 [M]⁺/[M+2]⁺.

### Step 4: 5′-bromospiro[cyclopropane-1,3′-isoindoline]-1′-one

To a solution of 5-bromo-3-methyleneisoindolin-1-one (1.0 g, 4.46 mmol) in DCM (25 mL) was added diethylzinc (27 mL, 27 mmol) at 0° C. under nitrogen. After 15 min stirring, diiodomethane (4.5 mL, 54 mmol) and diethylzinc (27 mL, 27 mmol) were added at 0° C. After 5 min stirring, additional diiodomethane (4.5 mL, 54 mmol) was added. The reaction mixture was then stirred at rt overnight, quenched with a saturated aqueous solution of NH₄Cl and extracted with DCM (100 mL×3). The combined organic layers were washed with brine, dried over Na₂SO₄ and concentrated under reduced pressure. Further purification by flash column chromatography eluting with DCM/MeOH (50:1) gave 5′-bromospiro[cyclopropane-1,3′-isoindoline]-1′-one (480 mg, 2.02 mmol, 45% yield).

LCMS (ES-API, Method C): 0.74 min, m/z 237.9/239.9 [M]⁺/[M+2].

### Step 5: tert-butyl N-(1′-oxospiro[cyclopropane-1,3′-isoindoline]-5′-yl)carbamate

To a solution of 5′-bromospiro[cyclopropane-1,3′-isoindoline]-1′-one (400 mg, 1.68 mmol, 1.0 eq.) and tert-butyl carbamate (394 mg, 3.36 mmol, 2.0 eq) in toluene (20 mL) were added Pd(OAc)₂ (38 mg, 0.168 mmol, 0.1 eq.) and Cs₂CO₃ (1.64 g, 5.04 mmol, 3.0 eq.) and Xant-Phos (194 mg, 0.336 mmol, 0.2 eq.). The mixture was then stirred at 110° C. under nitrogen overnight. The crude material was purified by flash column chromatography eluting with EtOAc/Pet. Ether (2:1) to give tert-butyl N-(1′-oxospiro[cyclopropane-1,3′-isoindoline]-5′-yl)carbamate (220 mg, 0.80 mmol, 48% yield).

LC-MS (ES-API, Method J): 0.62 min, m/z, 275.10 [M+H]⁺.

### Step 6: 5′-aminospiro[cyclopropane-1,3′-isoindoline]-1′-one

A mixture of tert-butyl N-(1′-oxospiro[cyclopropane-1,3′-isoindoline]-5′-yl)carbamate (1.6 g, 5.83 mmol, 1.0 eq) and TFA (10 mL, 5.83 mmol, 1.0 eq) in DCM was stirred at room temperature under N₂ for 1 h. The pH of the mixture was adjusted to pH 8 with NaHCO₃. The mixture was diluted with water (50 mL) and extracted with CHCl₃/iPrOH (3:1, 20 mL×3). The combined organic layers were washed with brine (50 mL), dried over Na₂SO₄ and concentrated in vacuum. The crude material was purified by flash column chromatography to give 5′-aminospiro[cyclopropane-1,3′-isoindoline]-1′-one (770 mg, 4.42 mmol, 76% yield).

LC-MS (ES-API, Method K): 0.40 min, m/z, 175.1 [M+H]⁺.

### Intermediate 5: 5-amino-3-methyl-isoindolin-1-one

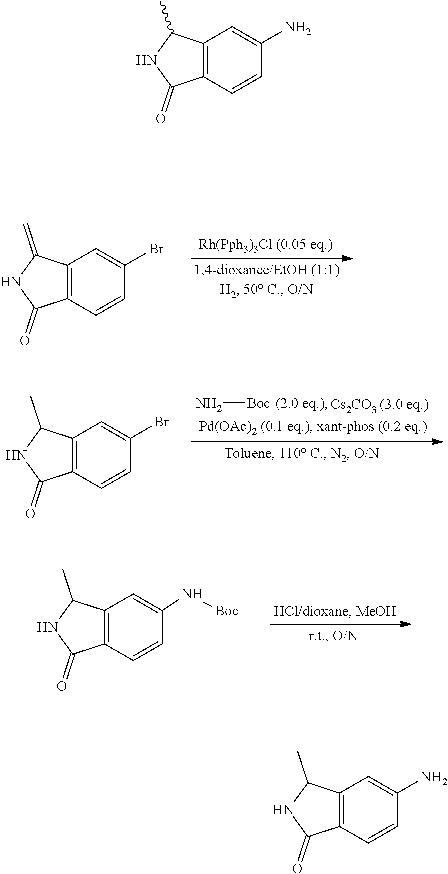 [SMILES: CC1NC(=O)c2ccc(N)cc21 ; C=C1NC(=O)c2ccc(Br)cc21 ; CC1NC(=O)c2ccc(Br)cc21 ; CC(C)(C)OC(N)=O ; CC1NC(=O)c2ccc(NC(=O)OC(C)(C)C)cc21 ; CC1NC(=O)c2ccc(N)cc21]

### Step 1: 5-bromo-3-methyl-isoindolin-1-one

To a solution of 5-bromo-3-methyleneisoindolin-1-one (11 g, 49.1 mmol, 1.0 eq.) in dioxane (275 mL) and EtOH (275 mL) was added (PPh₃)₃RhCl (2.27, 2.45 mmol, 0.05 eq), the mixture was stirred at 50° C. under H₂ atmosphere overnight. The mixture was concentrated under reduced pressure. The crude material was purified by flash column chromatography eluting with EtOAc in Pet. Ether (20:1 to 3:1) to give 5-bromo-3-methyl-isoindolin-1-one (9.1 g, 40.25 mmol, 82% yield).

LCMS (ES-API, Method D): 1.42 min, m/z 226.0 [M+H]⁺.

### Step 2: tert-butyl N-(3-methyl-1-oxo-isoindolin-5-yl)carbamate

To a solution of 5-bromo-3-methylisoindolin-1-one (6.8 g, 30.1 mmol) and tert-butyl carbamate (7.05 g, 60.2 mmol, 2.0 eq.) in toluene (200 mL) were added Pd(OAc)₂ (675 mg, 3.0 mmol, 0.1 eq.) and Cs₂CO₃ (29.4 g, 90.2 mmol, 3.0 eq,) and XantPhOS (3.5 g, 6.0 mmol, 0.2 eq.), the mixture was stirred at 110° C. under nitrogen overnight. The mixture was cooled, concentrated under reduced pressure. The crude material was purified by flash column chromatography eluting with EtOAc in Pet. Ether (50:1 to 20:1) to give tert-butyl N-(3-methyl-1-oxo-isoindolin-5-yl)carbamate.

LCMS (ES-API, Method D): 1.61 min, m/z 263.2 [M+H]+.

### Step 3: 5-amino-3-methyl-isoindolin-1-one

To a solution of tert-butyl N-(3-methyl-1-oxoisoindolin-5-yl)carbamate (2.3 g, 8.76 mmol, 1.0 eq.) in MeOH (2 mL) was added 4M HCl in 1,4-dioxane (6 mL). The mixture was stirred at rt overnight, adjusted to pH=8 with a saturated sodium bicarbonate solution and extracted with EtOAc (30 mL×5). The combined organic layer was washed with brine, dried over Na₂SO₄ to give 5-amino-3-methyl-isoindolin-1-one (1.28 g, 6.30 mmol, 72% yield).

LCMS (ES-API, Method C): 0.32 min, m/z 163.0 [M+H]+.

¹H NMR (400 MHz, DMSO-d₆) δ 7.99 (s, 1H), 7.28 (d, J=9.1 Hz, 1H), 6.62-6.58 (m, 2H), 5.73 (s, 2H), 4.42 (q, J=6.2 Hz, 1H), 1.30 (d, J=6.6 Hz, 3H).

### Intermediate 6: 5-amino-3,6-dimethylisoindolin-1-one

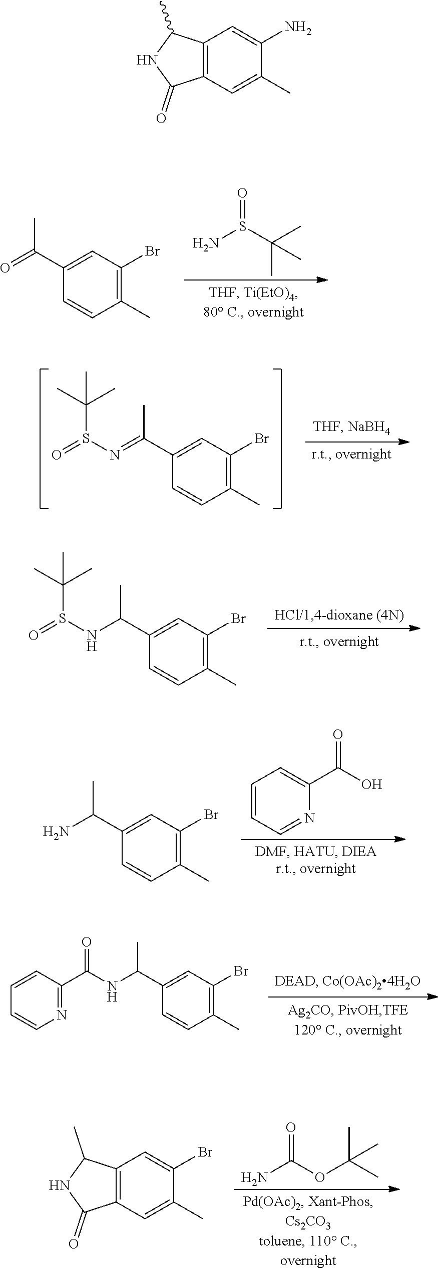

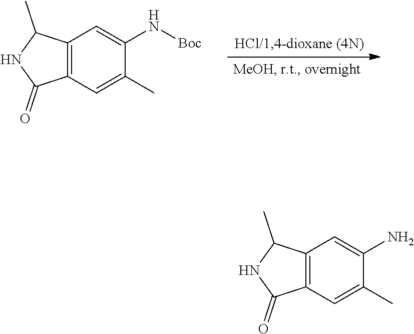 [SMILES: Cc1cc2c(cc1NC(=O)OC(C)(C)C)C(C)NC2=O ; Cc1cc2c(cc1N)C(C)NC2=O]

### Step 1a: (E)-N-(1-(3-bromo-4-methylphenyl)ethylidene)-2-methylpropane-2-sulfinamide

Titanium ethoxide (148 g, 422.4 mmol, 1.5 eq.) was added to a solution of 1-(3-bromo-4-methylphenyl)ethan-1-one (30 g, 140.8 mmol, 1.0 eq.) and 2-methylpropane-2-sulfinamide (34 g, 281.6 mmol, 2.0 eq.) in dry THF (600 mL) and the mixture was heated at 80° C. under N₂ atmosphere overnight, then allowed to cool to room temperature. The mixture was used directly in the next step without further process.

LC-MS (ES-API, Method C): 1.95 min, m/z 316.0/318.0 [M]⁺/[M+2]⁺.

### Step 1b: N-(1-(3-bromo-4-methylphenyl)ethyl)-2-methylpropane-2-sulfinamide

NaBH₄ (15.96 g, 422.4 mmol, 1.5 eq.) was added to the crude mixture (E)-N-(1-(3-bromo-4-methylphenyl)ethylidene)-2-methylpropane-2-sulfinamide in THF at 0° C. under N₂ atmosphere and then allowed to warm to r.t. and stirred overnight. The mixture was quenched with MeOH (250 mL), diluted with NaHCO₃ (sat. aq., 600 mL) and extracted with EtOAc (200 mL×3). The combined organic layers were washed with brine (100 mL), dried over Na₂SO₄, and concentrated in vacuo to give crude N-(1-(3-bromo-4-methylphenyl)ethyl)-2-methylpropane-2-sulfinamide (37.6 g, 118 mmol, 84% over two steps) as a yellow solid which was used directly in the next step without further purification.

LC-MS (ES-API, Method C): 1.72 min, m/z 318.1/320.1 [M]⁺/[M+2]⁺.

### Step 2: 1-(3-bromo-4-methy/phenyl)ethan-1-amine

To solution of N-(1-(3-bromo-4-methylphenyl)ethyl)-2-methylpropane-2-sulfinamide (44 g, 138.25 mmol) in MeOH (38 mL) was added a HCl solution in 1,4-dioxane (4M, 38 mL). The reaction mixture was stirred at room temperature overnight, adjusted to pH 8 with a solution of saturated sodium bicarbonate solution at 0° C., extracted by EtOAc (300 mL×5), washed with brine, dried over Na₂SO₄ and concentrated to give crude 1-(3-bromo-4-methylphenyl)ethan-1-amine (assumed quantitative) as a yellow solid, which was used as such for the next step.

¹H NMR (400 MHz, CDCl₃) δ 7.78 (s, 1H), 7.48-7.42 (m, 2H), 4.19-4.12 (m, 1H), 3.95-3.90 (m, 2H), 2.50 (s, 3H), 1.45-1.39 (d, J=6.6 Hz, 3H).

### Step 3: N-(1-(3-bromo-4-methylphenyl)ethyl)picolinamide

A solution of 1-(3-bromo-4-methylphenyl)ethan-1-amine (25 g, 116.8 mmol, 1.0 eq.), picolinic acid (14.4 g, 116.8 mmol, 1.0 eq.), HATU (53.3 g, 140.1 mmol, 1.2 eq.) and DIEA (45.3 g, 350.3 mmol, 3.0 eq.) in DMF (250 mL) was stirred at room temperature overnight, diluted with water (1,000 mL) and extracted with EtOAc (200 mL×3). The combined organic layers were washed with brine (200 mL), dried over Na₂SO₄ and then concentrated in vacuo. The residue was purified by column chromatography on silica gel eluting with 12.5% EtOAc in Pet. Ether to give crude N-(1-(3-bromo-4-methylphenyl)ethyl)picolinamide (37.3 g, 116.8 mmol, 62% yield over 4 steps) as a light yellow solid. LC-MS (ES-API, Method C): 1.75 min, m/z 319.1/321.1 [M]⁺/[M+2]⁺.

### Step 4: 5-bromo-3,6-dimethylisoindolin-1-one

A solution of N-(1-(3-bromo-4-methylphenyl)ethyl)picolinamide (5.0 g, 1.0 eq.), Co(OAc)₂·4H₂O (2.3 g, 0.6 eq.), Ag2CO₃ (25.9 g, 6.0 eq.), PivOH (9.6 g, 6.0 eq.) and DEAD (16.4 g, 6.0 eq.) in TFE (10 V) was heated at 120° C. in a sealed tube for 16 h. The reaction mixtures was then filtered through celite and washed with EtOAc (5 V) twice. Water (20 V) was added to the filtrate. The layers were separated and the organic layer was extracted with EtOAc (10 V) twice. The combined organic layers were washed with brine (10 V), dried over Na₂SO₄ and concentrated in vacuo. The residue was purified by column chromatography on silica gel, eluting with 5-35% EtOAc in n-heptane to give 5-bromo-3,6-dimethylisoindolin-1-one (2.4 g, 58% yield) as a yellow solid.

LC-MS (ES-API, Method D): 1.71 min, m/z=240.0/242.0 [M]⁺/[M+2]⁺

### Step 5: tert-butyl N-(3,6-dimethyl-1-oxoisoindolin-5-yl)carbamate

A solution of 5-bromo-3,6-dimethyl-isoindolin-1-one (4.0 g, 16.8 mmol, 1.0 eq.), tert-butyl carbamate (3.91 g, 33.6 mmol, 2.0 eq.), Cs₂CO₃ (16.32 g, 50.4 mmol, 3.0 eq.), Xant-Phos (1.93 g, 3.36 mmol, 0.2 eq.) and Pd(OAc)₂ (374 mg, 1.68 mmol, 0.1 eq.) in toluene (255 mL) was heated at 110° C. under N₂ atmosphere overnight. The reaction mixture was concentrated in vacuo. The residue was purified by column chromatography on silica gel eluting with 2-5% MeOH in DCM to give tert-butyl (3,6-dimethyl-1-oxoisoindolin-5-yl)carbamate (3.46 g, 12.52 mmol, 75% yield) as a yellow solid.

LC-MS (ES-API, Method D): 1.74 min, m/z 277.2 [M+H]⁺.

### Step 6: 5-amino-3,6-dimethylisoindolin-1-one

A mixture of tert-butyl N-(3,6-dimethyl-1-oxoisoindolin-5-yl)carbamate (3.16 g, 11.4 mmol, 1 eq.), MeOH (3 mL) and a 4 M solution of HCl in 1,4-dioxane (9 mL, 3 eq.) was stirred at 25° C. overnight.

The reaction was repeated using tert-butyl (3,6-dimethyl-1-oxoisoindolin-5-yl)carbamate (300 mg, 1.1 mmol) and the mixtures were combined, diluted with sat. aq. NaHCO₃ (50 mL) and extracted with EtOAc (50 mL×3). The combined organic layers were washed with brine (50 mL), dried over Na₂SO₄ and then concentrated in vacuo to give crude 5-amino-3,6-dimethylisoindolin-1-one (2.75 g, assumed quantitative) as a yellow solid.

LC-MS (ES-API, Method D): 0.39 min, m/z 177.1 [M+H]⁺.

¹H NMR (400 MHz, DMSO-d₆) δ 7.92 (s, 1H), 7.16 (s, 1H), 6.62 (s, 1H), 5.43 (s, 2H), 4.38 (q, J=6.6 Hz, 1H), 2.08 (s, 3H), 1.26 (d, J=6.6 Hz, 3H).

### Intermediate 7: 5-amino-6-fluoro-3-methylisoindolin-1-one

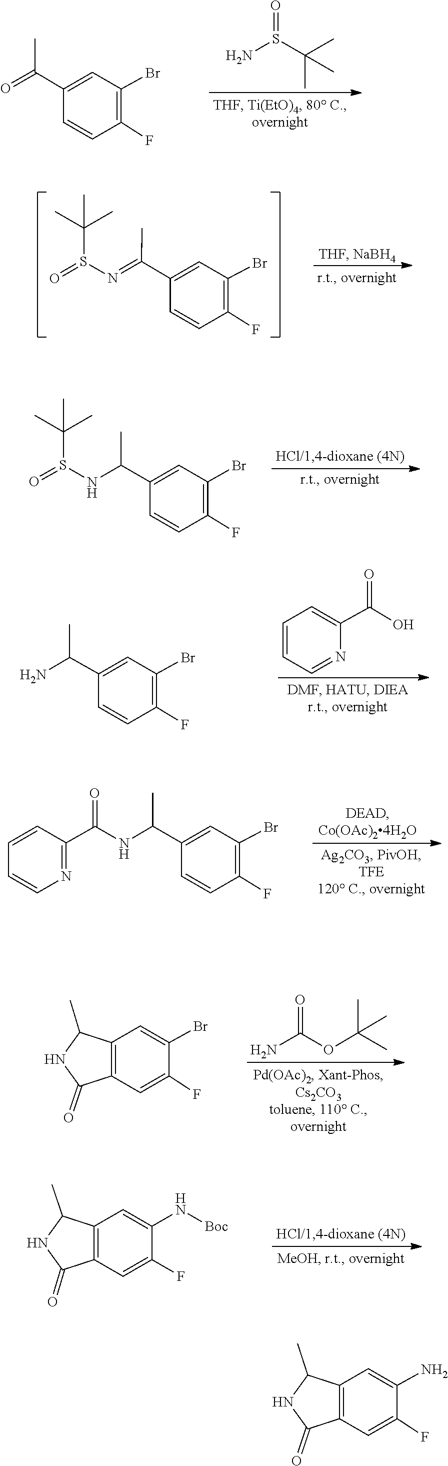

### Step 1: (E)-N-(1-(3-bromo-4-fluorophenyl)ethylidene)-2-methylpropane-2-sulfonamide

Titanium ethoxide (43.7 g, 124 mmol, 3.0 eq.) was added to a solution of 1-(3-bromo-4-fluorophenyl)ethan-1-one (9 g, 41.4 mmol, 1.0 eq.) and 2-methylpropane-2-sulfinamide (9.9 g, 82.8 mmol, 2 eq.) in THF (160 mL) and the mixture was heated at 80° C. under N₂ atmosphere overnight, then allowed to cool to room temperature. The mixture was used directly in the next step without further process.

LC-MS (ES-API, Method C): 1.57 min, m/z 322.1 [M+2]⁺

### Step 2: N-(1-(3-bromo-4-fluorophenyl)ethyl)-2-methylpropane-2-sulfinamide

NaBH₄ (4.7 g, 0.1 mol) was added to the mixture from the previous step at 0° C. under N₂ atmosphere and then allowed to warm to r.t. and stirred overnight. The reaction was quenched with MeOH (120 mL), diluted with NaHCO₃ (sat. aq., 300 mL) and extracted with EtOAc (100 mL×3). The combined organic layers were washed with brine (100 mL), dried over Na₂SO₄, and concentrated in vacuo to give crude N-(1-(3-bromo-4-fluorophenyl)ethyl)-2-methylpropane-2-sulfinamide (12.9 g, 40.0 mmol) as a light yellow solid which was used directly in the next step without further purification.

LC-MS (ES-API, Method C): 1.32 min, m/z 322.1 [M]⁺

¹H NMR (400 MHz, DMSO-d₆) δ 7.76 (dd, J=2.1, 6.8 Hz, 1H), 7.48-7.43 (m, 1H), 7.34 (t, J=8.6 Hz, 1H), 5.76 (d, J=8.0 Hz, 1H), 4.45-4.37 (m, 1H), 1.39 (d, J=7.2 Hz, 3H), 1.13 (s, 9H)

### Step 3: 1-(3-bromo-4-fluorophenyl)ethan-1-amine

HCl (4 M in 1,4-dioxane, 20 mL) was added to a solution of N-[1-(3-bromo-4-fluoro-phenyl)ethyl]-2-methyl-propane-2-sulfinamide (11.92 g, 37.0 mmol) in MeOH (20 mL) and the mixture was stirred at room temperature overnight. The mixture was diluted with NaHCO₃ (sat. aq., 100 mL) and extracted with EtOAc (100 mL×3). The combined organic layers were washed with brine (100 mL), dried over Na₂SO₄, and concentrated in vacuo to give crude 1-(3-bromo-4-fluorophenyl)ethan-1-amine (8.74 g) as an orange oil which was used in the next step without further purification.

LC-MS (ES-API, Method C): 0.35 min, m/z=218.0/220.0 [M]⁺/[M+2]⁺

¹H NMR (400 MHz, DMSO-d₆) δ 7.68 (dd, J=1.7, 6.9 Hz, 1H), 7.40-7.34 (m, 1H), 7.26 (t, J=8.7 Hz, 1H), 4.04-3.95 (m, 1H), 1.21 (d, J=6.6 Hz, 3H)

### Step 4: N-[1-(3-bromo-4-fluoro-phenyl)ethyl]pyridine-2-carboxamide

A solution of 1-(3-bromo-4-fluoro-phenyl)ethanamine (8.74 g, 40.1 mmol), picolinic acid (5.43 g, 44.1 mmol), HATU (18.26 g, 48.1 mmoL) and DIPEA (15.59 g, 120.3 mmol) in DMF (100 mL) was stirred at room temperature overnight. The mixture was diluted with water (1,000 mL), extracted with EtOAc (100 mL×3) and the combined organic layers washed with brine (50 mL), dried over Na₂SO₄ and concentrated in vacuo. The residue was purified by column chromatography on silica gel, eluting with 5% MeOH in DCM to give N-[1-(3-bromo-4-fluoro-phenyl)ethyl]pyridine-2-carboxamide (9.3 g, 69% yield over 4 steps) as a light yellow solid.

LC-MS (ES-API, Method C): 1.61 min, m/z=323.0/325.0 [M+H]⁺

¹H NMR (400 MHz, DMSO-d₆) δ 9.20 (d, J=8.5 Hz, 1H), 8.69 (d, J=4.7 Hz, 1H), 8.03-7.98 (m, 2H), 7.80 (dd, J=2.1, 6.9 Hz, 1H), 7.66-7.61 (m, 1H), 7.52-7.46 (m, 1H), 7.33 (t, J=8.8 Hz, 1H), 5.23-5.14 (m, 1H), 1.53 (d, J=7.9 Hz, 3H)

### Step 5: 5-bromo-6-fluoro-3-methylisoindolin-1-one

A solution of N-[1-(3-bromo-4-fluoro-phenyl)ethyl]pyridine-2-carboxamide (5.0 g, 1.0 eq.), Co(OAc)₂(H₂O)₄ (2.3 g, 0.6 eq.), Ag2CO₃ (25.6 g, 6.0 eq.), pivalic acid (9.5 g, 6.0 eq.) and DEAD (16.2 g, 6.0 eq.) in 2,2,2-trifluoroethanol (10 V) heated at 120° C. in a sealed tube for 16 h. The reaction mixtures was then filtered through celite and washed with EtOAc (5 V) twice. Water (20 V) was added to the filtrate. The layers were separated and the organic layer was extracted with EtOAc (10 V) twice. The combined organic layers were washed with brine (10 V), dried over Na₂SO₄ and concentrated in vacuo. The residue was purified by column chromatography on silica gel, eluting with 5-30% EtOAc in n-heptane to give 5-bromo-6-fluoro-3-methylisoindolin-1-one (2.1 g, 49% yield) as a light yellow solid

LC-MS (ES-API, Method C): 0.69 min, m/z=244.0/246.0 [M]⁺/[M+2]⁺

¹H NMR (400 MHz, DMSO-d₆) δ 8.87 (s, 1H), 8.04 (d, J=6.1 Hz, 1H), 7.57 (d, J=7.7 Hz, 1H), 4.64 (q, J=6.9 Hz, 1H), 1.38 (d, J=6.4 Hz, 3H)

### Step 6: tert-butyl N-(6-fluoro-3-methyl-1-oxoisoindolin-5-yl)carbamate

A solution of 5-bromo-6-fluoro-3-methyl-isoindolin-1-one (2.5 g, 10.2 mmol), tert-butyl carbamate (2.4 g, 20.4 mmol), Pd(OAc)₂ (230 mg, 1.02 mmol), Xantphos (1.2 g, 2.04 mmol) and Cs₂CO₃ (10 g, 30.6 mmol) in toluene (80 mL) was heated at 110° C. under N₂ atmosphere overnight. The mixture was concentrated in vacuo and the residue purified by column chromatography on silica gel, eluting with 2% MeOH in DCM to give tert-butyl (6-fluoro-3-methyl-1-oxoisoindolin-5-yl)carbamate (1.58 g, 60% over 2 steps) as an orange solid.

LC-MS (ES-API, Method D): 1.77 min, m/z=281.1 [M+H]⁺

¹H NMR (400 MHz, DMSO-d₆) δ 9.27 (s, 1H), 8.65 (s, 1H), 7.94 (d, J=6.8 Hz, 1H), 7.44-7.41 (m, 1H), 4.60 (q, J=6.4 Hz, 1H), 1.50 (s, 9H), 1.35 (d, J=7.1 Hz, 3H)

### Step 7: 5-amino-6-fluoro-3-methylisoindolin-1-one

A mixture of tert-butyl N-(6-fluoro-3-methyl-1-oxo-isoindolin-5-yl)carbamate (1.58 g, 5.6 mmol), MeOH (3 mL) and a 4M solution of HCl in 1,4-dioxane (9 mL) was stirred at 25° C. overnight. The mixture was diluted with NaHCO₃ (sat. aq., 100 mL), extracted with CHCl₃/iPrOH (3:1, v/v, 60 mL×3) and the combined organic layers washed with brine (20 mL), dried over Na₂SO₄ and concentrated in vacuo. The residue was slurried with EtOAc (6 mL) and collected by filtration to give 5-amino-6-fluoro-3-methylisoindolin-1-one (500 mg, 49%) as a brown solid.

LC-MS (ES-API, Method C): 0.42 min, m/z=181.1 [M+H]⁺

¹H NMR (400 MHz, DMSO-d₆) δ 8.17 (s, 1H), 7.15 (d, J=10.4 Hz, 1H), 6.79 (d, J=7.6 Hz, 1H), 5.76 (s, 2H), 4.43 (q, J=6.8 Hz, 1H), 1.27 (d, J=6.8 Hz, 3H)

Compounds prepared in a similar manner to that set out above are given in the table below:

TABLE 1

| Intermediate | | | | |
|---|---|---|---|---|
| N | Structure | NMR | MS [M + H]⁺ | Method |
| 8 | 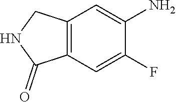 [SMILES: Nc1cc2c(cc1F)C(=O)NC2] | | 0.39 min, m/z = 167.1 [M + H]⁺ | Method D |

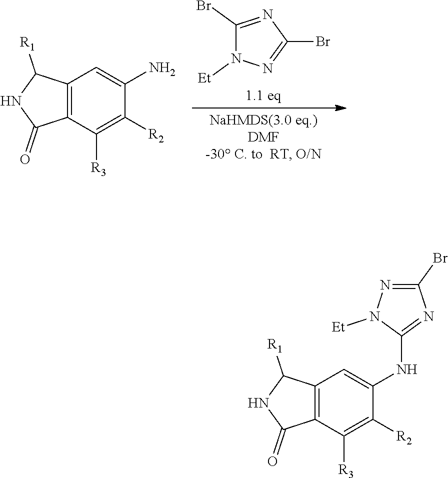

### Intermediate 9: 5-[(5-bromo-2-ethyl-1,2,4-triazol-3-yl)amino]isoindolin-1-one

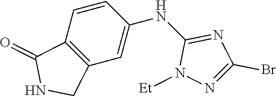 [SMILES: CCn1nc(Br)nc1Nc1ccc2c(c1)CNC2=O]

A solution of 5-aminoisoindolin-1-one (4.3 g, 29.02 mmol, 1.0 eq), 3,5-dibromo-1-ethyl-1H-1,2,4-triazole (7.77 g, 30.47 mmol, 1.05 eq), and NaHMDS (15.97 g, 87.07 mmol, 3.0 eq) in DMF (80 mL) was stirred at −30° C. over half an hour, and at r.t. under N₂ overnight. The mixture was diluted with NH₄Cl (300 mL) and extracted with EtOAc (200 mL). The combined organic layers were washed with brine (200 mL), dried over Na₂SO₄ and concentrated in vacuo to give 5-[(5-bromo-2-ethyl-1,2,4-triazol-3-yl)amino]isoindolin-1-one (5.5 g, 17.07 mmol, 59%).

LC-MS (ES-API, Method D): 1.20 min, m/z=322.1/324.0 [M]⁺/[M+2]⁺

¹H NMR (400 MHz, DMSO-d₆) δ 9.51 (s, 1H), 8.32 (s, 1H), 7.83 (s, 1H), 7.65-7.57 (m, 2H), 4.37 (s, 2H), 4.21-4.16 (m, 2H), 1.34 (t, J=7.0 Hz, 3H)

### Intermediate 10: 5-[(5-bromo-2-ethyl-1,2,4-triazol-3-yl)amino]-3,6-dimethyl-isoindolin-1-one

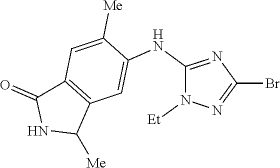 [SMILES: CCn1nc(Br)nc1Nc1cc2c(cc1C)C(=O)NC2C]

To a solution of 5-amino-3,6-dimethylisoindolin-1-one (5.5 g, 31.21 mmol) and 3,5-dibromo-1-ethyl-1H-1,2,4-triazole (7.96 mg, 31.21 mmol) in dry DMF (75 mL) was added NaHMDS (2 M in THF, 46.8 mL, 93.6 mmol). The mixture was stirred at −20° C. under N₂ for 15 min. Then the mixture was continued to react at r.t. under N₂ overnight. The mixture was diluted with water (800 mL) at 0° C., extracted with EtOAc (200 mL×5), washed with brine, dried over Na₂SO₄, concentrated in vacuo to give crude 5-[(5-bromo-2-ethyl-1,2,4-triazol-3-yl)amino]-3,6-dimethyl-isoindolin-1-one as a yellow solid (5.45 g, 80% purity).

LC-MS (ES-API, Method D): 1.33 min, m/z=350.1/352.1 [M]⁺/[M+2]⁺

¹H NMR (400 MHz, DMSO-d₆) δ 8.48 (s, 1H), 8.45 (s, 1H), 7.49 (s, 1H), 7.42 (s, 1H), 4.58 (q, J=6.5 Hz, 1H), 4.09 (q, J=7.5 Hz, 2H), 2.31 (s, 3H), 1.38-1.32 (m, 6H)

### Intermediate 11: 5-[(5-Bromo-2-ethyl-1,2,4-triazol-3-yl)amino]-3-methyl-isoindolin-1-one

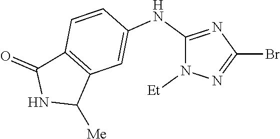 [SMILES: CCn1nc(Br)nc1Nc1ccc2c(c1)C(C)NC2=O]

To a solution of 5-amino-3-methylisoindolin-1-one (1.28 g, 7.88 mmol) and 3,5-dibromo-1-ethyl-1H-1,2,4-triazole (2.00 g, 7.88 mmol) in dry DMF (16 mL) was added NaHMDS (2 M in THF, 11.8 mL, 23.6 mmol). The mixture was stirred at −20° C. with N₂ for 15 min. then allow to return to r.t. with N₂ overnight. The mixture was concentrated and the residue was purified by flash column chromatography (SiO₂) eluting with MeOH in DCM (70:1 to 30:1) to give 5-[(5-bromo-2-ethyl-1,2,4-triazol-3-yl)amino]-3-methyl-isoindolin-1-one (1.98 g, 5.89 mmol, 75%) as light yellow solid LCMS (ES-API, Method D): 1.27 min, m/z=336.1/338.1 [M]⁺/[M+2]⁺

¹H NMR (400 MHz, DMSO-d₆) δ 9.47 (s, 1H), 8.42 (s, 1H), 7.69 (s, 1H), 7.64-7.56 (m, 2H), 4.62 (q, J=6.7 Hz, 1H), 4.16 (q, J=6.9 Hz, 2H), 1.38-1.33 (m, 6H)

### Intermediate 12: 5-[(5-bromo-2-ethyl-1,2,4-triazol-3-yl)amino]-6-fluoro-isoindolin-1-one

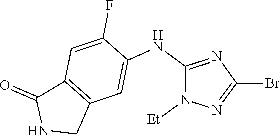 [SMILES: CCn1nc(Br)nc1Nc1cc2c(cc1F)C(=O)NC2]

To a solution of 5-amino-6-fluoro-isoindolin-1-one (73 mg, 0.44 mmol, 1.0 eq) and 3,5-dibromo-1-ethyl-1H-1,2,4-triazole (118 mg, 0.46 mmol) in DMF (3 mL) was added NaHMDS (2 M in THF, 0.7 mL, 1.4 mmol) at −30° C. under N₂, allowed to return to r.t. and stirred overnight. The mixture was diluted with sat.NH₄Cl (50 mL) and extracted with EtOAc (20 mL×3). The combined organic layers were washed with brine (10 mL), dried over Na₂SO₄ and concentrated in vacuum to give 5-[(5-bromo-2-ethyl-1,2,4-triazol-3-yl)amino]-6-fluoro-isoindolin-1-one as an orange solid (0.14 g, 0.421 mmol, 93%). LC-MS (ES-API, Method D): 1.14 min, m/z=340.1/342.1 [M]⁺/[M+2]⁺

### Intermediate 13: 5-[(5-bromo-2-ethyl-1,2,4-triazol-3-yl)amino]-6-fluoro-3-methyl-isoindolin-1-one

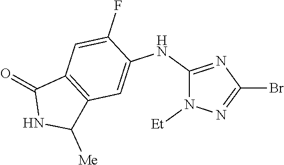 [SMILES: CCn1nc(Br)nc1Nc1cc2c(cc1F)C(=O)NC2C]

To a solution of 5-amino-6-fluoro-3-methyl-isoindolin-1-one (100 mg, 0.56 mmol, 1.0 eq) and 3,5-dibromo-1-ethyl-1H-1,2,4-triazole (148 mg, 0.58 mmol) in DMF (5 mL) was added NaHMDS (2 M in THF, 0.83 mL, 1.7 mmol) at −30° C. under N₂, allowed to return to r.t. and stirred overnight. The mixture was diluted with sat.NH₄Cl (50 mL) and extracted with EtOAc (10 mL×3), the combined organic layers were washed with brine (10 mL), dried over Na₂SO₄ and concentrated in vacuo to give 5-[(5-bromo-2-ethyl-1,2,4-triazol-3-yl)amino]-6-fluoro-3-methyl-isoindolin-1-one (0.17 g, 0.50 mmol, 90%).

LC-MS (ES-API, Method D): 1.33 min, m/z=354.1/356.1 [M]⁺/[M+2]⁺

¹H NMR (400 MHz, DMSO-d₆) δ 9.26 (s, 1H), 8.70 (s, 1H), 7.84 (d, J=7.6 Hz, 1H), 7.55 (d, J=10.1 Hz, 1H), 4.71 (q, J=6.4 Hz, 1H), 4.23 (q, J=7.5 Hz, 2H), 1.47-1.40 (m, 6H)

Compounds prepared in a similar manner to that set out above are given in Table 2 below:

TABLE 2

| Intermediate | | | MS | |
|---|---|---|---|---|
| N | Structure | NMR | [M + H⁺]+ | Method |
| 9 | 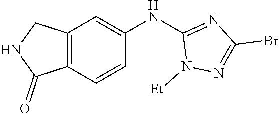 [SMILES: CCn1nc(Br)nc1Nc1ccc2c(c1)CNC2=O] | ¹H NMR (400 MHz, DMSO-d₆): δ 9.51 (s, 1H), 8.32 (s, 1H), 7.83 (s, 1H), 7.65-7.57 (m, 2H), 4.37 (s, 2H), 4.21-4.16 (m, 2H), 1.34 (t, J = 7.0 Hz, 3H) | 1.20 min, m/z = 322.1/324.0 [M]⁺/[M + 2]⁺ | Method D |
| 10 | 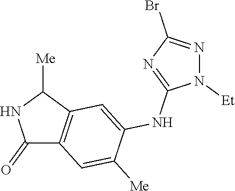 [SMILES: CCn1nc(Br)nc1Nc1cc2c(cc1C)C(=O)NC2C] | ¹H NMR (400 MHz, DMSO-d₆) δ 8.48 (s, 1H), 8.45 (s, 1H), 7.49 (s, 1H), 7.42 (s, 1H), 4.58 (q, J = 6.5 Hz, 1H), 4.09 (q, J = 7.5 Hz, 2H), 2.31 (s, 3H), 1.38-1.32 (m, 6H) | 1.33 min, m/z = 350.1/352.1 [M]⁺/[M + 2]⁺ | Method D |
| 11 | 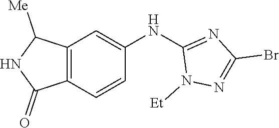 [SMILES: CCn1nc(Br)nc1Nc1ccc2c(c1)C(C)NC2=O] | ¹H NMR (400 MHz, DMSO-d₆): δ 9.47 (s, 1H), 8.42 (s, 1H), 7.69 (s, 1H), 7.64-7.56 (m, 2H), 4.62 (q, J = 6.7 Hz, 1H), 4.16 (q, J = 6.9 Hz, 2H), 1.38-1.33 (m, 6H) | 1.27 min, m/z = 336.1/338.1 [M]⁺/[M + 2]⁺ | Method D |
| 12 | 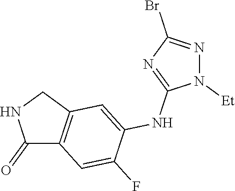 [SMILES: CCn1nc(Br)nc1Nc1cc2c(cc1F)C(=O)NC2] | | 1.14 min, m/z = 340.1/342.1 [M]⁺/[M + 2]⁺ | Method D |
| 13 | 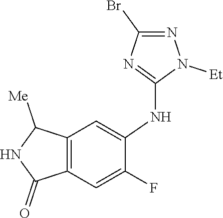 [SMILES: CCn1nc(Br)nc1Nc1cc2c(cc1F)C(=O)NC2C] | ¹H NMR (400 MHz, DMSO-d₆): δ 9.26 (s, 1H), 8.70 (s, 1H), 7.84 (d, J = 7.6 Hz, 1H), 7.55 (d, J = 10.1 Hz, 1H), 4.71 (q, J = 6.4 Hz, 1H), 4.23 (q, J = 7.5 Hz, 2H), 1.47-1.40 (m, 6H | 1.33 min, m/z 354.1/356.1 [M]⁺/[M + 2]⁺ | Method D |
| 14 | 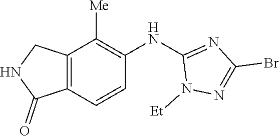 [SMILES: CCn1nc(Br)nc1Nc1ccc2c(c1C)CNC2=O] | ¹H NMR (400 MHz, DMSO-d₆) δ 8.63 (s, 1H), 8.46 (s, 1H), 7.49 (d, J = 8.3 Hz, 1H), 7.37 (d, J = 8.3 Hz, 1H), 4.34 (s, 2H), 4.10 (q, J = 7.1 Hz, 2H), 2.17 (s, 3H), 1.35 (t, J = 7.2 Hz, 3H) | 0.98 min, m/z = 335.9/337.9 [M]⁺/[M + 2]⁺ | Method D |
| 15 | 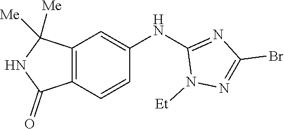 [SMILES: CCn1nc(Br)nc1Nc1ccc2c(c1)C(C)(C)NC2=O] | ¹H NMR (400 MHz, DMSO-d₆): δ 9.47 (s, 1H), 8.43 (s, 1H), 7.66 (dd, J = 1.9, 8.4 Hz, 1H), 7.61-7.54 (m, 2H), 4.19-4.13 (m, 2H), 1.44 (s, 6H), 1.34 (t, J = 7.6 Hz, 3H) | 1.30 min, m/z = 350.1/352.1 [M]⁺/[M + 2]⁺ | Method D |
| 16 | 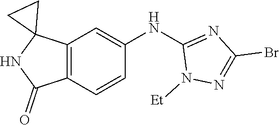 [SMILES: CCn1nc(Br)nc1Nc1ccc2c(c1)C1(CC1)NC2=O] | ¹H NMR (400 MHz, DMSO-d₆) δ 9.44 (s, 1H), 8.48 (s, 1H), 7.64 (s, 2H), 7.27 (s, 1H), 4.14 (q, J = 7.2 Hz, 2H), 1.51-1.45 (m, 2H), 1.36- 1.32 (m, 5H) | 1.36 min, m/z = 348.1/350.1 [M]⁺/[M + 2]⁺ | Method D |
| 17 | 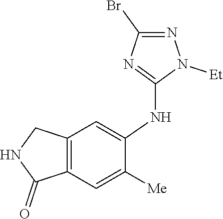 [SMILES: CCn1nc(Br)nc1Nc1cc2c(cc1C)C(=O)NC2] | | 1.11 min, m/z = 338.1 [M + 2]⁺ | Method D |

### General Procedures

### Intermediate 18: 5-methyl-2-(4-(4,4,5,5-tetramethyl-1,3,2-dioxaborolan-2-yl)phenyl) pyridine

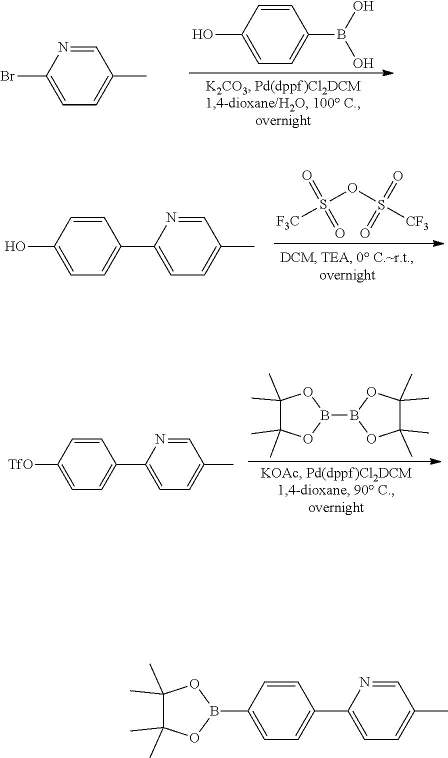

### Step 1: 4-(5-methylpyridin-2-yl)phenol

A solution of 2-bromo-5-methylpyridine (3.0 g, 17.4 mmol), (4-hydroxyphenyl)boronic acid (2.89 g, 20.9 mmol), Pd(dppf)Cl₂DCM (1.42 g, 1.74 mmol) and K₂CO₃ (3.62 g, 26.1 mmol) in 1,4-dioxane/H₂O (90 mL/10 mL) was stirred at 100° C. under N₂ atmosphere overnight. The mixture was cooled to room temperature and then concentrated in vacuo. The residue was purified by column chromatography on silica gel, eluting with 50% EtOAc in Pet. Ether to give 4-(5-methylpyridin-2-yl)phenol (1.44 g, 45%) as an off-white solid.

LC-MS (ES-API, Method C): 0.40 min, m/z=185.1 [M+H]+

¹H NMR (400 MHz, DMSO-d₆) δ 9.67 (s, 1H), 8.44 (s, 1H), 7.91 (d, J=9.4 Hz, 2H), 7.74 (d, J=7.8 Hz, 1H), 7.63 (dd, J=2.1, 8.0 Hz, 1H), 6.86 (d, J=8.6 Hz, 2H), 2.33 (s, 3H)

### Step 2: 4-(5-methylpyridin-2-yl)phenyl trifluoromethanesulfonate

To a mixture of 4-(5-methylpyridin-2-yl)phenol (1.34 g, 7.2 mmol) and TEA (2.2 g, 21.6 mmol) in DCM (30 mL) was added Trifluoromethanesulfonic anhydride (3.1 g, 11.0 mmol) slowly at 0° C. The resulting mixture was stirred at 0° C. for 0.5 h and then stirred at r.t. under N₂ atmosphere overnight. The mixture was diluted with water (100 mL) and extracted with DCM (20 mL×3). The combined organic layers were washed with brine (80 mL), dried over Na₂SO₄ and concentrated in vacuo. The residue was purified by column chromatography on silica gel, eluting with 25% EtOAc in Pet. Ether to give 4-(5-methylpyridin-2-yl)phenyl trifluoromethanesulfonate (1.94 g, 85%) as a brown oil.

LC-MS (ES-API, Method C): 2.00 min, m/z=318.1 [M+H]⁺

¹H NMR (400 MHz, DMSO-d₆) δ 8.55 (s, 1H), 8.25 (d, J=9.0 Hz, 2H), 7.95 (d, J=8.2 Hz, 1H), 7.76 (dd, J=1.8, 8.0 Hz, 1H), 7.61 (d, J=8.9 Hz, 2H), 2.37 (s, 3H) Step 3: 5-methyl-2-(4-(4,4,5,5-tetramethyl-1,3,2-dioxaborolan-2-yl)phenyl)pyridine A mixture of 4-(5-methylpyridin-2-yl)phenyl trifluoromethanesulfonate (1.7 g, 5.4 mmol), 4,4,4′,4′5,5,5′,5′-octamethyl-2,2′-bi(1,3,2-dioxaborolane) (2.72 g, 10.7 mmol), Pd(dppf)Cl₂DCM (438 mg, 0.54 mmol) and potassium acetate (1.58 g, 16.2 mmol) in 1,4-dioxane (75 mL) was stirred at 90° C. under N₂ atmosphere overnight. The mixture was concentrated in vacuo. and the crude was purified by column chromatography on silica gel, eluting with 25% EtOAc in Pet. Ether to give 5-methyl-2-(4-(4,4,5,5-tetramethyl-1,3,2-dioxaborolan-2-yl)phenyl)pyridine (1.8 g, assume quantitative) as a brown solid, which was used in the subsequent step without further purification.

LC-MS (ES-API, Method C): 1.70 min, m/z=296.2 [M+H]⁺

¹H NMR (400 MHz, DMSO-d₆) δ 8.51 (d, J=1.6 Hz, 1H), 8.07 (d, J=8.4 Hz, 2H), 7.88 (d, J=8.0 Hz, 1H), 7.77 (d, J=8.0 Hz, 2H), 7.71-7.69 (m, 1H), 2.34 (s, 3H), 1.31 (s, 12H)

### Intermediate 19: 2-methyl-5-[2-methyl-4-(4,4,5,5-tetramethyl-1,3,2-dioxaborolan-2-yl)phenyl]pyridine

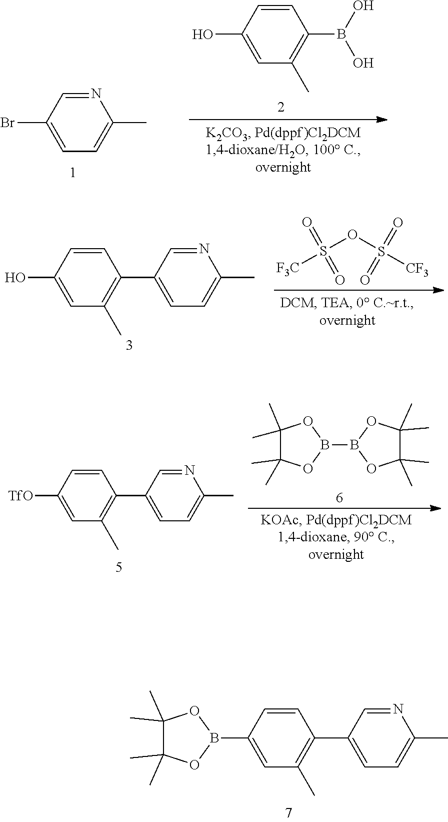

### Step 1: 3-methyl-4-(6-methyl-3-pyridyl)phenol

A solution of 5-bromo-2-methylpyridine (1.89 g, 10.99 mmol), (4-hydroxy-2-methylphenyl)boronic acid (2 g, 13.16 mmol), K₂CO₃ (2.3 g, 16.44 mmol) and Pd(dppf)Cl₂·DCM (897 mg, 1.10 mmol) in 1,4-dioxane/H₂O (45 mL/5 mL) was stirred at 100° C. under N₂ atmosphere overnight. The mixture was cooled to room temperature and then concentrated in vacuo. The residue was purified by column chromatography on silica gel, eluting with 1-2.5% MeOH in DCM to give 3-methyl-4-(6-methyl-3-pyridyl)phenol (1.16 g, 53%) as a yellow solid.

LC-MS (ES-API, Method D:) 0.33 min, m/z=200.1 [M+H]⁺

¹H NMR (400 MHz, DMSO-d₆) δ 9.42 (s, 1H), 8.35 (d, J=2.0 Hz, 1H), 7.60 (dd, J=2.3, 7.8 Hz, 1H), 7.27 (d, J=7.9 Hz, 1H), 7.02 (d, J=8.5 Hz, 1H), 6.72-6.70 (m, 1H), 6.69-6.66 (m, 1H), 2.49 (s, 3H), 2.15 (s, 3H)

### Step 2: [3-methyl-4-(6-methyl-3-pyridyl)phenyl]trifluoromethanesulfonate

To a mixture of 3-methyl-4-(6-methylpyridin-3-yl)phenol (1.1 g, 5.52 mmol) and TEA (1.67 g, 16.56 mmol) in dry DCM (15 mL) was added trifluoromethanesulfonic anhydride (2.3 g, 8.15 mmol) slowly at 0° C. The resulting mixture was stirred at 0° C. for 0.5 h and then stirred at room temperature under N₂ atmosphere overnight. The mixture was diluted with water (30 mL) and extracted with DCM (15 mL×2). The combined organic layers were washed with brine (50 mL), dried over Na₂SO₄ and concentrated in vacuo. The residue was purified by column chromatography on silica gel, eluting with 10% EtOAc in Pet. Ether to give [3-methyl-4-(6-methyl-3-pyridyl)phenyl]trifluoromethanesulfonate (716 mg, 39%) as a yellow oil.

LC-MS (ES-API, Method C): 1.09 min, m/z=332.1 [M+H]⁺

¹H NMR (400 MHz, DMSO-des) δ 8.44 (d, J=1.8 Hz, 1H), 7.72 (dd, J=2.5, 8.0 Hz, 1H), 7.49 (s, 1H), 7.42-7.39 (m, 2H), 7.34 (d, J=7.8 Hz, 1H), 2.52 (s, 3H), 2.28 (s, 3H)

### Step 3: 2-methyl-5-(2-methyl-4-(4,4,5,5-tetramethyl-1,3,2-dioxaborolan-2-yl) phenyl)pyridine

A mixture of 3-methyl-4-(6-methylpyridin-3-yl)phenyl trifluoromethanesulfonate (705 mg, 2.12 mmol), 4,4,4′,4′,5,5,5′,5′-octamethyl-2,2′-bi(1,3,2-dioxaborolane) (1.08 g, 4.25 mmol), Pd(dppf)Cl₂·DCM (173 mg, 0.212 mmol) and potassium acetate (626 mg, 6.38 mmol) in 1,4-dioxane (25 mL) was stirred at 90° C. under N₂ atmosphere overnight. The mixture was concentrated in vacuo. The residue was purified by column chromatography on silica gel, eluting with 10-25% EtOAc in Pet. Ether to give 2-methyl-5-(2-methyl-4-(4,4,5,5-tetramethyl-1,3,2-dioxaborolan-2-yl)phenyl)pyridine (707 mg, assumed quantitative) as a light yellow oil.

LC-MS (ES-API, Method C): 0.24 min, m/z=228.1 (boronic acid), 0.93 min, m/z=310.2 [M+H]⁺

¹H NMR (400 MHz, DMSO-d₆) δ 8.41 (d, J=2.3 Hz, 1H), 7.67 (dd, J=8.0, 2.4 Hz, 1H), 7.62 (s, 1H), 7.56 (d, J=7.5 Hz, 1H), 7.33 (d, J=8.0 Hz, 1H), 7.23 (d, J=7.5 Hz, 1H), 2.52 (s, 3H), 2.24 (s, 3H), 1.31 (s, 12H)

### Intermediate 20: 5-[4-(4,4,5,5-tetramethyl-1,3,2-dioxaborolan-2-yl)phenyl]-2-(trifluoromethyl)pyridine

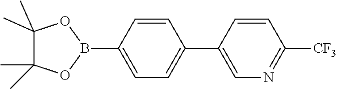 [SMILES: CC1(C)OB(c2ccc(-c3ccc(C(F)(F)F)nc3)cc2)OC1(C)C]

### Step 1: [4-[2-(trifluoromethyl)pyridine-4-yl]phenol

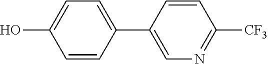 [SMILES: Oc1ccc(-c2ccc(C(F)(F)F)nc2)cc1]

To a solution of trifluoromethyl-5-bromo-2-pyridine (5.0 g, 22.1 mmol) and 4-hydroxybenzene boronic acid (3.36 g, 24.3 mmol) in 1,4-dioxane (110 mL) was added K₂CO₃ (9.17 g, 66.4 mmol) in water (33 mL) and the mixture was degassed with N₂ for 10 min. [1,1′-bis(diphenylphosphino)ferrocene]Palladium(II) chloride DCM complex (1.81 g, 2.21 mmol) was then added and the mixture was heated at 80° C. overnight. After cooling to r.t., the mixture was concentrated in vacuo and EtOAc (200 mL) was added. The mixture was filtered through celite and washed through with EtOAc. The filtrate was washed with water and brine (50 mL each), dried (Na₂SO₄), filtered and then concentrated in vacuo. The residue was purified by flash column chromatography (SiO₂) eluting with EtOAc in Pet. Ether (0-40%) to afford 4-[6-(trifluoromethyl)-3-pyridyl]phenol (5.13 g, 97%) as an off-white solid.

UPLC-MS (ES⁺, Method A): 1.57 min m/z=240.2 [M+H]⁺

¹H NMR (400 MHz, DMSO-d₆) δ 9.85 (s, 1H), 9.02 (d, J=2.1 Hz, 1H), 8.26 (dd, J=8.2, 2.1 Hz, 1H), 7.91 (d, J=8.2 Hz, 1H), 7.69-7.64 (m, 2H), 6.94-6.90 (m, 2H)

### Step 2: [4-[6-(trifluoromethyl)-3-pyridyl]phenyl]trifluoromethanesulfonate

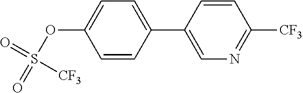 [SMILES: O=S(=O)(Oc1ccc(-c2ccc(C(F)(F)F)nc2)cc1)C(F)(F)F]

To a solution of 4-[6-(trifluoromethyl)-3-pyridyl]phenol (4.6 g, 19.2 mmol) in anhydrous pyridine (48 mL), under N₂, was added K₂CO₃ (2.66 g, 19.2 mmol) followed by N-phenyl bis-trifluoromethane sulfonimide (6.87 g, 19.2 mmol) portion-wise. The mixture was stirred at 25° C. for 3 days. The mixture was reduced in vacuo and the residue partitioned between sat. aq. (Na₂CO₃) solution (50 mL) and EtOAc (100 mL). The aqueous layer was extracted with EtOAc (50 mL). The combined organic phases were dried (Na₂SO₄), filtered and concentrated in vacuo. The residue was purified by silica chromatography eluting with EtOAc in Pet. Ether (0-30%) to afford [4-[6-(trifluoromethyl)-3-pyridyl]phenyl]trifluoromethanesulfonate (6.27 g, 88%) as a colourless oil that solidified upon standing.

UPLC-MS (ES⁴, Method A): 2.01 min, m/z=372.1 [M+H]⁺

¹H NMR (400 MHz, DMSO-d₆) δ 9.13 (d, J=2.0 Hz, 1H), 8.42 (dd, J=8.1, 2.0 Hz, 1H), 8.05-8.00 (m, 3H), 7.72-7.67 (m, 2H)

### Step 3: 5-[4-(4,4,5,5-tetramethyl-1,3,2-dioxaborolan-2-yl)phenyl]-2-(trifluoromethyl)pyridine

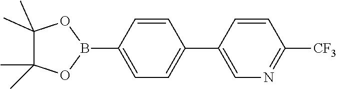 [SMILES: CC1(C)OB(c2ccc(-c3ccc(C(F)(F)F)nc3)cc2)OC1(C)C]

A solution of [4-[6-(trifluoromethyl)-3-pyridyl]phenyl]trifluoromethanesulfonate (6.27 g, 16.9 mmol), bis(pinacolato)diboron (6.44 g, 25.4 mmol) and KOAc (4.98 g, 50.7 mmol) in 1,4-dioxane (80 mL) was degassed with N₂ for 10 min then [1,1′-bis(diphenylphosphino)ferrocene]Palladium(II) chloride DCM complex (1.38 g, 1.69 mmol) was added and the mixture was heated at 80° C. for 2 hrs. After cooling to r.t., the mixture was filtered through celite and washed with EtOAc. The filtrate was reduced in vacuo and the residue purified by flash column chromatography (SiO₂) eluting with 0-100% DCM in Pet. Ether to afford 5-[4-(4,4,5,5-tetramethyl-1,3,2-dioxaborolan-2-yl)phenyl]-2-(trifluoromethyl)-pyridine (5.59 g, 16.0 mmol, 95%) as a white solid.

UPLC-MS (ES⁺, Method A): 2.15 min, m/z=350.0 [M+H]⁺

¹H NMR (400 MHz, DMSO-d₆) δ 9.11 (d, J=2.1 Hz, 1H), 8.39 (dd, J=8.2, 2.1 Hz, 1H), 8.00 (d, J=8.2 Hz, 1H), 7.87-7.81 (m, 4H), 1.32 (s, 12H)

### Intermediate 21: 4-[4-(4,4,5,5-tetramethyl-1,3,2-dioxaborolan-2-yl)phenyl]-2-(trifluoromethyl)pyridine

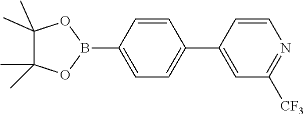 [SMILES: CC1(C)OB(c2ccc(-c3ccnc(C(F)(F)F)c3)cc2)OC1(C)C]

### Step 1: 4-[2-(trifluoromethyl)-4-pyridyl]phenol

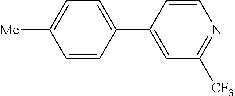 [SMILES: Cc1ccc(-c2ccnc(C(F)(F)F)c2)cc1]

4-Bromo-2-(trifluoromethyl)pyridine (9.5 g, 42.0 mmol), K₂CO₃ (17.5 g, 127 mmol) and 4-hydroxybenzene boronic acid (6.38 g, 46.3 mmol) were combined in 1,4-dioxane (100 mL) and water (20 mL). The reaction mixture was degassed with N₂ for 10 minutes then [1,1′-bis(diphenylphosphino)ferrocene]palladium(II) chloride DCM complex (3.43 g, 4.2 mmol) was added and the reaction mixture was heated at 80° C. for 1 hour. After cooling to r.t., the reaction mixture was concentrated in vacuo. The residue was diluted with EtOAc and passed through celite then evaporated to dryness in vacuo. The residue was purified by flash column chromatography (SiO₂) eluting with EtOAc in Pet. Ether (0-40%) to afford 4-[2-(trifluoromethyl)-4-pyridyl]phenol (9.19 g, 38.4 mmol, 91%) as a grey solid.

UPLC-MS (ES+, Method A): 1.59 min, m/z=240.1 [M+H]⁺

¹H NMR (400 MHz, DMSO-d₆) δ 10.00 (s, 1H), 8.73 (d, J=5.5 Hz, 1H), 8.08 (d, J=1.2 Hz, 1H), 7.96 (dd, J=1.2, 5.5 Hz, 1H), 7.83-7.79 (m, 2H), 6.94-6.90 (m, 2H)

### Step 2: [4-[2-(trifluoromethyl)-4-pyridyl]phenyl]trifluoromethanesulfonate

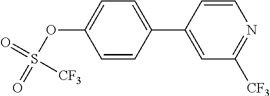 [SMILES: O=S(=O)(Oc1ccc(-c2ccnc(C(F)(F)F)c2)cc1)C(F)(F)F]

Trifluoromethanesulfonic anhydride (7.11 mL, 42.2 mmol) was added to a solution of 4-[2-(trifluoromethyl)-4-pyridyl]phenol (9.19 g, 38.4 mmol) and TEA (7.43 mL, 50 mmol) in DCM (200 mL) at 0° C., under N₂. The reaction mixture was allowed to warm to r.t. and after 30 minutes the reaction was poured into water. The organic layer was separated, washed with sat. aq. NaHCO₃ solution, sat. aq. NH₄Cl solution, dried (Na₂SO₄), filtered and concentrated in vacuo to afford [4-[2-(trifluoromethyl)-4-pyridyl]phenyl]trifluoromethanesulfonate (13.46 g, 36.3 mmol, 94%) as a yellow oil.

UPLC-MS (ES+, Method A): 2.01 min, m/z=372.2 [M+H]⁺

¹H NMR (400 MHz, CDCl₃) δ 8.82 (d, J=5.2 Hz, 1H), 7.88-7.86 (m, 1H), 7.77-7.73 (m, 2H), 7.69-7.66 (m, 1H), 7.48-7.44 (m, 2H)

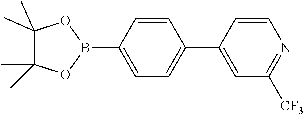 [SMILES: CC1(C)OB(c2ccc(-c3ccnc(C(F)(F)F)c3)cc2)OC1(C)C]

### Step 3: 4-[4-(4,4,5,5-tetramethyl-1,3,2-dioxaborolan-2-yl)phenyl]-2-(trifluoromethyl)pyridine CF₃

[4-[2-(Trifluoromethyl)-4-pyridyl]phenyl]trifluoromethanesulfonate (13.46 g, 36.3 mmol), bis(pinacolato)diboron (13.8 g, 54.4 mmol) and KOAc (10.7 g, 109 mmol) were combined in 1,4-dioxane (90 mL) and the reaction mixture was degassed with N₂. [1,1′-Bis(diphenylphosphino)ferrocene]palladium(II) chloride DCM complex (2.96 g, 3.63 mmol) was added and the mixture heated at 80° C. for 2 hours. After cooling to r.t., the mixture was filtered through celite and then washed with EtOAc. After evaporation of solvents, the residue was purified by flash column chromatography (SiO₂) eluting with EtOAc in Pet. Ether (0-100%) to afford 4-[4-(4,4,5,5-tetramethyl-1,3,2-dioxaborolan-2-yl)phenyl]-2-(trifluoromethyl)pyridine (12.6 g, 36.1 mmol, 99%) as a colourless oil.

UPLC-MS (ES⁺, Method A): 2.15 min, m/z=350.0 [M+H]⁺

¹H NMR (400 MHz, CDCl₃) δ 8.78 (d, J=4.4 Hz, 1H), 7.95 (d, J=8.4 Hz, 2H), 7.92-7.90 (m, 1H), 7.72-7.70 (m, 1H), 7.66 (d, J=8.4 Hz, 2H), 1.35 (s, 12H)

Compounds prepared in a similar manner to that set out above are given below in Table 3

TABLE 3

| Intermediate | | | MS | |
|---|---|---|---|---|
| N | Structure | NMR | [M + H⁺]+ | Method |
| 18 | 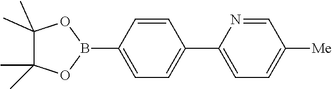 [SMILES: Cc1ccc(-c2ccc(B3OC(C)(C)C(C)(C)O3)cc2)nc1] | ¹H NMR (400 MHz, DMSO- d₆) δ 8.53 (s, 1H), 8.08 (d, J = 8.2 Hz, 2H),7.90 (d, J = 7.9 Hz, 1H), 7.78 (d, J = 7.9 Hz, 2H), 7.71 (dd, J = 1.8, 8.1 Hz, 1H), 2.35 (s, 3H), | 1.66 min, m/z = 296.2 | Method C |
| | | 1.32 (s, 12H) | | |
| 19 | 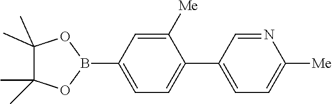 [SMILES: Cc1ccc(-c2ccc(B3OC(C)(C)C(C)(C)O3)cc2C)cn1] | ¹H NMR (400 MHz, DMSO- d₆) δ 8.39 (d, J = 1.8 Hz, 1H), 7.66 (dd, J =2.4, 8.0 Hz, 1H), 7.60 (s, 1H), 7.55 (d, J =7 .5 Hz, 1H), 7.31 (d, J = 8.1Hz, 1H), 7.21 (d, J = 7.5 Hz, 1H), 2.50 (s, 3H), | 0.93 min, m/z = 310.2 | Method C |
| | | 2.22 (s, 3H), 1.29 (s,12H) | | |
| 20 | 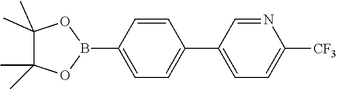 [SMILES: CC1(C)OB(c2ccc(-c3ccc(C(F)(F)F)nc3)cc2)OC1(C)C] | ¹H NMR (400 MHz, DMSO- d₆) δ 9.11 (d, J = 2.1 Hz, 1H), 8.39 (dd, J =8.2, 2.1 Hz, 1H), 8.00 (d, J = 8.2 Hz, 1H), 7.87-7.81 (m, 4H), 1.32 (s, 12H) | 2.15 min, m/z = 350.0 (84%) | Method A |
| 21 | 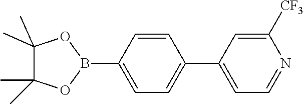 [SMILES: CC1(C)OB(c2ccc(-c3ccnc(C(F)(F)F)c3)cc2)OC1(C)C] | ¹H NMR (400 MHz, DMSO- d₆) δ 8.86 (d, J = 5.2 Hz, 1H), 8.22 (d, J = 1.2 Hz, 1H), 8.09 (dd, J = 1.4, 5.2 Hz, 1H), 7.96 (d, J = 8.2 Hz, 2H), 7.85 (d, J = 8.4 Hz, 2H), 1.34 (s, 12H) | 2.64 min, m/z = 350.0 | Method D |
| 22 | 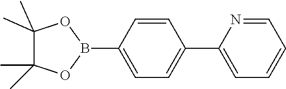 [SMILES: CC1(C)OB(c2ccc(-c3ccccn3)cc2)OC1(C)C] | | 1.79 min, m/z = 282.1 (92%) | Method A |
| 23 | 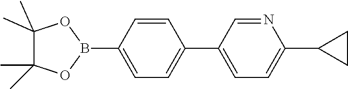 [SMILES: CC1(C)OB(c2ccc(-c3ccc(C4CC4)nc3)cc2)OC1(C)C] | | 1.84-1.87 min, m/z = 322.2 (95%) (35% boronic acid) | Method A |
| 24 | 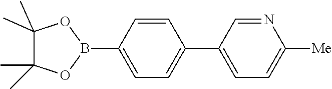 [SMILES: Cc1ccc(-c2ccc(B3OC(C)(C)C(C)(C)O3)cc2)cn1] | ¹H NMR (400 MHz, DMSO- d₆) δ 8.79 (d, J = 2.0 Hz, 1H), 8.00 (dd, J =2.5, 8.1 Hz, 1H), 7.79 (d, J = 8.2 Hz, 2H), 7.74 (d, J = 8.1 Hz, 2H), 7.37(d, J = 8.0 Hz, 1H), | 2.33 min, m/z = 296.2 | Method E |
| | | 2.53 (s, 3H), 1.33 (s, 12H) | | |
| 25 | 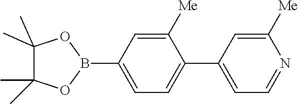 [SMILES: Cc1cc(-c2ccc(B3OC(C)(C)C(C)(C)O3)cc2C)ccn1] | | 1.49 min, m/z = 310.5 (85%) | Method A |
| 26 | 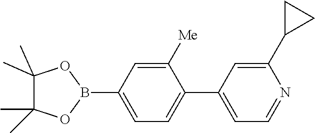 [SMILES: Cc1cc(B2OC(C)(C)C(C)(C)O2)ccc1-c1ccnc(C2CC2)c1] | | 1.71 min, m/z =336.6 (100%) (14% boronic acid) | Method A |
| 27 | 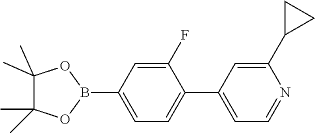 [SMILES: CC1(C)OB(c2ccc(-c3ccnc(C4CC4)c3)c(F)c2)OC1(C)C] | | 1.87 min, m/z = 340.4 (98%) (76% boronic acid) | Method A |
| 28 | 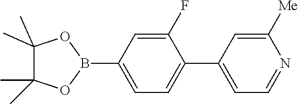 [SMILES: Cc1cc(-c2ccc(B3OC(C)(C)C(C)(C)O3)cc2F)ccn1] | | 1.57 min, m/z =314.0 (89%) (74% boronic acid) | Method A |
| 29 | 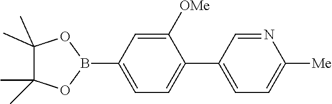 [SMILES: COc1cc(B2OC(C)(C)C(C)(C)O2)ccc1-c1ccc(C)nc1] | ¹H NMR (400 MHz, DMSO- d₆) δ 8.62 (d, J = 2.3 Hz, 1H), 7.86 (dd, J =2.3, 8.4 Hz, 1H), 7.47-7.42 (m, 2H), 7.40-7.36 (m, 2H), 3.88 (s, 3H), 2.59 (s, 3H), 1.40 (s, 12H) | 1.44 min, m/z = 326.1 | Method K |
| 30 | 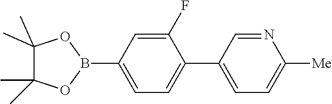 [SMILES: Cc1ccc(-c2ccc(B3OC(C)(C)C(C)(C)O3)cc2F)cn1] | ¹H NMR (400 MHz, DMSO- d₆) δ 8.60 (s, 1H), 7.84 (d, J = 8.1 Hz, 1H),7.58-7.54 (m, 2H), 7.43 (d, J = 11.0 Hz, 1H), 7.34 (d, J = 8.1 Hz, 1H), 2.49 (s, 3H), 1.28 (s, 12H) | 2.06 min, m/z = 314.2 | Method D |
| 31 | 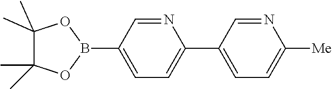 | | 0.29 min, m/z = 215.1 (boronic acid) | Method D |
| 32 | 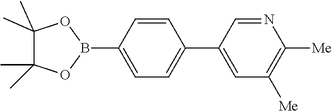 [SMILES: Cc1cc(-c2ccc(B3OC(C)(C)C(C)(C)O3)cc2)cnc1C] | ¹H NMR (400 MHz, DMSO- d₆) δ 8.60 (d, J = 2.1 Hz, 1H), 7.86 (d, J = 2.0 Hz, 1H), 7.79 (d, J = 8.1 Hz, 2H), 7.73 (d, J = 8.2 Hz, 2H), 2.48 (s, 3H), 2.35 (s, 3H), 1.33 (s, 12H) | 0.71 min, m/z = 310.1 | Method J |
| 33 | 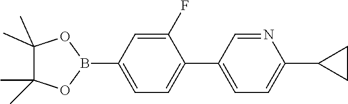 [SMILES: CC1(C)OB(c2ccc(-c3ccc(C4CC4)nc3)c(F)c2)OC1(C)C] | ¹H NMR (400 MHz, CDCl₃) δ 8.62 (s, 1H), 7.76-7.73 (m, 1H), 7.65-7.63 (m, 1H), 7.59-7.56 (m, 1H), 7.42 (t, J = 7.5 Hz, 1H), 7.20 (d, J = 8.1 Hz, 1H), 2.11-2.05 (m, 1H), 1.36 (s, 12H), 1.08-1.05 | 2.04 min, m/z = 340.3 (91%) 60% boronic acid) | Method A |
| | | (m, 2H), 1.05-1.01 (m, 2H) | | |
| 34 | 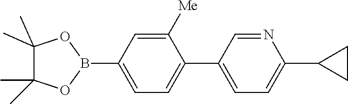 [SMILES: Cc1cc(B2OC(C)(C)C(C)(C)O2)ccc1-c1ccc(C2CC2)nc1] | ¹H NMR (400 MHz, CDCl₃) δ 8.38 (dd, J = 0.6, 2.3 Hz, 1H), 7.66 (dd, J = 2.3, 8.0 Hz, 1H), 7.62 (s, 1H), 7.57 (d, J = 7.7 Hz, 1H), 7.40- 7.37 (m, 1H), 7.23 (d, J = 7.6 Hz, 1H), 2.25 (s, 3H), 2.20- | 1.90 min, m/z = 336.3 (100%) (69% boronic acid) | Method A |
| | | 2.13 (m, 1H), 1.31 (s, 12H), | | |
| | | 1.02-0.95 (m, 4H) | | |
| 35 | 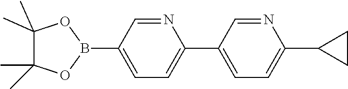 | ¹H NMR (400 MHz, DMSO- d₆) δ 9.21 (d, J = 1.8 Hz, 1H), 8.89 (s, 1H), 8.67 (dd, J = 2.1, 8.6 Hz, 1H), 8.19- 8.16 (m, 1H), 8.13 (d, J = 7.6 Hz,1H), 7.60 (d, J = 8.7 | 0.48 min, m/z = 241.1 (boronic acid) | Method K |
| | | Hz, 1H), 2.36-2.28 (m, 1H), | | |
| | | 1.35 (s, 12H), 1.26-1.20 (m, | | |
| | | 2H), 1.16-1.12 (m, 2H) | | |
| 36 | 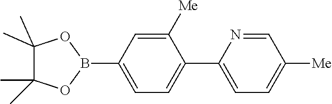 [SMILES: Cc1ccc(-c2ccc(B3OC(C)(C)C(C)(C)O3)cc2C)nc1] | ¹H NMR (400 MHz, DMSO- d₆) δ 8.53 (s, 1H), 7.73 (d, J = 8 .0 Hz, 1H),7.63 (s, 1H), 7.60 (d, J = 7.6 Hz, 1H), 7.45-7.39 (m, 2H), 2.38 (s, 3H), 2.36 (s, 3H), 1.35 (s, 12H) | 2.00 min, m/z = 310.2 | Method K |
| 37 | 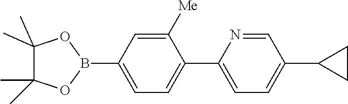 [SMILES: Cc1cc(B2OC(C)(C)C(C)(C)O2)ccc1-c1ccc(C2CC2)cn1] | ¹H NMR (400 MHz, DMSO- d₆) δ 8.51 (d, J = 1.8 Hz, 1H), 7.61 (s, 1H), 7.58 (d, J = 7.7 Hz, 1H), 7.51 (dd, J = 2.2, 8.2 Hz, 1H), 7.40 (t, J = 7.1 Hz, 2H), 2.34 (s, 3H), 2.08-1.98 (m, 1H), 1.33 (s, | 2.71 min, m/z = 336.3 | Method D |
| | | 12H), 1.07-1.03 (m, 2H), | | |
| | | 0.84-0.79 (m, 2H) | | |
| 38 | 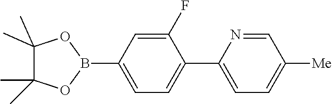 [SMILES: Cc1ccc(-c2ccc(B3OC(C)(C)C(C)(C)O3)cc2F)nc1] | ¹H NMR (400 MHz, DMSO- d₆) δ 8.59 (s, 1H), 8.00 (t, J = 7.9 Hz, 1H), 7.75 (s, 2H), 7.62 (d, J = 7.6 Hz, 1H), 7.48 (d, J = 11.6 Hz, 1H), 2.38 (s, 3H), 1.35 (s, 12H) | 2.28 min, m/z = 314.2 | Method C |
| 39 | 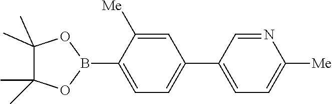 [SMILES: Cc1ccc(-c2ccc(B3OC(C)(C)C(C)(C)O3)c(C)c2)cn1] | ¹H NMR (400 MHz, DMSO- d₆) δ 8.77 (d, J = 2.2 Hz, 1H), 7.98 (dd, J = 2.5, 8.1 Hz, 1H), 7.73 (d, J = 7.7 Hz, 1H), 7.54 (s, 1H), 7.51 (d, J = 8.0 Hz, 1H), 7.36 (d, J = 7.3 Hz, 1H), 2.55 (s, 3H), | 2.02 min, m/z = 310.2 | Method D |
| | | 2.53 (s, 3H), 1.32 (s,12H) | | |
| 40 | 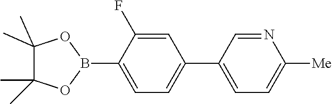 [SMILES: Cc1ccc(-c2ccc(B3OC(C)(C)C(C)(C)O3)c(F)c2)cn1] | ¹H NMR (400 MHz, DMSO-d₆) δ 8.84 (d, J = 2.1 Hz, 1H), 8.06 (dd, J =2.5, 8.2 Hz, 1H), 7.75 (t, J = 7.1 Hz, 1H), 7.58 (t, J = 10.2 Hz, 2H), 7.38(d, J = 8.0 Hz, 1H), 2.54 (s, 3H), 1.33 (s, 12H) | 1.83 min, m/z = 314.2 | Method D |
| 41 | 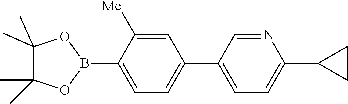 [SMILES: Cc1cc(-c2ccc(C3CC3)nc2)ccc1B1OC(C)(C)C(C)(C)O1] | ¹H NMR (400 MHz, DMSO- d₆) δ 8.73 (d, J = 1.9 Hz, 1H), 7.96 (d, J = 2.5Hz, 1H), 7.74 (d, J = 7.7 Hz, 1H), 7.52 (s, 1H), 7.49 (d, J = 7.7 Hz, 1H),7.39 (d, J = 8.0 Hz, 1H), 2.55 (s, 3H), 2.21-2.13 | 2.17 min, m/z-336.2 | Method C |
| | | (m, 1H), 1.34 (s, 12H), 1.02- | | |
| | | 0.96 (m, 4H) | | |
| 42 | 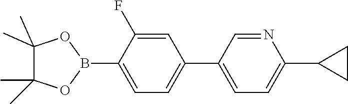 [SMILES: CC1(C)OB(c2ccc(-c3ccc(C4CC4)nc3)cc2F)OC1(C)C] | ¹H NMR (400 MHz, DMSO- d₆) δ 8.79 (d, J = 1.7 Hz, 1H), 8.02 (dd, J = 2.4, 8.2 Hz, 1H), 7.74 (t, J = 7.2 Hz, 1H), 7.56 (t, J = 10.4 Hz, 2H), 7.41(d, J = 8.2 Hz, 1H), 2.22-2.13 (m, 1H), 1.33 (s, | 2.03 min, m/z = 340.2 | Method C |
| | | 12H), 1.03-0.96 (m, 4H) | | |
| 43 | 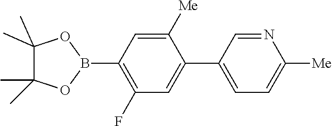 [SMILES: Cc1ccc(-c2cc(F)c(B3OC(C)(C)C(C)(C)O3)cc2C)cn1] | ¹H NMR (400 MHz, DMSO- d₆) δ 8.46 (d, J = 1.9 Hz, 1H), 7.72 (dd, J = 2.3, 7.9 Hz, 1H), 7.58 (d, J = 6.2 Hz, 1H), 7.40 (s, 1H), 7.06 (d, J = 9.9Hz, 1H), 2.54 (s, 3H), 2.22 (s, 3H), 1.33 (s, 12H) | 1.21 min, m/z = 328.2 | Method C |
| 44 | 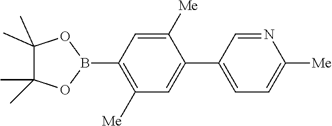 [SMILES: Cc1ccc(-c2cc(C)c(B3OC(C)(C)C(C)(C)O3)cc2C)cn1] | ¹H NMR (400 MHz, DMSO- d₆) δ 8.76 (d, J = 1.8 Hz, 1H), 8.29 (dd, J = 1.8, 8.1 Hz, 1H), 7.83 (d, J = 8.4 Hz, 1H), 7.61 (s, 1H), 7.15 (s, 1H), 2.72(s, 3H), 2.50 (s, 3H), 2.24 (s, 3H), 1.34 (s, 12H) | 1.35 min, m/z = 324.2 | Method C |
| 45 | 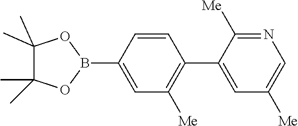 [SMILES: Cc1cnc(C)c(-c2ccc(B3OC(C)(C)C(C)(C)O3)cc2C)c1] | ¹H NMR (400 MHz, DMSO- d₆) δ 8.33 (d, J = 1.5 Hz, 1H), 7.65 (s, 1H), 7.58 (d, J = 7.5 Hz, 1H), 7.31 (d, J = 1.8 Hz, 1H), 7.12 (d, J = 7.4 Hz,1H), 2.31 (s, 3H), 2.14 (s, 3H), 2.04 (s, 3H), 1.34 (s, 12H) | 1.20 min, m/z = 324.2 | Method D |
| 46 | 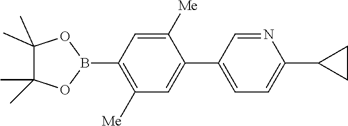 [SMILES: Cc1cc(-c2ccc(C3CC3)nc2)c(C)cc1B1OC(C)(C)C(C)(C)O1] | ¹H NMR (400 MHz, DMSO- d₆) δ 8.36 (d, J = 2.1 Hz, 1H), 7.63 (dd, J =2.3, 8.1 Hz, 1H), 7.56 (s, 1H), 7.37 (d, J = 8.0 Hz, 1H), 7.04 (s, 1H), 2.46 (s, 3H), 2.19 (s, 3H), 2.18-2.13 (m, 1H), 1.32 (s, 12H), 1.01-0.95 (m, 4H) | 2.31 min, m/z = 350.2 | Method C |
| 47 | 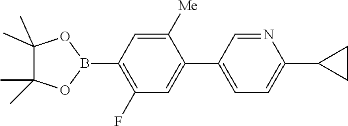 [SMILES: Cc1cc(B2OC(C)(C)C(C)(C)O2)c(F)cc1-c1ccc(C2CC2)nc1] | ¹H NMR (400 MHz, DMSO- d₆) δ 8.41 (d, J = 2.2 Hz, 1H), 7.68 (dd, J =2.6, 8.0 Hz, 1H), 7.57 (d, J = 6.6 Hz, 1H), 7.39 (d, J = 8.0 Hz, 1H), 7.05 (d, J = 9.8 Hz, 1H), 2.22 (s, 3H), 2.20-2.14 (m, 1H), 1.32 (s, 12H), 1.03- | 2.12 min, m/z = 354.3 | Method D |
| | | 0.96 (m, 4H) | | |
| 48 | 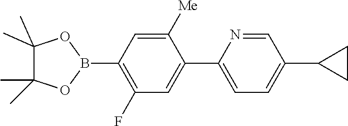 [SMILES: Cc1cc(B2OC(C)(C)C(C)(C)O2)c(F)cc1-c1ccc(C2CC2)cn1] | ¹H NMR (400 MHz, DMSO- d₆) δ 8.52 (d, J = 2.0 Hz, 1H), 7.57-7.50 (m, 2H), 7.46 (d, J = 8.0 Hz, 1H), 7.16 (d, J = 10.2 Hz, 1H), 2.31 (s, 3H), 2.08-2.00 (m, 1H), 1.34 (s, 12H), 1.08-1.04 (m, 2H), 0.84-0.80 (m, 2H) | 2.39 min, m/z = 354.2 | Method C |
| 49 | 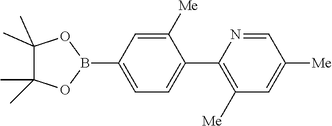 [SMILES: Cc1cnc(-c2ccc(B3OC(C)(C)C(C)(C)O3)cc2C)c(C)c1] | ¹H NMR (400 MHz, DMSO- d₆) δ 8.31 (s, 1H), 7.63 (s, 1H), 7.57-7.55 (m, 2H), 7.13 (d, J = 7.5 Hz, 1H), 2.33 (s, 3H), 2.02 (s, 3H), 2.00 (s, 3H), 1.33 (s, 12H) | 1.10 min, m/z = 324.2 | Method C |
| 50 | 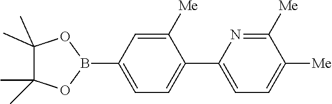 [SMILES: Cc1cc(B2OC(C)(C)C(C)(C)O2)ccc1-c1ccc(C)c(C)n1] | ¹H NMR (400 MHz, DMSO- d₆) δ 7.61-7.56 (m, 3H), 7.37 (d, J = 7.0 Hz, 1H), 7.24 (d, J = 8.4 Hz, 1H), 2.48 (s, 3H), 2.34 (s, 3H), 2.31 (s, 3H), 1.33 (s, 12H) | 1.74 min, m/z = 324.3 | Method D |
| 51 | 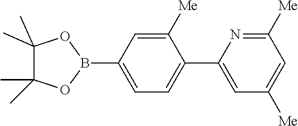 [SMILES: Cc1cc(C)nc(-c2ccc(B3OC(C)(C)C(C)(C)O3)cc2C)c1] | ¹H NMR (400 MHz, DMSO- d₆) δ 7.60 (s, 1H), 7.58 (d, J = 7.3 Hz, 1H), 7.36 (d, J = 7.6 Hz, 1H), 7.14 (s, 1H), 7.08 (s, 1H), 2.48 (s, 3H), 2.35 (s,3H), 2.33 (s, 3H), 1.33 (s, 12H) | 1.61 min, m/z = 324.2 | Method D |
| 52 | 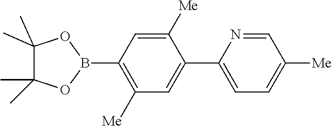 [SMILES: Cc1ccc(-c2cc(C)c(B3OC(C)(C)C(C)(C)O3)cc2C)nc1] | ¹H NMR (400 MHz, DMSO- d₆) δ 8.51 (s, 1H), 7.70 (dd, J = 2.2, 8.0 Hz, 1H), 7.55 (s, 1H), 7.41 (d, J = 8.1 Hz, 1H), 7.19 (s, 1H), 2.48 (s, 3H), 2.37 (s, 3H), 2.28 (s, 3H), 1.33 (s, 12H) | 1.75 min, m/z = 324.2 | Method C |
| 53 | 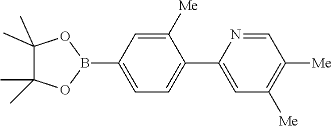 [SMILES: Cc1cnc(-c2ccc(B3OC(C)(C)C(C)(C)O3)cc2C)cc1C] | ¹H NMR (400 MHz, DMSO- d₆) δ 8.40 (s, 1H), 7.61 (s, 1H), 7.58 (d, J =7.7 Hz, 1H), 7.38 (d, J = 8.0 Hz, 1H), 7.32 (s, 1H), 2.34 (s, 3H), 2.33 (s, 3H), 2.28 (s, 3H), 1.34 (s, 12H) | 0.77 min, m/z = 324.2 | Method C |
| 54 | 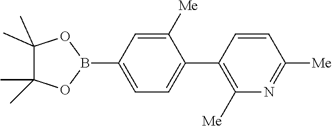 [SMILES: Cc1ccc(-c2ccc(B3OC(C)(C)C(C)(C)O3)cc2C)c(C)n1] | ¹H NMR (400MHZ, DMSO- d₆) δ 7.65 (s, 1H), 7.57 (d, J = 7.3 Hz, 1H),7.36 (d, J = 8.1 Hz, 1H), 7.16 (d, J = 6.9 Hz, 1H), 7.11 (d, J = 6.5 Hz, 1H), 2.50 (s, 3H), 2.15 (s, 3H), 2.03 (s, 3H), 1.32 (s, 12H) | 1.66 min, m/z = 324.3 | Method C |
| 55 | 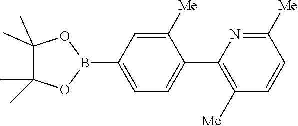 [SMILES: Cc1ccc(C)c(-c2ccc(B3OC(C)(C)C(C)(C)O3)cc2C)n1] | ¹H NMR (400 MHz, DMSO- d₆) δ 7.61-7.58 (m, 2H), 7.55 (d, J = 7.7 Hz, 1H), 7.16 (d, J = 8.1 Hz, 1H), 7.11 (d, J = 7.5 Hz, 1H), 2.44 (s, 3H), 2.00 (s, 3H), 1.97 (s, 3H), 1.32 (s, 12H) | 0.77 min, m/z = 324.2 | Method C |
| 56 | 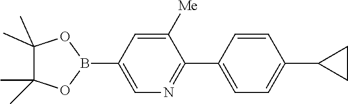 | | 0.52 min, m/z = 254.1 (boronic acid) | Method C |

### Intermediate 57: 4-methyl-1-[4-(4,4,5,5-tetramethyl-1,3,2-dioxaborolan-2-yl)phenyl]imidazole

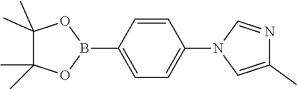

### Step 1: 1-(4-bromoanilino)propan-2-one

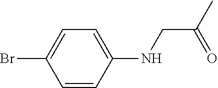

KI (1.59 g, 9.6 mmol) and K₂CO₃ (1.45 g, 10.5 mmol) were added to a solution of 4-bromoaniline (1.5 g, 8.7 mmol) in DMF (17.4 mL) under N₂, and the reaction mixture was stirred for 5 minutes at 20° C. before chloroacetone (1.56 mL, 19.6 mmol) was added. The reaction mixture was then stirred at 20° C. for 1 hour. The reaction mixture was concentrated in vacuo and the residue partitioned between EtOAc and water. The two phases were separated, and the water was re-extracted with EtOAc. The organic extracts were combined, washed with brine and filtered through phase separating filter paper. The crude material was purified by flash column chromatography (silica) eluting with EtOAc in Pet. Ether (0-60%) to afford 1-(4-bromoanilino)propan-2-one (701 mg, 3.07 mmol, 35% yield) as a yellow solid.

UPLC-MS (ES⁺, Method A): 1.62 min, m/z=227.9/229.8 [M]⁺/[M+2]⁺

¹H NMR (400 MHz, CDCl₃) δ 7.30-7.22 (m, 2H), 6.50-6.44 (m, 2H), 4.61 (br s, 1H), 3.97 (d, J=4.4 Hz, 2H), 2.26 (s, 3H)

### Step 2: N-acetonyl-N-(4-bromophenyl)formamide

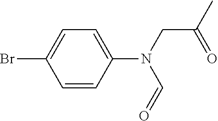

Formic acid (2.5 mL, 66.4 mmol) and acetic anhydride (6.3 mL, 66.4 mmol) were heated at 60° C. for 15 minutes. The solution was then cooled to 0° C. and 1-(4-bromoanilino)propan-2-one (0.13 mL, 3.07 mmol) was added. The mixture was heated at 60° C. for 1 hour. The reaction mixture was cooled to r.t. and concentrated in vacuo. The residue was partitioned between EtOAc and sat. aq. NaHCO₃ solution added. The two phases were separated and the aqueous phase was re-extracted with EtOAc. The combined organic extracts were washed with brine, filtered through phase separating filter paper and concentrated in vacuo to afford N-acetonyl-N-(4-bromophenyl)formamide (676 mg, 2.64 mmol, 86% yield) as a yellow oil.

UPLC-MS (ES⁺, Method A): 1.43 min, m/z=255.9/257.8 [M]⁺/[M+2]⁺

¹H NMR (400 MHz, CDCl₃) δ 8.46 (s, 1H), 7.57-7.50 (m, 2H), 7.10-7.02 (m, 2H), 4.52 (s, 2H), 2.22 (s, 3H)

### Step 3: 1-(4-bromophenyl)-4-methyl-imidazole

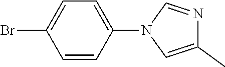

TEA (0.37 mL, 2.64 mmol) and NH₄OAc (2.04 g, 26.4 mmol) were successively added to a suspension of N-acetonyl-N-(4-bromophenyl)formamide (676 mg, 2.64 mmol) in 1-butanol (13 mL). The vial was sealed, and the mixture irradiated at 150° C. for 45 min. The reaction mixture was cooled to r.t. and then concentrated in vacuo. The residue was partitioned between EtOAc and water. The two phases were separated, and the aqueous phase re-extracted with EtOAc. The combined organic extracts were washed with brine, filtered through phase separating filter paper and then concentrated in vacuo. The crude material was purified by flash column chromatography (silica) eluting with EtOAc in Pet. Ether (0-100%) to afford 1-(4-bromophenyl)-4-methyl-imidazole (351 mg, 1.48 mmol, 56% yield) as a yellow solid.

UPLC-MS (ES⁺, Method A): 1.12 min, m/z=236.9/238.8 [M]⁺/[M+2]⁺

¹H NMR (400 MHz, CDCl₃) δ 7.76 (d, J=1.3 Hz, 1H), 7.62-7.56 (m, 2H), 7.27-7.21 (m, 2H), 6.98-6.96 (m, 1H), 2.29 (d, J=1.0 Hz, 3H)

### Step 4: 4-methyl-1-[4-(4,4,5,5-tetramethyl-1,3,2-dioxaborolan-2-yl)phenyl]imidazole

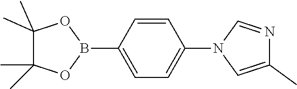

1-(4-Bromophenyl)-4-methyl-imidazole (351 mg, 1.48 mmol), bis(pinacolato)diboron (564 mg, 2.22 mmol) and KOAc (436 mg, 4.44 mmol) were combined in 1,4-dioxane (15 mL). The reaction mixture was degassed with N₂ then [1,1′-bis(diphenylphosphino)ferrocene]palladium(II) chloride DCM complex (60 mg, 0.07 mmol) was added. The reaction mixture was heated at 100° C. overnight. After cooling to r.t., the reaction mixture was filtered through celite and washed with EtOAc. The filtrate was concentrated in vacuo and the crude material was purified by flash column chromatography (silica) eluting with EtOAc in Pet. Ether (0-100%) to afford 4-methyl-1-[4-(4,4,5,5-tetramethyl-1,3,2-dioxaborolan-2-yl)phenyl]imidazole (189 mg, 0.66 mmol, 45% yield) as a brown oil.

UPLC-MS (ES⁺, Method A): 1.36 min, m/z—284.9 [M+H]⁺

¹H NMR (400 MHz, CDCl₃) δ 7.92-7.86 (m, 2H), 7.81 (d, J=1.4 Hz, 1H), 7.39-7.32 (m, 2H), 7.05-7.04 (m, 1H), 2.30 (d, J=1.0 Hz, 3H), 1.36 (s, 12H)

### Intermediate 58: 4-cyclopropyl-1-[2-methyl-4-(4,4,5,5-tetramethyl-1,3,2-dioxaborolan-2-yl)phenyl]imidazole

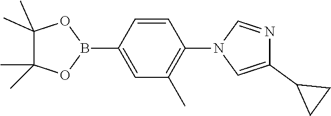 [SMILES: Cc1cc(B2OC(C)(C)C(C)(C)O2)ccc1-n1cnc(C2CC2)c1]

### Step 1: 2-(4-bromo-2-methyl-anilino)-1-cyclopropyl-ethanone

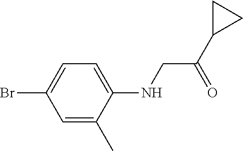

K₂CO₃ (446 mg, 3.2 mmol) and KI (491 mg, 3.0 mmol) were added to a solution of 4-bromo-2-methylaniline (500 mg, 2.7 mmol) in DMF (5.0 mL) under N₂, and the reaction mixture was stirred for 5 minutes at 20° C. before 2-bromo-1-cyclopropylethanone (0.59 mL, 6.05 mmol) was added. The reaction mixture was then stirred at 20° C. for 1 hour. The reaction mixture was concentrated in vacuo. The residue was partitioned between EtOAc and water. The two phases were separated, and the water was re-extracted with EtOAc. The organic extracts were combined, washed with brine and filtered through phase separating filter paper. The crude material was purified by flash column chromatography (silica) eluting with EtOAc in Pet. Ether (0-60%) to 2-(4-bromo-2-methyl-anilino)-1-cyclopropyl-ethanone (405 mg, 1.5 mmol, 56% yield) as a brown oil.

UPLC-MS (ES⁺, Method A): 1.90 min, m/z—267.9/269.9 [M]⁺/[M+2]⁺

¹H NMR (400 MHz, CDCl₃) δ 7.21 (dd, J=8.3, 2.5 Hz, 1H), 7.18-7.16 (m, 1H), 6.36 (d, J=8.4 Hz, 1H), 4.60 (br s, 1H), 4.17 (s, 2H), 2.17 (s, 3H), 2.05-1.98 (m, 1H), 1.20-1.15 (m, 2H), 1.05-0.99 (m, 2H)

### Step 2: N-(4-bromo-2-methyl-phenyl)-N-(2-cyclopropyl-2-oxo-ethyl)formamide

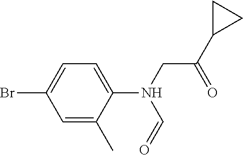 [SMILES: Cc1cc(Br)ccc1N(C=O)CC(=O)C1CC1]

Formic acid (1.23 mL, 32.6 mmol) and acetic anhydride (3.1 mL, 32.6 mmol) were heated at 60° C. for 15 minutes. The solution was then cooled to 0° C. and 2-(4-bromo-2-methyl-anilino)-1-cyclopropyl-ethanone (0.13 mL, 1.51 mmol) was added. The mixture was heated at 60° C. for 1 hour. The reaction mixture was cooled to r.t. and concentrated in vacuo. The residue was partitioned between EtOAc and sat. aq. NaHCQ₃ solution. The two phases were separated, and the aqueous phase was re-extracted with EtOAc. The combined organic extracts were washed with brine, filtered through phase separating filter paper and concentrated in vacuo to afford N-(4-bromo-2-methyl-phenyl)-N-(2-cyclopropyl-2-oxo-ethyl)formamide (423 mg, 1.43 mmol, 95% yield) as a yellow oil.

UPLC-MS (ES⁺, Method A): 1.67 min, m/z=295.9/297.9 [M]⁺/[M+2]⁺

¹H NMR (400 MHz, CDCl₃) δ 8.18 (s, 1H), 7.44 (d, J=2.0 Hz, 1H), 7.38-7.34 (m, 1H), 7.23 (d, J=8.4 Hz, 1H), 4.58 (s, 2H), 2.29 (s, 3H), 1.97-1.90 (m, 1H), 1.13-1.08 (m, 2H), 0.99-0.93 (m, 2H)

### Step 3: 1-(4-bromo-2-methylphenyl)-4-cyclopropyl-imidazole

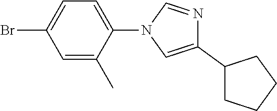 [SMILES: Cc1cc(Br)ccc1-n1cnc(C2CCCC2)c1]

TEA (0.2 mL, 1.43 mmol) and NH₄OAc (1.1 g, 14.3 mmol) were successively added to a suspension of N-(4-bromo-2-methyl-phenyl)-N-(2-cyclopropyl-2-oxo-ethyl)formamide (423 mg, 1.43 mmol) in 1-butanol (7 mL). The vial was sealed, and the mixture irradiated at 150° C. for 45 min. The reaction mixture was cooled to r.t. and concentrated in vacuo. The residue was partitioned between EtOAc and water. The two phases were separated, and the aqueous phase was re-extracted with EtOAc.

The combined organic extracts were washed with brine, filtered through phase separating filter paper and concentrated in vacuo. The crude material was purified by flash column chromatography (silica) eluting with EtOAc in Pet. Ether (0-50%) to afford 1-(4-bromo-2-methyl-phenyl)-4-cyclopropyl-imidazole (129 mg, 0.47 mmol, 33% yield) as a yellow oil.

UPLC-MS (ES⁴, Method A): 1.32 min, m/z—277.0/278.9 [M]⁺/[M+2]⁺

¹H NMR (400 MHz, CDCl₃) δ 7.47 (d, J=2.0 Hz, 1H), 7.42-7.38 (m, 2H), 7.07 (d, J=8.3 Hz, 1H), 6.75 (d, J=1.3 Hz, 1H), 2.18 (s, 3H), 1.94-1.86 (m, 1H), 0.92-0.85 (m, 2H), 0.84-0.79 (m, 2H)

### Step 4: 4-cyclopropyl-1-[2-methyl-4-(4,4,5,5-tetramethyl-1,3,2-dioxaborolan-2-yl)phenyl]imidazole

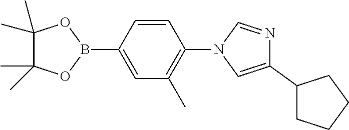 [SMILES: Cc1cc(B2OC(C)(C)C(C)(C)O2)ccc1-n1cnc(C2CCCC2)c1]

1-(4-Bromophenyl)-4-cyclopropyl-imidazole (129 mg, 0.47 mmol), bis(pinacolato)diboron (196 mg, 0.77 mmol) and KOAc (151 mg, 1.54 mmol) were combined in 1,4-dioxane (5 mL). The reaction mixture was degassed with N₂ then [1,1′-bis(diphenylphosphino)ferrocene]palladium(II) chloride DCM complex (21 mg, 0.03 mmol) was added. The reaction mixture was heated at 100° C. overnight. Cooled to r.t. and the reaction mixture was filtered through celite and washed through with EtOAc. The filtrate was concentrated in vacuo. The crude material was purified by flash column chromatography (silica) eluting with EtOAc in Pet. Ether (0-100%) to afford 4-cyclopropyl-1-[2-methyl-4-(4,4,5,5-tetramethyl-1,3,2-dioxaborolan-2-yl)phenyl]imidazole (quantitative) as a brown oil.

UPLC-MS (ES⁺, Method A): 1.52 min, m/z—325.0 [M+H]⁺

¹H NMR (400 MHz, CDCl₃) δ 7.76 (d, J=1.4 Hz, 1H), 7.70 (d, J=7.8 Hz, 1H), 7.44 (d, J=1.4 Hz, 1H), 7.20 (d, J=7.8 Hz, 1H), 6.79 (d, J=1.3 Hz, 1H), 2.23 (s, 3H), 1.94-1.87 (m, 1H), 1.36 (s, 12H), 0.91-0.85 (m, 2H), 0.84-0.80 (m, 2H)

### Intermediate 59: 4-cyclopropyl-1-[4-(4,4,5,5-tetramethyl-1,3,2-dioxaborolan-2-yl)phenyl]imidazole

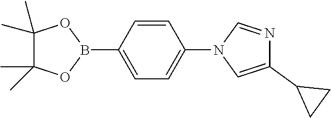 [SMILES: CC1(C)OB(c2ccc(-n3cnc(C4CC4)c3)cc2)OC1(C)C]

### Step 1: 2-(4-bromoanilino)-1-cyclopropyl-ethanone

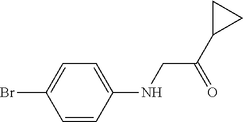 [SMILES: O=C(CNc1ccc(Br)cc1)C1CC1]

KI (3.4 g, 20.5 mmol) and K₂CO₃ (3.09 g, 22.4 mmol) were added to a solution of 4-bromoaniline (3.21 g, 18.7 mmol) in DMF (34 mL). The reaction was stirred for 5 minutes at 20° C., under N₂, before 2-bromo-1-cyclopropylethanone (4.1 mL, 41.9 mmol) was added. The reaction mixture was stirred at 20° C. for 1 hour. The reaction mixture was concentrated in vacuo and the residue was partitioned between EtOAc and water.and the two phases were separated. The aqueous layer was re-extracted with EtOAc. The organic extracts were combined, washed with brine, dried (Na₂SO₄), filtered and concentrated in vacuo. The crude material was purified by flash column chromatography (silica) eluting with EtOAc in Pet. Ether (0-60%) to afford 2-(4-bromoanilino)-1-cyclopropyl-ethanone (4.28 g, 16.8 mmol, 90% yield) as a yellow solid.

UPLC-MS (ES⁺, Method A): 1.80 min, m/z=253.9/255.9 [M]⁺/[M+2]⁺

¹H NMR (400 MHz, CDCl₃) δ 7.27 (d, J=8.9 Hz, 2H), 6.49 (d, J=8.9 Hz, 2H), 4.67 (s, 1H), 4.13 (s, 2H), 2.03-1.96 (m, 1H), 1.18-1.13 (m, 2H), 1.03-0.98 (m, 2H)

### Step 2: N-(4-bromophenyl)-N-(2-cyclopropyl-2-oxo-ethyl)formamide

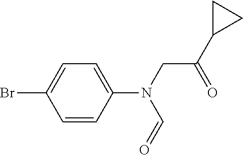 [SMILES: O=CN(CC(=O)C1CC1)c1ccc(Br)cc1]

Formic acid (13.7 mL, 364 mmol) and acetic anhydride (34 mL, 364 mmol) were heated at 60° C. for 15 minutes. The solution was then cooled to 0° C. and 2-(4-bromoanilino)-1-cyclopropyl-ethanone (4.28 g, 16.8 mmol) was added. The mixture was heated at 60° C. for 1 hour. The reaction mixture was cooled to r.t. and concentrated in vacuo. The residue was partitioned between EtOAc and sat. aq. NaHCO₃ solution. The two phases were separated, and the aqueous phase was re-extracted with EtOAc. The combined organic extracts were washed with brine, dried (Na₂SO₄), filtered and concentrated in vacuo to afford N-(4-bromophenyl)-N-(2-cyclopropyl-2-oxo-ethyl)formamide (4.75 g, 16.8 mmol, 99% yield) as a yellow oil.

UPLC-MS (ES⁴, Method A): 1.57 min, m/z=281.9/283.9 [M]⁺/[M+2]⁺

¹H NMR (400 MHz, CDCl₃) δ 8.47 (s, 1H), 7.51 (d, J=8.8 Hz, 2H), 7.06 (d, J=8.8 Hz, 2H), 4.70 (s, 2H), 2.02-1.94 (m, 1H), 1.15-1.10 (m, 2H), 1.01-0.95 (m, 2H)

### Step 3: 1-(4-bromophenyl)-4-cyclopropyl-imidazole

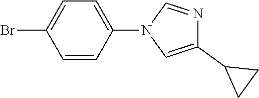 [SMILES: Brc1ccc(-n2cnc(C3CC3)c2)cc1]

N-(4-bromophenyl)-N-(2-cyclopropyl-2-oxo-ethyl)formamide (4.75 g, 16.8 mmol) in 1-butanol (40 mL), TEA (2.35 mL, 16.9 mmol) and NH₄OAc (13.0 g, 168 mmol) were split into 4 equal portions each and added to 4 microwave vials. The vials were sealed and the mixture irradiated at 150° C. for 1.5 hours. The reaction mixtures were combined and concentrated in vacuo. The residue was partitioned between EtOAc and water. The organic layer was washed with brine, dried (Na₂SO₄), filtered and concentrated. The crude material was purified by flash column chromatography (silica) eluting with EtOAc in Pet. Ether (0-50%) to afford 1-(4-bromophenyl)-4-cyclopropyl-imidazole (2.35 g, 8.93 mmol, 53% yield) as a yellow solid.

UPLC-MS (ES+, Method A): 1.21 min, m/z=262.9/264.9 [M]⁺/[M+2]⁺

¹H NMR (400 MHz, CDCl₃) δ 7.71 (d, J=1.2 Hz, 1H), 7.58 (d, J=8.8 Hz, 2H), 7.26 (d, J=8.8 Hz, 2H), 7.00 (d, 1H, J=1.2 Hz, 1H), 1.96-1.88 (m, 1H), 0.94-081 (m, 4H)

### Step 4: 4-cyclopropyl-1-[4-(4,4,5,5-tetramethyl-1,3,2-dioxaborolan-2-yl)phenyl]imidazole

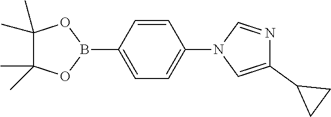 [SMILES: CC1(C)OB(c2ccc(-n3cnc(C4CC4)c3)cc2)OC1(C)C]

1-(4-Bromophenyl)-4-cyclopropyl-imidazole (2.35 g, 8.9 mmol), bis(pinacolato)diboron (2.49 g, 9.8 mmol) and KOAc (1.75 g, 17.9 mmol) were combined in 1,4-dioxane (50 mL) and the mixture was degassed with N₂ for 10 min. [1,1′-Bis(diphenylphosphino)ferrocene]Palladium(II) chloride DCM complex (729 mg, 0.89 mmol) was added and the reaction mixture was heated at 90° C. for 2 hours. After cooling to r.t., the mixture was filtered through celite washed with EtOAc. The filtrate was concentrated in vacuo and the residue purified by flash column chromatography (silica) eluting with EtOAc in Pet. Ether (0-100%) to afford 4-cyclopropyl-1-[4-(4,4,5,5-tetramethyl-1,3,2-dioxaborolan-2-yl)phenyl]imidazole (1.8 g, 5.8 mmol, 65% yield) as a colourless oil.

UPLC-MS (ES⁺, Method A): 1.45 min, m/z—311.0 [M+H]⁺.

¹H NMR (400 MHz, CDCl₃) δ 7.89 (d, J=8.4 Hz, 2H), 7.84-7.82 (m, 1H), 7.35 (d, J=8.4 Hz, 2H), 7.06-7.05 (m, 1H), 1.96-1.89 (m, 1H), 1.36 (s, 12H), 0.93-0.82 (m, 4H)

### Intermediate 60: 4-cyclopropyl-1-[3-(4,4,5,5-tetramethyl-1,3,2-dioxaborolan-2-yl)phenyl]imidazole

### Step 1: 1-(3-bromophenyl)-4-cyclopropyl-imidazole

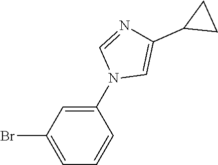 [SMILES: Brc1cccc(-n2cnc(C3CC3)c2)c1]

A mixture of 4-cyclopropyl-1H-imidazole (573 mg, 5.3 mmol), 1-bromo-3-iodo-benzene (2.25 g, 7.95 mmol), trans-1,2-diaminocyclohexane (363 mg, 3.18 mmol), CuI (303 mg, 1.59 mmol), Cs₂CO₃ (5.18 g, 15.9 mmol) in diethylene glycol dimethyl ether (5 mL) was degassed and the mixture was stirred at 90° C. for 18 h under N₂. The mixture was diluted with water (100 mL), extracted with EtOAc (250 mL). The organic layer was concentrated in vacuo and the residue purified by flash column chromatography (silica) eluting with MeOH in DCM (5%) containing 0.5% conc. NH₄OH to afford 1-(3-bromophenyl)-4-cyclopropyl-imidazole (700 mg, 2.66 mmol, 50% yield) as a dark green solid.

LC-MS (ES-API, Method D): 0.88 min, m/z=263.0/265.0 [M]⁺/[M+2]⁺

### Step 2: 4-cyclopropyl-1-[3-(4,4,5,5-tetramethyl-1,3,2-dioxaborolan-2-yl)phenyl]imidazole

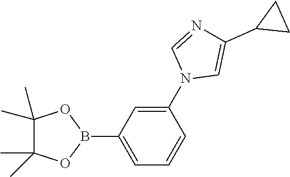 [SMILES: CC1(C)OB(c2cccc(-n3cnc(C4CC4)c3)c2)OC1(C)C]

Pd₂(dba)₃ (61 mg, 0.066 mmol) was added to a mixture of 1-(3-bromophenyl)-4-cyclopropyl-imidazole (350 mg, 1.33 mmol), bis(pinacolato)diboron (507 mg, 2.0 mmol), KOAc (392 mg, 3.99 mmol) and XPhos (63 mg, 0.13 mmol) in THF (10 mL) and the mixture was stirred at 75° C. under N₂ for 18 h. The mixture was concentrated in vacuo and purified by reverse phase column chromatography (C18 silica, 40-60 μm, 0-100% water/MeCN containing 0.1% TFA) to afford 4-cyclopropyl-1-[3-(4,4,5,5-tetramethyl-1,3,2-dioxaborolan-2-yl)phenyl]imidazole (400 mg, 1.29 mmol, 97% yield) as a yellow oil.

LC-MS (ES-API, Method D): 1.18 min, m/z=311.2 [M+H]⁺.

### Intermediate 61: 2,5-dimethyl-1-[4-(4,4,5,5-tetramethyl-1,3,2-dioxaborolan-2-yl)phenyl]imidazole

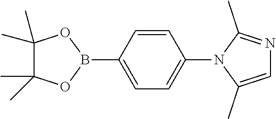 [SMILES: Cc1cnc(C)n1-c1ccc(B2OC(C)(C)C(C)(C)O2)cc1]

Step 1: N-prop-2-ynylacetamide

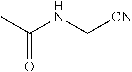

To a solution of prop-2-yn-1-amine (2.0 g, 36.3 mmol) and NEt₃ (11 g, 109 mmol) in DCM (50 mL) was added acetyl chloride (2.8 mL, 40 mmol) at 0° C. The mixture was stirred and allowed to warm to r.t., overnight under N₂. The mixture was concentrated in vacuo and purified by flash column chromatography (silica) eluting with EtOAc in Pet. Ether (35%) to afford N-prop-2-ynylacetamide (3.3 g, 34 mmol, 94% yield).

¹H NMR (400 MHz, DMSO-d₆) δ 8.26, (s, 1H), 3.82 (dd, J=5.6, 2.4 Hz, 2H), 3.07 (s, 1H); 1.81, (s, 3H)

Step 2: 1-(4-bromophenyl)-2,5-dimethyl-imidazole

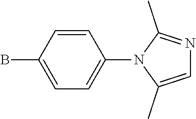

To a solution of N-(prop-2-yn-1-yl)acetamide (300 mg, 3.09 mmol) and 4-bromoaniline (797 mg, 4.64 mmol) in toluene (3 mL) was added zinc trifluoromethanesulfonate (56 mg, 0.15 mmol). The mixture was sealed in a vial and heated at 140° C. under N₂ in the microwave for 1 h. The mixture was concentrated in vacuo and purified by flash column chromatography (silica) eluting with MeOH in DCM (5%) to afford 1-(4-bromophenyl)-2,5-dimethyl-imidazole (490 mg, 1.95 mmol, 63% yield).

LCMS (ES-API, Method C): 0.31 min, m/z=251.0/253.0 [M]⁺/[M+2]⁺

### Step 3: 2,5-dimethyl-1-[4-(4,4,5,5-tetramethyl-1,3,2-dioxaborolan-2-yl)phenyl]imidazole

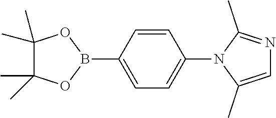 [SMILES: Cc1cnc(C)n1-c1ccc(B2OC(C)(C)C(C)(C)O2)cc1]

Pd₂dba₃ (160 mg, 0.18 mmol) and XPhos (167 mg, 0.35 mmol) were added to a solution of 1-(4-bromophenyl)-2,5-dimethyl-1H-imidazole (440 mg, 1.75 mmol), bis(pinacolato)diboron (890 mg, 3.5 mmol) and KOAc (516 mg, 5.25 mmol) in THF (10 mL). The mixture was heated at 75° C. for 18 h under N₂. The mixture was filtered and concentrated in vacuo and purified by reverse phase-column (C18 spherical 40-60 μm 100 A 40 g) chromatography to afford 2,5-dimethyl-1-[4-(4,4,5,5-tetramethyl-1,3,2-dioxaborolan-2-yl)phenyl]imidazole (450 mg, 1.51 mmol, 86% yield).

LC-MS (ES-API, Method D): 1.16 min, m/z=299.1 [M+H]⁺

### Intermediate 62: 2,4-dimethyl-1-[4-(4,4,5,5-tetramethyl-1,3,2-dioxaborolan-2-yl)phenyl]imidazole

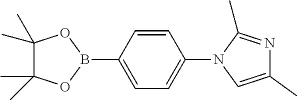

### Step 1: 1-(4-bromophenyl)-2,4-dimethyl-imidazole

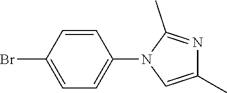

CuI (606 mg, 3.18 mmol) and (+/−)-trans-1,2-diaminocyclohexane (727 mg, 6.4 mmol) were added to 1-bromo-4-iodobenzene (3.0 g, 10.6 mmol), 2,5-dimethyl-1H-imidazole (2.04 g, 21.2 mmol) and Cs₂CO₃ (10.4 g, 31.8 mmol) in diethylene glycol dimethyl ether (10 mL). The mixture was degassed with N₂ for 10 minutes. The vial was then sealed and heated at 150° C. for 18 h. The mixture was extracted with EtOAc and concentrated in vacuo and purified by flash column chromatography (silica) eluting with MeOH in DCM (2%) to afford 1-(4-bromophenyl)-2,4-dimethyl-imidazole (470 mg, 1.87 mmol, 17% yield) as an orange oil.

LCMS (ES-API, Method C): 0.30 min, m/z=251.0/253.0 [M]⁺/[M+2]⁺

### Step 2: 2,4-dimethyl-1-[4-(4,4,5,5-tetramethyl-1,3,2-dioxaborolan-2-yl)phenyl]imidazole

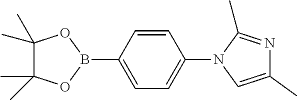

Pd₂dba₃ (157 mg, 0.17 mmol) and XPhos (163 mg, 0.34 mmol) were added to a solution of 1-(4-bromophenyl)-2,4-dimethyl-1H-imidazole (430 mg, 1.7 mmol), 4,4,5,5-tetramethyl-2-(3,3,4,4-tetramethylborolan-1-yl)-1,3,2-dioxaborolane (870 mg, 3.4 mmol) and KOAc (504 mg, 5.1 mmol) in THF (10 mL) The mixture was heated to 75° C. for 18 h under N₂. The mixture was filtered and concentrated in vacuo and purified by reverse phase column (C18 spherical 40-60 um 100 A 40 g; eluent: MeCN/water; gradient: 0-30%) to afford 2,4-dimethyl-1-[4-(4,4,5,5-tetramethyl-1,3,2-dioxaborolan-2-yl)phenyl]imidazole (300 mg, 1.0 mmol, 59% yield).

LCMS (ES-API, Method D): 1.12 min, m/z=299.1 [M+H]⁺

### Intermediate 63: 2,4,5-trimethyl-1-[4-(4,4,5,5-tetramethyl-1,3,2-dioxaborolan-2-yl)phenyl]imidazole

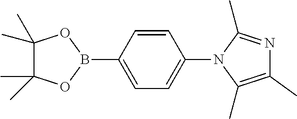

### Step 1: 1-(4-bromophenyl)-2,4,5-trimethyl-imidazole

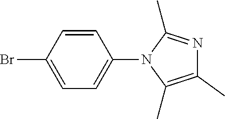

Butane-2,3-dione (1.0 g, 11.6 mmol) was added to a solution of acetaldehyde (512 mg, 11.6 mmol), 4-bromoaniline (999 mg, 5.8 mmol) and NH₄OAc (895 mg, 11.6 mmol) in MeOH (4.6 mL). The reaction was heated and stirred for 18 h at 80° C. The reaction mixture was extracted with toluene and the solvent was removed in vacuo. The crude product was purified by flash column chromatography (silica) eluting with EtOAc in Pet. Ether (50%) to afford 1-(4-bromophenyl)-2,4,5-trimethyl-imidazole (400 mg, 1.51 mmol, 26% yield).

LCMS (ES-API, Agilent, Method C) 0.38 min, m/z=265.0/267.0 [M]⁺/[M+2]⁺

### Step 2: 4,5-trimethyl-1-[4-(4,4,5,5-tetramethyl-1,3,2-dioxaborolan-2-yl)phenyl]imidazole

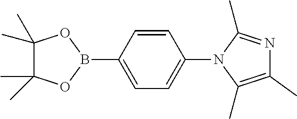

Pd₂(dba)₃ (121 mg, 0.13 mmol) and XPhos (126 mg, 0.27 mmol) was added to a solution of 1-(4-bromophenyl)-2,4,5-trimethyl-imidazole (350 mg, 1.33 mmol), bis(pinacolato)diboron (525 mg, 1.99 mmol) and KOAc (390 mg, 3.98 mmol) in THF (15 mL). The solution was heated and stirred at 75° C. for 18 h. The mixture was filtered and the solvent was removed in vacuo. The residue was purified by reverse phase chromatography (C18) eluting with MeCN/water gradient 0-100% to afford 2,4,5-trimethyl-1-[4-(4,4,5,5-tetramethyl-1,3,2-dioxaborolan-2-yl)phenyl]imidazole (334 mg, 1.07 mmol, 81% yield).

LCMS (ES-API, Method D): 0.35 min, m/z=231.0 [M+H]⁺ (boronic acid) & 1.18 min, m/z=313.2 [M+H]⁺ (boronic ester)

### Intermediate 64: 4-ethyl-5-methyl-1-[4-(4,4,5,5-tetramethyl-1,3,2-dioxaborolan-2-yl)phenyl]imidazole

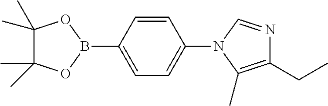 [SMILES: CCc1ncn(-c2ccc(B3OC(C)(C)C(C)(C)O3)cc2)c1C]

### Step 1: 2-(4-bromoanilino)pentan-3-one

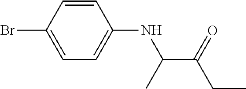

LiBr (4.17 g, 48.0 mmol) was added to a solution of 4-bromoaniline (5.5 g, 32.0 mmol), 2-bromopentan-3-one (5.84 g, 35.4 mmol) and NaHCO₃ (5.41 g, 64.4 mmol) in EtOH (60 mL). The mixture was heated at 90° C. for 18 h under N₂. The mixture was purified by flash column chromatography (silica) eluting with EtOAc in Pet. Ether (20%) to afford 2-(4-bromoanilino)pentan-3-one (7.6 g, 29.7 mmol, 93% yield) as an orange solid.

LCMS (ES-API, Method D): 2.36 min, m/z=256.0/258.0 [M]⁺/[M+2]⁺

### Step 2: N-(4-bromophenyl)-N-(1-methyl-2-oxo-butyl)formamide

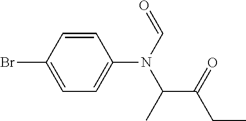 [SMILES: CCC(=O)C(C)N(C=O)c1ccc(Br)cc1]

Acetic anhydride (40 mL) was added to a solution of 2-((4-bromophenyl)amino)pentan-3-one (7.6 g, 29.7 mmol) in formic acid (120 mL) at 0° C. The mixture was stirred at r.t. for 2 h. The mixture was extracted with EtOAc (3×100 mL), the combined organic layer was washed with sat. aq. NaHCO₃ solution and saturated brine, dried (Na₂SO₄) and concentrated in vacuo to afford N-(4-bromophenyl)-N-(1-methyl-2-oxo-butyl)formamide (7.7 g, 27.0 mmol, 92% yield) as an orange oil.

LCMS (ES-API, Method D): 2.00 min, m/z=283.9/285.9 [M]⁺/[M+2]⁺

### Step 3: 1-(4-bromophenyl)-4-ethyl-5-methyl-imidazole

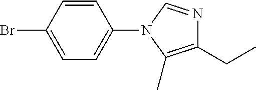

NH₄OAc (2.71 g, 35.2 mmol) was added to a solution of N-(4-bromophenyl)-N-(3-oxopentan-2-yl)formamide (2.0 g, 7.0 mmol) in acetic acid (glacial) (20 mL). The mixture was heated at 130° C. for 1 h under N₂. The reaction mixture was extracted with EtOAc (3×150 mL) and the combined organic layers were washed with sat. aq. NaHCO₃ solution, brine, dried (Na₂SO₄) and concentrated in vacuo and purified by flash column chromatography (silica) eluting with MeOH in DCM (5%) to afford 1-(4-bromophenyl)-4-ethyl-5-methyl-imidazole (1.7 g, 6.4 mmol, 91% yield) as an orange solid.

LCMS (ES-API, Method D): 0.87 min, m/z=265.0/266.9 [M]⁺/[M+2]⁺

### Step 4: 4-ethyl-5-methyl-1-[4-(4,4,5,5-tetramethyl-1,3,2-dioxaborolan-2-yl)phenyl]imidazole

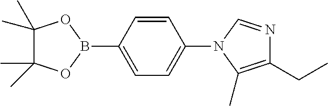 [SMILES: CCc1ncn(-c2ccc(B3OC(C)(C)C(C)(C)O3)cc2)c1C]

Pd₂dba₃ (173 mg, 0.19 mmol) and XPhos (180 mg, 0.38 mmol) were added to a solution of 1-(4-bromophenyl)-4-ethyl-5-methyl-1H-imidazole (500 mg, 1.89 mmol), 4,4,5,5-tetramethyl-2-(3,3,4,4-tetramethylborolan-1-yl)-1,3,2-dioxaborolane (958 mg, 3.78 mmol) and KOAc (555 mg, 5.7 mmol) in THF (10 mL). The mixture was heated at 75° C. for 18 h under N₂. The mixture was filtered and solvent removed in vacuo and purified by reverse phase chromatography (C18 spherical 40-60 μm 100 A 40 g) to afford 4-ethyl-5-methyl-1-[4-(4,4,5,5-tetramethyl-1,3,2-dioxaborolan-2-yl)phenyl]imidazole (450 mg, 1.44 mmol, 76% yield) as an orange solid.

LCMS (ES-API, Method D): 1.34 min, m/z=313.2 [M+H]⁺

### Intermediate 65: 4-cyclopropyl-1-[2-fluoro-4-(4,4,5,5-tetramethyl-1,3,2-dioxaborolan-2-yl)phenyl]imidazole

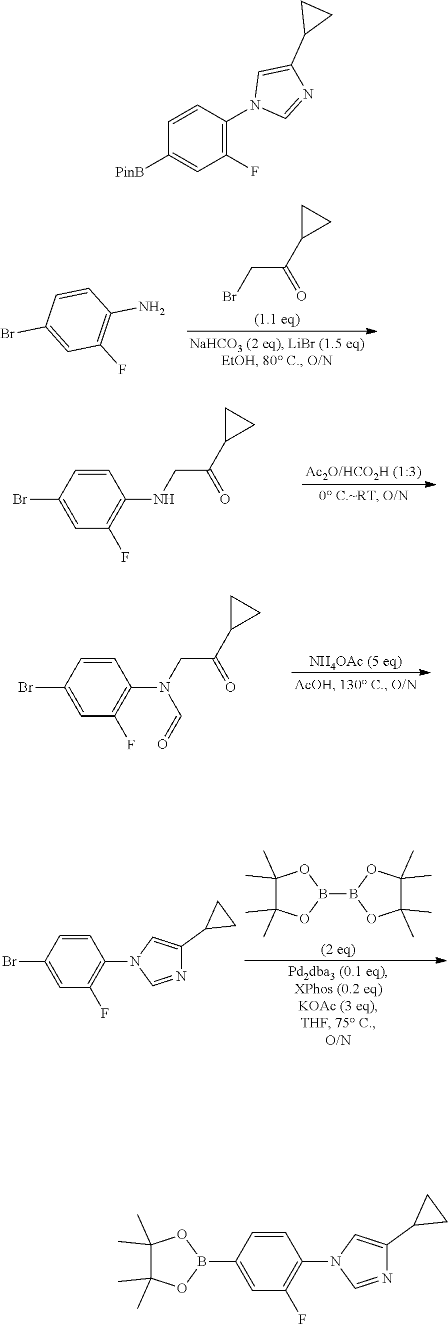

### Step 1: 2-(4-bromo-2-fluoro-anilino)-1-cyclopropyl-ethanone

To a solution of 4-bromo-2-fluoroaniline (3.0 g, 15.8 mmol) in EtOH (60 ml) were added 2-bromo-1-cyclopropylethan-1-one (2.83 g, 17.4 mmol), LiBr (2.06 g, 23.7 mmol) and NaHCO3 (2.65 g, 31.6 mmol), the mixture was stirred at 80° C. under nitrogen overnight. The mixture was concentrated and purified by flash column chromatography (Pet.Ether/EtOAc=10/1) to give 2-(4-bromo-2-fluoro-anilino)-1-cyclopropyl-ethanone (2.78 g, 10.21 mmol, 65%).

LCMS (ES-API, Method C): 1.62 min, m/z=272/274 [M]⁺/[M+2]⁺

### Step 2: N-(4-bromo-2-fluoro-phenyl)-N-(2-cyclopropyl-2-oxo-ethyl)formamide

To a solution of 2-((4-bromo-2-fluorophenyl)amino)-1-cyclopropylethan-1-one (2.72 g, 10 mmol) in formic acid (21 mL) was added acetic anhydride (7 mL), the mixture was stirred at r.t. overnight. The mixture was concentrated and adjusted to pH=9 with NaHCO₃ and extracted with EtOAc, washed with brine and dried over Na₂SO₄ and concentrated to give N-(4-bromo-2-fluoro-phenyl)-N-(2-cyclopropyl-2-oxo-ethyl)formamide (2.74 g, 9.13 mmol, 91%).

LCMS (ES-API, Method C): 1.22 min, m/z=300/302 [M]⁺/[M+2]⁺

### Step 3: 1-(4-bromo-2-fluoro-phenyl)-4-cyclopropyl-imidazole

To a solution of N-(4-bromo-2-fluorophenyl)-N-(2-cyclopropyl-2-oxoethyl)formamide (2.56 g, 8.5 mmol) in acetic acid (64 mL) was added NH₄OAc (3.29 g, 42.5 mmol), the mixture was stirred at 130° C. under nitrogen overnight. The mixture was concentrated and adjusted to pH=9 with NaHCO₃ and extracted with EtOAc, washed with brine and dried over Na₂SO₄, concentrated and purified by flash column chromatography (DCM/MeOH=20/1) to give 1-(4-bromo-2-fluoro-phenyl)-4-cyclopropyl-imidazole (0.95 g, 3.38 mmol, 40%).

LCMS (ES-API, Method C): 0.40 min, m/z=281/283 [M]⁺/[M+2]⁺

### Step 4: 4-cyclopropyl-1-[2-fluoro-4-(4,4,5,5-tetramethyl-1,3,2-dioxaborolan-2-yl)phenyl]imidazole

To a solution of 1-(4-bromo-2-fluorophenyl)-4-cyclopropyl-1H-imidazole (900 mg, 3.2 mmol) in THF (50 mL) were added 4,4,4′,4′,5,5,5′,5′-octamethyl-2,2′-bi(1,3,2-dioxaborolane) (1.63 g, 6.4 mmol), Pd₂dba₃ (360 mg, 0.32 mmol) and XPhos (378 mg, 0.64 mmol) and KOAc (1.08 g, 9.6 mmol), the mixture was stirred at 75° C. under nitrogen overnight. The mixture was concentrated and purified by reverse phase chromatography (30% MeCN in water) to give 4-cyclopropyl-1-[2-fluoro-4-(4,4,5,5-tetramethyl-1,3,2-dioxaborolan-2-yl)phenyl]imidazole (0.63 g, 1.92 mmol, 60%).

LCMS (ES-API, Method D): 0.29 min, m/z=247.1 [M+H]⁺ boronic acid

### Intermediate 66: 2-(4-cyclopropyl-1H-imidazol-1-yl)-5-(4,4,5,5-tetramethyl-1,3,2-dioxaborolan-2-yl)benzonitrile

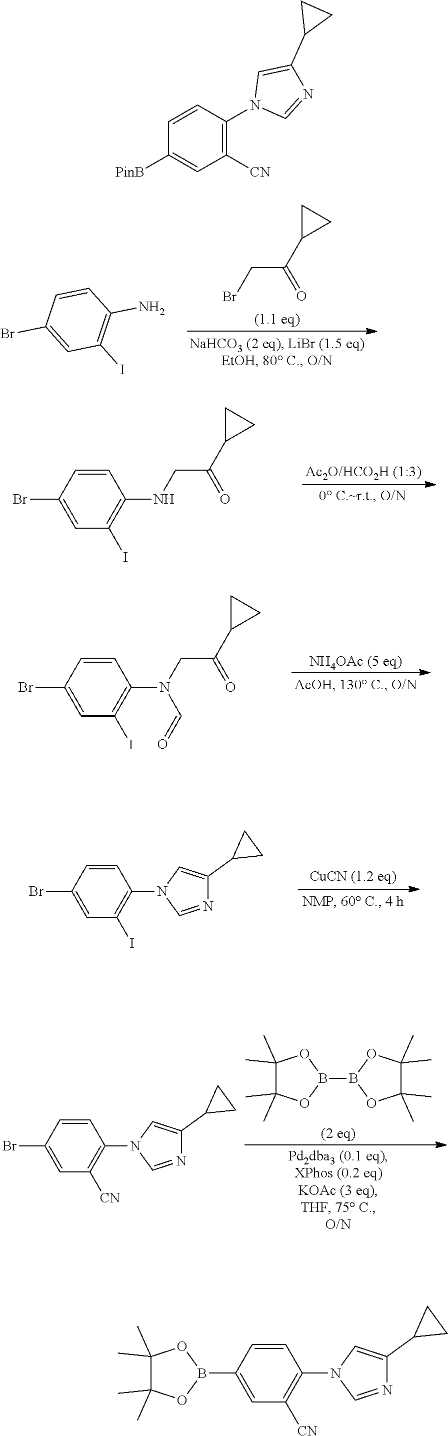

### Step 1: 2-(4-bromo-2-iodo-anilino)-1-cyclopropyl-ethanone

To a solution of 4-bromo-2-iodoaniline (16.5 g, 55.57 mmol, 1.0 eq.) in EtOH (80 mL) was added 2-bromo-1-cyclopropylethan-1-one (9.9 g, 61.12 mmol, 1.1 eq.), LiBr (9.31 g, 83.36 mmol, 1.5 eq.) and NaHCO₃ (7.26 g, 111.14 mmol, 2.0 eq.). The reaction mixture was stirred at 80° C. under N₂ overnight. The solution was concentrated and purified by flash column chromatography to give 2-(4-bromo-2-iodo-anilino)-1-cyclopropyl-ethanone (21.3 g, 56.0 mmol, 100%).

LC-MS (ES-API, Method C): 2.27 min, m/z=380.0/381.9 [M]⁺/[M+2]⁺

### Step 2: N-(4-bromo-2-iodo-phenyl)-N-(2-cyclopropyl-2-oxo-ethyl)formamide

To a solution of 2-((4-bromo-2-iodophenyl)amino)-1-cyclopropylethan-1-one (19 g, 50.13 mmol, 1.0 eq.) stirred at 0° C. under N₂ was added acetic anhydride (28.8 mL) and formic acid (86.3 mL), the reaction mixture was warmed to r.t. overnight. The solution was concentrated, basified to pH 9 and diluted with water (1000 mL). The aqueous phase was extracted with EtOAc (300 mL×4) and concentrated. The crude was purified by flash column chromatography (Pet.Ether/EtOAc=100:1 to 20:1) to give N-(4-bromo-2-iodo-phenyl)-N-(2-cyclopropyl-2-oxo-ethyl)formamide (23.0 g, 56.36 mmol, assumed quantitative).

LC-MS (ES-API, Method D): 2.39 min, m/z=407.9/409.9 [M]⁺/[M+2]⁺

### Step 3: 1-(4-bromo-2-iodo-phenyl)-4-cyclopropyl-imidazole

To a solution of N-(4-bromo-2-iodophenyl)-N-(2-cyclopropyl-2-oxo-ethyl)formamide (21.0 g, 51.47 mmol, 1.0 eq.) in acetic acid (20 mL) was added NH₄OAc (1.87 g, 24.52 mmol, 5.0 e.q). The reaction mixture was stirred at 130° C. under N₂ overnight. The solution was concentrated and basified to pH 9.

The crude was purified by flash column chromatography (DCM/MeOH 100:1 to 30:1) to give 1-(4-bromo-2-iodo-phenyl)-4-cyclopropyl-imidazole (5.5 g, 14.14 mmol, 25%).

¹H NMR (400 MHz, DMSO-d₆) δ 8.18 (d, J=2.1 Hz, 1H), 7.68 (dd, J=2.2, 8.3 Hz, 1H), 7.58 (s, 1H), 7.31 (d, J=8.2 Hz, 1H), 7.03 (s, 1H), 1.86-1.78 (m, 1H), 0.80-0.73 (m, 2H), 0.69-0.64 (m, 2H)

### Step 4: 5-bromo-2-(4-cyclopropylimidazol-1-yl)benzonitrile

A solution of 1-(4-bromo-2-iodophenyl)-4-cyclopropyl-1H-imidazole (1.0 g, 2.57 mmol) and CuCN (4.6 g, 51.41 mmol) in NMP (10 mL) was stirred at 100° C. with N₂ overnight. The mixture was diluted with water (100 mL) and extracted with EtOAc (20 mL×10), washed with brine and dried over Na₂SO₄, concentrated and purified by flash column chromatography (Pet. Ether/EtOAc=6/1) to give 5-bromo-2-(4-cyclopropylimidazol-1-yl)benzonitrile (0.23 g, 0.79 mmol, 31%).

LC-MS (ES-API, Method K): 1.07 min, m/z=288.00 [M]⁺/[M+2]⁺

### Step 5: 2-(4-cyclopropyl-1H-imidazol-1-yl)-5-(4,4,5,5-tetramethyl-1,3,2-dioxaborolan-2-yl)benzonitrile

A solution of 5-bromo-2-(4-cyclopropyl-1H-imidazol-1-yl)benzonitrile (1.3 g, 4.51 mmol), 4,4,4′,4′,5,5,5′,5′-octamethyl-2,2′-bi(1,3,2-dioxaborolane) (11.46 g, 45.12 mmol), XPhos (2.15 g, 4.51 mmol), KOAc (1.33 g, 13.55 mmol) and Pd₂(dba)₃ (826.3 mg, 0.90 mmol) in THF (180 mL) was stirred at 75° C. under N₂ overnight. The mixture was concentrated and purified by flash column chromatography (Pet.Ether/EtOAc 2:1) to give [3-cyano-4-(4-cyclopropylimidazol-1-yl)phenyl]boronic acid.

¹H NMR (400 MHz, DMSO-d₆) δ 8.15 (s, 1H), 8.08 (d, J=8.4 Hz, 1H), 8.03 (s, 1H), 7.73 (d, J=7.6 Hz, 1H), 7.46 (s, 1H), 1.97-1.89 (m, 1H), 1.38 (s, 12H), 0.91-0.84 (m, 2H), 0.79-0.75 (m, 2H)

Compounds prepared in a similar manner to that set out above are given below in Table 4

TABLE 4

| Intermediate | | | MS | |
|---|---|---|---|---|
| N | Structure | NMR | [M + H⁺]+ | Method |
| 57 | 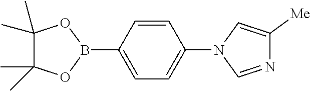 [SMILES: Cc1cn(-c2ccc(B3OC(C)(C)C(C)(C)O3)cc2)cn1] | ¹H NMR (400 MHz, CDCl₃) δ 7.89 (d, J = 8.5 Hz, 2H), 7.81 (d, J = 1.4 Hz, 1H), 7.36 (d, J = 8.5 Hz, 2H), 7.05-7.04 (m, 1H), 2.30 (d, J = 1.0 Hz, 3H), 1.36 (s, 12H) | 1.36 min, m/z = 284.9 [M + H]⁺ | Method A |
| 58 | 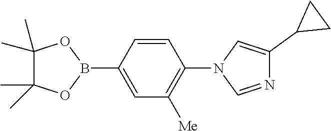 [SMILES: Cc1cc(B2OC(C)(C)C(C)(C)O2)ccc1-n1cnc(C2CC2)c1] | ¹H NMR (400 MHz, DMSO-d₆) δ 7.66 (d, J=12.8 Hz, 2H), 7.58 (d, J = 7.6Hz, 1H), 7.26 (d, J = 8.1 Hz, 1H), 7.13 (d, J = 0.9 Hz, 1H), 2.20 (s, 3H), 1.88-1.80 (m, 1H), 1.30 (s, 12H), 0.82-0.76 (m, 2H), 0.72-0.67 (m, 2H) | 1.52 min, m/z = 325.2 [M + H]⁺ | Method D |
| 59 | 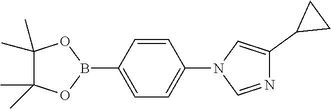 [SMILES: CC1(C)OB(c2ccc(-n3cnc(C4CC4)c3)cc2)OC1(C)C] | ¹H NMR (400 MHz, DMSO-d₆) δ 8.02 (d, J = 1.3 Hz, 1H), 7.62 (d, J =8.5 Hz, 2H), 7.49 (d, J = 8.4 Hz, 2H), 7.40 (d, J = 1.4 Hz, 1H), 1.74-1.67 (m, 1H), 1.17 (s, 12H), 0.70-0.64 (m, 2H), 0.59- 0.53 (m, 2H) | 1.42 min, m/z = 311.2 [M + H]⁺ | Method D |
| 65 | 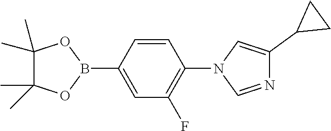 [SMILES: CC1(C)OB(c2ccc(-n3cnc(C4CC4)c3)c(F)c2)OC1(C)C] | ¹H NMR (400 MHz, CDCl₃) δ 7.77 (s, 1H), 7.67-7.63 (m, 2H), 7.35 (t, J= 7.7 Hz, 1H), 7.02 (s, 1H), 1.94-1.87 (m, 1H), 1.35 (s, 12H), 0.93-0.85(m, 2H), 0.84- 0.80 (m, 2H) | 1.54 min, m/z = 329.1 [M + H]⁺ | Method A |
| 67 | 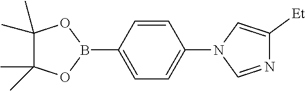 [SMILES: CCc1cn(-c2ccc(B3OC(C)(C)C(C)(C)O3)cc2)cn1] | ¹H NMR (400 MHz, CDCl₃) δ 7.93-7.89 (m, 3H), 7.38 (d, J = 8.4 Hz, 2H),7.07-7.05 (m, 1H), 2.70 (qd, J = 7.6 Hz, 1.2 Hz, 2H), 1.33 (s, 12H), 1.30 (t, J = 7.6 Hz, 3H) | 1.46 min, m/z = 299.0 [M + H]⁺ | Method A |
| 68 | 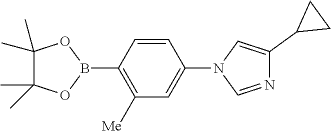 [SMILES: Cc1cc(-n2cnc(C3CC3)c2)ccc1B1OC(C)(C)C(C)(C)O1] | | 1.47 min, m/z = 325.1 [M + H]⁺ | Method K |
| 69 | 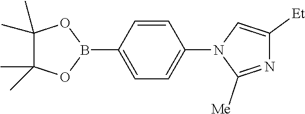 [SMILES: CCc1cn(-c2ccc(B3OC(C)(C)C(C)(C)O3)cc2)c(C)n1] | ¹H NMR (400 MHz, DMSO-d₆) δ 7.66 (d, J = 8.1 Hz, 2H), 7.30 (d, J = 8.4 Hz, 2H), 6.87 (s, 1H), 2.35-2.30 (m, 2H), 2.15 (s, 3H), 1.18 (s,12H), 0.94 (s, 3H) | 1.67 min, m/z = 313.0 [M + H]+ | Method D |
| 70 | 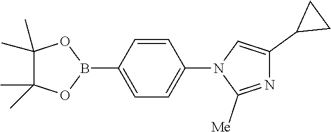 [SMILES: Cc1nc(C2CC2)cn1-c1ccc(B2OC(C)(C)C(C)(C)O2)cc1] | ¹H NMR (400 MHz, DMSO-d₆) δ 7.91 (d, J = 9.3 Hz, 2H), 7.53 (d, J = 8.2 Hz, 2H), 7.17 (s, 1H), 2.38 (s, 3H), 1.99-1.94 (m, 1H), 1.45 (s,12H), 0.92-0.84 (m, 2H), 0.82-0.74 (m, 2H) | 1.67 min, m/z 325.0 [M + H]⁺ | Method D |

### General Method for the Synthesis of Intermediates 71-74

A method for preparing Intermediate 71 is given below. Further intermediates that were prepared in a similar manner from commercially available bromophenols are given in Table 5.

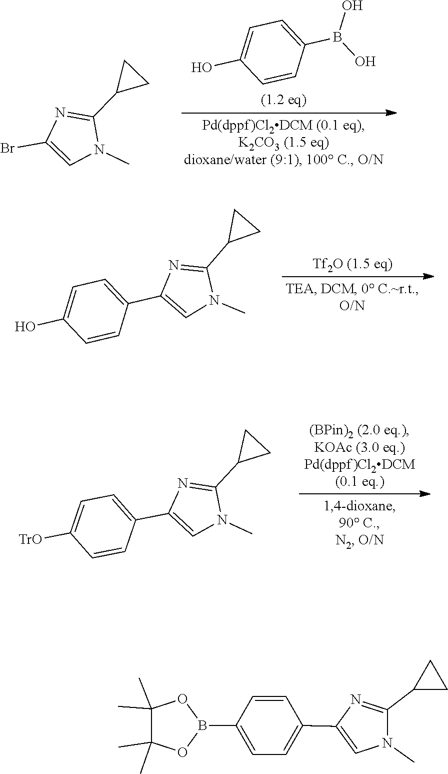

### Intermediate 71: 2-cyclopropyl-1-methyl-4-[4-(4,4,5,5-tetramethyl-1,3,2-dioxaborolan-2-yl)phenyl]imidazole

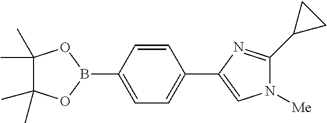 [SMILES: Cn1cc(-c2ccc(B3OC(C)(C)C(C)(C)O3)cc2)nc1C1CC1]

### Step 1: 4-(2-cyclopropyl-1-methyl-imidazol-4-yl)phenol

To a solution of 4-bromo-2-cyclopropyl-1-methyl-1H-imidazole (20 g, 0.1 mol) in 1,4-dioxane (470 mL) and water (53 mL) were added (4-hydroxyphenyl)boronic acid (17.81 g, 0.12 mol), Pd(dppf)Cl₂CH₂Cl₂ (8.13 g, 0.01 mol) and K₂CO₃ (20.63 g, 0.15 mol), the mixture was stirred at 100° C. under nitrogen overnight. The mixture was concentrated and purified by column chromatography (Pet.Ether/EtOAc 2:1) to give 4-(2-cyclopropyl-1-methyl-imidazol-4-yl)phenol (19.86 g, 92.69 mmol, 93%).

LCMS (ES-API, Method C): 0.32 min, m/z=215.2 [M+H]⁺

¹H NMR (400 MHz, DMSO-d₆) δ 9.27 (s, 1H), 7.48 (d, J=8.5 Hz, 2H), 7.26 (s, 1H), 6.73 (d, J=8.7 Hz, 2H), 3.67 (s, 3H), 2.01-1.94 (m, 1H), 0.96-0.85 (m, 4H)

### Step 2: [4-(2-cyclopropyl-1-methyl-imidazol-4-yl)phenyl]trifluoromethanesulfonate

To a solution of 4-(2-cyclopropyl-1-methyl-1H-imidazol-4-yl)phenol (19.86 g, 92.691 mmol) in DCM was added Tf₂O (23.4 mL, 139.04 mmol) and TEA (38.6 mL, 278.07 mmol) at 0° C. and then at room temperature overnight. The mixture was concentrated and purified by flash column chromatography (Pet.Ether/EtOAc 4:1) to give [4-(2-cyclopropyl-1-methyl-imidazol-4-yl)phenyl]trifluoromethanesulfonate (13.0 g, 37.54 mmol, 41%).

LCMS (ES-API, Method D): 1.23 min, m/z=347.0 [M+H]⁺

¹H NMR (400 MHz, DMSO-d) δ 7.82 (d, J=9.0 Hz, 2H), 7.60 (s, 1H), 7.43 (d, J=8.9 Hz, 2H), 3.71 (s, 3H), 2.04-1.96 (m, 1H), 0.98-0.93 (m, 2H), 0.91-0.86 (m, 2H)

### Step 3: 2-cyclopropyl-1-methyl-4-[4-(4,4,5,5-tetramethyl-1,3,2-dioxaborolan-2-yl)phenyl]imidazole

To a solution of 4-(2-cyclopropyl-1-methyl-1H-imidazol-4-yl)phenyl trifluoromethanesulfonate (13.56 g, 39.2 mmol) in dioxane (90 mL) were added bis(pinacolato)diboron (19.89 g, 78.3 mmol), Pd(dppf)Cl₂CH₂Cl₂ (3.2 g, 3.9 mmol) and KOAc (11.53 g, 117 mmol), the mixture was stirred at 90° C. under nitrogen overnight. The mixture was concentrated and purified by column (Pet.Ether/EtOAc 2:1, v/v) to give 2-cyclopropyl-1-methyl-4-[4-(4,4,5,5-tetramethyl-1,3,2-dioxaborolan-2-yl)phenyl]imidazole (17.8 g, 54.9 mmol, 100%)

TABLE 5

| Example | | | MS | |
|---|---|---|---|---|
| N | Structure | NMR | [M + H⁺]+ | Method |
| 71 | 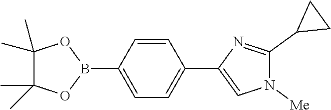 [SMILES: Cn1cc(-c2ccc(B3OC(C)(C)C(C)(C)O3)cc2)nc1C1CC1] | ¹H NMR (400 MHz, DMSO-d₆) δ 7.78 (d, J = 8.0 Hz, 2H), 7.72 (d, J = 7.9Hz, 2H), 7.65 (s, 1H), 3.79 (s, 3H), 2.13-2.04 (m, 1H), 1.41 (s, 12H), 1.07-0.98 (m, 4H) | 1.27 min, m/z = 325.1 [M + H]⁺ | Method D |
| 72 | 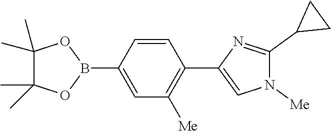 [SMILES: Cc1cc(B2OC(C)(C)C(C)(C)O2)ccc1-c1cn(C)c(C2CC2)n1] | ¹H NMR (400 MHz, DMSO-d₆) δ 7.81 (d, J = 8.0 Hz, 1H), 7.47- 7.42 (m, 2H), 7.30 (s, 1H), 3.69 (s, 3H), 2.38 (s, 3H), 2.00-1.93 (m, 1H), 1.26 (s,12H), 0.94- 0.87 (m, 2H), 0.87-0.83 (m, 2H) | 1.39 min, m/z = 339.2 [M + H]⁺ | Method D |
| 73 | 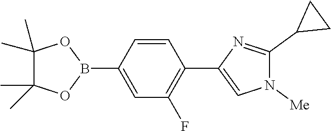 [SMILES: Cn1cc(-c2ccc(B3OC(C)(C)C(C)(C)O3)cc2F)nc1C1CC1] | ¹H NMR (400 MHz, DMSO-d₆) δ 8.02 (t, J = 7.6 Hz, 1H), 7.52- 7.47 (m, 2H),7.36 (d, J = 12.6 Hz, 1H), 3.74 (s, 3H), 2.07-1.98 (m, 1H), 1.32 (s, 12H),1.01-0.94 (m, 2H), 0.94-0.90 (m, 2H) | 2.32 min, m/z = 343.2 [M + H]⁺ | Method E |
| 74 | 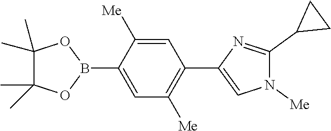 [SMILES: Cc1cc(-c2cn(C)c(C3CC3)n2)c(C)cc1B1OC(C)(C)C(C)(C)O1] | ¹H NMR (400 MHz, DMSO-d₆) δ 7.64 (s, 1H), 7.45 (s, 1H), 7.32 (s, 1H), 3.72 (s, 3H), 2.44 (s, 3H), 2.37 (s, 3H), 2.04-1.96 (m, 1H), 1.32 (s, 12H), 0.98-0.93 (m, 2H), 0.91-0.87 (m, 2H) | 0.85 min, m/z = 353.3 [M + H]⁺ | Method C |
| 75 | 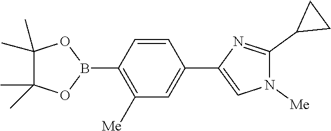 [SMILES: Cc1cc(-c2cn(C)c(C3CC3)n2)ccc1B1OC(C)(C)C(C)(C)O1] | ¹H NMR (400 MHz, DMSO-d₆) δ 7.97 (s, 1H), 7.60 (d, J = 7.2 Hz, 1H), 7.54 (s, 1H), 7.48 (d, J = 7.8 Hz, 1H), 3.70 (s, 3H), 2.49 (s, 3H), 2.05-1.97 (m, 1H), 1.33 (s, 12H), 0.98-0.86 (m, 4H) | 1.67 min, m/z = 339.3 [M + H]⁺ | Method D |

### General Method for the Synthesis of Intermediates 76-82

A method for preparing Intermediate 76 is given below. Further intermediates that were prepared in a similar manner from commercially available bromophenols are given in Table 6.

### Intermediate 76: 4-[4-(4,4,5,5-tetramethyl-1,3,2-dioxaborolan-2-yl)phenyl]-1-(2,2,2-trifluoro-1-methyl-ethyl)piperidine

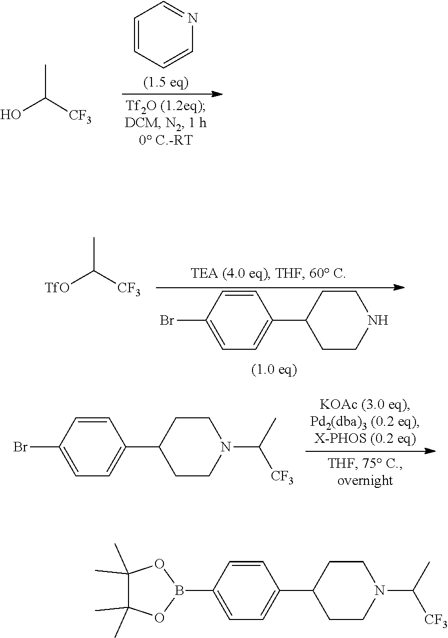

### Step 1: (2,2,2-trifluoro-1-methyl-ethyl) trifluoromethanesulfonate

To a solution of 1,1,1-trifluoro-2-propanol (1.0 g, 8.77 mmol) and pyridine (1.06 mL, 13.15 mmol) in DCM (10 mL) was added trifluoromethanesulfonic anhydride (1.77 mL, 10.52 mmol) at 0° C. under nitrogen atmosphere and was stirred at r.t. overnight. The crude reaction mixture was directly used in the next step without purification.

### Step 2: 4-(4-bromophenyl)-1-(2,2,2-trifluoro-1-methyl-ethyl)piperidine

To a solution of 4-(4-bromophenyl)piperidine (500 mg, 2.08 mmol) and TEA (1.16 mL, 8.32 mmol) in THF (20 mL) was added (2,2,2-trifluoro-1-methyl-ethyl) trifluoromethanesulfonate as a crude solution from previous step at r.t. and stirred at 60° C. overnight. The mixture was concentrated and purified by flash column chromatography (Pet.Ether/EtOAc 50:1) to give 4-(4-bromophenyl)-1-(2,2,2-trifluoro-1-methyl-ethyl)piperidine (120 mg, 17%) as a white oil.

LCMS (ES-API, Method C): 2.59 min, m/z=336.0/338.0 [M]⁺/[M+2]⁺

¹H NMR (400 MHz, DMSO-d₆) δ 7.48 (d, J=8.8 Hz, 2H), 7.23 (d, J=8.6 Hz, 2H), 3.54-3.45 (m, 1H), 2.99 (t, J=13.0 Hz, 2H), 2.60 (t, J=10.7 Hz, 1H), 2.51-2.45 (m, 1H), 1.80-1.70 (m, 2H), 1.62-1.52 (m, 2H), 1.20 (d, J=6.9 Hz, 3H).

### Step 3: 4-[4-(4,4,5,5-tetramethyl-1,3,2-dioxaborolan-2-yl)phenyl]-1-(2,2,2-trifluoro-1-methyl-ethyl)piperidine

To a solution of 4-(4-bromophenyl)-1-(2,2,2-trifluoro-1-methyl-ethyl)piperidine (320 mg, 0.95 mmol) in THF (15 mL) were added bis(pinacolato)diboron (483 mg, 1.90 mmol), Pd₂(dba)₃ (87.17 mg, 0.10 mmol), XPhos (90.75 mg, 0.19 mmol) and KOAc (280 mg, 2.86 mmol). The mixture was stirred at 70° C. under nitrogen overnight. The mixture was concentrated and purified by flash column chromatography (Pet.Ether/EtOAc 40:1) to give 4-[4-(4,4,5,5-tetramethyl-1,3,2-dioxaborolan-2-yl)phenyl]-1-(2,2,2-trifluoro-1-methyl-ethyl)piperidine.

LCMS (ES-API, Method C): 2.89 min. m/z=384.3 [M+H]⁺

TABLE 6

| Intermediate | | | MS | |
|---|---|---|---|---|
| N | Structure | NMR | [M + H⁺]+ | Method |
| 76 | 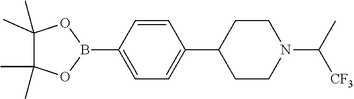 [SMILES: CC(N1CCC(c2ccc(B3OC(C)(C)C(C)(C)O3)cc2)CC1)C(F)(F)F] | | 2.89 min, m/z 384.3 [M + H]⁺ | Method C |
| 77 | 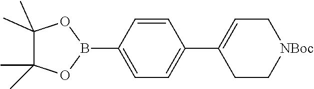 [SMILES: CC(C)(C)OC(=O)N1CC=C(c2ccc(B3OC(C)(C)C(C)(C)O3)cc2)CC1] | ¹H NMR (400 MHz, DMSO-d₆) δ 7.67 (d, J = 8.2 Hz, 2H), 7.47 (d, J = 7.8 Hz, 2H), 6.26 (s, 1H), 4.02 (s, 2H), 3.56 (s, 2H), 2.48 | 1.87 min, m/z 330.1 (M − tBu) | Method D |
| | | (s, 2H), 1.45 (s, 12H), | | |
| | | 1.30 (s, 9H) | | |
| 78 | 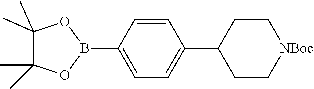 [SMILES: CC(C)(C)OC(=O)N1CCC(c2ccc(B3OC(C)(C)C(C)(C)O3)cc2)CC1] | | 2.84 min, m/z 388.3 [M + H]⁺ | Method C |
| 78 | 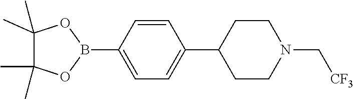 [SMILES: CC1(C)OB(c2ccc(C3CCN(CC(F)(F)F)CC3)cc2)OC1(C)C] | | 2.53 min, m/z 370.2[M + H]⁺ | Method C |
| 79 | 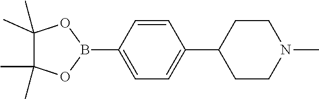 | | 1.52 min, m/z 302.2 [M + H]⁺ | Method D |
| 80 | 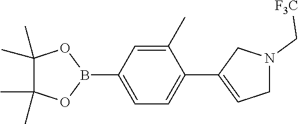 [SMILES: Cc1cc(B2OC(C)(C)C(C)(C)O2)ccc1C1=CCN(CC(F)(F)F)C1] | ¹H NMR (400 MHz, DMSO-d₆) δ 7.83-7.81 (m, 2H), 7.54 (s, 1H),, 6.02 (s, 1H), 4.01-3.9 (m, 2H), 3.88-3.80 (m, 2H), 2.38 (s, 2H), 1.30 (s, 12H), 1.25 (s, 3H). | 2.65 min, m/z 368.2 [M + H]⁺ | Method C |
| 81 | 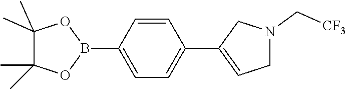 [SMILES: CC1(C)OB(c2ccc(C3=CCN(CC(F)(F)F)C3)cc2)OC1(C)C] | | 1.39 min, m/z 354.2 [M + H]⁺ | Method R |

| Intermediate | | |
|---|---|---|
| N | Structure | MS [M + H⁺]+ |
| 82 | 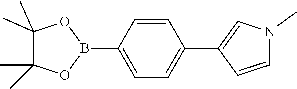 | LC-MS (ES-API, Method R): 1.074 min, m/z, 284.1 [M + H] |
| 83 | 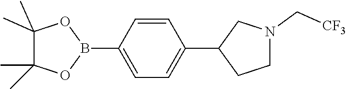 [SMILES: CC1(C)OB(c2ccc(C3CCN(CC(F)(F)F)C3)cc2)OC1(C)C] | LC-MS (ES-API, Method C): 2.031 min, m/z, 356.21 [M + H]. |
| 84 | 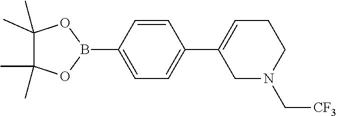 [SMILES: CC1(C)OB(c2ccc(C3=CCCN(CC(F)(F)F)C3)cc2)OC1(C)C] | LC-MS (ES-API, Method R): 1.606 min, m/z, 368.2 [M + H] |
| 85 | 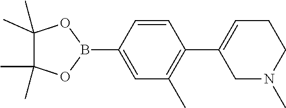 | LC-MS (ES-API, Method D): 1.578 min, m/z, 314.3 [M + H]+ (%) |
| 86 | 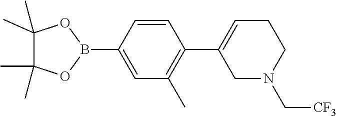 | LC-MS(ES-API, Method D): 1.831 min, m/z, 300.2 [M + H] |
| 87 | 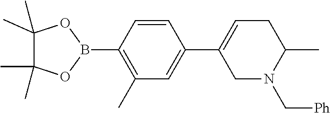 [SMILES: Cc1cc(C2=CCC(C)N(Cc3ccccc3)C2)ccc1B1OC(C)(C)C(C)(C)O1] | LC-MS (ES-API, Method D): 1.962 min, m/z, 404.3 [M + H] |
| 88 | 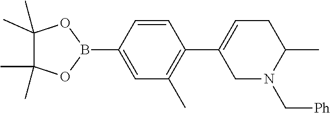 [SMILES: Cc1cc(B2OC(C)(C)C(C)(C)O2)ccc1C1=CCC(C)N(Cc2ccccc2)C1] | LC-MS (ES-API, Method C): 1.075 min, m/z, 404.3 M + H]+ (%) |
| 89 | 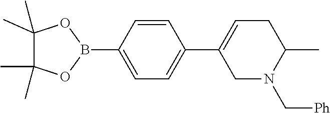 [SMILES: CC1CC=C(c2ccc(B3OC(C)(C)C(C)(C)O3)cc2)CN1Cc1ccccc1] | LCMS (ES-API, Method C): 0.940 min, m/z = 390.3 [M + H]+ |
| 90 | 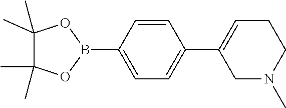 | LC-MS(ES-API, Method D): 1.203 min, m/z, 300.30 [M + H] |
| 91 | 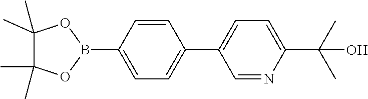 [SMILES: CC(C)(O)c1ccc(-c2ccc(B3OC(C)(C)C(C)(C)O3)cc2)cn1] | LC-MS (ES-API, Method C): 1.214 min, m/z, 340.2 [M + H]. |
| 92 | 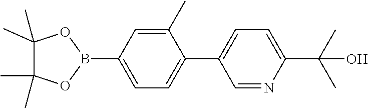 [SMILES: Cc1cc(B2OC(C)(C)C(C)(C)O2)ccc1-c1ccc(C(C)(C)O)nc1] | LC-MS (ES-API, Method C): 1.298 min, m/z, 354.2 [M + H]+ (%) |
| 93 | 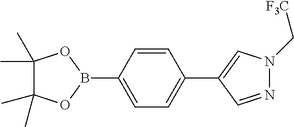 [SMILES: CC1(C)OB(c2ccc(-c3cnn(CC(F)(F)F)c3)cc2)OC1(C)C] | LC-MS (ES-API, Method C): 1.868 min, m/z. [M + H] = 353.2 |
| 94 | 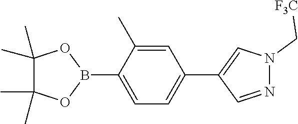 [SMILES: Cc1cc(-c2cnn(CC(F)(F)F)c2)ccc1B1OC(C)(C)C(C)(C)O1] | LC-MS (ES-API, Method C): 2.193 min, m/z, 367.2 M + H]+ |

Intermediate 93 and analogues described above were prepared following the scheme and procedures below:

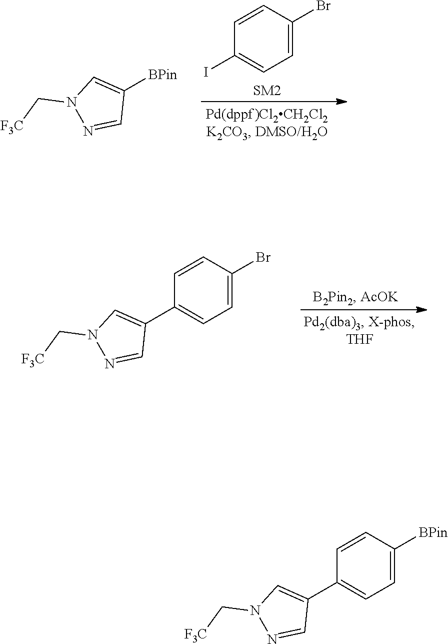

### 4-(4-bromophenyl)-1-(2,2,2-trifluoroethyl)pyrazole

A solution of 4-(4,4,5,5-tetramethyl-1,3,2-dioxaborolan-2-yl)-1-(2,2,2-trifluoroethyl)-1H-pyrazole (1.00 g, 3.62 mmol), 1,4-dibromobenzene (1.7 mg, 7.25 mmol), Pd(dppf)Cl₂DCM (296 mg, 0.36 mmol) and K₂CO₃ (751 mg, 5.43 mmol) in 1,4-dioxane (90 mL) and water (10 mL) was stirred at 80° C. under inert atmosphere overnight. The mixture was concentrated and purified by column chromatograpy eluting with Pet. Ether/EtOAc (10:1) to give 4-[4-(4,4,5,5-tetramethyl-1,3,2-dioxaborolan-2-yl)phenyl]-1-(2,2,2-trifluoroethyl)pyrazole (530 mg).

LC-MS (Method C): 1.60 min, m/z 304.9 [M+H]⁺

### 4-[4-(4,4,5,5-tetramethyl-1,3,2-dioxaborolan-2-yl)phenyl]-1-(2,2,2-trifluoroethyl)pyrazole

A solution of 4-(4-bromophenyl)-1-(2,2,2-trifluoroethyl)-1H-pyrazole (480 mg, 1.57 mmol), 4,4,4′,4′,5,5,5′,5′-octamethyl-2,2′-bi(1,3,2-dioxaborolane) (799 mg, 3.15 mmol), KOAc (463 mg, 4.72 mmol), Pd2(dba)3 (144 mg, 0.16 mmol) and X-PhOS (150 mg, 0.31 mmol) in THF (20 mL) was stirred at 75° C. with N2 overnight. Further purification by flash column chromatography eluting with DCM/MeOH (20:1) gave 4-[4-(4,4,5,5-tetramethyl-1,3,2-dioxaborolan-2-yl)phenyl]-1-(2,2,2-trifluoroethyl)pyrazole (assumed quantitative).

LC-MS (Method C): 1.87 min, m/z 353.2 [M+H]⁺

TABLE 7
Example Compounds

| Example | | | | |
|---|---|---|---|---|
| Number | Structure | NMR | MS [M + H⁺]+ | Method |
| 1 | 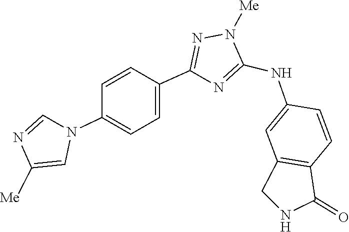 [SMILES: Cc1cn(-c2ccc(-c3nc(Nc4ccc5c(c4)CNC5=O)n(C)n3)cc2)cn1] | ¹H NMR (400 MHz, DMSO-d₆) δ 9.39 (s, 1H), 8.28 (s, 1H), 8.19 (d, J = 1.1 Hz, 1H), 7.70 (d, J = 8.7 Hz, 2H), 7.99 (s, 1H), 7.72-7.67 (m, 3H), 7.62-7.59 (m, 1H), 7.49 (s, 1H), 4.38 (s, 2H), 3.84 (s, 3H), 2.18 (s, 3H) | 0.94 min, m/z = 386.1 [M + H]⁺ | Method D |
| 2 | 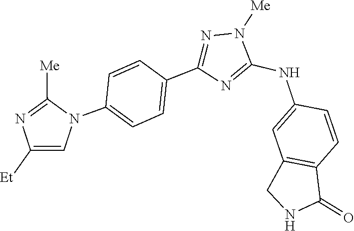 [SMILES: CCc1cn(-c2ccc(-c3nc(Nc4ccc5c(c4)CNC5=O)n(C)n3)cc2)c(C)n1] | ¹H NMR (400 MHz, DMSO-d₆) δ 9.40 (br s, 1H), 8.28 (s, 1H), 8.08 (d, J = 8.5 Hz, 2H), 8.00 (s, 1H), 7.70 (dd, J = 8.4, 1.4 Hz, 1H), 7.63-7.58 (m, 1H), 7.50 (d, J= 8.4 Hz, 2H), 7.02 (s, 1H), 4.37 (s, 2H), 3.85 (s, 3H), 2.50-2.44 (m, 2H), 2.29 (s, 3H), 1.17 (t, J = 7.5 Hz, 3H) | 1.68 min, m/z = 414.0 [M + H]⁺ | Method N |
| 3 | 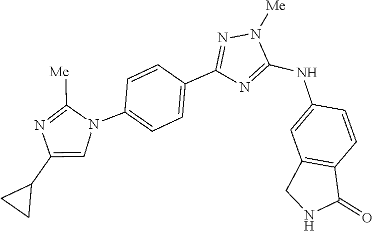 [SMILES: Cc1nc(C2CC2)cn1-c1ccc(-c2nc(Nc3ccc4c(c3)CNC4=O)n(C)n2)cc1] | ¹H NMR (400 MHz, DMSO-d₆) δ 9.39 (s, 1H), 8.28 (s, 1H), 8.08 (d, J = 8.5 Hz, 2H), 8.00 (s, 1H), 7.69 (dd, J = 8.4, 1.6 Hz, 1H), 7.63-7.59 (m, 1H), 7.49 (d, J = 8.6 Hz, 2H), 7.06 (s, 1H), 4.37 (s, 2H), 3.85 (s, 3H), 2.27 (s, 3H), 1.82-1.74 (m, 1H), 0.78-0.73 (m, 2H), 0.69-0.65 (m, 2H) | 1.67 min, m/z = 425.9 [M + H]⁺ | Method N |
| 4 | 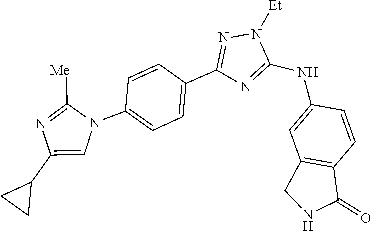 [SMILES: CCn1nc(-c2ccc(-n3cc(C4CC4)nc3C)cc2)nc1Nc1ccc2c(c1)CNC2=O] | ¹H NMR (400 MHz, DMSO-d₆) δ 9.35 (s, 1H), 8.28 (s, 1H), 8.09 (d, J = 8.5 Hz, 2H), 8.00 (s, 1H), 7.70 (dd, J = 8.3, 1.7 Hz, 1H), 7.62-7.58 (m, 1H), 7.49 (d, J = 8.5 Hz, 2H), 7.07 (s, 1H), 4.37 (s, 2H), 4.24 (q, J = 7.1 Hz, 2H), 2.27 (s, 3H), 1.82-1.74 (m, 1H), 1.39 (t, J = 7.2 Hz, 3H), 0.78-0.73 (m, 2H), 0.69-0.65 (m, 2H) | 0.85 min, m/z = 440.0 [M + H]⁺ | Method D |
| 5 | 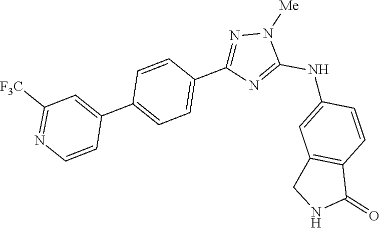 [SMILES: Cn1nc(-c2ccc(-c3ccnc(C(F)(F)F)c3)cc2)nc1Nc1ccc2c(c1)CNC2=O] | ¹H NMR (400 MHz, DMSO-d₆) δ 9.41 (s, 1H), 8.84 (d, J = 5.1 Hz, 1H), 8.29 (s, 1H), 8.24 (s, 1H), 8.17-8.13 (m, 2H), 8.11 (d, J = 4.9 Hz, 1H), 8.07-8.02 (m, 2H), 8.00 (s, 1H), 7.72-7.68 (m, 1H), 7.64-7.60 (m, 1H), 4.38 (s, 2H), 3.87 (s, 3H) | 3.64 min, m/z = 451.25 [M + H]⁺ | Method G |
| 6 | 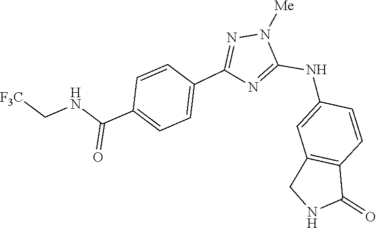 [SMILES: Cn1nc(-c2ccc(C(=O)NCC(F)(F)F)cc2)nc1Nc1ccc2c(c1)CNC2=O] | ¹H NMR (400 MHz, DMSO-d₆) δ 9.42 (s, 1H), 9.16 (t, J = 6.3 Hz, 1H), 8.30 (s, 1H), 8.12-8.08 (m, 2H), 8.00-7.97 (m, 3H), 7.72-7.68 (m, 1H), 7.63-7.59 (m, 1H), 4.38 (s, 2H), 4.17-4.06 (m, 2H), 3.86 (s, 3H) | 3.18 min, m/z = 431.1 [M + H]⁺ | Method B |
| 7 | 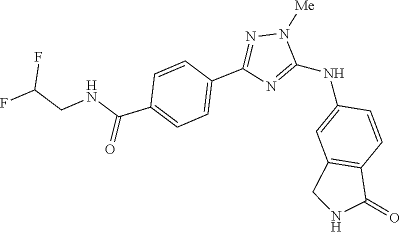 [SMILES: Cn1nc(-c2ccc(C(=O)NCC(F)F)cc2)nc1Nc1ccc2c(c1)CNC2=O] | ¹H NMR (400 MHz, DMSO-d₆) δ 9.40 (s, 1H), 8.93 (t, J = 5.9 Hz, 1H), 8.30 (s, 1H), 8.11-8.07 (m, 2H), 8.00-7.95 (m, 3H), 7.72-7.68 (m, 1H), 7.63-7.59 (m, 1H), 6.15 (tt, J = 4.0 Hz, 56.1 Hz, 1H), 4.38 (s, 2H), 3.86 (s, 3H), 3.76- 3.64 (m, 2H) | 2.98 min, m/z = 413.2 [M + H]⁺ | Method B |
| 8 | 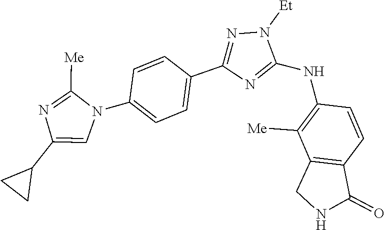 [SMILES: CCn1nc(-c2ccc(-n3cc(C4CC4)nc3C)cc2)nc1Nc1ccc2c(c1C)CNC2=O] | ¹H NMR (400 MHz, DMSO-d₆) δ 8.41-8.38 (m, 2H), 7.99 (d, J = 8.5 Hz, 2H), 7.47-7.43 (m, 4H), 7.04 (s, 1H), 4.33 (s, 2H), 4.17 (q, J = 7.2 Hz, 2H), 2.24 (s, 3H), 2.21 (s, 3H), 1.81-1.73 (m, 1H), 1.40 (t, J = 7.2 Hz, 3H), 0.78-0.72 (m, 2H), 0.68-0.64 (m, 2H) | 0.23 min, m/z = 454.30 [M + H]⁺ | Method K |
| 9 | 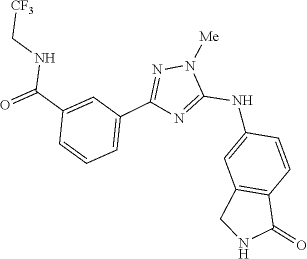 [SMILES: Cn1nc(-c2cccc(C(=O)NCC(F)(F)F)c2)nc1Nc1ccc2c(c1)CNC2=O] | ¹H NMR (400 MHz, DMSO-d₆) δ 9.41 (s, 1H), 9.28 (t, J = 6.1 Hz, 1H), 8.50 (t, J = 1.5 Hz, 1H), 8.29 (s, 1H), 8.19 (dt, J = 2.7 Hz, 7.8 Hz, 1H), 8.00-7.99 (m, 1H), 7.93-7.90 (m, 1H), 7.71-7.67 (m, 1H), 7.63-7.58 (m, 2H), 4.38 (s, 2H), 4.18-4.07 (m, 2H), 3.87 (s, 3H) | 3.31 min, m/z = 431.1 [M + H]⁺ | Method B |
| 10 | 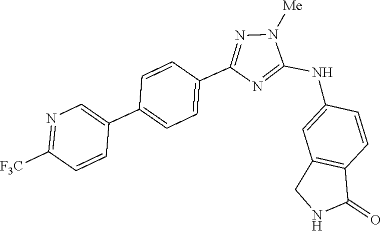 [SMILES: Cn1nc(-c2ccc(-c3ccc(C(F)(F)F)nc3)cc2)nc1Nc1ccc2c(c1)CNC2=O] | ¹H NMR (400 MHz, DMSO-d₆) δ 9.41 (s, 1H), 9.17 (d, J = 2.1 Hz, 1H), 8.46- 8.42 (m, 1H), 8.30 (s, 1H), 8.14-8.14 (m, 2H), 8.04-8.00 (m, 2H), 7.98-7.94 (m, 2H), 7.74-7.70 (m, 1H), 7.64-7.61 (m, 1H), 4.39 (s, 2H), 3.87 (s, 3H) | 3.98 min, m/z = 451.1 [M + H]⁺ | Method B |
| 11 | 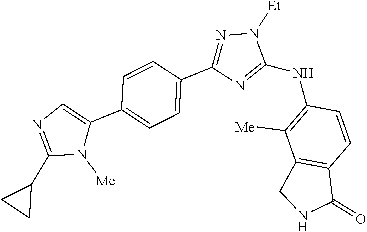 [SMILES: CCn1nc(-c2ccc(-c3cnc(C4CC4)n3C)cc2)nc1Nc1ccc2c(c1C)CNC2=O] | ¹H NMR (400 MHz, DMSO-d₆) δ 8.42-8.38 (m, 2H), 7.85 (d, J = 8.4 Hz, 2H), 7.71 (d, J = 8.3 Hz, 2H), 7.55 (s, 1H), 7.48-7.40 (m, 2H), 4.33 (s, 2H), 4.16 (q, J = 7.2 Hz, 2H), 3.69 (s, 3H), 2.21 (s, 3H), 2.04-1.96 (m, 1H), 1.38 (t, J = 7.2 Hz, 3H), 0.97-0.89 (m, 4H) | 2.42 min, m/z = 454.2 [M + H]⁺ | Method M |
| 12 | 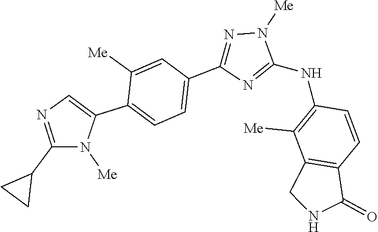 [SMILES: Cc1cc(-c2nc(Nc3ccc4c(c3C)CNC4=O)n(C)n2)ccc1-c1cnc(C2CC2)n1C] | ¹H NMR (400 MHz, DMSO-d₆) δ 8.36 (s, 1H), 8.32 (s, 1H), 7.84-7.80 (m, 1H), 7.74-7.69 (m, 2H), 7.46 (s, 2H), 7.32 (s, 1H), 4.33 (s, 2H), 3.77 (s, 3H), 3.72 (s, 3H), 2.44 (s, 3H), 2.22 (s, 3H), 2.04-1.97 (m, 1H), 0.97-0.88 (m, 4H) | 1.49 min, m/z - 454.2 [M + H]⁺ | Method E |
| 13 | 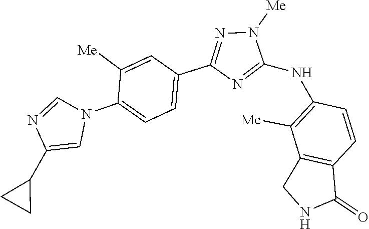 [SMILES: Cc1cc(-c2nc(Nc3ccc4c(c3C)CNC4=O)n(C)n2)ccc1-n1cnc(C2CC2)c1] | ¹H NMR (400 MHz, DMSO-d₆) δ 8.39 (s, 2H), 7.89 (s, 1H), 7.84-7.79 (m, 1H), 7.66 (s, 1H), 7.53-7.45 (m, 2H), 7.31 (d, J = 8.1 Hz, 1H), 7.15 (s, 1H), 4.33 (s, 2H), 3.79 (s, 3H), 2.22 (s, 6H), 1.88-1.80 (m, 1H), 0.82-0.76 (m, 2H), 0.72-0.67 (2H) | 1.58 min, m/z = 440.2 [M + H]⁺ | Method E |
| 14 | 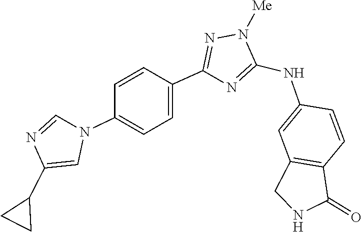 [SMILES: Cn1nc(-c2ccc(-n3cnc(C4CC4)c3)cc2)nc1Nc1ccc2c(c1)CNC2=O] | ¹H NMR (400 MHz, DMSO-d₆) δ 9.39 (s, 1H), 8.29 (s, 1H), 8.16 (d, J = 1.3 Hz, 1H), 8.09-8.04 (m, 2H), 8.00-7.99 (m, 1H), 7.73-7.67 (m, 3H), 7.63-7.60 (m, 1H), 7.54 (d, J = 1.4 Hz, 1H), 4.38 (s, 2H), 3.85 (s, 3H), 1.90-1.82 (m, 1H), 0.86-0.79 (m, 2H), 0.74-0.68 (m, 2H) | 2.82 min, m/z = 412.5 [M + H]⁺ | Method B |
| 15 | 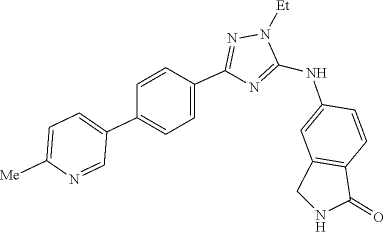 [SMILES: CCn1nc(-c2ccc(-c3ccc(C)nc3)cc2)nc1Nc1ccc2c(c1)CNC2=O] | ¹H NMR (400 MHz, DMSO-d₆) δ 9.33 (s, 1H), 8.82 (s, 1H), 8.27 (s, 1H), 8.10 (d, J = 8.0 Hz, 2H), 8.04-7.98 (m, 2H), 7.80 (d, J = 8.0 Hz, 2H), 7.74- 7.67 (m, 1H), 7.64-7.58 (m, 1H), 7.36 (d, J = 8.1 Hz, 1H), 4.38 (s, 2H), 4.29- 4.20 (m, 2H), 2.52 (s, 3H), 1.40 (t, J = 6.9 Hz, 3H) | 0.99 min, m/z - 411.2 [M + H]⁺ | Method D |
| 16 | 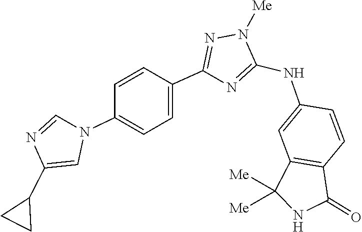 [SMILES: Cn1nc(-c2ccc(-n3cnc(C4CC4)c3)cc2)nc1Nc1ccc2c(c1)C(C)(C)NC2=O] | ¹H NMR (400 MHz, DMSO-d₆) δ 9.38 (s, 1H), 8.36 (s, 1H), 8.15 (s, 1H), 8.05 (d, J = 8.6 Hz, 2H), 7.85 (s, 1H), 7.73-7.68 (m, 3H), 7.57-7.52 (m, 2H), 3.84 (s, 3H), 1.90-1.82 (m, 1H), 1.46 (s, 6H), 0.84-0.79 (m, 2H), 0.73-0.68 (m, 2H) | 1.30 min, m/z = 440.2 [M + H]⁺ | Method D |
| 17 | 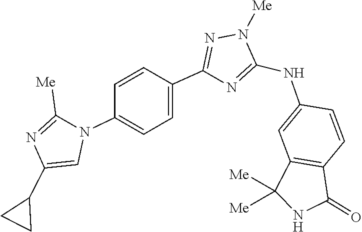 [SMILES: Cc1nc(C2CC2)cn1-c1ccc(-c2nc(Nc3ccc4c(c3)C(C)(C)NC4=O)n(C)n2)cc1] | ¹H NMR (400 MHz, DMSO-d₆) δ 9.49 (s, 1H), 8.39 (s, 1H), 8.08 (d, J = 8.4 Hz, 2H), 7.86-7.83 (m, 1H), 7.79-7.74 (m, 1H), 7.57-7.49 (m, 3H), 7.12 (s, 1H), 3.86 (s, 3H), 2.29 (s, 3H), 1.84-1.76 (m, 1H), 1.45 (s, 6H), 0.81-0.75 (m, 2H), 0.71-0.67 (m, 2H) | 1.29 min, m/z = 454.2 [M + H]⁺ | Method D |
| 18 | 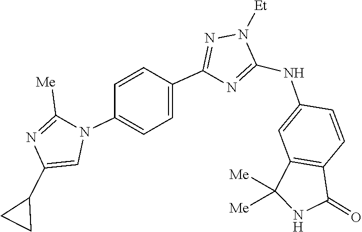 [SMILES: CCn1nc(-c2ccc(-n3cc(C4CC4)nc3C)cc2)nc1Nc1ccc2c(c1)C(C)(C)NC2=O] | ¹H NMR (400 MHz, DMSO-d₆) δ 9.42 (s, 1H), 8.40 (s, 1H), 8.10 (d, J = 9.6 Hz, 2H), 7.86 (s, 1H), 7.77 (d, J = 9.6 Hz, 1H), 7.57-7.52 (m, 3H), 7.17 (s, 1H), 4.26 (q, J = 7.1 Hz, 2H), 2.32 (s, 3H), 1.86-1.78 (m, 1H), 1.46 (s, 6H), 1.40 (t, J = 6.8 Hz, 3H), 0.84-0.80 (m, 2H), 0.73-0.67 (m, 2H) | 1.30 min, m/z = 468.2 [M + H]⁺ | Method D |
| 19 | 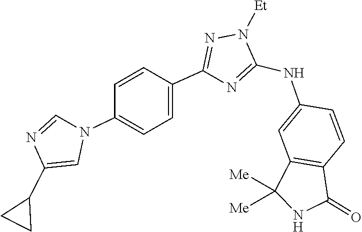 [SMILES: CCn1nc(-c2ccc(-n3cnc(C4CC4)c3)cc2)nc1Nc1ccc2c(c1)C(C)(C)NC2=O] | ¹H NMR (400 MHz, DMSO-d₆) δ 9.37 (s, 1H), 8.40 (s, 1H), 8.16 (s, 1H), 8.06 (d, J = 8.3 Hz, 2H), 7.87 (s, 1H), 7.75-7.69 (m, 3H), 7.57-7.53 (m, 2H), 4.23 (q, J = 7.0 Hz, 2H), 1.91-1.83 (m, 1H), 1.46 (s, 6H), 1.39 (t, J = 7.1 Hz, 3H), 0.84-0.79 (m, 2H), 0.73-0.68 (m, 2H) | 1.27 min, m/z = 454.2 [M + H]⁺ | Method D |
| 20 | 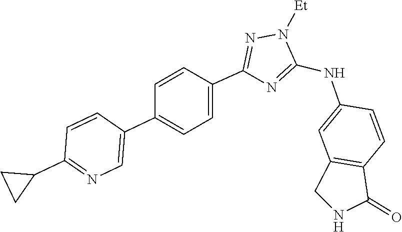 [SMILES: CCn1nc(-c2ccc(-c3ccc(C4CC4)nc3)cc2)nc1Nc1ccc2c(c1)CNC2=O] | ¹H NMR (400 MHz, DMSO-d₆) δ 9.33 (s, 1H), 8.77 (d, J = 2.1 Hz, 1H), 8.27 (s, 1H), 8.09 (d, J = 8.3 Hz, 2H), 8.02-8.00 (m, 1H), 7.98 (dd, J = 8.2, 2.4 Hz, 1H), 7.79 (d, J = 8.4 Hz, 2H), 7.72-7.68 (m, 1H), 7.62-7.59 (m, 1H), 7.40 (d, J = 8.0 Hz, 1H), 4.38 (s, 2H), 4.24 (q, J = 7.1 Hz, 2H), 2.20-2.12 (m, 1H), 1.40 (t, J = 7.1 Hz, 3H), 1.01-0.95 (m, 4H) | 1.51 min, m/z = 437.2 [M + H]⁺ | Method D |
| 21 | 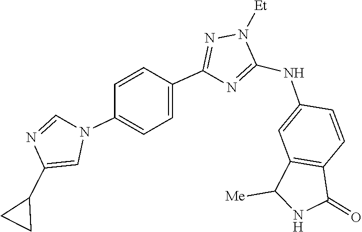 [SMILES: CCn1nc(-c2ccc(-n3cnc(C4CC4)c3)cc2)nc1Nc1ccc2c(c1)C(C)NC2=O] | ¹H NMR (400 MHz, DMSO-d₆) δ 9.34 (s, 1H), 8.37 (s, 1H), 8.16-8.14 (m, 1H), 8.06 (d, J = 8.6 Hz, 2H), 7.93 (s, 1H), 7.75-7.68 (m, 3H), 7.58 (d, J = 8.3 Hz, 1H), 7.54-7.52 (m, 1H), 4.62 (q, J = 6.6 Hz, 1H), 4.23 (q, J = 7.1 Hz, 2H), 1.89-1.81 (m, 1H), 1.42-1.36 (m, 6H), 0.84-0.78 (m, 2H), 0.73-0.68 (m, 2H) | 1.59 min, m/z = 440.2 [M + H]⁺ | Method O |
| 22 | 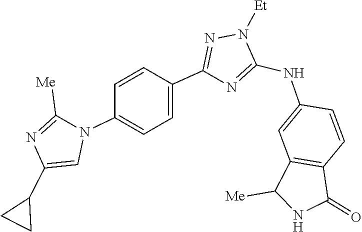 [SMILES: CCn1nc(-c2ccc(-n3cc(C4CC4)nc3C)cc2)nc1Nc1ccc2c(c1)C(C)NC2=O] | ¹H NMR (400 MHz, DMSO-d₆) δ 9.35 (s, 1H), 8.37 (s, 1H), 8.09 (d, J = 8.4 Hz, 2H), 7.91 (s, 1H), 7.77-7.73 (m, 1H), 7.58 (d, J = 8.3 Hz, 1H), 7.50 (d, J = 8.4 Hz, 2H), 7.06 (s, 1H), 4.61 (q, J = 6.5 Hz, 1H), 4.24 (q, J = 7.1 Hz, 2H), 2.27 (s, 3H), 1.82-1.74 (m, 1H), 1.42-1.36 (m, 6H), 0.79-0.73 (m, 2H), 0.70-0.65 (m, 2H) | 1.52 min, 454.2 = [M + H]⁺ | Method O |
| 23 | 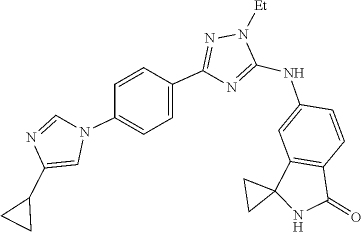 [SMILES: CCn1nc(-c2ccc(-n3cnc(C4CC4)c3)cc2)nc1Nc1ccc2c(c1)C1(CC1)NC2=O] | ¹H NMR (400 MHz, DMSO-d₆) δ 9.34 (s, 1H), 8.43 (s, 1H), 8.16 (s, 1H), 8.04 (d, J = 8.5 Hz, 2H), 7.74-7.68 (m, 3H), 7.64-7.60 (m, 1H), 7.56 (s, 1H), 7.54 (s, 1H), 4.22 (q, J = 7.1 Hz, 2H), 1.90-1.82 (m, 1H), 1.51-1.46 (m, 2H), 1.41-1.33 (m, 5H), 0.84-0.79 (m, 2H), 0.73-0.68 (m, 2H) | 0.45 min, m/z = 452.2 [M + H]⁺ | Method J |
| 24 | 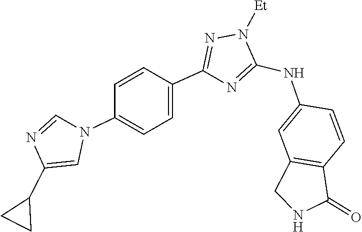 [SMILES: CCn1nc(-c2ccc(-n3cnc(C4CC4)c3)cc2)nc1Nc1ccc2c(c1)CNC2=O] | ¹H NMR (400 MHz, DMSO-d₆) δ 9.33 (s, 1H), 8.28 (s, 1H), 8.15 (d, J = 1.3 Hz, 1H), 8.09-8.04 (m, 2H), 8.00-7.98 (m, 1H), 7.72-7.67 (m, 3H), 7.60 (d, J = 8.4 Hz, 1H), 7.54 (d, J = 1.3 Hz, 1H), 4.37 (s, 2H), 4.23 (q, J = 7.1 Hz, 2H), 1.89-1.81 (m, 1H), 1.39 (t, J = 7.1 Hz, 3H), 0.84-0.79 (m, 2H), 0.73- 0.68 (m, 2H) | 2.49 min m/z = 426.8 [M + H]⁺ | Method B |
| 25 | 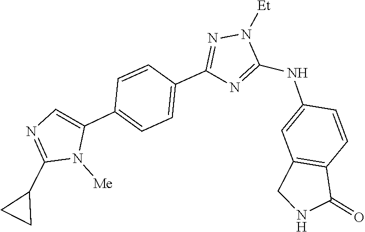 [SMILES: CCn1nc(-c2ccc(-c3cnc(C4CC4)n3C)cc2)nc1Nc1ccc2c(c1)CNC2=O] | ¹H NMR (400 MHz, DMSO-d₆) δ 9.27 (s, 1H), 8.26 (s, 1H), 7.97-7.95 (m, 1H), 7.94-7.90 (m, 2H), 7.75-7.71 (m, 2H), 7.67 (dd, J = 8.5, 2.0 Hz, 1H), 7.59 (d, J = 8.5 Hz, 1H), 7.53 (s, 1H), 4.37 (s, 2H), 4.21 (q, J = 7.3 Hz, 2H), 3.70 (s, 3H), 2.02-1.94 (m, 1H), 1.38 (t, J = 7.3 Hz, 3H), 0.95-0.86 (m, 4H) | 2.43 min m/z = 440.8 [M + H]⁺ | Method B |
| 26 | 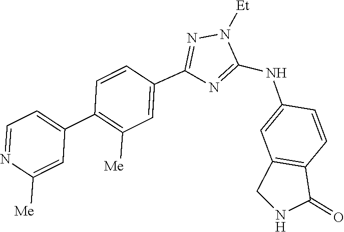 [SMILES: CCn1nc(-c2ccc(-c3ccnc(C)c3)c(C)c2)nc1Nc1ccc2c(c1)CNC2=O] | ¹H NMR (400 MHz, DMSO-d₆) δ 9.32 (s, 1H), 8.52 (d, J = 5.2 Hz, 1H), 8.28 (s, 1H), 7.99-7.94 (m, 2H), 7.93-7.90 (m, 1H), 7.71 (dd, J = 8.4, 1.9 Hz, 1H), 7.61 (d, J = 8.4 Hz, 1H), 7.34 (d, J = 7.9 Hz, 1H), 7.31-7.29 (m, 1H), 7.25- 7.21 (m, 1H), 4.38 (s, 2H), 4.25 (q, J = 7.2 Hz, 2H), 2.54 (s, 3H), 2.34 (s, 3H), 1.41 (t, J = 7.2 Hz, 3H) | 2.46 min, m/z = 425.1 [M + H]⁺ | Method B |
| 27 | 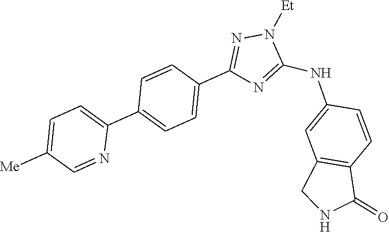 [SMILES: CCn1nc(-c2ccc(-c3ccc(C)cn3)cc2)nc1Nc1ccc2c(c1)CNC2=O] | ¹H NMR (400 MHz, DMSO-d₆) δ 9.32 (s, 1H), 8.52 (s, 1H), 8.27 (s, 1H), 8.17 (d, J = 8.4 Hz, 2H), 8.10 (d, J = 8.1 Hz 2H), 7.99 (s, 1H), 7.91 (d, J = 8.1 Hz, 1H), 7.73-7.69 (m, 2H), 7.62 (d, J = 8.7 Hz, 1H), 4.38 (s, 2H), 4.24 (q, J = 7.1 Hz, 2H), 2.35 (s, 3H), 1.40 (t, J = 7.1 Hz, 3H) | 2.80 min, m/z = 411.2 [M + H]⁺ | Method M |
| 28 | 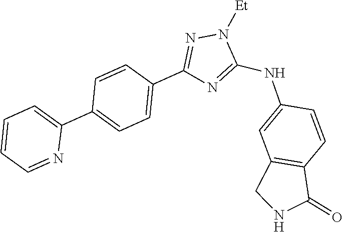 [SMILES: CCn1nc(-c2ccc(-c3ccccn3)cc2)nc1Nc1ccc2c(c1)CNC2=O] | ¹H NMR (400 MHz, DMSO-d₆) δ 9.33 (s, 1H), 8.69 (d, J = 4.0 Hz, 1H), 8.27 (s, 1H), 8.20 (d, J = 8.3 Hz, 2H), 8.12 (d, J = 8.3 Hz, 2H), 8.03-7.99 (m, 2H), 7.93-7.88 (m, 1H), 7.72 (dd, J = 8.3, 1.3 Hz, 1H), 7.61 (d, J = 8.3 Hz, 1H), 7.39-7.35 (m, 1H), 4.38 (s, 2H), 4.25 (q, J = 7.1 Hz, 2H), 1.41 (t, J = 7.1 Hz, 3H) | 1.22 min, m/z = 397.2 [M + H]⁺ | Method D |
| 29 | 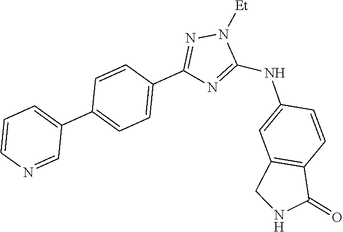 [SMILES: CCn1nc(-c2ccc(-c3cccnc3)cc2)nc1Nc1ccc2c(c1)CNC2=O] | ¹H NMR (400 MHz, DMSO-d₆) δ 9.33 (s, 1H), 8.96 (d, J = 2.0 Hz, 1H), 8.60- 8.58 (m, 1H), 8.27 (s, 1H), 8.16-8.10 (m, 3H), 8.01 (s, 1H), 7.84 (d, J = 8.3 Hz, 2H), 7.71 (dd, J = 8.3,1.6 Hz, 1H), 7.61 (d, J = 8.7 Hz, 1H), 7.53-7.49 (m, 1H), 4.38 (s, 2H), 4.25 (q, J = 7.1 Hz, 2H), 1.40 (t, J = 7.2 Hz, 3H) | 1.03 min, m/z = 397.2 [M + H]⁺ | Method D |
| 30 | 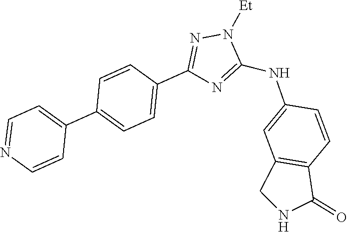 [SMILES: CCn1nc(-c2ccc(-c3ccncc3)cc2)nc1Nc1ccc2c(c1)CNC2=O] | ¹H NMR (400 MHz, DMSO-d₆) δ 9.35 (s, 1H), 8.69-8.63 (m, 2H), 8.27 (s, 1H), 8.13 (d, J = 8.3 Hz, 2H), 8.00 (s, 1H), 7.92 (d, J = 8.2 Hz, 2H), 7.79- 7.74 (m, 2H), 7.73-7.69 (m, 1H), 7.61 (d, J = 8.2 Hz, 1H), 4.38 (s, 2H), 4.25 (q, J = 7.1 Hz, 2H), 1.40 (t, J = 7.1 Hz, 3H) | 1.54 min, m/z = 397.3 [M + H]⁺ | Method E |
| 31 | 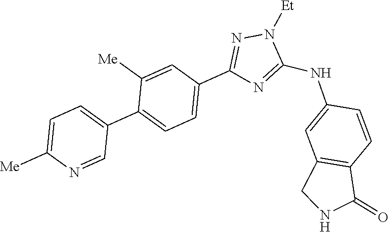 [SMILES: CCn1nc(-c2ccc(-c3ccc(C)nc3)c(C)c2)nc1Nc1ccc2c(c1)CNC2=O] | ¹H NMR (400 MHz, DMSO-d₆) δ 9.30 (s, 1H), 8.47 (d, J = 1.9 Hz, 1H), 8.26 (s, 1H), 7.98 (s, 1H), 7.94 (s, 1H), 7.92-7.88 (m, 1H), 7.75-7.67 (m, 2H), 7.60 (d, J = 8.3 Hz, 1H), 7.36-7.30 (m, 2H), 4.37 (s, 2H), 4.23 (q, J = 7.1 Hz, 2H), 2.53 (s, 3H), 2.32 (s, 3H), 1.40 (t, J = 7.2 Hz, 3H) | 0.30 min, m/z = 425.2 [M + H]⁺ | Method D |
| 32 | 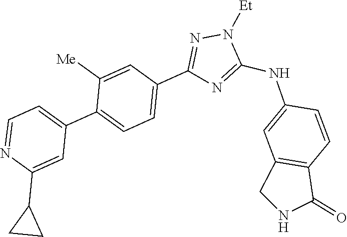 [SMILES: CCn1nc(-c2ccc(-c3ccnc(C4CC4)c3)c(C)c2)nc1Nc1ccc2c(c1)CNC2=O] | ¹H NMR (400 MHz, DMSO-d₆) δ 9.33 (s, 1H), 8.47-8.44 (m, 1H), 8.28 (s, 1H), 7.99-7.97 (m, 1H), 7.96-7.94 (m, 1H), 7.93-7.90 (m, 1H), 7.71 (dd, J = 7.4, 1.9 Hz, 1H), 7.61 (d, J = 8.3 Hz, 1H), 7.37-7.32 (m, 2H), 7.16 (dd, J = 5.1, 1.7 Hz, 1H), 4.38 (s, 2H), 4.25 (q, J = 7.2 Hz, 2H), 2.35 (s, 3H), 2.21- 2.13 (m, 1H), 1.41 (t, J = 7.1 Hz, 3H), 1.00-0.95 (m, 4H) | 2.75 min, m/z = 451.7 [M + H]⁺ | Method B |
| 33 | 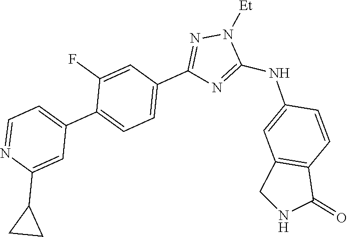 [SMILES: CCn1nc(-c2ccc(-c3ccnc(C4CC4)c3)c(F)c2)nc1Nc1ccc2c(c1)CNC2=O] | ¹H NMR (400 MHz, DMSO-d₆) δ 9.39 (s, 1H), 8.50 (d, J = 8.5 Hz, 1H), 8.30 (s, 1H), 8.02-7.99 (m, 1H), 7.96 (dd, J = 8.0, 1.6 Hz, 1H), 7.86 (dd, J = 12.0, 1.5 Hz, 1H), 7.78-7.73 (m, 1H), 7.71 (dd, J = 8.4, 1.9 Hz, 1H), 7.62 (d, J = 8.4 Hz, 1H), 7.54 (s, 1H), 7.38-7.34 (m, 1H), 4.39 (s, 2H), 4.26 (q, J = 7.2 Hz, 2H), 2.24-2.16 (m, 1H), 1.41 (t, J = 7.1 Hz, 3H), 1.01-0.97 (m, 4H) | 2.89 min, m/z = 455.8 [M + H]⁺ | Method B |
| 34 | 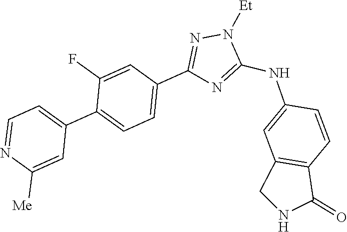 [SMILES: CCn1nc(-c2ccc(-c3ccnc(C)c3)c(F)c2)nc1Nc1ccc2c(c1)CNC2=O] | ¹H NMR (400 MHz, DMSO-d₆) δ 9.38 (s, 1H), 8.56 (d, J = 5.1 Hz, 1H), 8.29 (s, 1H), 8.02-7.99 (m, 1H), 7.96 (dd, J = 8.1, 1.5 Hz 1H), 7.86 (dd, J = 12.0, 1.5 Hz 1H), 7.77-7.69 (m, 2H), 7.62 (d, J = 8.4 Hz, 1H), 7.51 (s, 1H), 7.45- 7.42 (m, 1H), 4.39 (s, 2H), 4.26 (q, J = 7.2 Hz, 2H), 2.56 (s, 3H), 1.41 (t, J = 7.2 Hz, 3H) | 2.52 min, m/z = 429.8 [M + H]⁺ | Method B |
| 35 | 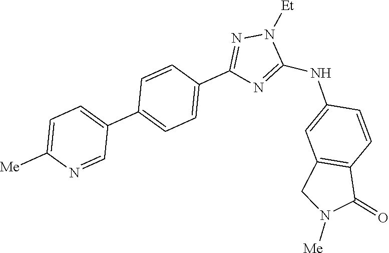 [SMILES: CCn1nc(-c2ccc(-c3ccc(C)nc3)cc2)nc1Nc1ccc2c(c1)CN(C)C2=O] | ¹H NMR (400 MHz, DMSO-d₆) δ 9.33 (s, 1H), 8.82 (d, J = 2.1 Hz, 1H), 8.10 (d, J = 8.3 Hz, 2H), 8.02 (dd, J = 8.1 Hz, 2.3 Hz, 1H), 7.99 (s, 1H), 7.81 (d, J = 8.3 Hz, 2H), 7.73-7.70 (m, 1H), 7.60 (d, J = 8.3 Hz, 1H), 7.37 (d, J = 8.1 Hz, 1H), 4.46 (s, 2H), 4.24 (q, J = 7.1 Hz, 2H), 3.05 (s, 3H), 2.52 (s, 3H), 1.40 (t, J = 7.1 Hz, 3H) | 1.66 min, m/z = 425.2 [M + H]⁺ | Method O |
| 36 | 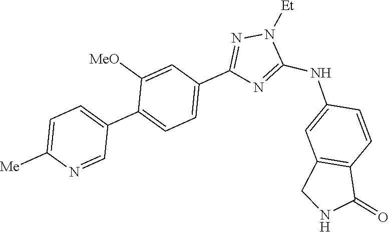 [SMILES: CCn1nc(-c2ccc(-c3ccc(C)nc3)c(OC)c2)nc1Nc1ccc2c(c1)CNC2=O] | ¹H NMR (400 MHz, DMSO-d₆) δ 9.32 (s, 1H), 8.60-8.57 (m, 1H), 8.26 (s, 1H), 7.97 (s, 1H), 7.82 (dd, J = 8.0, 2.0 Hz, 1H), 7.72-7.65 (m, 3H), 7.60 (d, J = 10.0 Hz, 1H), 7.43 (d, J = 7.8 Hz, 1H), 7.30 (d, J = 8.0 Hz, 1H), 4.37 (s, 2H), 4.24 (q, J = 7.1 Hz, 2H), 3.86 (s, 3H), 2.49 (s, 3H), 1.40 (t, J = 7.1 Hz, 3H) | 1.58 min, m/z = 441.2 [M + H]⁺ | Method O |
| 37 | 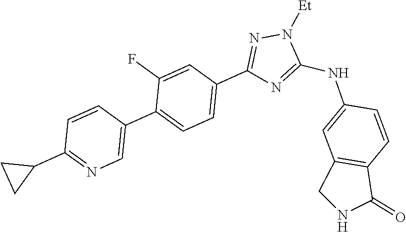 [SMILES: CCn1nc(-c2ccc(-c3ccc(C4CC4)nc3)c(F)c2)nc1Nc1ccc2c(c1)CNC2=O] | ¹H NMR (400 MHz, DMSO-d₆) δ 9.38 (s, 1H), 8.64 (s, 1H), 8.29 (s, 1H), 8.03-8.00 (m, 1H), 7.93 (dd, J = 8.1, 1.6 Hz, 1H), 7.90-7.87 (m, 1H), 7.83 (dd, J = 11.9, 1.5 Hz, 1H), 7.72-7.67 (m, 2H), 7.62 (d, J = 8.3 Hz, 1H), 7.45- 7.42 (m, 1H), 4.39 (s, 2H), 4.26 (q, J = 7.2 Hz, 2H), 2.19-2.16 (m, 1H), 1.41 (t, J = 7.2 Hz, 3H), 1.02-0.98 (m, 4H) | 3.17 min, m/z = 455.3 [M + H]⁺ (100%) | Method B |
| 38 | 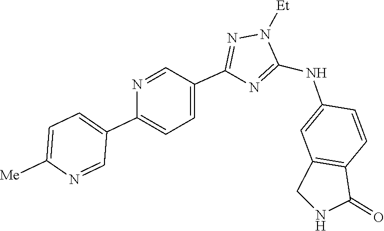 [SMILES: CCn1nc(-c2ccc(-c3ccc(C)nc3)nc2)nc1Nc1ccc2c(c1)CNC2=O] | ¹H NMR (400 MHz, DMSO-d₆) δ 9.36 (s, 1H), 8.68 (s, 1H), 8.27 (s, 1H), 8.00 (s, 1H), 7.95-7.89 (m, 2H), 7.83 (dd, J = 11.8, 1.2 Hz, 1H), 7.72-7.66 (m, 2H), 7.61 (d, J = 8.7 Hz, 1H), 7.39 (d, J = 8.1 Hz, 1H), 4.38 (s, 2H), 4.25 (q, J = 7.1 Hz, 2H), 2.54 (s, 3H), 1.40 (t, J = 7.1 Hz, 3H) | 1.77 min, m/z = 429.2 [M + H]⁺ | Method E |
| 39 | 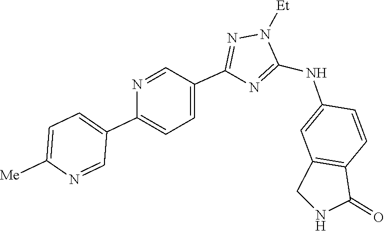 [SMILES: CCn1nc(-c2ccc(-c3ccc(C)nc3)nc2)nc1Nc1ccc2c(c1)CNC2=O] | ¹H NMR (400 MHz, DMSO-d₆) δ 9.39 (s, 1H), 9.27 (d, J = 1.6 Hz, 1H), 9.19 (d, J = 1.9 Hz, 1H), 8.41-8.36 (m, 2H), 8.28 (s, 1H), 8.12 (d, J = 8.3 Hz, 1H), 8.05 (s, 1H), 7.70 (dd, J = 8.3 Hz, 1.5 Hz, 1H), 7.62 (d, J = 8.5 Hz, 1H), 7.40 (d, J = 8.1 Hz, 1H), 4.38 (s, 2H), 4.27 (q, J = 7.1 Hz, 2H), 2.54 (s, 3H), 1.41 (t, J = 7.1 Hz, 3H) | 0.90 min, m/z = 412.2 [M + H]⁺ | Method D |
| 40 | 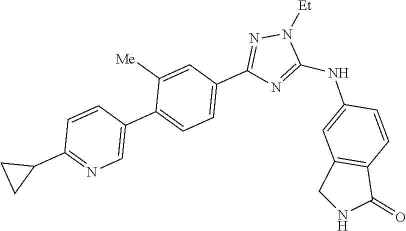 [SMILES: CCn1nc(-c2ccc(-c3ccc(C4CC4)nc3)c(C)c2)nc1Nc1ccc2c(c1)CNC2=O] | ¹H NMR (400 MHz, DMSO-d₆) δ 9.32 (s, 1H), 8.44-8.42 (m, 1H), 8.27 (s, 1H), 7.99-7.97 (m, 1H), 7.95-7.92 (m, 1H), 7.90-7.87 (m, 1H), 7.71-7.69 (m, 2H), 7.61 (d, J = 8.3 Hz, 1H), 7.40-7.37 (m, 1H), 7.32 (d, J = 7.9 Hz, 1H), 4.38 (s, 2H), 4.24 (q, J = 7.2 Hz, 2H), 2.33 (s, 3H), 2.20-2.14 (m, 1H), 1.40 (t, J = 7.2 Hz, 3H), 1.00-0.98 (m, 4H) | 2.92 min, m/z = 451.4 [M + H]⁺ | Method B |
| 41 | 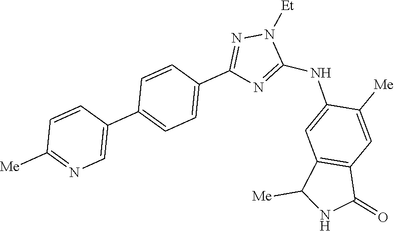 [SMILES: CCn1nc(-c2ccc(-c3ccc(C)nc3)cc2)nc1Nc1cc2c(cc1C)C(=O)NC2C] | ¹H NMR (400 MHz, DMSO-d₆) δ 8.80 (d, J = 2.1 Hz, 1H), 8.37 (s, 1H), 8.25 (s, 1H), 8.05-7.98 (m, 3H), 7.78 (d, J = 8.4 Hz, 2H), 7.55 (s, 1H), 7.47 (s, 1H), 7.35 (d, J = 8.1 Hz, 1H), 4.55 (q, J = 6.5 Hz, 1H), 4.16 (q, J = 7.2 Hz, 2H), 2.51 (s, 3H), 2.35 (s, 3H), 1.40 (t, J = 7.2 Hz, 3H), 1.33 (d, J = 6.6 Hz, 3H) | 1.87 min, m/z = 439.3 [M + H]⁺ | Method D |
| 42 | 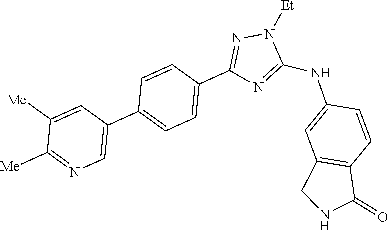 [SMILES: CCn1nc(-c2ccc(-c3cnc(C)c(C)c3)cc2)nc1Nc1ccc2c(c1)CNC2=O] | ¹H NMR (400 MHz, DMSO-d₆) δ 9.33 (br s, 1H), 8.62 (d, J = 1.7 Hz, 1H), 8.26 (s, 1H), 8.09 (d, J = 8.3 Hz, 2H), 8.00 (s, 1H), 7.89-7.85 (m, 1H), 7.80 (d, J = 8.3 Hz, 2H), 7.72-7.68 (m, 1H), 7.61 (d, J = 8.2 Hz, 1H), 4.38 (s, 2H), 4.24 (q, J = 7.1 Hz, 2H), 2.47 (s, 3H), 2.34 (s, 3H), 1.40 (t, J = 7.1 Hz, 3H) | 2.56 min, m/z = 425.4 [M + H]⁺ | Method G |
| 43 | 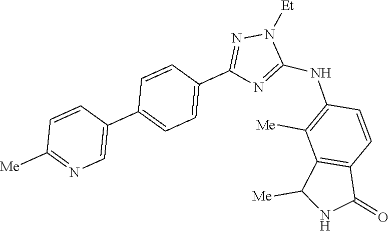 [SMILES: CCn1nc(-c2ccc(-c3ccc(C)nc3)cc2)nc1Nc1ccc2c(c1C)C(C)NC2=O] | ¹H NMR (400 MHz, DMSO-d₆) δ 8.78 (d, J = 1.8 Hz, 1H), 8.42 (s, 1H), 8.31 (s, 1H), 8.02-7.97 (m, 3H), 7.75 (d, J = 8.3 Hz, 2H), 7.46-7.42 (m, 2H), 7.33 (d, J = 8.1 Hz, 1H), 4.68 (q, J = 6.5 Hz, 1H), 4.16 (q, J = 7.1 Hz, 2H), 2.51 (s, 3H), 2.27 (s, 3H), 1.42-1.36 (m, 6H) | 1.03 min, m/z = 439.3 [M + H]⁺ | Method D |
| 44 | 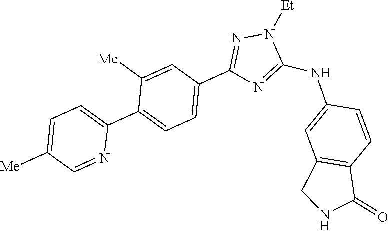 [SMILES: CCn1nc(-c2ccc(-c3ccc(C)cn3)c(C)c2)nc1Nc1ccc2c(c1)CNC2=O] | ¹H NMR (400 MHz, DMSO-d₆) δ 9.35 (s, 1H), 8.52 (s, 1H), 8.27 (s, 1H), 7.98 (s, 1H), 7.93-7.88 (m, 2H), 7.73-7.69 (m, 2H), 7.60 (d, J= 8.5 Hz, 1H), 7.47 (s, 1H), 7.45 (s, 1H), 4.37 (s, 2H), 4.25 (q, J = 7.5 Hz, 2H), 2.41 (s, 3H), 2.36 (s, 3H), 1.40 (t, J = 7.1 Hz, 3H) | 2.17 min, m/z = 425.3 [M + H]⁺ | Method D |
| 45 | 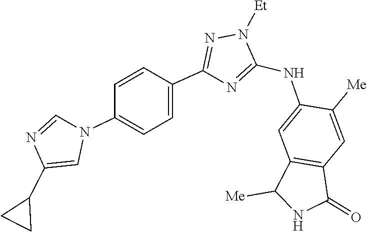 [SMILES: CCn1nc(-c2ccc(-n3cnc(C4CC4)c3)cc2)nc1Nc1cc2c(cc1C)C(=O)NC2C] | ¹H NMR (400 MHz, DMSO-d₆) δ 8.38 (s, 1H), 8.26 (s, 1H), 8.14-8.12 (m, 1H), 8.00 (d, J = 8.3 Hz, 2H), 7.68 (d, J = 8.3 Hz, 2H), 7.54 (s, 1H), 7.53- 7.51 (m, 1H), 7.47 (s, 1H), 4.55 (q, J = 6.5 Hz, 1H), 4.16 (q, J = 7.2 Hz, 2H), 2.35 (s, 3H), 1.88-1.81 (m, 1H), 1.39 (t, J = 7.3 Hz, 3H), 1.32 (d, J = 7.0 Hz, 3H), 0.84-0.77 (m, 2H), 0.72-0.67 (m, 2H) | 0.30 min, m/z = 454.3 [M + H]⁺ | Method D |
| 46 | 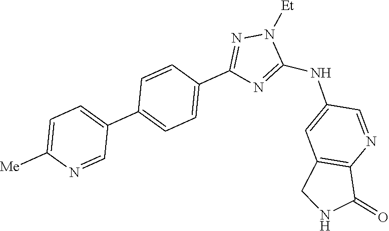 [SMILES: CCn1nc(-c2ccc(-c3ccc(C)nc3)cc2)nc1Nc1cnc2c(c1)CNC2=O] | ¹H NMR (400 MHz, DMSO-d₆) δ 9.63 (s, 1H), 8.87 (d, J = 2.4 Hz, 1H), 8.82 (d, J = 2.4 Hz, 1H), 8.67 (s, 1H), 8.58 (d, J = 2.4 Hz, 1H), 8.13-8.09 (m, 2H), 8.03 (dd, J = 7.8, 2.4 Hz, 1H), 7.83-7.79 (m, 2H), 7.37 (d, J = 7.8 Hz, 1H), 4.43 (s, 2H), 4.27 (q, J = 7.2 Hz, 2H), 2.52 (s, 3H), 1.42 (t, J = 7.2 Hz, 3H) | 2.38 min, m/z - 412.5 [M + H]⁺ | Method B |
| 47 | 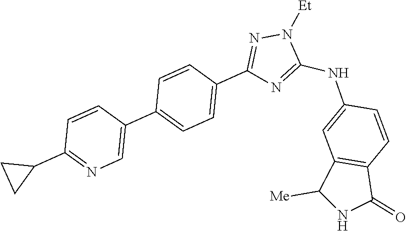 [SMILES: CCn1nc(-c2ccc(-c3ccc(C4CC4)nc3)cc2)nc1Nc1ccc2c(c1)C(C)NC2=O] | ¹H NMR (400 MHz, DMSO-d₆) δ 9.37 (s, 1H), 8.79 (d, J = 1.5 Hz, 1H), 8.39 (s, 1H), 8.11 (d, J = 8.1 Hz, 2H), 8.00 (dd, J = 8.4, 2.6 Hz, 1H), 7.95 (s, 1H), 7.82 (d, J = 7.9 Hz, 2H), 7.79-7.74 (m, 1H), 7.60 (d, J = 8.2 Hz, 1H), 7.42 (d, J = 8.2 Hz, 1H), 4.65 (q, J = 6.8 Hz, 1H), 4.27 (q, J = 6.9 Hz, 2H), 2.22-2.15 (m, 1H), 1.45-1.40 (m, 6H), 1.03-0.97 (m, 4H) | 1.33 min, m/z = 451.1 [M + H]⁺ | Method K |
| 48 | 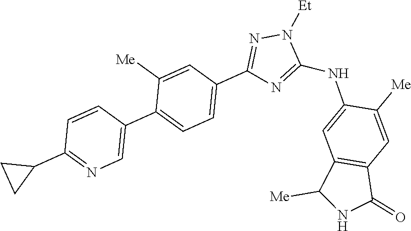 [SMILES: CCn1nc(-c2ccc(-c3ccc(C4CC4)nc3)c(C)c2)nc1Nc1cc2c(cc1C)C(=O)NC2C] | ¹H NMR (400 MHz, DMSO-d₆) δ 8.42 (d, J = 2.2 Hz, 1H), 8.39 (s, 1H), 8.26 (s, 1H), 7.89 (s, 1H), 7.85-7.81 (m, 1H), 7.69 (dd, J = 8.2, 2.4 Hz, 1H), 7.53 (s, 1H), 7.49 (s, 1H), 7.39 (d, J = 8.4 Hz, 1H), 7.30 (d, J = 8.2 Hz, 1H), 4.57 (q, J = 6.7 Hz, 1H), 4.18 (q, J = 7.1 Hz, 2H), 2.36 (s, 3H), 2.31 (s, 3H), 2.21- 2.14 (m, 1H), 1.42 (t, J = 7.2 Hz, 3H), 1.34 (d, J = 6.5 Hz, 3H), 1.02-0.97 (m, 4H) | 1.54 min, m/z = 479.3 [M + H]⁺ | Method D |
| 49 | 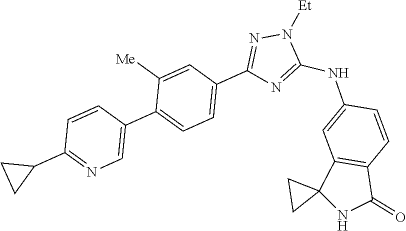 [SMILES: CCn1nc(-c2ccc(-c3ccc(C4CC4)nc3)c(C)c2)nc1Nc1ccc2c(c1)C1(CC1)NC2=O] | ¹H NMR (400 MHz, DMSO-d₆) δ 9.35 (s, 1H), 8.46-8.42 (m, 2H), 7.94 (s, 1H), 7.90-7.85 (m, 1H), 7.75 (dd, J = 8.2, 1.8 Hz, 1H), 7.71 (dd, J = 8.1, 2.4 Hz, 1H), 7.64 (d, J = 8.7 Hz, 1H), 7.56-7.54 (m, 1H), 7.40 (d, J = 8.3 Hz, 1H), 7.33 (d, J = 8.6 Hz, 1H), 4.24 (q, J = 7.1 Hz, 2H), 2.34 (s, 3H), 2.21- 2.15 (m, 1H), 1.53-1.47 (m, 2H), 1.43-1.35 (m, 5H), 1.01-0.98 (m, 4H) | 1.53 min, m/z = 477.3 [M + H]⁺ | Method D |
| 50 | 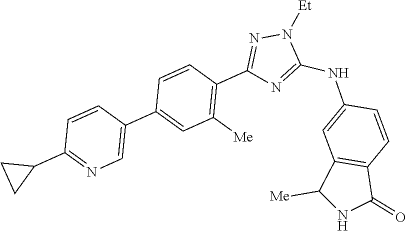 [SMILES: CCn1nc(-c2ccc(-c3ccc(C4CC4)nc3)cc2C)nc1Nc1ccc2c(c1)C(C)NC2=O] | ¹H NMR (400 MHz, DMSO-d₆) δ 9.48 (s, 1H), 8.87 (s, 1H), 8.46 (s, 1H), 8.11-7.89 (m, 3H), 7.74-7.48 (m, 4H), 7.43-7.30 (m, 1H), 4.68-4.52 (m, 1H), 4.32-4.17 (m, 2H), 2.85 (s, 3H), 2.26-2.21 (m, 1H), 1.52-1.49 (m, 6H), 1.15- 1.02 (m, 4H) | 1.58 min, m/z = 465.3 [M + H]⁺ | Method D |
| 51 | 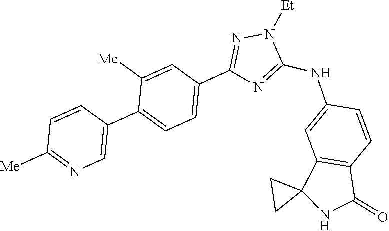 [SMILES: CCn1nc(-c2ccc(-c3ccc(C)nc3)c(C)c2)nc1Nc1ccc2c(c1)C1(CC1)NC2=O] | ¹H NMR (400 MHz, DMSO-d₆) δ 9.36 (s, 1H), 8.50 (s, 1H), 8.45 (s, 1H), 7.95 (s, 1H), 7.90 (d, J = 7.8 Hz, 1H), 7.79-7.74 (m, 2H), 7.65 (d, J = 8.3 Hz, 1H), 7.57 (s, 1H), 7.37 (m, 2H), 4.26 (q, J = 7.0 Hz, 2H), 2.57 (s, 3H), 2.35 (s, 3H), 1.53-1.50 (m, 2H), 1.46-1.35 (m, 5H) | 1.18 min, m/z = 451.2 [M + H]⁺ | Method D |
| 52 | 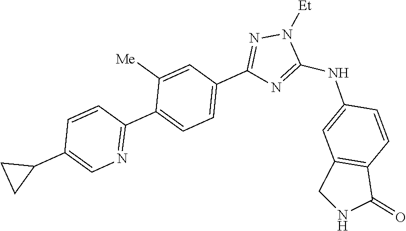 [SMILES: CCn1nc(-c2ccc(-c3ccc(C4CC4)cn3)c(C)c2)nc1Nc1ccc2c(c1)CNC2=O] | ¹H NMR (400 MHz, DMSO-d₆) δ 9.33 (s, 1H), 8.53 (d, J = 2.0 Hz, 1H), 8.29 (s, 1H), 8.00 (s, 1H), 7.95-7.90 (m, 2H), 7.72 (dd, J = 1.6, 8.3 Hz, 1H), 7.63 (d, J = 8.4 Hz, 1H), 7.53 (dd, J = 2.1, 7.9 Hz, 1H), 7.50-7.43 (m, 2H), 4.40 (s, 2H), 4.26 (q, J = 7.2 Hz, 2H), 2.43 (s, 3H), 2.08-2.00 (m, 1H), 1.42 (t, J = 7.2 Hz, 3H), 1.10-1.03 (m, 2H), 0.85-0.79 (m, 2H) | 2.15 min, m/z = 451.3 [M + H]⁺ | Method E |
| 53 | 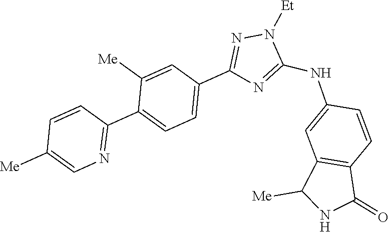 [SMILES: CCn1nc(-c2ccc(-c3ccc(C)cn3)c(C)c2)nc1Nc1ccc2c(c1)C(C)NC2=O] | ¹H NMR (400 MHz, DMSO-d₆) δ 9.35 (s, 1H), 8.54 (s, 1H), 8.39 (s, 1H), 7.94-7.89 (m, 3H), 7.77 (dd, J = 1.7, 8.5 Hz, 1H), 7.73 (dd, J = 1.7, 8.2 Hz, 1H), 7.60 (d, J = 8.3 Hz, 1H), 7.51-7.47 (m, 2H), 4.64 (q, J = 6.5 Hz, 1H), 4.26 (q, J = 7.2 Hz, 2H), 2.42 (s, 3H), 2.38 (s, 3H), 1.45-1.39 (m, 6H) | 2.28 min, m/z = 439.3 [M + H]⁺ | Method D |
| 54 | 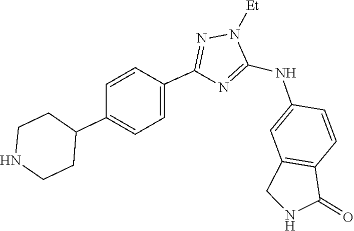 [SMILES: CCn1nc(-c2ccc(C3CCNCC3)cc2)nc1Nc1ccc2c(c1)CNC2=O] | ¹H NMR (400 MHz, DMSO-d₆) δ 9.35 (s, 1H), 8.27 (s, 1H), 7.99 (s, 1H), 7.94 (d, J = 8.4 Hz, 2H), 7.71 (d, J = 7.6 Hz, 1H), 7.61 (d, J = 8.4 Hz, 1H), 7.33 (d, J = 8.4 Hz, 2H), 4.39 (s, 2H), 4.24 (q, J = 7.3 Hz, 2H), 3.13-3.05 (m, 2H), 2.71-2.61 (m, 3H), 1.79-1.70 (m, 2H), 1.59 (m, 2H), 1.40 (t, J = 7.0 Hz, 3H). | 0.32 min, m/z = 403.3 [M + H]⁺ | Method D |
| 55 | 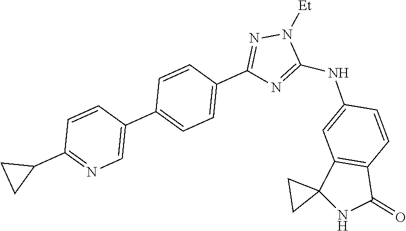 [SMILES: CCn1nc(-c2ccc(-c3ccc(C4CC4)nc3)cc2)nc1Nc1ccc2c(c1)C1(CC1)NC2=O] | ¹H NMR (400 MHz, DMSO-d₆) δ 9.48 (br s, 1H), 8.77 (s, 1H), 8.44 (s, 1H), 8.04 (m, 3H), 7.81-7.76 (m, 3H), 7.63-7.58 (m, 2H), 7.40 (m, 1H), 4.26 (m, 2H), 2.18 (m, 1H), 1.56-1.25 (m, 7H), 0.97 (m, 4H) | 3.11 min, m/z = 463.3 [M + H]⁺ | Method G |
| 56 | 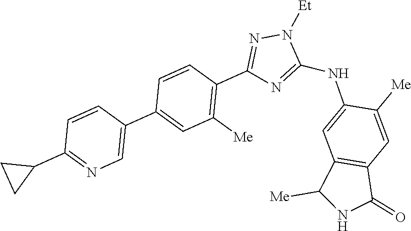 [SMILES: CCn1nc(-c2ccc(-c3ccc(C4CC4)nc3)cc2C)nc1Nc1cc2c(cc1C)C(=O)NC2C] | ¹H NMR (400 MHz, DMSO-d₆) δ 8.79 (d, J = 1.5 Hz, 1H), 8.39 (s, 1H), 8.22 (s, 1H), 8.02-7.98 (m, 2H), 7.68 (s, 1H), 7.65 (s, 1H), 7.60 (d, J = 8.0 Hz, 1H), 7.50 (s, 1H), 7.41 (d, J = 8.6 Hz, 1H), 4.59 (q, J = 6.6 Hz, 1H), 4.23 (q, J = 7.2 Hz, 2H), 2.73 (s, 3H), 2.41 (s, 3H), 2.22-2.15 (m, 1H), 1.45 (t, J = 7.5 Hz, 3H), 1.37 (d, J = 6.8 Hz, 3H), 1.04-0.97 (m, 4H) | 1.63 min, m/z = 479.3 [M + H]⁺ | Method D |
| 57 | 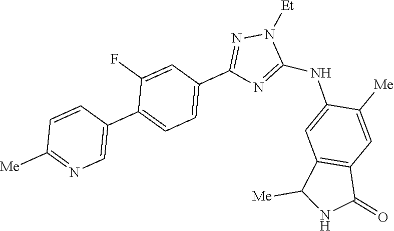 [SMILES: CCn1nc(-c2ccc(-c3ccc(C)nc3)c(F)c2)nc1Nc1cc2c(cc1C)C(=O)NC2C] | ¹H NMR (400 MHz, DMSO-d₆) δ 8.68 (s, 1H), 8.40 (s, 1H), 8.33 (s, 1H), 7.93 (d, J = 8.1 Hz, 1H), 7.88 (dd, J = 1.3, 8.0 Hz, 1H), 7.76 (dd, J = 1.1, 11.9 Hz, 1H), 7.68 (m, 1H), 7.59 (s, 1H), 7.50 (s, 1H), 7.40 (d, J = 8.6 Hz, 1H), 4.59 (q, J = 6.6 Hz, 1H), 4.20 (q, J = 7.2 Hz, 2H), 2.55 (s, 3H), 2.37 (s, 3H), 1.42 (t, J = 7.4 Hz, 3H), 1.36 (d, J = 6.4 Hz, 3H) | 2.26 min, m/z = 457.3 [M + H]⁺ | Method P |
| 58 | 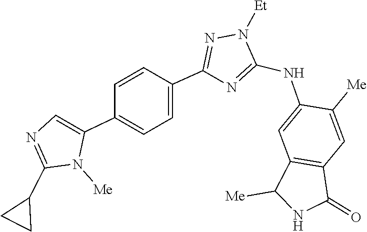 [SMILES: CCn1nc(-c2ccc(-c3cnc(C4CC4)n3C)cc2)nc1Nc1cc2c(cc1C)C(=O)NC2C] | ¹H NMR (400 MHz, DMSO-d₆) δ 8.38 (s, 1H), 8.22 (s, 1H), 7.88 (d, J = 8.3 Hz, 2H), 7.73 (d, J = 8.3 Hz, 2H), 7.53-7.51 ( m, 2H), 7.48 (s, 1H), 4.55 (q, J = 6.6 Hz, 1H), 4.15 (q, J = 6.9 Hz, 2H), 3.70 (s, 3H), 2.36 (s, 3H), 2.04-1.95 (m, 1H), 1.40 (t, J = 6.8 Hz, 3H), 1.35 (d, J = 6.6 Hz, 3H), 0.98-0.88 (m, 4H) | 2.56 min, m/z = 468.3 [M + H]⁺ | Method M |
| 59 | 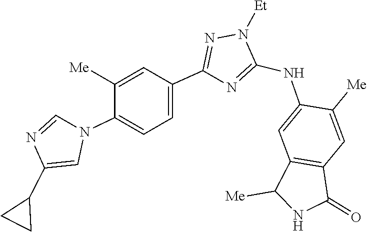 [SMILES: CCn1nc(-c2ccc(-n3cnc(C4CC4)c3)c(C)c2)nc1Nc1cc2c(cc1C)C(=O)NC2C] | ¹H NMR (400 MHz, DMSO-d₆) δ 8.41 (s, 1H), 8.30 (s, 1H), 7.94 (s, 1H), 7.86 (d, J = 7.7 Hz, 1H), 7.69 (s, 1H), 7.54 (s, 1H), 7.49 (s, 1H), 7.34 (d, J = 8.5 Hz, 1H), 7.18 (s, 1H), 4.56 (q, J = 6.2 Hz, 1H), 4.18 (q, J = 7.2 Hz, 2H), 2.37 (s, 3H), 2.25 (s, 3H), 1.90-1.82 (m, 1H), 1.42 (t, J = 6.7 Hz, 3H), 1.34 (d, J = 6.1 Hz, 3H), 0.85-0.78 (m, 2H), 0.75-0.69 (m, 2H) | 2.70 min, m/z = 468.3 [M + H]⁺ | Method M |
| 60 | 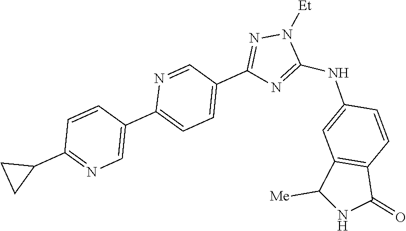 [SMILES: CCn1nc(-c2ccc(-c3ccc(C4CC4)nc3)nc2)nc1Nc1ccc2c(c1)C(C)NC2=O] | ¹H NMR (400 MHz, DMSO-d₆) δ 9.43 (s, 1H), 9.27 (d, J = 1.4 Hz, 1H), 9.16 (d, J = 1.4 Hz, 1H), 8.42-8.34 (m, 3H), 8.12 (d, J = 8.5 Hz, 1H), 8.00 (s, 1H), 7.75 (dd, J = 1.3, 8.2 Hz, 1H), 7.61 (d, J = 8.3 Hz, 1H), 7.45 (d, J = 8.3 Hz, 1H), 4.65 (q, J = 6.7 Hz, 1H), 4.29 (q, J = 7.0 Hz, 2H), 2.24-2.17 (m, 1H), 1.46-1.39 (m, 6H), 1.06-1.00 (m, 4H) | 1.21 min, m/z = 452.2 [M + H]⁺ | Method K |
| 61 | 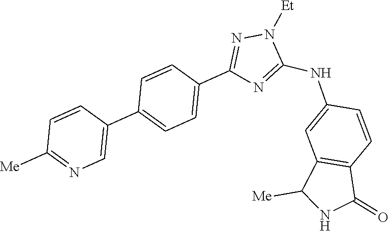 [SMILES: CCn1nc(-c2ccc(-c3ccc(C)nc3)cc2)nc1Nc1ccc2c(c1)C(C)NC2=O] | ¹H NMR (400 MHz, DMSO-d₆) δ 9.36 (s, 1H), 8.84 (d, J = 1.5 Hz, 1H), 8.38 (s, 1H), 8.12 (d, J = 8.3 Hz, 2H), 8.04 (dd, J = 2.1, 8.0 Hz, 1H), 7.95 (s, 1H), 7.83 (d, J= 8.1 Hz, 2H), 7.77 (d, J = 8.5 Hz, 1H), 7.61 (d, J = 8.4 Hz, 1H), 7.38 (d, J = 8.0 Hz, 1H), 4.64 (q, J = 6.6 Hz, 1H), 4.26 (q, J = 7.2 Hz, 2H), 2.55 (s, 3H), 1.46-1.40 (m, 6H) | 2.68 min, m/z = 425.3 [M + H]⁺ | Method G |
| 62 | 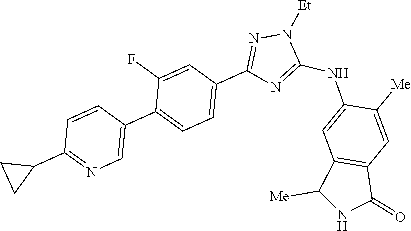 [SMILES: CCn1nc(-c2ccc(-c3ccc(C4CC4)nc3)c(F)c2)nc1Nc1cc2c(cc1C)C(=O)NC2C] | ¹H NMR (400 MHz, DMSO-d₆) δ 8.63 (s, 1H), 8.40 (s, 1H), 8.31 (s, 1H), 7.90-7.85 (m, 2H), 7.75 (dd, J = 0.9, 12.0 Hz, 1H), 7.67 (m, 1H), 7.58 (s, 1H), 7.50 (s, 1H), 7.43 (d, J = 8.1 Hz, 1H), 4.58 (q, J = 6.4 Hz, 1H), 4.20 (q, J= 7.2 Hz, 2H), 2.37 (s, 3H), 2.22-2.14 (m, 1H), 1.42 (t, J = 7.1 Hz, 3H), 1.35 (d, J = 6.5 Hz, 3H), 1.04-0.98 (m, 4H) | 3.46 min, m/z = 483.2 [M + H]⁺ | Method E |
| 63 | 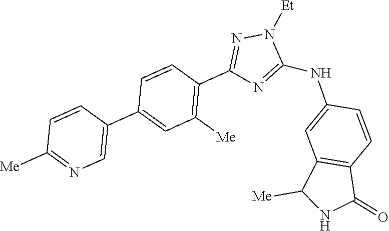 [SMILES: CCn1nc(-c2ccc(-c3ccc(C)nc3)cc2C)nc1Nc1ccc2c(c1)C(C)NC2=O] | ¹H NMR (400 MHz, DMSO-d₆) δ 9.38 (s, 1H), 8.82 (d, J = 1.8 Hz, 1H), 8.38 (s, 1H), 8.08-8.01 (m, 2H), 7.98 (s, 1H), 7.70-7.62 (m, 3H), 7.59 (d, J = 8.5 Hz, 1H), 7.37 (d, J= 7.9 Hz, 1H), 4.62 (q, J = 6.6 Hz, 1H), 4.25 (q, J = 7.1 Hz, 2H), 2.75 (s, 3H), 2.54 (s, 3H), 1.43 (t, J = 7.2 Hz, 3H), 1.38 (d, J = 6.7 Hz, 3H) | 2.77 min, m/z = 439.3 [M + H]⁺ | Method G |
| 64 | 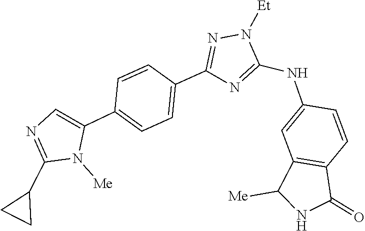 [SMILES: CCn1nc(-c2ccc(-c3cnc(C4CC4)n3C)cc2)nc1Nc1ccc2c(c1)C(C)NC2=O] | ¹H NMR (400 MHz, DMSO-d₆) δ 9.31 (s, 1H), 8.38 (s, 1H), 7.97-7.92 (m, 3H), 7.78-7.73 (m, 3H), 7.59 (d, J = 8.3 Hz, 1H), 7.55 (s, 1H), 4.64 (q, J = 6.6 Hz, 1H), 4.23 (q, J = 7.1 Hz, 2H), 3.72 (s, 3H), 2.05-1.98 (m, 1H), 1.43- 1.38 (m, 6H), 0.99-0.91 (m, 4H) | 0.77 min, m/z = 454.2 [M + H]⁺ | Method K |
| 65 | 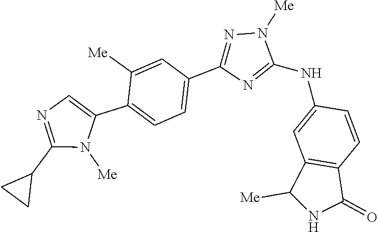 [SMILES: Cc1cc(-c2nc(Nc3ccc4c(c3)C(C)NC4=O)n(C)n2)ccc1-c1cnc(C2CC2)n1C] | ¹H NMR (400 MHz, DMSO-d₆) δ 9.33 (s, 1H), 8.41 (s, 1H), 7.97-7.92 (m, 2H), 7.87-7.82 (m, 2H), 7.76 (dd, J = 1.4, 8.3 Hz, 1H), 7.62 (d, J = 8.4 Hz, 1H), 7.38 (s, 1H), 4.68 (q, J = 6.7 Hz, 1H), 4.27 (q, J = 7.0 Hz, 2H), 3.78 (s, 3H), 2.53 (s, 3H), 2.10-2.02 (m, 1H), 1.47-1.41 (m, 6H), 1.02-0.93 (m, 4H) | 2.03 min, m/z = 468.3 [M + H]⁺ | Method P |
| 66 | 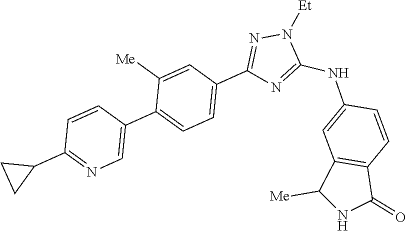 [SMILES: CCn1nc(-c2ccc(-c3ccc(C4CC4)nc3)c(C)c2)nc1Nc1ccc2c(c1)C(C)NC2=O] | ¹H NMR (400 MHz, DMSO-d₆) δ 9.36 (s, 1H), 8.44 (d, J = 1.5 Hz, 1H), 8.38 (s, 1H), 7.96-7.88 (m, 3H), 7.77 (dd, J= 1.4, 8.2 Hz, 1H), 7.71 (dd, J = 2.3, 8.0 Hz, 1H), 7.60 (d, J = 8.4 Hz, 1H), 7.40 (d, J = 8.0 Hz, 1H), 7.33 (d, J = 7.8 Hz, 1H), 4.64 (q, J = 6.7 Hz, 1H), 4.25 (q, J = 7.0 Hz, 2H), 2.34 (s, 3H), 2.22-2.14 (m, 1H), 1.45-1.39 (m, 6H), 1.03-0.98 (m, 4H) | 2.51 min, m/z = 465.3 [M + H]⁺ | Method N |
| 67 | 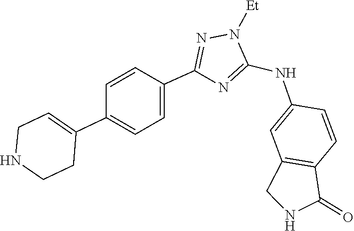 [SMILES: CCn1nc(-c2ccc(C3=CCNCC3)cc2)nc1Nc1ccc2c(c1)CNC2=O] | ¹H NMR (400 MHz, DMSO-d₆) δ 9.35 (s, 1H), 8.29 (s, 1H), 8.02-7.95 (m, 3H), 7.72 (d, J = 7.7 Hz, 1H), 7.62 (d, J = 8.0 Hz, 1H), 7.53 (d, J = 8.6 Hz, 2H), 6.33 (s, 1H), 4.39 (s, 2H), 4.24 (q, J = 7.1 Hz, 2H), 3.43-3.38 (br s, 4H), 2.99 (br s, 1H), 2.41 (br s, 1H), 1.41 (t, J = 6.7 Hz, 3H). | 2.36 min, m/z = 401.2 [M + H]⁺ | Method G |
| 68 | 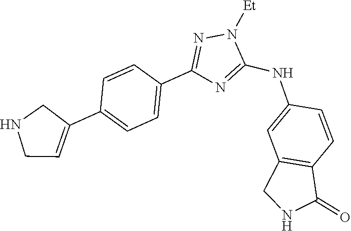 [SMILES: CCn1nc(-c2ccc(C3=CCNC3)cc2)nc1Nc1ccc2c(c1)CNC2=O] | ¹H NMR (400 MHz, DMSO-d₆) δ 9.32 (s, 1H), 8.28 (s, 1H), 8.01-7.96 (m, 3H), 7.69 (m, 1H), 7.62-7.56 (m, 2H), 7.52 (d, J = 8.3 Hz, 1H), 6.47 (s, 1H), 4.47 (s, 1H), 4.37 (s, 2H), 4.23 (m, 3H), 4.02 (s, 1H), 3.83 (s, 1H), 1.39 (t, J = 7.2 Hz, 3H). | 0.31 min, m/z = 387.3 [M + H]⁺ | Method D |
| 69 | 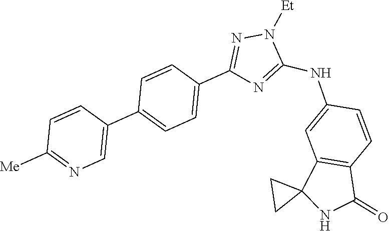 [SMILES: CCn1nc(-c2ccc(-c3ccc(C)nc3)cc2)nc1Nc1ccc2c(c1)C1(CC1)NC2=O] | ¹H NMR (400 MHz, DMSO-d₆) δ 9.37 (br s, 1H), 8.84 (d, J = 2.0 Hz, 1H), 8.45 (s, 1H), 8.09 (d, J = 8.3 Hz, 2H), 8.04 (dd, J = 2.4, 8.1 Hz, 1H), 7.83 (d, J = 8.3 Hz, 2H), 7.75 (dd, J = 1.7, 8.3 Hz, 1H), 7.64 (d, J = 8.3 Hz, 1H), 7.58 (d, J = 1.3 Hz, 1H), 7.38 (d, J = 8.1 Hz, 1H), 4.24 (q, J = 7.2 Hz, 2H), 2.54 (s, 3H), 1.54-1.49 (m, 2H), 1.41 (t, J = 7.3 Hz, 3H), 1.39-1.35 (m, 2H) | 1.08 min, m/z = 437.1 [M + H]⁺ | Method K |
| 70 | 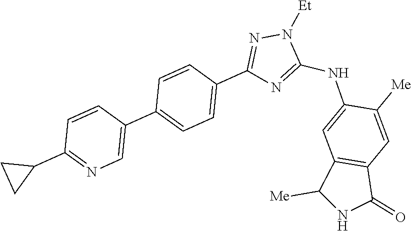 [SMILES: CCn1nc(-c2ccc(-c3ccc(C4CC4)nc3)cc2)nc1Nc1cc2c(cc1C)C(=O)NC2C] | ¹H NMR (400 MHz, DMSO-d₆) δ 8.77 (d, J = 2.1 Hz, 1H), 8.40 (s, 1H), 8.27 (s, 1H), 8.03 (d, J = 8.3 Hz, 2H), 7.99 (dd, J = 2.5, 8.2 Hz, 1H), 7.78 (d, J = 8.4 Hz, 2H), 7.57 (s, 1H), 7.49 (s, 1H), 7.41 (d, J = 8.0 Hz, 1H), 4.58 (q, J = 6.7 Hz, 1H), 4.18 (q, J = 7.3 Hz, 2H), 2.37 (s, 3H), 2.21-2.12 (m, 1H), 1.42 (t, J = 7.2 Hz, 3H), 1.36 (m, 3H), 1.02-0.96 (m, 4H) | 1.38 min, m/z = 465.1 [M + H]⁺ | Method K |
| 71 | 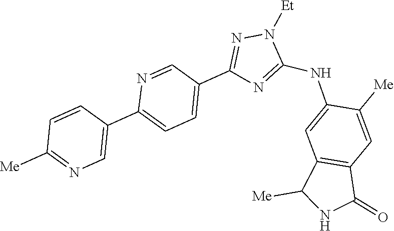 [SMILES: CCn1nc(-c2ccc(-c3ccc(C)nc3)nc2)nc1Nc1cc2c(cc1C)C(=O)NC2C] | ¹H NMR (400 MHz, DMSO-d₆) δ 9.19 (m, 2H), 8.42-8.38 (m, 1H), 8.34 (m, 3H), 8.11 (d, J = 8.4 Hz, 1H), 7.62 (s, 1H), 7.50 (s, 1H), 7.40 (d, J = 7.8 Hz, 1H), 4.58 (q, J = 6.9 Hz, 1H), 4.21 (q, J = 7.2 Hz, 2H), 2.55 (s, 3H), 2.38 (s, 3H), 1.43 (t, J = 7.2 Hz, 3H), 1.36 (d, J = 6.7 Hz, 3H) | 2.01 min, m/z = 440.3 [M + H]⁺ | Method P |
| 72 | 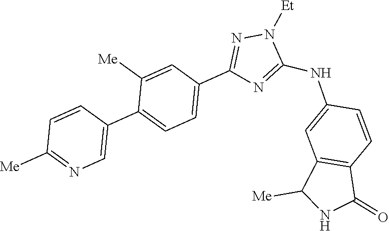 [SMILES: CCn1nc(-c2ccc(-c3ccc(C)nc3)c(C)c2)nc1Nc1ccc2c(c1)C(C)NC2=O] | ¹H NMR (400 MHz, DMSO-d₆) δ 9.34 (s, 1H), 8.50 (d, J =1.9 Hz, 1H), 8.39 (s, 1H), 7.97 (s, 1H), 7.94-7.90 (m, 2H), 7.79-7.73 (m, 2H), 7.61 (d, J = 8.3 Hz, 1H), 7.37 (m, 2H), 4.64 (q, J = 6.5 Hz, 1H), 4.27 (q, J = 7.0 Hz, 2H), 2.56 (s, 3H), 2.34 (s, 3H), 1.46-1.40 (m, 6H) | 2.10 min, m/z = 439.2 [M + H]⁺ | Method D |
| 73 | 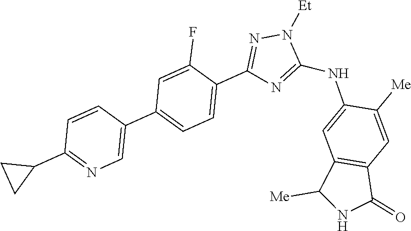 [SMILES: CCn1nc(-c2ccc(-c3ccc(C4CC4)nc3)cc2F)nc1Nc1cc2c(cc1C)C(=O)NC2C] | ¹H NMR (400 MHz, DMSO-d₆) δ 8.84 (s, 1H), 8.39 (s, 1H), 8.30 (s, 1H), 8.09-8.01 (m, 2H), 7.72 (d, J = 12.4 Hz, 1H), 7.66 (d, J = 8.1 Hz, 1H), 7.61 (s, 1H), 7.50 (s, 1H), 7.42 (d, J = 8.1 Hz, 1H), 4.62-4.53 (m, 1H), 4.27-4.16 (m, 2H), 2.40 (s, 3H), 2.24-2.14 (m, 1H), 1.43 (t, J = 7.1 Hz, 3H), 1.37 (d, J = 5.4 Hz, 3H), 1.06-0.96 (m, 4H) | 1.58 min, m/z = 483.3 [M + H]⁺ | Method D |
| 74 | 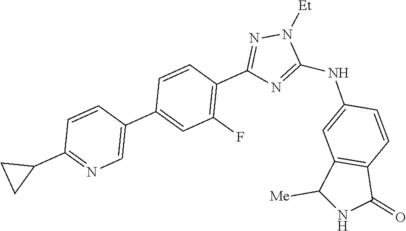 [SMILES: CCn1nc(-c2ccc(-c3ccc(C4CC4)nc3)cc2F)nc1Nc1ccc2c(c1)C(C)NC2=O] | ¹H NMR (400 MHz, DMSO-d₆) δ 9.41 (s, 1H), 8.84 (d, J = 2.0 Hz, 1H), 8.40 (s, 1H), 8.12 (m, 1H), 8.07 (dd, J = 2.5, 8.1 Hz, 1H), 7.98 (s, 1H), 7.76-7.68 (m, 3H), 7.59 (d, J = 8.3 Hz, 1H), 7.43 (d, J = 8.1 Hz, 1H), 4.64 (q, J = 6.7 Hz, 1H), 4.28 (q, J = 7.2 Hz, 2H), 2.23-2.16 (m, 1H), 1.44-1.38 (m, 6H), 1.04-0.98 (m, 4H) | 1.45 min, m/z = 469.1 [M + H]⁺ | Method K |
| 75 | 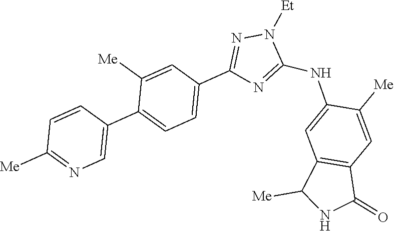 [SMILES: CCn1nc(-c2ccc(-c3ccc(C)nc3)c(C)c2)nc1Nc1cc2c(cc1C)C(=O)NC2C] | ¹H NMR (400 MHz, DMSO-d₆) δ 8.47 (d, J = 1.8 Hz, 1H), 8.38 (s, 1H), 8.26 (s, 1H), 7.90 (s, 1H), 7.84 (d, J = 7.8 Hz, 1H), 7.73 (dd, J = 2.2, 7.8 Hz, 1H), 7.54 (s, 1H), 7.49 (s, 1H), 7.36 (d, J = 8.1 Hz, 1H), 7.31 (d, J = 8.0 Hz, 1H), 4.58 (q, J = 6.7 Hz, 1H), 4.18 (q, J = 7.1 Hz, 2H), 2.55 (s, 3H), 2.37 (s, 3H), 2.31 (s, 3H), 1.42 (t, J = 7.2 Hz, 3H), 1.35 (d, J = 6.9 Hz, 3H) | 1.36 min, m/z = 453.3 [M + H]⁺ | Method N |
| 76 | 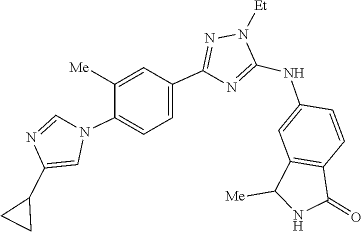 [SMILES: CCn1nc(-c2ccc(-n3cnc(C4CC4)c3)c(C)c2)nc1Nc1ccc2c(c1)C(C)NC2=O] | ¹H NMR (400 MHz, DMSO-d₆) δ 9.37 (s, 1H), 8.40 (s, 1H), 8.01 (s, 1H), 7.95-7.90 (m, 2H), 7.77 (dd, J = 1.4, 8.4 Hz, 1H), 7.69 (s, 1H), 7.60 (d, J = 8.2 Hz, 1H), 7.38 (d, J = 8.0 Hz, 1H), 7.19 (s, 1H), 4.64 (q, J = 6.6 Hz, 1H), 4.25 (q, J = 7.2 Hz, 2H), 2.27 (s, 3H), 1.91-1.83 (m, 1H), 1.44-1.38 (m, 6H), 0.85-0.79 (m, 2H), 0.75-0.70 (m, 2H) | 2.03 min, m/z = 454.2 [M + H]⁺ | Method S |
| 77 | 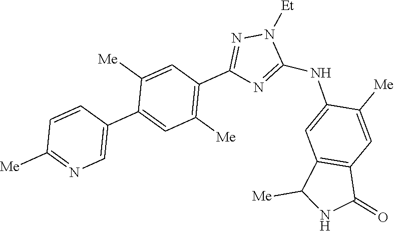 [SMILES: CCn1nc(-c2cc(C)c(-c3ccc(C)nc3)cc2C)nc1Nc1cc2c(cc1C)C(=O)NC2C] | ¹H NMR (400 MHz, DMSO-d₆) δ 8.47 (s, 1H), 8.39 (s, 1H), 8.20 (s, 1H), 7.84 (s, 1H), 7.72 (d, J = 7.9 Hz, 1H), 7.66 (s, 1H), 7.48 (s, 1H), 7.35 (d, J = 8.0 Hz, 1H), 7.16 (s, 1H), 4.60-4.52 (m, 1H), 4.21 (q, J = 7.2 Hz, 2H), 2.64 (s, 3H), 2.55 (s, 3H), 2.39 (s, 3H), 2.26 (s, 3H), 1.44 (t, J = 7.0 Hz, 3H), 1.34 (d, J = 6.3 Hz, 3H) | 2.81 min, m/z = 467.3 [M + H]⁺ | Method E |
| 78 | 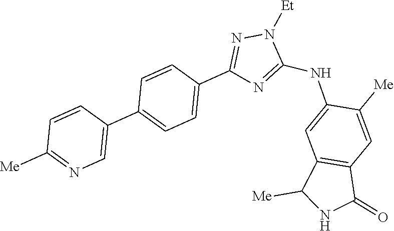 [SMILES: CCn1nc(-c2ccc(-c3ccc(C)nc3)cc2)nc1Nc1cc2c(cc1C)C(=O)NC2C] | ¹H NMR (400 MHz, DMSO-d₆) δ 8.67 (d, J = 1.8 Hz, 1H), 8.25 (s, 1H), 8.13 (s, 1H), 7.91-7.86 (m, 3H), 7.66 (d, J = 8.2 Hz, 2H), 7.43 (s, 1H), 7.34 (s, 1H), 7.22 (d, J = 8.2 Hz, 1H), 4.43 (q, J = 6.4 Hz, 1H), 4.03 (q, J = 7.3 Hz, 2H), 2.39 (s, 3H), 2.22 (s, 3H), 1.27 (t, J = 7.4 Hz, 3H), 1.20 (d, J = 6.3 Hz, 3H) | 2.06 min, m/z = 439.2 [M + H]⁺ 12.60 min | Method L SFC A |
| | Enantiomer 2 | | | |
| 79 | 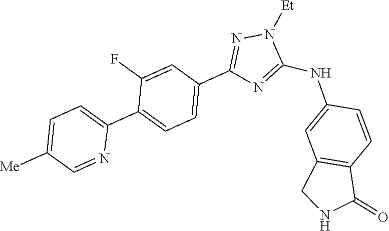 [SMILES: CCn1nc(-c2ccc(-c3ccc(C)cn3)c(F)c2)nc1Nc1ccc2c(c1)CNC2=O] | ¹H NMR (400 MHz, DMSO-d₆) δ 9.39 (s, 1H), 8.60 (s, 1H), 8.29 (s, 1H), 8.08 (m, 1H), 8.02 (s, 1H), 7.96 (d, J = 8.5 Hz, 1H), 7.85 (d, J = 12.1 Hz, 1H), 7.78 (s, 2H), 7.72 (d, J = 8.1 Hz, 1H), 7.64 (d, J = 7.9 Hz, 1H), 4.40 (s, 2H), 4.28 (q, J = 7.0 Hz, 2H), 2.39 (s, 3H), 1.42 (t, J = 7.0 Hz, 3H) | 1.65 min, m/z = 429.2 [M + H]⁺ | Method D |
| 80 | 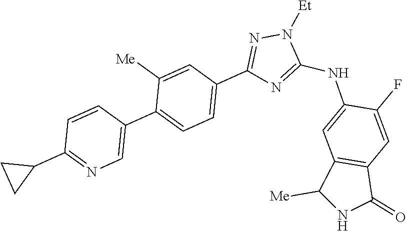 [SMILES: CCn1nc(-c2ccc(-c3ccc(C4CC4)nc3)c(C)c2)nc1Nc1cc2c(cc1F)C(=O)NC2C] | ¹H NMR (400 MHz, DMSO-d₆) 9.02 (s, 1H), 8.58 (s, 1H), 8.43 (d, J = 1.8 Hz, 1H), 8.01 (d, J = 7.0 Hz, 1H), 7.92 (s, 1H), 7.87 (d, J = 7.9 Hz, 1H), 7.70 (dd, J = 2.3, 8.1 Hz, 1H), 7.47 (d, J = 10.2 Hz, 1H), 7.39 (d, J = 8.0 Hz, 1H), 7.32 (d, J = 8.3 Hz, 1H), 4.63 (q, J = 6.7 Hz, 1H), 4.24 (q, J = 7.2 Hz, 2H), 2.32 (s, 3H), 2.21-2.14 (m, 1H), 1.42 (t, J = 7.3 Hz, 3H), 1.38 (d, J = 6.7 Hz, 3H), 1.03-0.97 (m, 4H) | 3.19 min, m/z = 483.3 [M + H]⁺ | Method M |
| 81 | 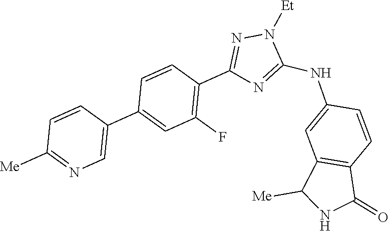 [SMILES: CCn1nc(-c2ccc(-c3ccc(C)nc3)cc2F)nc1Nc1ccc2c(c1)C(C)NC2=O] | ¹H NMR (400 MHz, DMSO-d₆) δ 9.40 (s, 1H), 8.89 (d, J = 2.3 Hz, 1H), 8.40 (s, 1H), 8.16-8.08 (m, 2H), 7.98 (s, 1H), 7.78-7.69 ((m, 3H), 7.59 (d, J = 8.5 Hz, 1H), 7.39 (d, J = 8.1 Hz, 1H), 4.63 (q, J = 6.7 Hz, 1H), 4.27 (q, J = 7.0 Hz, 2H), 2.55 (s, 3H), 1.44-1.38 (m, 6H) | 1.76 min, m/z = 443.2 [M + H]⁺ | Method D |
| 82 | 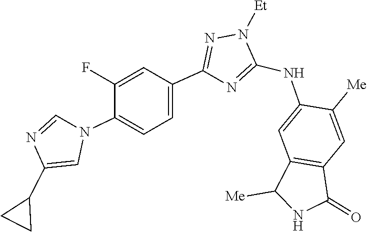 [SMILES: CCn1nc(-c2ccc(-n3cnc(C4CC4)c3)c(F)c2)nc1Nc1cc2c(cc1C)C(=O)NC2C] | ¹H NMR (400 MHz, DMSO-d₆) δ 8.37 (s, 1H), 8.30 (s, 1H), 7.87 (s, 1H), 7.83-7.77 (m, 2H), 7.65 (m, 1H), 7.53 (s, 1H), 7.45 (s, 1H), 7.32 (s, 1H), 4.54 (q, J = 6.5 Hz, 1H), 4.15 (q, J = 7.1 Hz, 2H), 2.32 (s, 3H), 1.88-1.80 (m, 1H), 1.37 (t, J = 7.2 Hz, 3H), 1.30 (d, J = 6.5 Hz, 3H), 0.81-0.76 (m, 2H), 0.71-0.65 (m, 2H) | 2.77 min, m/z = 472.3 [M + H]⁺ | Method E |
| 83 | 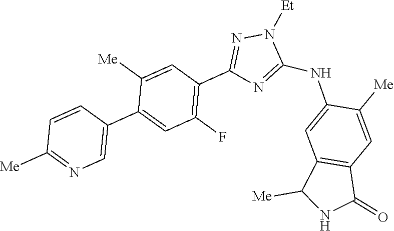 [SMILES: CCn1nc(-c2cc(C)c(-c3ccc(C)nc3)cc2F)nc1Nc1cc2c(cc1C)C(=O)NC2C] | 1H NMR (400 MHz, DMSO-d6) d 8.51 (s, 1H), 8.39 (s, 1H), 8.30 (s, 1H), 7.87 (d, J = 7.3 Hz, 1H), 7.77 (d, J = 7.4 Hz, 1H), 7.57 (s, 1H), 7.49 (s, 1H), 7.37 (d, J = 7.8 Hz, 1H), 7.22 (d, J = 11.2 Hz, 1H), 4.61-4.53 (m, 1H), 4.25- 4.16 (m, 2H), 2.56 (s, 3H), 2.38 (s, 3H), 2.28 (s, 3H), 1.43 (t, J = 6.7 Hz, 3H), 1.35 (d, J = 6.7 Hz, 3H) | 2.83 min, m/z = 471.3 [M + H]⁺ | Method G |
| 84 | 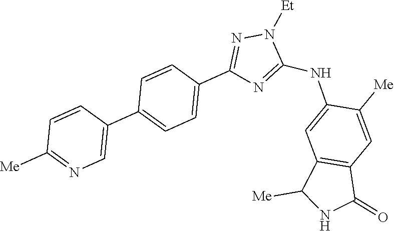 [SMILES: CCn1nc(-c2ccc(-c3ccc(C)nc3)cc2)nc1Nc1cc2c(cc1C)C(=O)NC2C] | ¹H NMR (400 MHz, DMSO-d₆) δ 8.83 (d, J = 1.9 Hz, 1H), 8.40 (s, 1H), 8.28 (s, 1H), 8.07-8.01 (m, 3H), 7.81 (d, J = 8.3 Hz, 2H), 7.58 (s, 1H), 7.50 (s, 1H), 7.38 (d, J = 8.1 Hz, 1H), 4.58 (q, J = 6.7 Hz, 1H), 4.19 (q, J = 7.2 Hz, 2H), 2.54 (s, 3H), 2.38 (s, 3H), 1.43 (t, J = 7.1 Hz, 3H), 1.35 (d, J = 6.7 Hz, 3H | 2.11 min, m/z = 439.3 [M + H]⁺ 10.31 min | Method N SFC A |
| | Enantiomer 1 | | | |
| 85 | 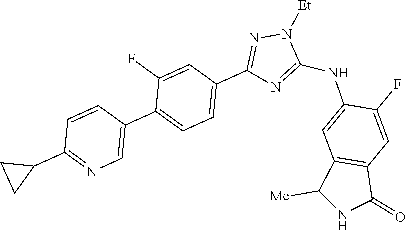 [SMILES: CCn1nc(-c2ccc(-c3ccc(C4CC4)nc3)c(F)c2)nc1Nc1cc2c(cc1F)C(=O)NC2C] | ¹H NMR (400 MHz, DMSO-d₆) δ 9.09 (s, 1H), 8.65 (s, 1H), 8.61 (s, 1H), 8.05 (d, J = 6.7 Hz, 1H), 7.92-7.88 (m, 2H), 7.80 (d, J = 11.8 Hz, 1H), 7.70 (m, 1H), 7.47 (m, 2H), 4.66 (q, J = 6.7 Hz, 1H), 4.27 (q, J = 7.1 Hz, 2H), 2.23-2.17 (m, 1H), 1.44 (t, J = 7.2 Hz, 3H), 1.39 (d, J = 6.5 Hz, 3H), 1.05- 0.99 (m, 4H) | 3.52 min, m/z = 487.2 [M + H]⁺ | Method M |
| 86 | 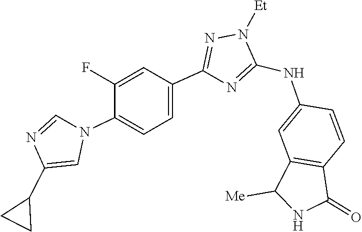 [SMILES: CCn1nc(-c2ccc(-n3cnc(C4CC4)c3)c(F)c2)nc1Nc1ccc2c(c1)C(C)NC2=O] | ¹H NMR (400 MHz, DMSO-d₆) δ 9.40 (s, 1H), 8.39 (s, 1H), 7.96-7.90 (m, 4H), 7.77-7.68 (m, 2H), 7.58 (d, J = 8.3 Hz, 1H), 7.38 (s, 1H), 4.62 (q, J = 6.7 Hz, 1H), 4.24 (q, J = 7.2 Hz, 2H), 1.94-1.85 (m, 1H), 1.43-1.39 (m, 6H), 0.87-0.82 (m, 2H), 0.77-0.72 (m, 2H) | 1.12 min, m/z = 458.3 [M + H]⁺ | Method D |
| 87 | 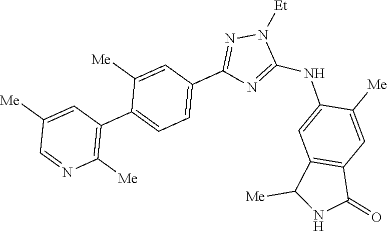 [SMILES: CCn1nc(-c2ccc(-c3cc(C)cnc3C)c(C)c2)nc1Nc1cc2c(cc1C)C(=O)NC2C] | ¹H NMR (400 MHz, DMSO-d₆) δ 8.37 (s, 1H), 8.31 (d, J = 2.2 Hz, 1H), 8.25 (s, 1H), 7.88 (s, 1H), 7.83 (d, J = 7.9 Hz, 1H), 7.52 (s, 1H), 7.48 (s, 1H), 7.33 (d, J = 2.2 Hz, 1H), 7.15 (d, J = 7.9 Hz, 1H), 4.55 (q, J = 6.7 Hz, 1H), 4.16 (q, J = 7.2 Hz, 2H), 2.35 (s, 3H), 2.30 (s, 3H), 2.16 (s, 3H), 2.06 (s, 3H), 1.39 (t, J = 7.2 Hz, 3H), 1.32 (d, J = 6.7 Hz, 3H) | 1.07 min, m/z = 467.1 [M + H]⁺ | Method K |
| 88 | 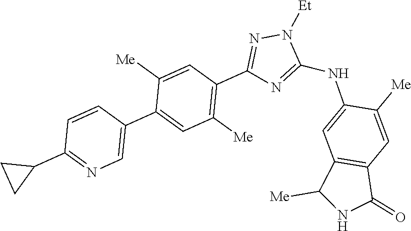 [SMILES: CCn1nc(-c2cc(C)c(-c3ccc(C4CC4)nc3)cc2C)nc1Nc1cc2c(cc1C)C(=O)NC2C] | ¹H NMR (400 MHz, DMSO-d₆) δ 8.40 (d, J = 2.2 Hz, 1H), 8.35 (s, 1H), 8.17 (s, 1H), 7.80 (s, 1H), 7.68 (dd, J = 2.0, 8.5 Hz, 1H), 7.65 (s, 1H), 7.46 (s, 1H), 7.36 (d, J = 8.0 Hz, 1H), 7.13 (s, 1H), 4.54 (q, J = 6.6 Hz, 1H), 4.18 (q, J = 7.2 Hz, 2H), 2.60 (s, 3H), 2.37 (s, 3H), 2.23 (s, 3H), 2.18-2.11 (m, 1H), 1.41 (t, J = 7.2 Hz, 3H), 1.32 (d, J = 6.6 Hz, 3H), 1.01-0.97 (m, 4H) | 1.70 min, m/z = 493.3 [M + H]⁺ | Method D |
| 89 | 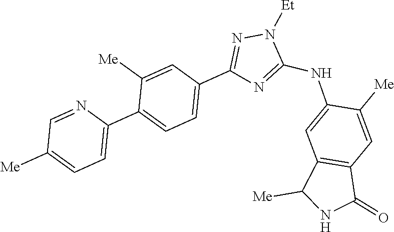 [SMILES: CCn1nc(-c2ccc(-c3ccc(C)cn3)c(C)c2)nc1Nc1cc2c(cc1C)C(=O)NC2C] | ¹H NMR (400 MHz, DMSO-d₆) δ 8.55 (s, 1H), 8.42 (s, 1H), 8.30 (s, 1H), 7.89 (s, 1H), 7.86 (d, J = 8.0 Hz, 1H), 7.74 (d, J = 8.0 Hz, 1H), 7.55 (s, 1H), 7.52 (s, 1H), 7.49 (m, 2H), 4.60 (q, J = 6.7 Hz, 1H), 4.20 (q, J = 7.3 Hz, 2H), 2.42 (s, 3H), 2.39 (m, 6H), 1.44 (t, J = 7.3 Hz, 3H), 1.37 (d, J = 6.5 Hz, 3H) | 2.86 min, m/z = 453.3 [M + H]⁺ | Method M |
| 90 | 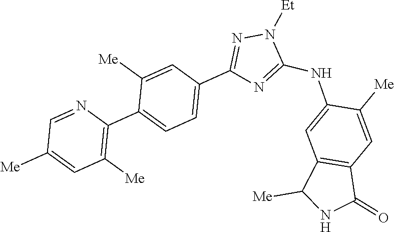 [SMILES: CCn1nc(-c2ccc(-c3ncc(C)cc3C)c(C)c2)nc1Nc1cc2c(cc1C)C(=O)NC2C] | ¹H NMR (400 MHz, DMSO-d₆) δ 8.43 (s, 1H), 8.35 (s, 1H), 8.30 (s, 1H), 7.91 (s, 1H), 7.85 (d, J = 8.6 Hz, 1H), 7.61-7.55 (m, 2H), 7.53 (s, 1H), 7.22 (d, J = 8.3 Hz, 1H), 4.60 (q, J = 6.8 Hz, 1H), 4.22 (q, J = 6.9 Hz, 2H), 3.40 (s, 3H), 2.40 (s, 3H), 2.10-2.08 (m, 6H), 1.45 (t, J = 7.3 Hz, 3H), 1.37 (d, J = 7.0 Hz, 3H) | 2.79 min, m/z = 467.3 [M + H]⁺ | Method M |
| 91 | 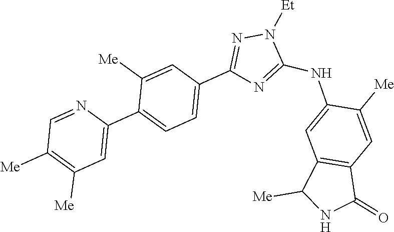 [SMILES: CCn1nc(-c2ccc(-c3cc(C)c(C)cn3)c(C)c2)nc1Nc1cc2c(cc1C)C(=O)NC2C] | ¹H NMR (400 MHz, DMSO-d₆) δ 8.42-8.39 (m, 2H), 8.28 (s, 1H), 7.87 (s, 1H), 7.83 (d, J = 8.0 Hz, 1H), 7.53 (s, 1H), 7.50 (s, 1H), 7.45 (d, J = 8.0 Hz, 1H), 7.37 (s, 1H), 4.58 (q, J = 6.6 Hz, 1H), 4.19 (q, J = 7.1 Hz, 2H), 2.40 (s, 3H), 2.37 (s, 3H), 2.33 (s, 3H), 2.29 (s, 3H), 1.43 (t, J = 7.1 Hz, 3H), 1.35 (d, J = 6.6 Hz, 3H) | 2.23 min, m/z = 467.3 [M + H]⁺ | Method N |
| 92 | 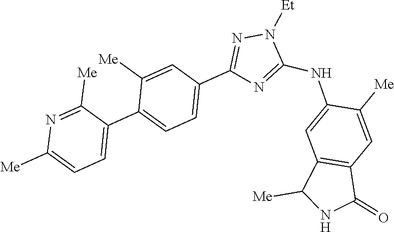 [SMILES: CCn1nc(-c2ccc(-c3ccc(C)nc3C)c(C)c2)nc1Nc1cc2c(cc1C)C(=O)NC2C] | ¹H NMR (400 MHz, DMSO-d₆) δ 8.41 (s, 1H), 8.27 (s, 1H), 7.89 (s, 1H), 7.82 (d, J = 7.4 Hz, 1H), 7.52 (s, 1H), 7.49 (s, 1H), 7.42 (d, J = 8.0 Hz, 1H), 7.20-7.14 (m, 2H), 4.58 (q, J = 6.3 Hz, 1H), 4.19 (q, J = 7.1 Hz, 2H), 2.50 (s, 3H), 2.37 (s, 3H), 2.20 (s, 3H), 2.07 (s, 3H), 1.41 (t, J = 7.3 Hz, 3H), 1.34 (d, J = 6.6 Hz, 3H) | 2.62 min, m/z = 467.3 [M + H]⁺ | Method M |
| 93 | 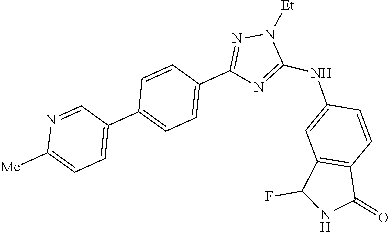 [SMILES: CCn1nc(-c2ccc(-c3ccc(C)nc3)cc2)nc1Nc1ccc2c(c1)C(F)NC2=O] | ¹H NMR (400 MHz, DMSO-d₆) δ 9.11 (s, 1H), 8.80 (d, J = 2.0 Hz, 1H), 8.49 (s, 1H), 8.06 (d, J = 8.4 Hz, 2H), 8.03-8.00 (m, 2H), 7.79 (d, J = 8.4 Hz, 2H), 7.48 (d, J = 10.0 Hz, 1H), 7.36 (d, J = 8.0 Hz, 1H), 4.35 (s, 2H), 4.27-4.22 (q, J = 7.2 Hz, 2H), 2.52 (s, 3H), 1.40 (t, J = 14.4 Hz, 3H) | 2.51 min, m/z = 429.1 [M + H]⁺ | Method M |
| 94 | 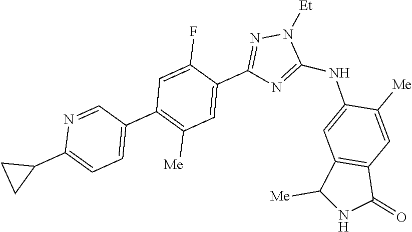 [SMILES: CCn1nc(-c2cc(C)c(-c3ccc(C4CC4)nc3)cc2F)nc1Nc1cc2c(cc1C)C(=O)NC2C] | ¹H NMR (400 MHz, DMSO-d₆) δ 8.43 (s, 1H), 8.41 s, 1H) (), 8.30 (s, 1H), 7.83 (d, J = 7.5 Hz, 1H), 7.70 (d, J = 8.0 Hz, 1H), 7.60-7.58 (m, 1H), 7.47 (s, 1H), 7.38 (d, J = 8.1 Hz, 1H), 7.19 (d, J = 11.3 Hz, 1H), 4.54 (q, J = 6.8 Hz, 1H), 4.18 (q, J = 7.4 Hz, 2H), 2.35 (s, 3H), 2.24 (s, 3H), 2.22-2.13 (m, 1H), 1.39 (t, J = 7.2 Hz, 3H), 1.32 (d, J = 6.6 Hz, 3H), 1.05-0.94 (m, 4H) | 1.66 min, m/z = 497.3 [M + H]⁺ | Method D |
| 95 | 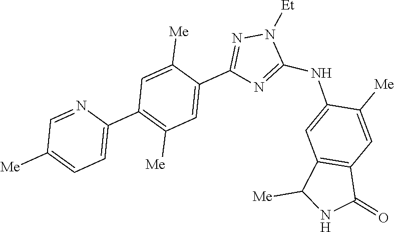 [SMILES: CCn1nc(-c2cc(C)c(-c3ccc(C)cn3)cc2C)nc1Nc1cc2c(cc1C)C(=O)NC2C] | ¹H NMR (400 MHz, DMSO-d₆) δ 8.50 (s, 1H), 8.36 (s, 1H), 8.31 (s, 1H), 7.77 (s, 1H), 7.69 (d, J = 8.0 Hz, 1H), 7.64 (s, 1H), 7.50-7.46 (m, 2H), 7.28 (s, 1H), 4.55 (q, J = 6.7 Hz, 1H), 4.23 (q, J = 7.1 Hz, 2H), 2.60 (s, 3H), 2.40 (s, 3H), 2.36 (s, 3H), 2.31 (s, 3H), 1.44 (t, J = 7.1Hz, 3H), 1.32 (d, J = 6.6 Hz, 3H) | 2.39 min, m/z = 467.3 [M + H]⁺ | Method N |
| 96 | 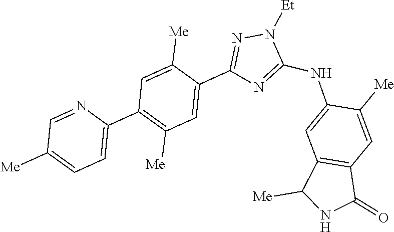 [SMILES: CCn1nc(-c2cc(C)c(-c3ccc(C)cn3)cc2C)nc1Nc1cc2c(cc1C)C(=O)NC2C] | ¹H NMR (400 MHz, DMSO-d₆) δ 9.40 (s, 1H), 8.50 (d, J = 1.5 Hz, 1H), 8.41 (s, 1H), 8.05 (s, 1H), 7.91 (s, 1H), 7.76 (dd, J = 2.2, 8.1 Hz, 1H), 7.68 (dd, J = 0.8, 8.2 Hz, 1H), 7.60 (d, J = 8.2 Hz, 1H), 7.38 (d, J = 8.0 Hz, 1H), 7.20 (s, 1H), 4.64 (q, J = 6.7 Hz, 1H), 4.28 (q, J = 7.2 Hz, 2H), 2.69 (s, 3H), 2.58 (s, 3H), 2.31 (s, 3H), 1.45 (t, J = 7.2 Hz, 3H), 1.41 (d, J = 6.5 Hz, 3H) | 2.77 min, m/z = 453.3 [M + H]⁺ | Method M |
| 97 | 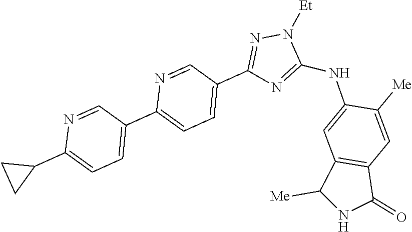 [SMILES: CCn1nc(-c2ccc(-c3ccc(C4CC4)nc3)nc2)nc1Nc1cc2c(cc1C)C(=O)NC2C] | ¹H NMR (400 MHz, DMSO-d₆) δ 9.17 (d, J = 1.7 Hz, 1H), 9.12 (d, J = 1.8 Hz, 1H), 8.39 (s, 1H), 8.34-8.29 (m, 3H), 8.07 (d, J = 8,2 Hz, 1H), 7.60 (s, 1H), 7.48 (s, 1H), 7.41 (d, J = 8.5 Hz, 1H), 4.56 (q, J = 6.6 Hz, 1H), 4.19 (q, J = 7.2 Hz, 2H), 2.36 (s, 3H), 2.19-2.15 (m, 1H), 1.43 (t, J = 7.1 Hz, 3H), 1.34 (d, J = 6.6 Hz, 3H), 1.02-0.98 (m, 4H) | 2.96 min, m/z = 466.2 [M + H]⁺ | Method M |
| 98 | 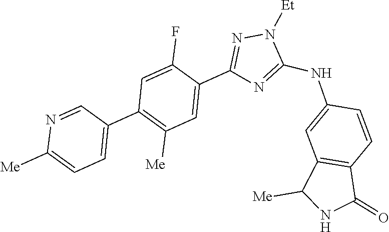 [SMILES: CCn1nc(-c2cc(C)c(-c3ccc(C)nc3)cc2F)nc1Nc1ccc2c(c1)C(C)NC2=O] | ¹H NMR (400 MHz, DMSO-d₆) δ 9.40 (s, 1H), 8.52 (d, J = 1.8 Hz, 1H), 8.39 (s, 1H), 7.97 (s, 1H), 7.94 (d, J = 8.0 Hz, 1H), 7.79 (dd, J = 2.1, 7.9 Hz, 1H), 7.72 (dd, J = 1.3, 8.5 Hz, 1H), 7.59 (d, J = 8.1 Hz, 1H), 7.39 (d, J = 7.5 Hz, 1H), 7.24 (d, J = 11.7 Hz, 1H), 4.64 (q, J = 6.4 Hz, 1H), 4.28 (q, J = 7 .0 Hz, 2H), 2.56 (s, 3H), 2.30 (s, 3H), 1.44-1.38 (m, 6H) | 1.56 min, m/z = 457.2 [M + H]⁺ | Method K |
| 99 | 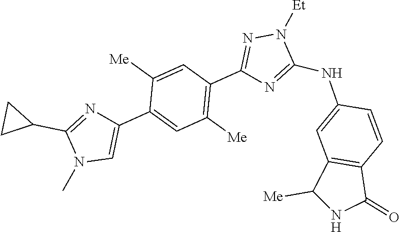 [SMILES: CCn1nc(-c2cc(C)c(-c3cn(C)c(C4CC4)n3)cc2C)nc1Nc1ccc2c(c1)C(C)NC2=O] | ¹H NMR (400 MHz, DMSO-d₆) δ 9.31 (s, 1H), 8.35 (s, 1H), 7.98 (s, 1H), 7.74 (s, 1H), 7.70 (s, 1H), 7.63 (dd, J = 8.3, 1.9 Hz, 1H), 7.55 (d, J = 8.3 Hz, 1H), 7.30 (s, 1H), 4.62 (q, J = 6.6 Hz, 1H), 4.24 (q, J = 7.2 Hz, 2H), 3.72 (s, 3H), 2.61 (s, 3H), 2.42 (s, 3H), 2.03-1.96 (m, 1H), 1.44-1.29 (m, 6H), 0.99- 0.83 (m, 4H) | 2.11 min, m/z = 482.3 [M + H]⁺ | Method N |
| 100 | 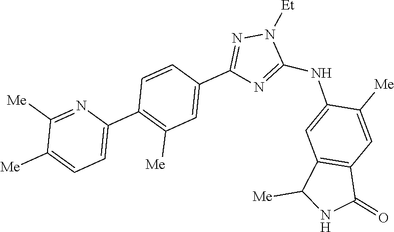 [SMILES: CCn1nc(-c2ccc(-c3ccc(C)c(C)n3)c(C)c2)nc1Nc1cc2c(cc1C)C(=O)NC2C] | ¹H NMR (400 MHz, DMSO-d₆) δ 8.37 (s, 1H), 8.25 (s, 1H), 7.83 (s, 1H), 7.81 (d, J = 8.1 Hz, 1H), 7.59 (d, J = 7.8 Hz, 1H), 7.54 (s, 1H), 7.50 (s, 1H), 7.42 (d, J = 7.9 Hz, 1H), 7.27 (d, J = 7.8 Hz, 1H), 4.59 (q, J = 7.1Hz, 1H), 4.19 (q, J = 7.4 Hz, 2H), 2.47 (s, 3H), 2.41 (s, 3H), 2.38 (s, 3H), 2.30 (s, 3H), 1.40 (t, J = 7.2 Hz, 3H), 1.33 (d, J = 6.6 Hz, 3H) | 2.15 min, m/z = 467.4 [M + H]⁺ | Method N |
| 101 | 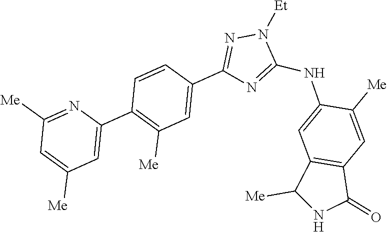 [SMILES: CCn1nc(-c2ccc(-c3cc(C)cc(C)n3)c(C)c2)nc1Nc1cc2c(cc1C)C(=O)NC2C] | ¹H NMR (400 MHz, DMSO-d₆) δ 8.37 (s, 1H), 8.28 (s, 1H), 7.86 (s, 1H), 7.83 (d, J = 8.2 Hz, 1H), 7.55-7.52 (m, 1H), 7.47 (s, 1H), 7.41 (d, J = 7.9 Hz, 1H), 7.16 (s, 1H), 7.06 (s, 1H), 4.55 (q, J = 6.7 Hz, 1H), 4.16 (q, J = 7.2 Hz, 2H), 2.47 (s, 3H), 2.36 (s, 3H), 2.35 (s, 3H), 2.33 (s, 3H), 1.40 (t, J = 7.2 Hz, 3H), 1.33 (d, J = 6.6 Hz, 3H) | 1.19 min, m/z = 467.3 [M + H]⁺ | Method D |
| 102 | 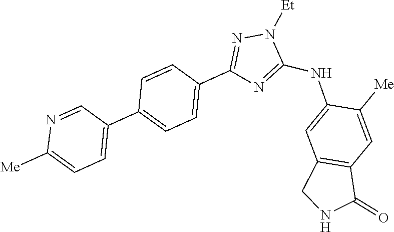 [SMILES: CCn1nc(-c2ccc(-c3ccc(C)nc3)cc2)nc1Nc1cc2c(cc1C)C(=O)NC2] | ¹H NMR (400 MHz, DMSO-d₆) δ 8.80 (s, 1H), 8.25 (s, 1H), 8.19 (s, 1H), 8.04-7.99 (m, 3H), 7.78 (d, J = 8.1 Hz, 2H), 7.54 (s, 1H), 7.49 (s, 1H), 7.36 (d, J = 8.0 Hz, 1H), 4.29 (s, 2H), 4.17 (q, J = 7.2 Hz, 2H), 2.37 (s, 3H), 1.40 (t, J = 7.2 Hz, 3H), 1.23 (s, 3H) | 1.98 min, m/z = 425.3 [M + H]⁺ | Method N |
| 103 | 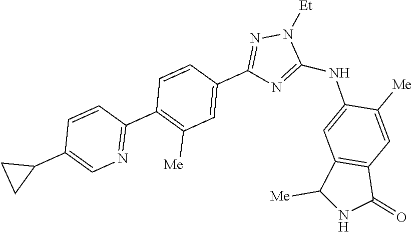 [SMILES: CCn1nc(-c2ccc(-c3ccc(C4CC4)cn3)c(C)c2)nc1Nc1cc2c(cc1C)C(=O)NC2C] | ¹H NMR (400 MHz, DMSO-d₆) δ 8.50 (d, J = 2.2 Hz, 1H), 8.37 (s, 1H), 8.25 (s, 1H), 7.84 (s, 1H), 7.81 (d, J = 7.9 Hz, 1H), 7.53-7.46 (m, 3H), 7.43 (d, J = 8.1, 3.2 Hz, 2H), 4.54 (m, 1H), 4.16 (m, 2H), 2.37 (s, 3H), 2.34 (s, 3H), 2.04- 1.97 (m, 1H), 1.40 (t, J = 7.2 Hz, 3H), 1.33 (d, J = 6.7 Hz, 3H), 1.07-1.00 (m, 2H), 0.82-0.77 (m, 2H) | 2.65 min, m/z = 479.3 [M + H]⁺ | Method N |
| 104 | 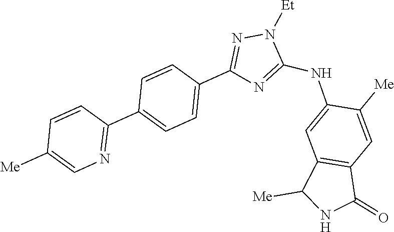 [SMILES: CCn1nc(-c2ccc(-c3ccc(C)cn3)cc2)nc1Nc1cc2c(cc1C)C(=O)NC2C] | ¹H NMR (400 MHz, DMSO-d₆) δ 8.52 (s, 1H), 8.37 (s, 1H), 8.26 (s, 1H), 8.14 (d, J = 8.4 Hz, 2H), 8.02 (d, J = 8.5 Hz, 2H), 7.91 (d, J = 8.1 Hz, 1H), 7.73-7.69 (m, 1H), 7.54 (s, 1H), 7.48 (s, 1H), 4.55 (q, J = 6.7 Hz, 1H), 4.16 (q, J = 7.2 Hz, 2H), 2.35 (d, J = 2.2 Hz, 6H), 1.40 (t, J = 7.2 Hz, 3H), 1.33 (d, J = 6.7 Hz, 3H) | 2.48 min, m/z = 439.3 [M + H]⁺ | Method N |
| 105 | 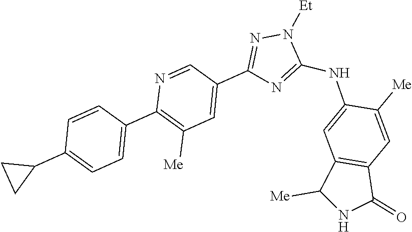 [SMILES: CCn1nc(-c2cnc(-c3ccc(C4CC4)cc3)c(C)c2)nc1Nc1cc2c(cc1C)C(=O)NC2C] | ¹H NMR (400 MHz, DMSO-d₆) δ 8.96 (s, 1H), 8.39 (s, 1H), 8.32 (s, 1H), 8.12 (s, 1H), 7.56 (s, 1H), 7.51-7.45 (m, 3H), 7.16 (d, J = 8.0 Hz, 2H), 4.56 (q, J = 6.2 Hz, 1H), 4.18 (q, J = 7.4 Hz, 2H), 2.39 (s, 3H), 2.35(s, 3H), 2.01- 1.95 (m, 1H), 1.40 (t, J = 14.0 Hz, 3H), 1.33 (d, J= 6.8 Hz, 3H), 1.02-0.97 (m, 2H), 0.75-0.71 (m, 2H) | 3.74 min, m/z = 479.3 [M + H]⁺ | Method M |
| 106 | 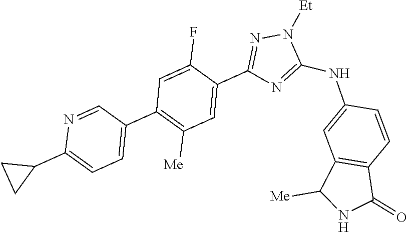 [SMILES: CCn1nc(-c2cc(C)c(-c3ccc(C4CC4)nc3)cc2F)nc1Nc1ccc2c(c1)C(C)NC2=O] | ¹H NMR (400 MHz, DMSO-d₆) δ 9.37 (s, 1H), 8.45 (s, 1H), 8.38 (s, 1H), 7.95 (s, 1H), 7.91 (d, J = 6.1 Hz, 1H), 7.78-7.66 (m, 2H), 7.57 (d, J = 8.3 Hz, 1H), 7.39 (d, J = 8.0 Hz, 1H), 7.20 (d, J = 11.4 Hz, 1H), 4.61 (q, J = 6.6 Hz, 1H), 4.25 (q, J = 7.2 Hz, 2H), 2.28 (s, 3H), 2.20-2.11 (m, 1H), 1.42-1.35 (m, 6H), 0.98 (m, 4H). | 1.64 min, m/z = 483.2 [M + H]⁺ | Method D |
| 107 | 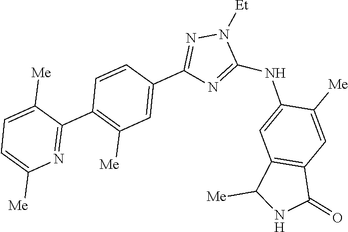 [SMILES: CCn1nc(-c2ccc(-c3nc(C)ccc3C)c(C)c2)nc1Nc1cc2c(cc1C)C(=O)NC2C] | ¹H NMR (400 MHz, CDCl₃) δ 8.01 (s, 1H), 7.97 (d, J = 8.0 Hz, 1H), 7.65 (s, 1H), 7.57 (s, 1H), 7.47 (d, J = 7.8 Hz, 1H), 7.24 (s, 1H), 7.07 (d, J = 7.8 Hz, 1H), 6.30 (s, 1H), 6.15 (s, 1H), 4.63 (q, J = 6.5 Hz, 1H), 4.09 (q, J = 7.3 Hz, 2H), 2.57 (s, 3H), 2.42 (s, 3H), 2.15 (s, 3H), 2.05 (s, 3H), 1.53 (t, J = 7.2 Hz, 3H), 1.46 (d, J = 6.7 Hz, 3H) | 2.66 min, m/z = 467.3 [M + H]⁺ | Method M |
| 108 | 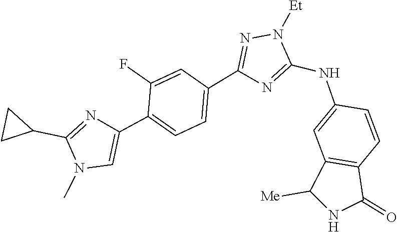 [SMILES: CCn1nc(-c2ccc(-c3cn(C)c(C4CC4)n3)c(F)c2)nc1Nc1ccc2c(c1)C(C)NC2=O] | ¹H NMR (400 MHz, DMSO-d₆) δ 9.33 (s, 1H), 8.37 (s, 1H), 8.04 (m, 1H), 7.90 (s, 1H), 7.81 (dd, J = 8.1, 1.6 Hz, 1H), 7.73-7.67 (m, 2H), 7.58 (d, J = 8.7 Hz. 1H), 7.45 (d, J = 4.2 Hz, 1H), 4.62 (q, J = 6.7 Hz, 1H), 4.22 (q, J = 7.2 Hz, 2H), 3.73 (s, 3H), 2.06-1.97 (m, 1H), 1.41-1.35 (m, 6H), 0.99-0.87 (m, 4H) | 1.16 min, m/z = 472.3 [M + H]⁺ | Method D |
| 109 | 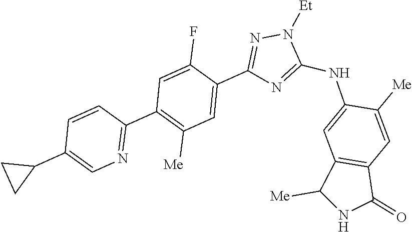 [SMILES: CCn1nc(-c2cc(C)c(-c3ccc(C4CC4)cn3)cc2F)nc1Nc1cc2c(cc1C)C(=O)NC2C] | ¹H NMR (400 MHz, DMSO-d₆) δ 8.52 (s, 1H), 8.37 (s, 1H), 8.28 (s, 1H), 7.81 (d, J = 7.5 Hz, 1H), 7.54 (s, 1H), 7.51 (m, 2H), 7.47 (s, 1H), 7.30 (d, J = 11.6 Hz, 1H), 4.54 (q, J = 6.6 Hz, 1H), 4.18 (q, J = 7.2 Hz, 2H), 2.37 (s, 3H), 2.36 (s, 3H), 2.08-2.00 (m, 1H), 1.39 (t, J = 7.2 Hz, 3H), 1.32 (d, J = 6.6 Hz, 3H), 1.10-1.04 (m, 2H), 0.86-0.80 (m, 2H) | 2.93 min, m/z = 497.3 [M + H]⁺ | Method N |
| 110 | 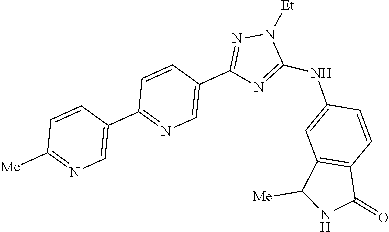 [SMILES: CCn1nc(-c2ccc(-c3ccc(C)nc3)nc2)nc1Nc1ccc2c(c1)C(C)NC2=O] | ¹H NMR (400 MHz, DMSO-d₆) δ 9.42 (s, 1H), 9.26 (s, 1H), 9.20 (d, J = 2.3 Hz, 1H), 8.44-8.32 (m, 3H), 8.13 (d, J = 8.3 Hz, 1H), 7.97 (s, 1H), 7.73 (d, J = 8.3 Hz, 1H), 7.59 (d, J = 8.3 Hz, 1H), 7.41 (d, J = 8.1 Hz, 1H), 4.63 (q, J = 6.7 Hz, 1H), 4.27 (q, J = 7.2 Hz, 2H), 2.55 (s, 3H), 1.44-1.36 (m, 6H) | 1.99 min, m/z = 426.2 [M + H]⁺ | Method N |
| 111 | 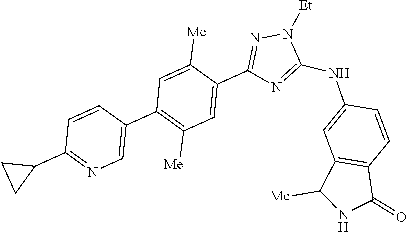 [SMILES: CCn1nc(-c2cc(C)c(-c3ccc(C4CC4)nc3)cc2C)nc1Nc1ccc2c(c1)C(C)NC2=O] | ¹H NMR (400 MHz, DMSO-d₆) δ 9.35 (s, 1H), 8.41 (d, J = 2.0 Hz, 1H), 8.36 (s, 1H), 8.00 (s, 1H), 7.86 (s, 1H), 7.67 (dd, J = 2.2, 8.0 Hz, 1H), 7.64 (dd, J = 1.6, 8.5 Hz, 1H), 7.56 (d, J = 8.3 Hz, 1H), 7.37 (d, J = 8.0 Hz, 1H), 7.15 (s, 1H), 4.60 (q, J = 6.7 Hz, 1H), 4.24 (q, J = 7.2 Hz, 2H), 2.63 (s, 3H), 2.26 (s, 3H), 2.19-2.11 (m, 1H), 1.40 (t, J = 7.3 Hz, 3H), 1.36 (d, J = 6.7 Hz, 3H), 1.01-0.94 (m, 4H) | 1.67 min, m/z = 479.3 [M + H]⁺ | Method D |
| 112 | 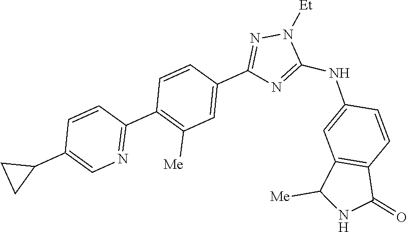 [SMILES: CCn1nc(-c2ccc(-c3ccc(C4CC4)cn3)c(C)c2)nc1Nc1ccc2c(c1)C(C)NC2=O] | ¹H NMR (400 MHz, DMSO-d₆) δ 9.34 (s, 1H), 8.51 (d, J = 1.8 Hz, 1H), 8.37 (s, 1H), 7.95-7.89 (m, 3H), 7.75 (d, J = 8.6 Hz, 1H), 7.60 (d, J = 8.4 Hz, 1H), 7.55-7.45 (m, 3H), 4.64 (q, J = 6.4 Hz, 1H), 4.25 (q, J = 7.1 Hz, 2H), 2.40 (s, 3H), 2.08-1.99 (m, 1H), 1.45-1.39 (m, 6H), 1.10-1.03 (m, 2H), 0.85-0.80 (m, 2H) | 1.57 min, m/z = 465.3 [M + H]⁺ | Method D |
| 113 | 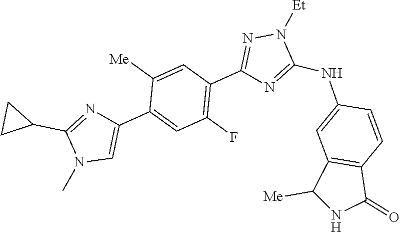 [SMILES: CCn1nc(-c2cc(C)c(-c3cn(C)c(C4CC4)n3)cc2F)nc1Nc1ccc2c(c1)C(C)NC2=O] | ¹H NMR (400 MHz, DMSO-d₆) δ 9.38 (s, 1H), 8.41 (s, 1H), 7.94 (s, 1H), 7.81 (d, J = 7.9 Hz, 1H), 7.72 (s, 1H), 7.69 (d, J = 4.9 Hz, 1H), 7.59 (d, J = 8.8 Hz, 1H), 7.45 (s, 1H), 4.63 (q, J = 6.9 Hz, 1H), 4.24 (q, J = 7.2 Hz, 2H), 3.75 (s, 3H), 2.47 (s, 3H), 2.06-1.98 (m, 1H), 1.43-1.36 (m, 6H), 1.01-0.89 (m, 4H) | 2.04 min, m/z = 486.3 [M + H]⁺ | Method N |
| 114 | 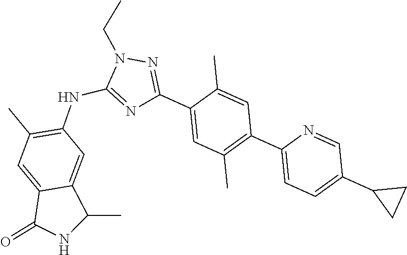 [SMILES: CCn1nc(-c2cc(C)c(-c3ccc(C4CC4)cn3)cc2C)nc1Nc1cc2c(cc1C)C(=O)NC2C] | ¹H NMR (400 MHz, DMSO-d₆) δ 8.49 (d, J = 1.9 Hz, 1H), 8.35 (s, 1H), 8.17 (s, 1H), 7.78 (s, 1H), 7.63 (s, 1H), 7.51-7.39 (m, 3H), 7.28 (s, 1H), 4.54 (q, J = 6.6 Hz, 1H), 4.18 (q, J = 7.3 Hz, 2H), 2.61 (s, 3H), 2.37 (s, 3H), 2.32 (s, 3H), 2.05-1.93 (m, 1H), 1.41 (t, J = 7.2 Hz, 3H), 1.31 (d, J = 6.2 Hz, 3H), 1.07-1.00 (m, 2H), 0.82-0.75 (m, 2H) | 2.76 min, m/z = 493.3 [M + H]⁺ | Method L |
| 115 | 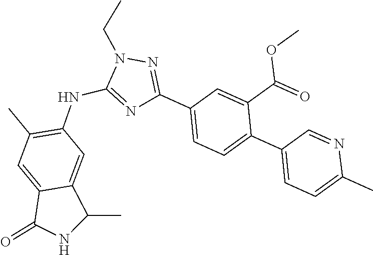 [SMILES: CCn1nc(-c2ccc(-c3ccc(C)nc3)c(C(=O)OC)c2)nc1Nc1cc2c(cc1C)C(=O)NC2C] | ¹H NMR (400 MHz, DMSO-d₆) δ 8.40-8.30 (m, 4H), 8.15 (d, J = 8.0 Hz, 1H), 7.64 (dd, J = 2.0, 6.0 Hz, 1H), 7.56 (s, 1H), 7.52 (d, J = 7.8 Hz, 1H), 7.51 (s, 1H), 7.31 (d, J = 8.0 Hz, 1H), 4.56 (q, J = 6.6 Hz, 1H), 4.19 (q, J = 7.1 Hz, 2H), 3.68 (s, 3H), 2.51 (s, 3H), 2.35 (s, 3H), 1.41 (t, J = 7.1 Hz, 3H), 1.33 (d, J = 6.4 Hz, 3H) | 2.72 min, m/z = 497.3 [M + H]⁺ | Method M |
| 116 | 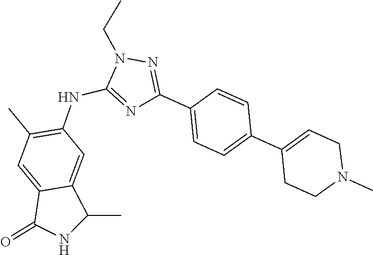 [SMILES: CCn1nc(-c2ccc(C3=CCN(C)CC3)cc2)nc1Nc1cc2c(cc1C)C(=O)NC2C] | ¹H NMR (400 MHz, DMSO-d₆) δ 8.35 (s, 1H), 8.21 (s, 1H), 7.88 (d, J = 8.1 Hz, 2H), 7.51 (s, 2H), 7.47 (d, J = 10.7 Hz, 2H), 6.23 (s, 1H), 4.54 (q, J = 6.6 Hz, 1H), 4.13 (q, J = 7.0 Hz, 2H), 3.02 (s, 2H), 2.62-2.54 (m, 2H), 2.48 (s, 2H), 2.34 (s, 3H), 2.28 (s, 3H), 1.38 (t, J = 7.2 Hz, 3H), 1.31 (d, J = 6.6 Hz, 3H) | 1.95 min, m/z = 443.3 [M + H]⁺ | Method L |
| 117 | 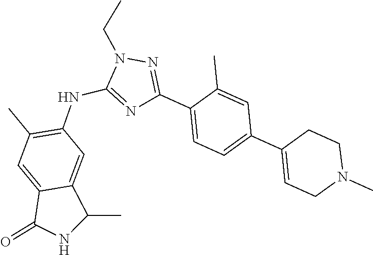 [SMILES: CCn1nc(-c2ccc(C3=CCN(C)CC3)cc2C)nc1Nc1cc2c(cc1C)C(=O)NC2C] | ¹H NMR (400 MHz, DMSO-d₆) o 8.35 (s, 1H), 8.16 (s, 1H), 7.82 (d, J = 8.4 Hz, 1H), 7.61 (s, 1H), 7.45 (s, 1H), 7.36-7.28 (m, 2H), 6.21 (s, 1H), 4.52 (q, J = 7.5 Hz, 1H), 4.16 (q, J = 7.3 Hz, 2H), 3.02 (s, 2H), 2.61 (s, 3H), 2.59- 2.54 (m, 2H), 2.47 (s, 2H), 2.36 (s, 3H), 2.27 (s, 3H), 1.39 (t, J = 7.2 Hz, 3H), 1.31 (d, J = 6.8 Hz, 3H) | 1.01 min, m/z= 457.4 [M + H]⁺ | Method D |
| 118 | 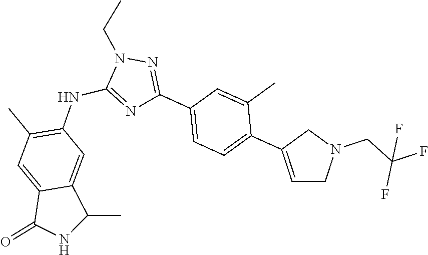 [SMILES: CCn1nc(-c2ccc(C3=CCN(CC(F)(F)F)C3)c(C)c2)nc1Nc1cc2c(cc1C)C(=O)NC2C] | ¹H NMR (400 MHz, DMSO-d₆) δ 8.37 (s, 1H), 8.23 (s, 1H), 7.78 (s, 1H), 7.72 (d, J = 7.9 Hz, 1H), 7.47 (d, J = 10.3 Hz, 2H), 7.23 (d, J = 8.1 Hz, 1H), 6.00 (s, 1H), 4.54 (q, J = 6.5 Hz, 1H), 4.14 (q, J = 6.4 Hz, 2H), 4.00 (s, 2H), 3.86 (s, 2H), 3.50 (q, J = 9.9 Hz, 2H), 2.41 (s, 3H), 2.33 (s, 3H), 1.38 (t, J = 7.4 Hz, 3H), 1.32 (d, J = 6.4 Hz, 3H) | 3.99 min, m/z = 511.3 [M + H]⁺ | Method M |
| 119 | 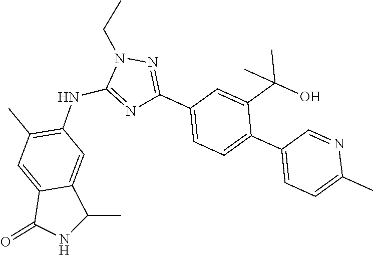 [SMILES: CCn1nc(-c2ccc(-c3ccc(C)nc3)c(C(C)(C)O)c2)nc1Nc1cc2c(cc1C)C(=O)NC2C] | ¹H NMR (400 MHz, DMSO-d₆) δ 8.40 (s, 1H), 8.35 (s, 1H), 8.32 (s, 1H), 8.24 (s, 1H), 7.78 (d, J = 7.2 Hz, 1H), 7.58 (d, J = 7.9 Hz, 1H), 7.52 (s, 1H), 7.46 (s, 1H), 7.25 (d, J = 7.8 Hz, 1H), 7.03 (d, J = 7.8 Hz, 1H), 4.99 (s, 1H), 4.53 (q, J = 6.6 Hz, 1H), 4.16 (q, J = 7.2 Hz, 2H), 2.51 (s, 3H), 2.35 (s, 3H), 1.39 (t, J = 7.0 Hz, 3H), 1.32 (d, J = 6.5 Hz, 3H), 1.26 (s, 6H) | 1.98 min, m/z = 497.3 [M + H]⁺ | Method L |
| 120 | 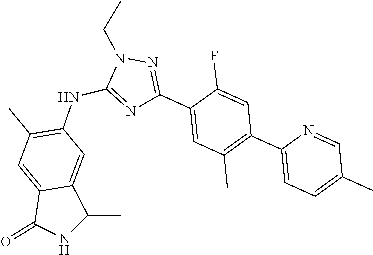 [SMILES: CCn1nc(-c2cc(C)c(-c3ccc(C)cn3)cc2F)nc1Nc1cc2c(cc1C)C(=O)NC2C] | ¹H NMR (400 MHz, DMSO-d₆) δ 8.52 (s, 1H), 8.36 (s, 1H), 8.28 (s, 1H), 7.82 (d, J = 8.0 Hz, 1H), 7.71 (d, J = 8.8 Hz, 1H), 7.58-7.41 (m, 3H), 7.30 (d, J= 11.2 Hz, 1H), 4.54 (q, J = 7.2 Hz, 1H), 4.18 (q, J = 7.0 Hz, 2H), 2.42- 2.25 (m, 9H), 1.39 (t, J = 6.9 Hz, 3H), 1.32 (d, J = 5.3 Hz, 3H) | 2.59 min, m/z = 471.3 [M + H]⁺ | Method L |
| 121 | 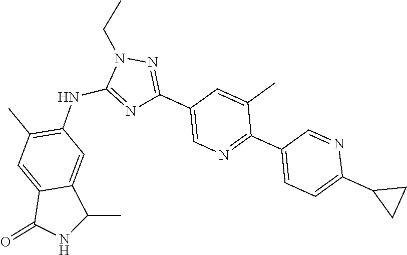 [SMILES: CCn1nc(-c2cnc(-c3ccc(C4CC4)nc3)c(C)c2)nc1Nc1cc2c(cc1C)C(=O)NC2C] | ¹H NMR (400 MHz, DMSO-d₆) δ 9.00 (s, 1H), 8.62 (s, 1H), 8.32 (s, 2H), 8.16 (s, 1H), 7.88 (dd, J = 2.4, 7.7 Hz, 1H), 7.62 (s, 1H), 7.45 (s, 1H), 7.39 (d, J = 7.7 Hz, 1H), 4.54 (q, J = 6.6 Hz, 1H), 4.18 (q, J = 7.3 Hz, 2H), 2.41 (s, 3H), 2.34 (s, 3H), 2.22-2.12 (m, 1H), 1.40 (t, J = 7.0 Hz, 3H), 1.33 (d, J = 6.2 Hz, 3H), 1.03-0.94 (m, 4H) | 2.36 min, m/z = 480.3 [M + H]⁺ | Method L |
| 122 | 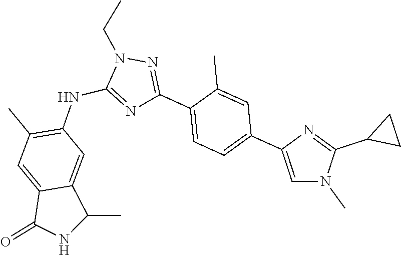 [SMILES: CCn1nc(-c2ccc(-c3cn(C)c(C4CC4)n3)cc2C)nc1Nc1cc2c(cc1C)C(=O)NC2C] | ¹H NMR (400 MHz, DMSO-d₆) δ 8.34 (s, 1H), 8.14 (s, 1H), 7.80 (d, J = 7.8 Hz, 1H), 7.61 (s, 1H), 7.57 (s, 1H), 7.52 (d, J = 8.2 Hz, 1H), 7.50 (s, 1H), 7.45 (s, 1H), 4.53 (q, J = 6.0 Hz, 1H), 4.16 (q, J = 7.6 Hz, 2H), 3.68 (s, 3H), 2.61 (s, 3H), 2.36 (s, 3H), 2.01-1.92 (m, 1H), 1.39 (t, J = 7.2 Hz, 3H), 1.31 (d, J = 6.1 Hz, 3H), 0.96-0.81 (m, 4H) | 2.60 min, m/z = 482.2 [M + H]⁺ | Method G |
| 123 | 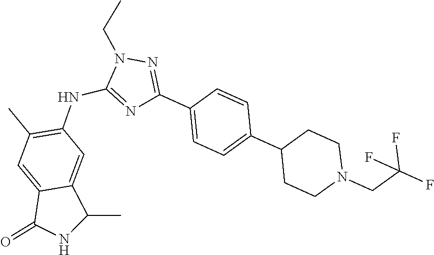 [SMILES: CCn1nc(-c2ccc(C3CCN(CC(F)(F)F)CC3)cc2)nc1Nc1cc2c(cc1C)C(=O)NC2C] | ¹H NMR (400 MHz, DMSO-d₆) δ 8.35 (s, 1H), 8.19 (s, 1H), 7.84 (d, J = 8.2 Hz, 2H), 7.49 (s, 1H), 7.46 (s, 1H), 7.31 (d, J = 7.8 Hz, 2H), 4.53 (q, J = 7.0 Hz, 1H), 4.13 (q, J = 7.0 Hz, 2H), 3.19 (q, J = 10.6 Hz, 2H), 3.02 (d, J = 11.2 Hz, 2H), 2.56-2.51 (m, 1H), 2.44 (d, J = 11.3 Hz, 2H), 2.34 (s, 3H), 1.80-1.60 (m, 4H), 1.38 (t, J = 7.0 Hz, 3H), 1.31 (d, J = 6.4 Hz, 3H) | 3.68 min, m/z = 513.3 [M + H]⁺ | Method G |
| 124 | 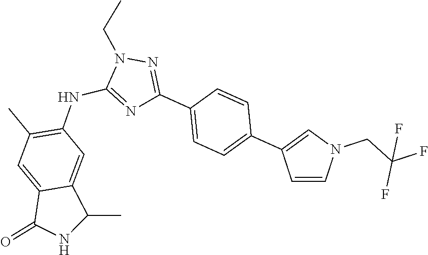 [SMILES: CCn1nc(-c2ccc(-c3ccn(CC(F)(F)F)c3)cc2)nc1Nc1cc2c(cc1C)C(=O)NC2C] | ¹H NMR (400 MHz, DMSO-d₆) δ 8.36 (s, 1H), 8.20 (s, 1H), 7.88 (d, J = 8.2 Hz, 2H), 7.58 (d, J = 8.2 Hz, 2H), 7.52 (s, 1H), 7.46 (s, 1H), 7.34 (s, 1H), 6.91 (s, 1H), 6.57 (s, 1H), 4.91 (q, J = 9.5 Hz, 2H), 4.54 (q, J = 6.7 Hz, 1H), 4.13 (q, J = 6.7 Hz, 2H), 2.35 (s, 3H), 1.38 (t, J = 7.4 Hz, 3H), 1.32 (d, J = 6.6 Hz, 3H) | 1.40 min, m/z = 495.3 [M + H]⁺ | Method C |
| 125 | 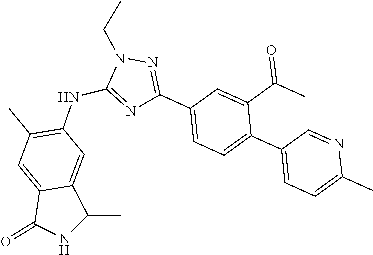 [SMILES: CCn1nc(-c2ccc(-c3ccc(C)nc3)c(C(C)=O)c2)nc1Nc1cc2c(cc1C)C(=O)NC2C] | ¹H NMR (400 MHz, DMSO-d6) 0 8.38 (s, 2H), 8.31 (s, 1H), 8.18 (s, 1H), 8.11 (d, J = 8.0 Hz, 1H), 7.65 (dd, J = 2.0, 7.9 Hz, 1H), 7.55 (s, 1H), 7.51 (d, J = 8.0 Hz, 1H), 7.47 (s, 1H), 7.32 (d, J = 8.0 Hz, 1H), 4.55 (q, J = 6.5 Hz, 1H), 4.18 (q, J = 7.3 Hz, 2H), 2.52 (s, 3H), 2.35 (s, 3H), 2.31 (s, 3H), 1.41 (t, J = 7.1 Hz, 3H), 1.33 (d, J = 6.6 Hz, 3H) | 2.08 min, m/z = 481.2 [M + H]⁺ | Method L |
| 126 | 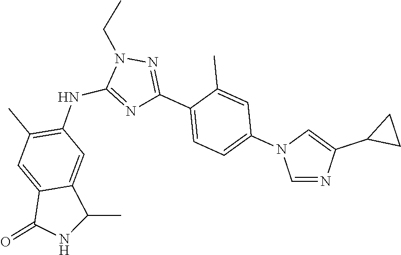 [SMILES: CCn1nc(-c2ccc(-n3cnc(C4CC4)c3)cc2C)nc1Nc1cc2c(cc1C)C(=O)NC2C] | ¹H NMR (400 MHz, DMSO-d₆) δ 8.35 (s, 1H), 8.18 (s, 1H), 8.12 (s, 1H), 7.94 (d, J = 8.4 Hz, 1H), 7.63 (s, 1H), 7.56 (s, 1H), 7.53-7.43 (m, 3H), 4.53 (q, J = 6.7 Hz, 1H), 4.17 (q, J = 7.2 Hz, 2H), 2.66 (s, 3H), 2.36 (s, 3H), 1.88-1.79 (m, 1H), 1.40 (t, J = 7.2 Hz, 3H), 1.32 (d, J = 6.6 Hz, 3H), 0.84-0.77 (m, 2H), 0.72-0.66 (m, 2H) | 2.21 min, m/z = 468.3 [M + H]⁺ | Method L |
| 127 | 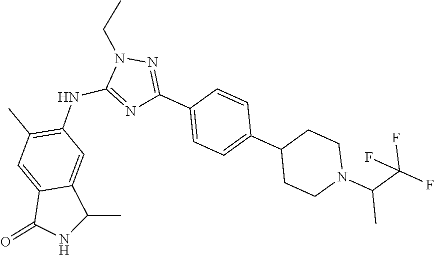 [SMILES: CCn1nc(-c2ccc(C3CCN(C(C)C(F)(F)F)CC3)cc2)nc1Nc1cc2c(cc1C)C(=O)NC2C] | ¹H NMR (400 MHz, DMSO-d₆) δ 8.35 (s, 1H), 8.20 (s, 1H), 7.84 (d, J = 8.2 Hz, 2H), 7.49 (s, 1H), 7.45 (s, 1H), 7.29 (d, J = 8.2 Hz, 2H), 4.53 (q, J = 6.7 Hz, 1H), 4.13 (q, J = 7.2 Hz, 2H), 3.55-3.45 (m, 1H), 2.98 (t, J = 13.0 Hz, 2H), 2.64-2.53 (m, 2H), 2.34 (s, 3H), 1.82-1.71 (m, 2H), 1.68- 1.52 (m, 2H), 1.38 (t, J = 7.2 H, 3H), 1.31 (d, J = 6.6 Hz, 3H), 1.19 (d, J = 7.0 Hz, 3H) | 3.13 min, m/z = 527.3 [M + H]⁺ | Method L |
| 128 | 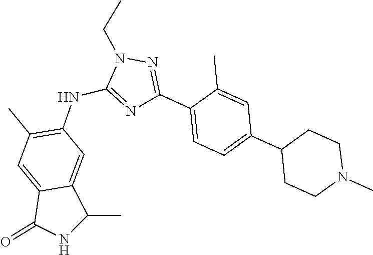 [SMILES: CCn1nc(-c2ccc(C3CCN(C)CC3)cc2C)nc1Nc1cc2c(cc1C)C(=O)NC2C] | ¹H NMR (400 MHz, DMSO-d₆) δ 8.34 (s, 1H), 8.12 (s, 1H), 7.76 (d, J = 7.6 Hz, 1H), 7.61 (s, 1H), 7.45 (s, 1H), 7.15-7.06 (m, 2H), 4.52 (q, J = 6.2 Hz, 1H), 4.16 (q, J = 7.0 Hz, 2H), 2.85 (d, J = 10.9 Hz, 2H), 2.57 (s, 3H), 2.45-2.38 (m, 1H), 2.36 (s, 3H), 2.19 (s, 3H), 1.95 (t, J = 10.9 Hz, 2H), 1.77-1.58 (m, 4H), 1.38 (t, J = 7.2 Hz, 3H), 1.30 (d, J = 6.6 Hz, 3H) | 2.48 min, m/z = 459.3 [M + H]⁺ | Method G |
| 129 | 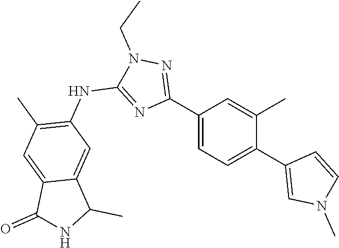 [SMILES: CCn1nc(-c2ccc(-c3ccn(C)c3)c(C)c2)nc1Nc1cc2c(cc1C)C(=O)NC2C] | ¹H NMR (400 MHz, DMSO-d₆) δ 8.35 (s, 1H), 8.19 (s, 1H), 7.75 (s, 1H), 7.69 (d, J = 8.0 Hz, 1H), 7.49 (s, 1H), 7.46 (s, 1H), 7.38 (d, J = 8.0 Hz, 1H), 6.98 (s, 1H), 6.77 (s, 1H), 6.28 (s, 1H), 4.55 (q, J = 6.5 Hz, 1H), 4.13 (q, J = 7.2 Hz, 2H), 3.67 (s, 3H), 2.43 (s, 3H), 2.34 (s, 3H), 1.38 (t, J = 7.1 Hz, 3H), 1.32 (d, J = 6.6 Hz, 3H) | 3.15 min, m/z = 441.2 [M + H]⁺ | Method L |
| 130 | 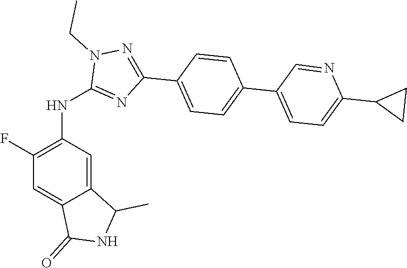 [SMILES: CCn1nc(-c2ccc(-c3ccc(C4CC4)nc3)cc2)nc1Nc1cc2c(cc1F)C(=O)NC2C] | ¹H NMR (400 MHz, DMSO-d₆) δ 9.02 (s, 1H), 8.76 (s, 1H), 8.56 (s, 1H), 8.10-8.02 (m, 3H), 7.98 (d, J = 8.2 Hz, 1H), 7.78 (d, J = 8.1 Hz, 2H), 7.46 (d, J = 10.0 Hz, 1H), 7.39 (d, J = 8.0 Hz, 1H), 4.61 (q, J = 6.5 Hz, 1H), 4.23 (q, J = 7.4 Hz, 2H), 2.21-2.10 (m, 1H), 1.40 (t, J = 6.8 Hz, 3H), 1.36 (d, J = 6.6 Hz, 3H), 1.03-0.91 (m, 4H) | 2.53 min, m/z = 469.2 [M + H]⁺ | Method L |
| 131 | 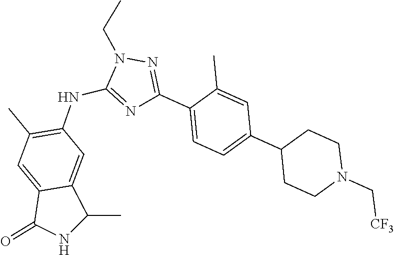 [SMILES: CCn1nc(-c2ccc(C3CCN(CC(F)(F)F)CC3)cc2C)nc1Nc1cc2c(cc1C)C(=O)NC2C] | ¹H NMR (400 MHz, DMSO-d₆) δ 8.36 (s, 1H), 8.15 (s, 1H), 7.79-7.77 (m, 1H), 7.62 (s, 1H), 7.47 (s, 1H), 7.17-7.13 (m, 2H), 4.59-4.51 (m, 1H), 4.21-4.14 (m, 2H), 3.26-3.17 (m, 2H), 3.05-3.01 (m, 2H), 2.59 (s, 3H), 2.48 (s, 2H), 2.44 (s, 1H), 2.38 (s, 3H), 1.76-1.66 (m, 4H), 1.42-1.38 (m, 3H), 1.33-1.31 (m, 3H) | 1.16 min, m/z = 527.3 [M + H]⁺ | Method D |
| 132 | 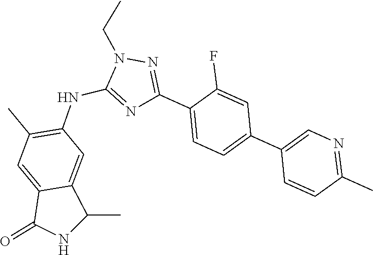 [SMILES: CCn1nc(-c2ccc(-c3ccc(C)nc3)cc2F)nc1Nc1cc2c(cc1C)C(=O)NC2C] | ¹H NMR (400 MHz, DMSO-d₆) δ 8.88-8.86 (m, 1H), 8.39 (s, 1H), 8.31 (s, 1H), 8.11-8.08 (m, 1H), 8.06-8.02 (m, 1H), 7.76-7.71 (m, 1H), 7.69-7.66 (m, 1H), 7.60 (s, 1H), 7.49 (s, 1H), 7.39-7.37 (m, 1H), 4.59-4.54 (m, 1H), 4.24-4.18(m, 2H), 2.54 (s, 3H), 2.38 (s, 3H), 1.42 (t, J = 7.2 Hz, 3H), 1.36-1.34 (d, J = 6.6 Hz, 3H) | 2.78 min, m/z = 457.2 [M + H]⁺ | Method G |

| | | | |
|---|---|---|---|
| 133 | 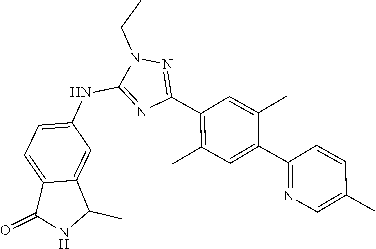 [SMILES: CCn1nc(-c2cc(C)c(-c3ccc(C)cn3)cc2C)nc1Nc1ccc2c(c1)C(C)NC2=O] | ¹H NMR (400 MHz, DMSO-d₆) δ 9.34 (s, 1H), 8.51 (s, 1H), 8.36 (s, 1H), 8.00 (s, 1H), 7.85 (s, 1H), 7.69 (d, J = 8.3 Hz, 1H), 7.65 (d, J = 8.4 Hz, 1H), 7.56 (d, J = 8.3 Hz, 1H), 7.46 (d, J = 8.0 Hz, 1H), 7.31 (s, 1H), 4.60 (q, J = 6.7 Hz, 1H), 4.24 (q, J = 7.2 Hz, 2H), 2.65 (s, 3H), 2.35 (d, J = 3.4 Hz, 6H), 1.41 (t, J = 7.1 Hz, 3H), 1.37 (d, J = 6.7 Hz, 3H). | LC-MS (ES-API, Method G): 3.017 min, m/z, 453.25 [M + H]+ |
| 134 | 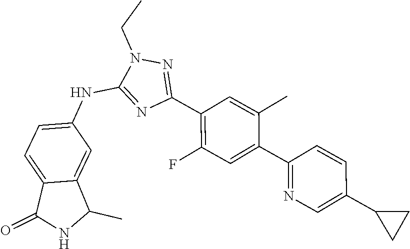 [SMILES: CCn1nc(-c2cc(C)c(-c3ccc(C4CC4)cn3)cc2F)nc1Nc1ccc2c(c1)C(C)NC2=O] | ¹H NMR (400 MHz, DMSO-d₆) δ 9.37 (s, 1H), 8.52 (s, 1H), 8.37 (s, 1H), 7.93 (s, 1H), 7.90 (d, J = 7.2 Hz, 1H), 7.70 (d, J = 8.1 Hz, 1H), 7.57 (d, J = 8.4 Hz, 1H), 7.56-7.48 (m, 2H), 7.31 (d, J = 11.5 Hz, 1H), 4.61 (q, J = 6.2 Hz, 1H), 4.29- 4.21 (m, 2H), 2.37 (s, 3H), 2.07-1.98 (m, 1H), 1.43-1.34 (m, 6H), 1.07-1.02 (m, 2H), 0.84-0.78 (m, 2H). | LC-MS (ES-API, Method L): 2.725 min, m/z, [M + H] = 483.3 |
| 135 | 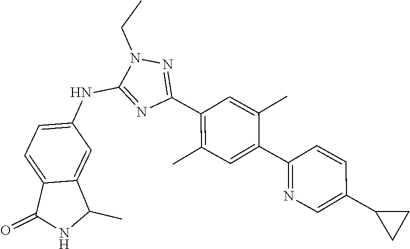 [SMILES: CCn1nc(-c2cc(C)c(-c3ccc(C4CC4)cn3)cc2C)nc1Nc1ccc2c(c1)C(C)NC2=O] | ¹H NMR (400 MHz, DMSO-d₆) δ 9.34 (s, 1H), 8.50 (s, 1H), 8.36 (s, 1H), 8.00 (s, 1H), 7.84 (s, 1H), 7.64 (d, J = 8.4 Hz, 1H), 7.56 (d, J = 8.3 Hz, 1H), 7.50 (d, J = 7.8 Hz, 1H), 7.44 (d, J = 8.1 Hz, 1H), 7.30 (s, 1H), 4.60 (q, J = 6.7 Hz, 1H), 4.24 (q, J = 7.2 Hz, 2H), 2.65 (s, 3H), 2.35 (s, 3H), 2.05-1.98 (m, 1H), 1.41 (t, J = 7.1 Hz, 3H), 1.37 (d, J = 6.6 Hz, 3H), 1.04 (q, J = 4.2 Hz, 2H), 0.81 (q, J = 5.4 Hz, 2H). | LC-MS (ES-API, Method G): 3.283 min, m/z, 479.20 [M + H]+ (%) |
| 136 | 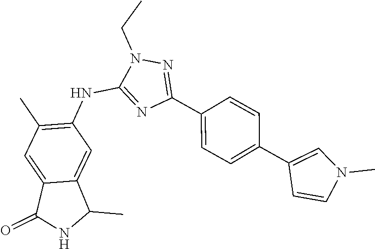 [SMILES: CCn1nc(-c2ccc(-c3ccn(C)c3)cc2)nc1Nc1cc2c(cc1C)C(=O)NC2C] | ¹H NMR (400 MHz, DMSO-d₆) δ 8.35 (s, 1H), 8.19 (s, 1H), 7.84 (d, J = 8.2 Hz, 2H), 7.54 (d, J = 8.2 Hz, 2H), 7.51 (s, 1H), 7.46 (s, 1H), 7.22 (s, 1H), 6.75 (s, 1H), 6.43 (s, 1H), 4.54 (q, J = 6.1 Hz, 1H), 4.13 (q, J = 7.0 Hz, 2H), 3.64 (s, 3H), 2.35 (s, 3H), 1.38 (t, J = 7.1 Hz, 3H), 1.32 (d, J = 6.6 Hz, 3H). | LC-MS (ES-API, Method C): 1.074 min, m/z, 427.3 [M + H]. |
| 137 | 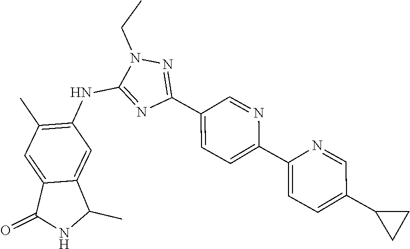 [SMILES: CCn1nc(-c2ccc(-c3ccc(C4CC4)cn3)nc2)nc1Nc1cc2c(cc1C)C(=O)NC2C] | ¹H NMR (400 MHz, DMSO-d₆) δ 9.15 (s, 1H), 8.52 (s, 1H), 8.44-8.37 (m, 2H), 8.37-8.33 (m, 2H), 8.29 (d, J = 8.3 Hz, 1H), 7.60-7.54 (m, 2H), 7.48 (s, 1H), 4.60-4.52 (m, 1H), 4.19 (q, J = 7.2 Hz, 2H), 2.36 (s, 3H), 2.08-2.00 (m, 1H), 1.41 (t, J = 7.1 Hz, 3H), 1.34 (d, J = 6.4 Hz, 3H), 1.09-1.03 (m, 2H), 0.85-0.79 (m, 2H). | LC-MS (ES-API, Method D): 1.641 min, m/z, 466.3 [M + H]. |
| 138 | 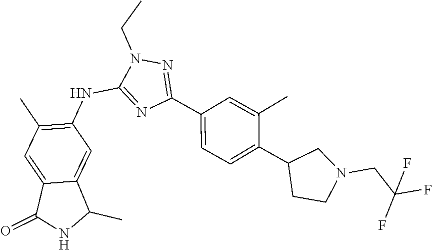 [SMILES: CCn1nc(-c2ccc(C3CCN(CC(F)(F)F)C3)c(C)c2)nc1Nc1cc2c(cc1C)C(=O)NC2C] | ¹H NMR (400 MHz, DMSO-d₆) δ 8.35 (s, 1H), 8.19 (s, 1H), 7.73-7.68 (m, 2H), 7.46 (d, J = 4.7 Hz, 2H), 7.37 (d, J = 7.9 Hz, 1H), 4.57-4.50 (m, 1H), 4.12 (q, J = 7.0 Hz, 2H), 3.58-3.49 (m , 1H), 3.43-3.33 (m,2H), 3.07 (t, J = 8.4 Hz, 1H), 2.96- 2.84 (m, 2H), 2.76 (t, J = 7.9 Hz, 1H), 2.34 (s, 3H), 2.33 (s, 3H), 2.28-2.20 (m, 1H), 1.79-1.69 (m, 1H), 1.37 (t, J = 7.2 Hz, 3H), 1.31 (d, J = 6.6 Hz, 3H). | LC-MS(ES-API, Method L):2.640 min, m/z, 513.2 [M + H]. |
| 139 | 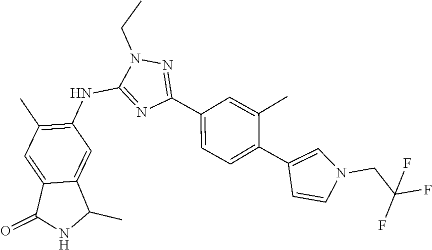 [SMILES: CCn1nc(-c2ccc(-c3ccn(CC(F)(F)F)c3)c(C)c2)nc1Nc1cc2c(cc1C)C(=O)NC2C] | ¹H NMR (400 MHz, DMSO-d₆) δ 8.35 (s, 1H), 8.20 (s, 1H), 7.78 (s, 1H), 7.72 (d, J = 8.0 Hz, 1H), 7.48 (d, J = 10.9 Hz, 2H), 7.40 (d, J = 8.0 Hz, 1H), 7.10 (s, 1H), 6.92 (s, 1H), 6.41 (s, 1H), 4.93 (q, J = 9.3 Hz, 2H), 4.58-4.51 (m, 1H), 4.13 (q, J = 7.1 Hz, 2H), 2.42 (s, 3H), 2.34 (s, 3H), 1.38 (t, J = 7.1 Hz, 3H), 1.32 (d, J = 6.4 Hz, 3H). | TM2: LC-MS(ES- API, Method L):3.261 min, m/z, 509.3 [M + H]. |
| 140 | 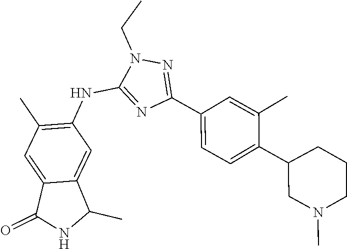 [SMILES: CCn1nc(-c2ccc(C3CCCN(C)C3)c(C)c2)nc1Nc1cc2c(cc1C)C(=O)NC2C] | ¹H NMR (400 MHz, DMSO-d₆) δ 8.35 (s, 1H), 8.18 (s, 1H), 7.71-7.68 (m, 2H), 7.46 (d, J = 3.2 Hz, 2H), 7.28 (d, J = 8.1 Hz, 1H), 4.53 (q, J = 6.7 Hz, 1H), 4.12 (q, J = 7.2 Hz, 2H), 2.93 (t, J = 11.4 Hz, 1H), 2.77 (dd, J = 23.6, 11.3 Hz, 2H), 2.33 (d, J = 4.1 Hz, 6H), 2.18 (s, 3H), 1.89 (t, J = 10.9 Hz, 2H), 1.74-1.60 (m, 3H), 1.37 (t, J = 7.2 Hz, 4H), 1.31 (d, J = 6.6 Hz, 3H). | LC-MS (ES-API, Method G): 2.517 min, m/z, 459.30 [M + H]+ |
| 141 | 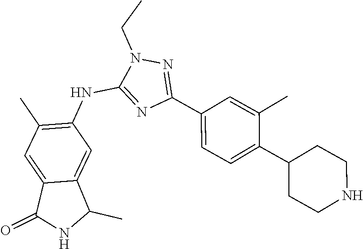 [SMILES: CCn1nc(-c2ccc(C3CCNCC3)c(C)c2)nc1Nc1cc2c(cc1C)C(=O)NC2C] | ¹H NMR (400 MHz, DMSO-d₆) δ 8.36 (s, 1H), 8.19 (br s, 1H), 7.73-7.68 (m, 2H), 7.46 (d, J = 3.5 Hz, 2H), 7.24 (d, J = 8.0 Hz, 1H), 4.53 (q, J = 6.7 Hz, 1H), 4.12 (q, J = 7.2 Hz, 2H), 3.03 (d, J = 11.8 Hz, 1H), 2.84-2.74 (m, 1H), 2.61 (t, J = 12.0 Hz, 2H), 2.33 (s, 6H), 1.65-1.58 (m, 2H), 1.56-1.44 (m, 2H), 1.37 (t, J = 7.2 Hz, 3H), 1.31 (d, J = 6.6 Hz, 3H). | LC-MS (ES-API, Method G): 2.517 min, m/z, 445.30 [M + H]+ (%) |
| 142 | 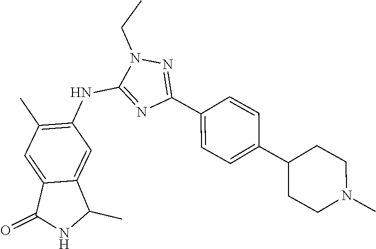 [SMILES: CCn1nc(-c2ccc(C3CCN(C)CC3)cc2)nc1Nc1cc2c(cc1C)C(=O)NC2C] | ¹H NMR (400 MHz, DMSO-d₆) δ 8.35 (s, 1H), 8.19 (s, 1H), 7.84 (d, J = 7.9 Hz, 2H), 7.49 (s, 1H), 7.45 (s, 1H), 7.29 (d, J = 8.0 Hz, 2H), 4.53 (q, J = 6.7 Hz, 1H), 4.12 (q, J = 7.2 Hz, 2H), 2.87 (d, J = 11.0 Hz, 2H), 2.48-2.43 (m, 1H), 2.33 (s, 3H), 2.19 (s, 3H), 1.97 (t, J = 10.7 Hz, 2H), 1.76-1.61 (m, 4H), 1.37 (t, J = 7.2 Hz, 3H), 1.31 (d, J = 6.6 Hz, 3H). | LCMS (ES-API, Method L): 1.802 min, m/z= 445.3 [M + H]⁺ |
| 143 | 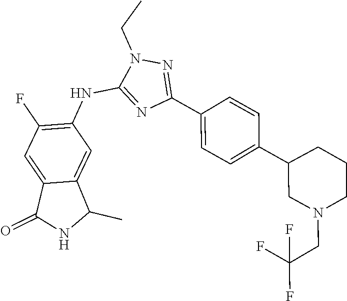 [SMILES: CCn1nc(-c2ccc(C3CCCN(CC(F)(F)F)C3)cc2)nc1Nc1cc2c(cc1F)C(=O)NC2C] | ¹HNMR (400 MHz, DMSO-d₆, ppm) δ 8.96 (s, 1H), 8.54 (s, 1H), 7.97 (d, J = 7.2 Hz, 1H), 7.87 (d, J = 8.4 Hz, 2H), 7.44 (d, J = 10.0 Hz, 1H), 7.33 (d, J = 8.0 Hz, 2H), 4.59 (q, J = 6.5 Hz, 1H), 4.19 (q, J = 7.1 Hz, 2H), 3.19 (q, J = 10.3 Hz, 2H), 3.02 (d, J = 11.6 Hz, 2H), 2.56-2.53 (m, 1H), 2.46-2.44 (m, 2H), 1.76-1.64 (m, 4H), 1.38 (t, J = 7.3 Hz, 3H), 1.34 (d, J = 6.8 Hz, 3H). | LC-MS (ES-API, Method L): 2.864 min, m/z, 517.3 [M + H] |
| 144 | 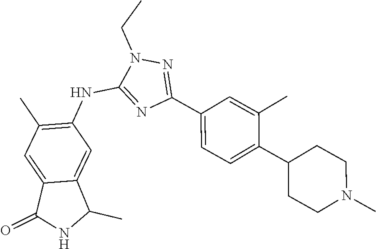 [SMILES: CCn1nc(-c2ccc(C3CCN(C)CC3)c(C)c2)nc1Nc1cc2c(cc1C)C(=O)NC2C] | ¹H NMR (400 MHz, DMSO-d₆) δ 8.35 (s, 1H), 8.18 (s, 1H), 7.73-7.66 (m, 2H), 7.46 (d, J = 4.9 Hz, 2H), 7.26 (d, J = 8.1 Hz, 1H), 4.57-4.50 (m, 1H), 4.12 (q, J = 7.2 Hz, 2H), 2.87 (d, J = 10.9 Hz, 2H), 2.69-2.60 (m, 1H), 2.33 (s, 6H), 2.19 (s, 3H), 2.02-1.92 (m, 2H), 1.68-1.62 (m, 4H), 1.37 (t, J = 7.2 Hz, 3H), 1.31 (d, J = 6.6 Hz, 3H). | LC-MS(ES-API, Method D): 0.920 min, m/z, 459.4 [M + H] |
| 145 | 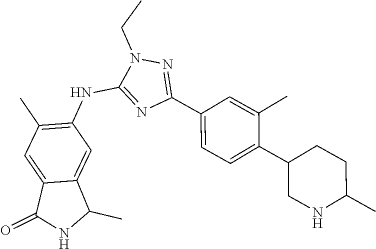 [SMILES: CCn1nc(-c2ccc(C3CCC(C)NC3)c(C)c2)nc1Nc1cc2c(cc1C)C(=O)NC2C] | ¹H NMR (400 MHz, DMSO-d₆) δ 8.36 (s, 1H), 8.18 (s, 1H), 7.74 (d, J = 8.4 Hz, 1H), 7.69 (d, J = 4.7 Hz, 2H), 7.47-7.43 (m, 2H), 7.23 (d, J = 8.0 Hz, 1H), 4.53 (q, J = 6.7 Hz, 1H), 4.12 (q, J = 7.2 Hz, 2H), 3.00-2.86 (m, 2H), 2.82-2.74 (m, 1H), 2.61-2.54 (m, 1H), 2.5-2.31 (m, 6H), 1.83-1.74(m, 1H), 1.73-1.57 (m, 2H), 1.37 (t, J = 7.2 Hz, 3H), 1.31 (d, J = 6.6 Hz, 3H), 1.23-1.16 (m, 1H), 1.12 (d, J = 6.6 Hz, 2H), 1.02 (d, J = 6.2 Hz, 1H). | LC-MS (ES-API, Method G): 0.833 min, m/z, 459.30 [M + H]+ (%) |
| 146 | 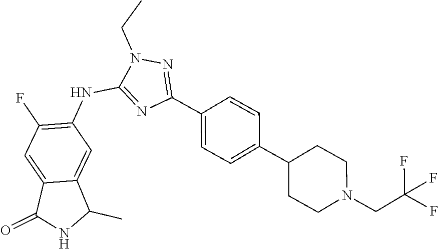 [SMILES: CCn1nc(-c2ccc(C3CCN(CC(F)(F)F)CC3)cc2)nc1Nc1cc2c(cc1F)C(=O)NC2C] | ¹H NMR (400 MHz, DMSO) δ 8.96 (s, 1H), 8.54 (s, 1H), 7.98 (d, J = 7.1 Hz, 1H), 7.88 (d, J = 8.0 Hz, 1H), 7.44 (d, J = 10.1 Hz, 1H), 7.33 (d, J = 8.1 Hz, 1H), 4.59 (q, J = 4.6 Hz, 1H), 4.19 (q, J = 7.1 Hz, 2H), 3.26-3.16 (m, 2H), 2.99-2.92 (m, , 2H), 2.84-2.75 (m, 1H), 2.45-2.34 (m, 2H), 1.88-1.79 (m, , 1H), 1.74-1.65 (m, , 1H), 1.66-1.56 (m, 1H), 1.52-1.42 (m, , 1H), 1.40-1.33 (m, 5H). | LC-MS (ES-API, Method D): 2.112 min, m/z, 517.3 [M + H]. |
| 147 | 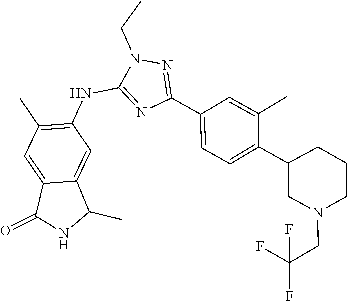 [SMILES: CCn1nc(-c2ccc(C3CCCN(CC(F)(F)F)C3)c(C)c2)nc1Nc1cc2c(cc1C)C(=O)NC2C] | ¹H NMR (400 MHz, DMSO-d₆) δ 8.31 (s, 1H), 8.18 (br s, 1H), 7.75-7.68 (m, 2H), 7.50 (s, 1H), 7.44 (s, 1H), 7.27 (d, J = 8.0 Hz, 1H), 4.52 (q, J = 6.6 Hz, 1H), 4.12 (q, J = 7.2 Hz, 2H), 3.20 (q, J = 10.3 Hz, 2H), 2.97-2.88 (m, 3H), 2.42-2.37 (m, 3H), 2.36-2.31 (m, 6H), 1.77-1.60 (m, 3H), 1.37 (t, J = 7.1 Hz, 3H), 1.31 (d, J = 6.7 Hz, 3H). | LC-MS(ES-API, Method D): 2.082 min, m/z, 527.3 [M + H] |
| 148 | 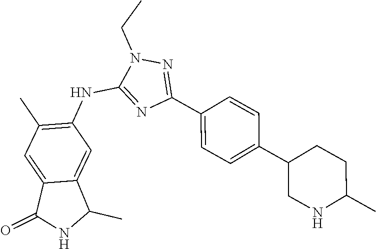 [SMILES: CCn1nc(-c2ccc(C3CCC(C)NC3)cc2)nc1Nc1cc2c(cc1C)C(=O)NC2C] | ¹H NMR (400 MHz, DMSO-d₆) δ 8.35 (s, 1H), 8.18 (s, 1H), 7.83 (d, J = 8.4 Hz, 2H), 7.49 (s, 1H), 7.45 (s, 1H), 7.27 (d, J = 8.1 Hz, 2H), 4.53 (q, J = 6.7 Hz, 1H), 4.12 (q, J = 7.2 Hz, 2H), 3.17-2.83 (m, 2H), 2.62-2.53 (m, 2H), 2.33 (s, 3H), 2.08-1.97 (m, 2H), 1.89-1.81 (m, 1H), 1.71-1.65 (m, 1H), 1.63-1.51 (m, 1H), 1.37 (t, J = 7.2 Hz, 3H), 1.31 (d, J = 6.7 Hz, 3H), 1.06 (d, J = 6.4 Hz, 1H), 1.00 (d, J = 6.2 Hz, 2H). | LCMS (ES-API, Method D): 0.921 min, m/z= 445.3 [M + H]+ |
| 149 | 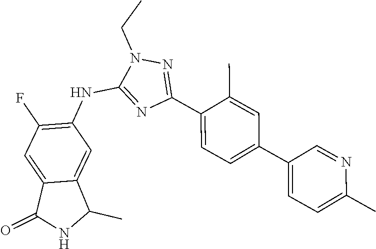 [SMILES: CCn1nc(-c2ccc(-c3ccc(C)nc3)cc2C)nc1Nc1cc2c(cc1F)C(=O)NC2C] | ¹H NMR (400 MHz, DMSO-d₆) δ 9.02 (s, 1H), 8.81 (d, J = 2.3 Hz, 1H), 8.55 (s, 1H), 8.08 (d, J = 7.1 Hz, 1H), 8.03-7.99 (m, 2H), 7.65 (s, 1H), 7.61 (d, J = 8.2 Hz, 1H), 7.45 (d, J = 10.1 Hz, 1H), 7.35 (d, J = 8.1 Hz, 1H), 4.59 (q, J = 6.7 Hz, 1H), 4.24 (q, J = 7.1 Hz, 2H), 2.71 (s, 3H), 2.51 (s, 3H), 1.41 (t, J = 7.1 Hz, 3H), 1.35 (d, J = 6.6 Hz, 3H). | LC-MS (ES-API, Method G): 2.783 min, m/z, 457.25 [M + H]+ (%) |
| 150 | 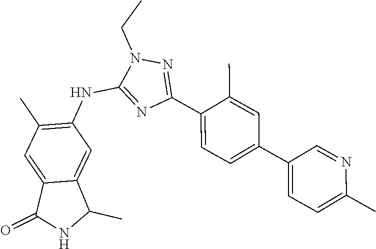 [SMILES: CCn1nc(-c2ccc(-c3ccc(C)nc3)cc2C)nc1Nc1cc2c(cc1C)C(=O)NC2C] | ¹H NMR (400 MHz, DMSO-d₆) δ 8.80 (d, J = 2.3 Hz, 1H), 8.35 (s, 1H), 8.18 (s, 1H), 8.00 (dd, J = 8.1, 2.1 Hz, 1H), 7.96 (d, J = 8.2 Hz, 1H), 7.67-7.63 (m, 2H), 7.57 (d, J = 8.3Hz, 1H), 7.46 (s, 1H), 7.34 (d, J = 8.2 Hz, 1H), 4.54 (q, J = 6.6 Hz, 1H), 4.19 (q, J = 7.2 Hz, 2H), 2.69 (s, 3H), 2.50 (s, 3H), 2.37 (s, 3H), 1.41 (t, J = 7.2 Hz, 3H), 1.32 (d, J = 6.6 Hz, 3H). | LCMS (ES-API, Method D): 1.077 min, m/z 453.3 [M + H]+ |
| 151 | 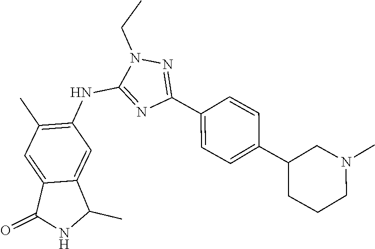 [SMILES: CCn1nc(-c2ccc(C3CCCN(C)C3)cc2)nc1Nc1cc2c(cc1C)C(=O)NC2C] | ¹H NMR (400 MHz, DMSO-d₆) δ 8.36 (s, 1H), 8.24 (s, 1H), 7.85 (d, J = 7.7 Hz, 2H), 7.49 (s, 1H), 7.45 (s, 1H), 7.30 (d, J = 7.8 Hz, 2H), 4.57-4.50 (m, 1H), 4.18- 4.10 (m, 2H), 2.90-2.74 (m, 3H), 2.33 (s, 3H), 2.23 (s, 3H), 2.05-1.92 (m, 2H), 1.86-1.78 (m, 1H), 1.75-1.68 (m, 1H), 1.68-1.56 (m, 1H), 1.47-1.40 (m, 1H), 1.37 (t, J = 7.0 Hz, 3H), 1.31 (d, J = 6.3 Hz, 3H). | LC-MS(ES-API, Method L): 1.801 min, m/z, 445.4 [M + H]. |
| 152 | 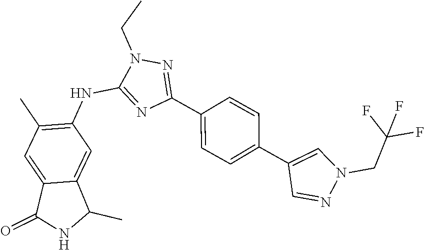 [SMILES: CCn1nc(-c2ccc(-c3cnn(CC(F)(F)F)c3)cc2)nc1Nc1cc2c(cc1C)C(=O)NC2C] | ¹H NMR (400 MHz, DMSO-d₆) δ 8.36 (s, 1H), 8.33 (s, 1H), 8.22 (s, 1H), 8.08 (s, 1H), 7.92 (d, J = 8.3 Hz, 2H), 7.68 (d, J = 8.3 Hz, 2H), 7.53 (s, 1H), 7.47 (s, 1H), 5.16 (q, J = 9.1 Hz, 2H), 4.55 (q, J = 6.7 Hz, 1H), 4.14 (q, J = 7.2 Hz, 2H), 2.35 (s, 3H), 1.39 (t, J = 7.2 Hz, 3H), 1.33 (d, J = 6.7 Hz, 3H). | LC-MS (ES-API, Method L): 2.786min, m/z .[M + H] = 496.2 |
| 153 | 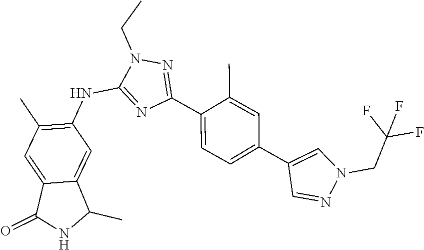 [SMILES: CCn1nc(-c2ccc(-c3cnn(CC(F)(F)F)c3)cc2C)nc1Nc1cc2c(cc1C)C(=O)NC2C] | ¹H NMR (400 MHz, DMSO-d₆) δ 8.35 (s, , 1H), 8.31 (s, 1H), 8.16 (s, 1H), 8.07 (s, 1H), 7.87 (d, J = 8.1 Hz, 1H), 7.63 (s, 1H), 7.54 (s, 1H), 7.51-7.45 (m, 2H), 5.15 (q, J = 9.1 Hz, 2H), 4.54 (q, J = 6.7 Hz, 1H), 4.17 (q, J = 7.2 Hz, 2H), 2.64 (s, 3H), 2.37 (s, 3H), 1.40 (t, J = 7.2 Hz, 3H), 1.32 (d, J = 6.6 Hz, 3H). | LC-MS (ES-API, Method G): 3.713 min, m/z, 510.15 [M + H]+ |
| 154 | 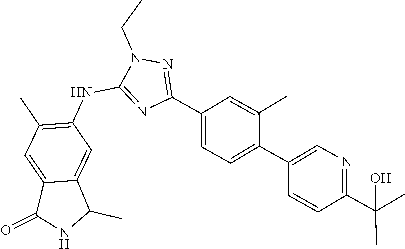 [SMILES: CCn1nc(-c2ccc(-c3ccc(C(C)(C)O)nc3)c(C)c2)nc1Nc1cc2c(cc1C)C(=O)NC2C] | ¹H NMR (400 MHz, DMSO-d₆) δ 8.50 (d, J = 2.3 Hz, 1H), 8.36 (s, 1H), 8.24 (s, 1H), 7.88 (s, 1H), 7.84-7.79 (m, 2H), 7.73 (d, J = 8.2 Hz, 1H), 7.51 (s, 1H), 7.47 (s, 1H), 7.31 (d, J = 7.9 Hz, 1H), 5.25 (s, 1H), 4.55 (q, J = 6.4 Hz, 1H), 4.16 (q, J = 7.2 Hz, 2H), 2.35 (s, 3H), 2.30 (s, 3H), 1.49 (s, 6H), 1.40 (t, J = 7.2 Hz, 3H), 1.33 (d, J = 6.6 Hz, 3H). | LCMS (ES-API, Method D): 1.203 min, m/z = 497.3 [M + H]+. |
| 155 | 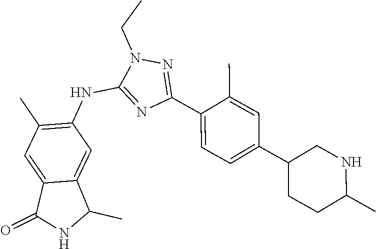 [SMILES: CCn1nc(-c2ccc(C3CCC(C)NC3)cc2C)nc1Nc1cc2c(cc1C)C(=O)NC2C] | ¹H NMR (400 MHz, DMSO-d₆) δ 8.36 (s, 1H), 8.19 (s, 1H), 7.81 (d, J = 7.9 Hz, 1H), 7.61 (s, 1H), 7.45 (s, 1H), 7.19-7.15 (m, 2H), 4.52 (q, J = 6.9 Hz, 1H), 4.17 (q, J = 7.2 Hz, 2H), 3.20-3.13 (m, 1H), 3.05 (t, J = 12.2 Hz, 1H), 2.97-2.87 (m, 1H), 2.59 (s, 3H), 2.35 (s, 3H), 2.03-1.97 (m, 2H), 1.95- 1.88 (m, 2H), 1.82-1.71 (m, 2H), 1.39 (t, J = 7.2 Hz, 3H), 1.30 (d, J = 6.7 Hz, 3H), 1.27 (d, J = 6.5 Hz, 3H). | LC-MS (ES-API, Method D): 0.983 min, m/z, 459.4 [M + H]+ (%) |
| 156 | 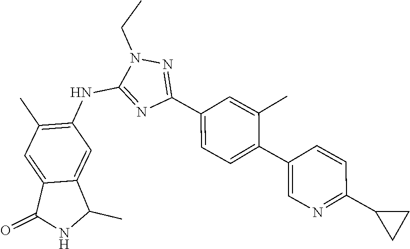 [SMILES: CCn1nc(-c2ccc(-c3ccc(C4CC4)nc3)c(C)c2)nc1Nc1cc2c(cc1C)C(=O)NC2C] | ¹H NMR (400 MHz, DMSO-d₆) δ 9.34 (s, 1H), 8.42 (s, 1H), 8.25 (s, 1H), 7.92 s, 1H), 7.87 (d, J = 7.9 Hz, 1H), 7.77 (s, 1H), 7.72-7.66 (m, 1H), 7.46 (m, 1H), 7.37 (d, J = 8.0 Hz, 1H), 7.31 (d, J = 7.9 Hz, 1H), 4.53 (q, J = 6.6 Hz, 1H), 4.29-4.20 (m, 2H), 2.57 (s, 3H), 2.31 (s, 3H), 2.20-2.12 (m, 1H), 1.41-1.34 (m, 6H), 1.02- 0.95 (m, 4H). | LCMS (ES-API, Method D): 1.546 min, m/z = 479.3 [M + H]+. |
| 157 | 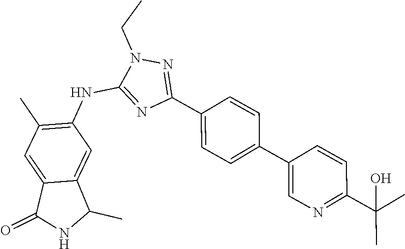 [SMILES: CCn1nc(-c2ccc(-c3ccc(C(C)(C)O)nc3)cc2)nc1Nc1cc2c(cc1C)C(=O)NC2C] | ¹H NMR (400 MHz, DMSO) δ 8.83 (d, J = 2.3 Hz, 1H), 8.37 (s, 1H), 8.25 (s, 1H), 8.09 (dd, J = 8.3, 2.4 Hz, 2H), 8.03 (d, J = 8.1 Hz, 1H), 7.80 (d, J = 8.1 Hz, 2H), 7.74 (d, J = 8.3 Hz, 1H), 7.56 (s, 1H), 7.47 (s, 1H), 5.26 (s, 1H), 4.56 (q, J = 6.6 Hz, 1H), 4.17 (q, J = 7.2 Hz, 2H), 2.36 (s, 3H), 1.48 (s, 6H), 1.40 (t, J = 7.2 Hz, 2H), 1.33 (d, J = 6.7 Hz, 2H). | LC-MS (ES-API, Method D): 1.109 min, m/z, 483.3 [M + H]. |
| 158 | 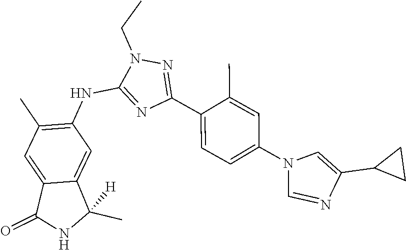 [SMILES: CCn1nc(-c2ccc(-n3cnc(C4CC4)c3)cc2C)nc1Nc1cc2c(cc1C)C(=O)N[C@@H]2C] | ¹H NMR (400 MHz, CDCI₃) δ: 8.08 (d, J = 8 .. 1 Hz, 1H), 7.87 (s, 1H), 7.77 (br s, 1H), 7.67 (s, 1H), 7.29-7.26 (m, 2H), 7.06 (s, 1H), 6.14 (d, J = 16.3 Hz, 2H), 4.64 (q, J = 6.7 Hz, 1H), 4.12 (q, J = 7.3 Hz, 2H), 2.75 (s, 3H), 2.43 (s, 3H), 1.97-1.87 (m, 1H), 1.56 (t, J = 7.3 Hz, 3H), 1.47 (d, J = 6.7 Hz, 3H), 0.93-0.78 (m, 4H). | Agilent 8 min: 3.884 min, m/z 468.10 [M + H]+ |
| 159 | 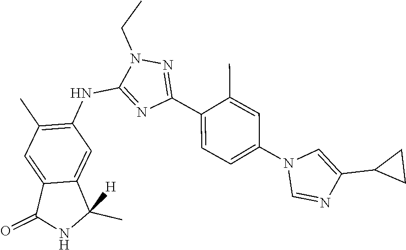 [SMILES: CCn1nc(-c2ccc(-n3cnc(C4CC4)c3)cc2C)nc1Nc1cc2c(cc1C)C(=O)N[C@H]2C] | ¹H NMR (400 MHz, CDCI₃) δ: 8.07 (d, J = 8.0 Hz, 2H), 7.82 (s, 1H), 7.78 (br s, 1H), 7.66 (s, 1H), 7.28-7.24 (m, 2H), 7.06 (s, 1H), 6.56 (s, 1H), 6.25 (s, 1H), 4.63 (q, J = 6.6 Hz, 1H), 4.12 (q, J = 7.3 Hz, 2H), 2.75 (s, 3H), 2.43 (s, 3H), 1.97-1.88 (m, 1H), 1.55 (t, J = 7.3 Hz, 3H), 1.47 (d, J = 6.7 Hz, 3H), 0.93-0.85 (m, 2H), 0.85-0.79 (m, 2H). | Agilent 8 min: 3.86 min, m/z 468.20 [M + H]+ |
| 160 | 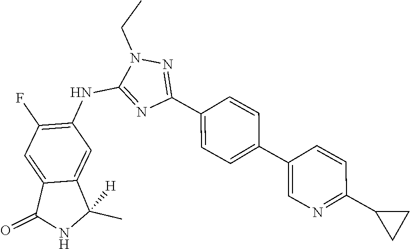 [SMILES: CCn1nc(-c2ccc(-c3ccc(C4CC4)nc3)cc2)nc1Nc1cc2c(cc1F)C(=O)N[C@@H]2C] | ¹H NMR (400 MHz, DMSO-d₆) δ 9.01 (s, 1H), 8.77 (s, 1H), 8.56 (s, 1H), 8.10-7.90 (m, 4H), 7.85-7.73 (m, 2H), 7.54-7.34 (m, 2H), 4.69-4.56 (m, 1H), 4.30-4.17 (m, 2H), 2.23-2.09 (m, 1H), 1.47-1.33 (m, 6H), 1.01-0.94 (m, 4H) | HPLC (Agilent 17 min): 10.3 min |
| 161 | 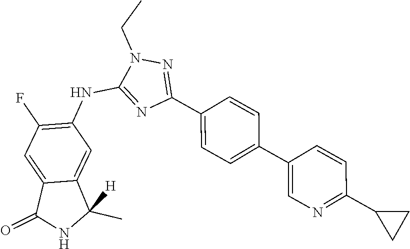 [SMILES: CCn1nc(-c2ccc(-c3ccc(C4CC4)nc3)cc2)nc1Nc1cc2c(cc1F)C(=O)N[C@H]2C] | ¹H NMR (400 MHz, DMSO-d₆) δ 9.02 (s, 1H), 8.77 (s, 1H), 8.56 (s, 1H), 8.12-7.94 (m, 4H), 7.84-7.74 (m, 2H), 7.52-7.35 (m, 2H), 4.68-4.56 (m, 1H), 4.31-4.16 (m, 2H), 2.22-2.09 (m, 1H), 1.49-1.31 (m, 6H), 1.03-0.92 (m, 4H) | HPLC (Agilent 17 min): 9.3 min |
| 162 | 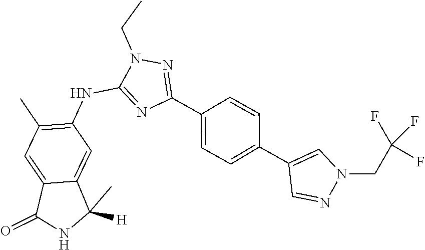 [SMILES: CCn1nc(-c2ccc(-c3cnn(CC(F)(F)F)c3)cc2)nc1Nc1cc2c(cc1C)C(=O)N[C@H]2C] | 1H NMR (400 MHz, DMSO-d6) δ 8.36 (s, 1H), 8.33 (s, 1H), 8.22 (s, 1H), 8.08 (s, 1H), 7.92 (d, J = 8.3 Hz, 2H), 7.68 (d, J = 8.3 Hz, 2H), 7.53 (s, 1H), 7.47 (s, 1H), 5.16 (q, J = 9.1 Hz, 2H), 4.55 (q, J = 6.7 Hz, 1H), 4.14 (q, J = 7.2 Hz, 2H), 2.35 (s, 3H), 1.39 (t, J = 7.2 Hz, 3H), 1.33 (d, J = 6.7 Hz, 3H) | LC-MS (ES-API, Method D): 1.717 min, m/z [M + H] = 497.2 |
| 163 | 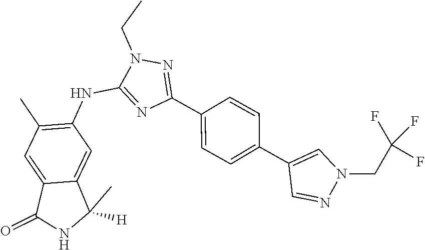 [SMILES: CCn1nc(-c2ccc(-c3cnn(CC(F)(F)F)c3)cc2)nc1Nc1cc2c(cc1C)C(=O)N[C@@H]2C] | 1H NMR (400 MHz, DMSO-d6) δ 8.36 (s, 1H), 8.33 (s, 1H), 8.22 (s, 1H), 8.08 (s, 1H), 7.92 (d, J = 8.3 Hz, 2H), 7.68 (d, J = 8.3 Hz, 2H), 7.53 (s, 1H), 7.47 (s, 1H), 5.16 (q, J = 9.1 Hz, 2H), 4.55 (q, J = 6.7 Hz, 1H), 4.14 (q, J = 7.2 Hz, 2H), 2.35 (s, 3H), 1.39 (t, J = 7.2 Hz, 3H), 1.33 (d, J = 6.7 Hz, 3H) | LC-MS (ES-API, Method D): 1.717 min, m/z [M + H] = 497.2 |

Compound 145 and analogues were prepared according to the following procedure.

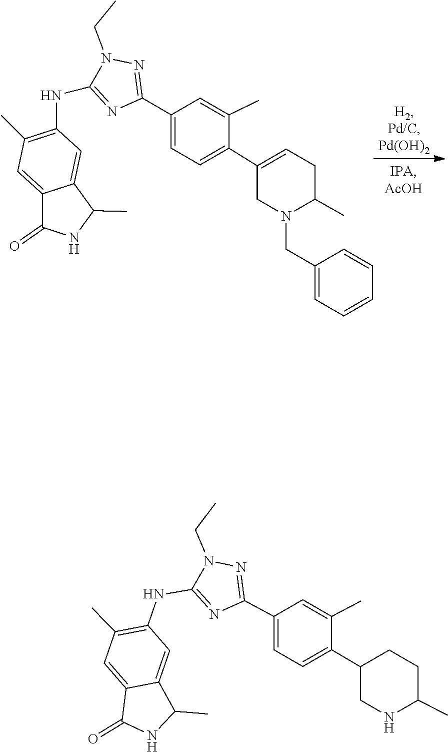

A mixture of 5-((3-(4-(1-benzyl-6-methyl-1,2,5,6-tetrahydropyridin-3-yl)-3-methylphenyl)-1-ethyl-1H-1,2,4-triazol-5-yl)amino)-3,6-dimethylisoindolin-1-one (180 mg, 0.329 mmol, 1.0 eq) in IPA (27 mL), Pd/C (180 mg), Pd(OH)₂ (180 mg) and acetic acid (1.97 mg, 0.0329, 0.1 eq) was stirred at 80° C. under H₂ (0.4 MPa) overnight. The mixture was concentrated in vacuum to give the crude which was purified by filtration to give crude product. Further purification by preparative-HPLC gave 5-[[2-ethyl-5-[3-methyl-4-(6-methyl-3-piperidyl)phenyl]-1,2,4-triazol-3-yl]amino]-3,6-dimethyl-isoindolin-1-one (20 mg, 0.0436 mmol). LC-MS (ES-API, Method G): 0.833 min, m/z, 459.30 [M+H]+(%). ¹H NMR (400 MHz, DMSO-d₆) δ 8.36 (s, 1H), 8.18 (s, 1H), 7.74 (d, J=8.4 Hz, 1H), 7.69 (d, J=4.7 Hz, 2H), 7.47-7.43 (m, 2H), 7.23 (d, J=8.0 Hz, 1H), 4.53 (q, J=6.7 Hz, 1H), 4.12 (q, J=7.2 Hz, 2H), 3.00-2.86 (m, 2H), 2.82-2.74 (m, 1H), 2.61-2.54 (m, 1H), 2.34 (s, 3H), 2.32 (s, 3H), 1.83-1.74 (m, 1H), 1.71-1.57 (m, 2H), 1.37 (t, J=7.2 Hz, 3H), 1.31 (d, J=6.6 Hz, 3H), 1.23-1.16 (m, 1H), 1.12 (d, J=6.6 Hz, 2H), 1.02 (d, J=6.2 Hz, 1H).

### ROCK2 Binding Activity:

Assay for ROCK2 inhibition was performed using the protein construct N-terminal 6His-tagged ROCK2 catalytic domain 11-552 (Dundee University, UK). Protein was purified from a baculovirus expression system. Long S6 peptide (KEAKEKRQEQIAKRRRLSSLRASTSKSGGSQK) was used as substrate. Kinase reactions were carried out in 15 μl volume in a 96-well plate (black, half area) using 1.25 nM constitutively active ROCK2 kinase, 100 μM long S6 peptide, 20 μM ATP and test compound in DMSO (or DMSO only for controls). The final concentration of DMSO was 1%. Assay buffer was 50 mM HEPES pH 7.5 supplemented with 0.2 mM EDTA, 10 mM magnesium acetate, 0.01% Tween-20, 1 mM DTT and 0.01% BSA. Test compounds were pre-incubated with ROCK2 kinase for 1 hour before addition of ATP and long S6 peptide. After incubation for a further 1 hour, the amount of ADP produced was measured using ADP-Glo Kinase Assay (Promega) as per manufacturer's instructions. The luminescence was measured on a PHERAstar FS (BMG Labtech). The concentration of test compound required to inhibit ADP production by 50% (the IC₅₀) was calculated using a four-parameter logistic function with software by Dotmatics.

Table 8 shows the ROCK2 or ROCK1 binding activity, as determined by the assay described above, for certain compounds of the formula, categorised based on the ROCK2 or ROCK1 IC₅₀ value of the compound as “+”, “++”, “+++” and “++++”. The category “+” refers to compounds with a ROCK2 or ROCK1 IC₅₀ value of >10 μM. The category “++” refers to compounds with a ROCK2 or ROCK1 IC₅₀ value of 10 to 3 μM. The category “+++” refers to compounds with a ROCK1 or ROCK2 IC₅₀ value of 3 to 0.3 μM. The category “++++” refers to compounds with a ROCK1 or ROCK2 IC₅₀ value of <0.3 μM.

TABLE 8

| Example | | |
|---|---|---|
| Number | ROCK 2 | ROCK 1 |
| 1 | ++++ | + |
| 2 | ++++ | + |
| 3 | ++++ | + |
| 4 | ++++ | ++ |
| 5 | ++++ | ++ |
| 6 | ++++ | + |
| 7 | ++ | + |
| 8 | ++++ | + |
| 9 | ++ | + |
| 10 | ++++ | + |
| 11 | ++ | + |
| 12 | ++ | + |
| 13 | ++++ | + |
| 14 | ++++ | + |
| 15 | ++++ | ++ |
| 16 | ++++ | + |
| 17 | ++ | + |
| 18 | ++ | + |
| 19 | ++++ | + |
| 20 | ++++ | ++ |
| 21 | ++++ | ++ |
| 22 | ++++ | ++ |
| 23 | ++++ | + |
| 24 | ++++ | ++ |
| 25 | ++++ | ++ |
| 26 | ++++ | ++ |
| 27 | ++++ | ++ |
| 28 | ++++ | ++ |
| 29 | ++++ | ++ |
| 30 | ++++ | ++ |
| 31 | ++++ | ++ |
| 32 | ++++ | ++++ |
| 33 | ++++ | ++ |
| 34 | ++++ | ++ |
| 35 | ++ | + |
| 36 | ++++ | ++++ |
| 37 | ++++ | + |
| 38 | ++++ | + |
| 39 | ++++ | + |
| 40 | ++++ | + |
| 41 | ++++ | ++ |
| 42 | ++++ | ++++ |
| 43 | ++ | + |
| 44 | ++++ | + |
| 45 | ++++ | ++ |
| 46 | +++ | + |
| 47 | ++++ | ++ |
| 48 | ++++ | ++ |
| 49 | ++++ | + |
| 50 | ++++ | + |
| 51 | ++++ | + |
| 52 | ++++ | + |
| 53 | ++++ | + |
| 54 | ++ | + |
| 55 | ++++ | + |
| 56 | ++++ | ++ |
| 57 | ++++ | + |
| 58 | ++++ | ++ |
| 59 | ++++ | ++ |
| 60 | ++++ | + |
| 61 | ++++ | ++ |
| 62 | ++++ | + |
| 63 | ++++ | ++ |
| 64 | ++++ | ++ |
| 65 | ++++ | ++ |
| 66 | ++++ | + |
| 67 | ++ | ++ |
| 68 | ++ | + |
| 69 | ++++ | + |
| 70 | ++++ | ++ |
| 71 | ++++ | + |
| 72 | ++++ | + |
| 73 | ++++ | ++ |
| 74 | ++++ | ++ |
| 75 | ++++ | ++ |
| 76 | ++++ | ++ |
| 77 | ++++ | ++ |
| 78 | ++++ | ++ |
| 79 | ++++ | + |
| 80 | ++++ | + |
| 81 | ++++ | + |
| 82 | ++++ | ++ |
| 83 | ++++ | ++ |
| 84 | ++++ | ++ |
| 85 | ++++ | + |
| 86 | ++++ | ++ |
| 87 | ++ | + |
| 88 | ++++ | + |
| 89 | ++++ | + |
| 90 | ++ | + |
| 91 | ++++ | ++ |
| 92 | ++++ | + |
| 93 | ++++ | ++ |
| 94 | ++++ | ++ |
| 95 | ++++ | + |
| 96 | ++++ | + |
| 97 | ++++ | ++ |
| 98 | ++++ | ++ |
| 99 | ++++ | ++ |
| 200 | ++++ | + |
| 201 | ++++ | ++ |
| 102 | ++++ | +++ |
| 103 | ++++ | ++ |
| 104 | ++++ | ++ |
| 105 | ++++ | ++ |
| 106 | ++++ | ++ |
| 107 | ++ | + |
| 108 | ++++ | ++ |
| 109 | ++++ | ++ |
| 110 | ++++ | + |
| 111 | ++++ | + |
| 112 | ++++ | + |
| 113 | ++++ | ++ |
| 114 | ++++ | + |
| 115 | ++++ | + |
| 116 | ++ | + |
| 117 | +++ | + |
| 118 | ++++ | + |
| 119 | +++ | + |
| 120 | ++++ | + |
| 121 | ++++ | + |
| 122 | ++++ | ++ |
| 123 | +++ | + |
| 124 | ++++ | ++ |
| 125 | ++++ | + |
| 126 | ++++ | ++ |
| 127 | +++ | +++ |
| 128 | + | + |
| 129 | ++++ | ++ |
| 130 | ++++ | ++ |
| 131 | ++++ | + |
| 132 | ++++ | ++ |
| 133 | ++++ | + |
| 134 | ++++ | + |
| 135 | ++++ | + |
| 136 | ++++ | ++ |
| 137 | ++++ | +++ |
| 138 | ++++ | + |
| 139 | ++++ | +++ |
| 140 | + | + |
| 141 | ++ | + |
| 142 | + | + |
| 143 | +++ | + |
| 144 | + | + |
| 145 | ++ | + |
| 146 | +++ | + |
| 147 | +++ | + |
| 148 | + | + |
| 149 | ++++ | + |
| 150 | ++++ | ++ |
| 151 | + | + |
| 152 | ++++ | ++ |
| 153 | ++++ | +++ |
| 154 | ++++ | +++ |
| 155 | ++ | + |
| 156 | +++ | + |
| 157 | ++++ | +++ |
| 158 | ++++ | ++ |
| 159 | ++++ | +++ |
| 160 | ++++ | ++ |
| 161 | ++++ | ++ |
| 162 | ++++ | ++ |
| 163 | ++++ | +++ |

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A compound of formula (I) and pharmaceutically acceptable salts thereof: 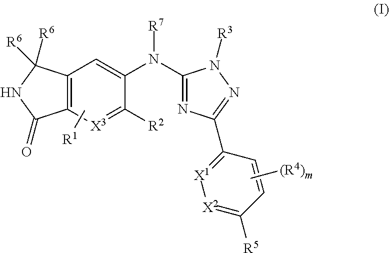
X¹ and X² are each independently selected from carbon and nitrogen; wherein at least one of X¹ and X² is carbon;
X³ is selected from carbon and nitrogen;
R¹ and R² are each independently selected from H, halo, nitro, cyano, NR⁸R⁸, OR¹⁰, SR⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl, C₁-C₄-alkyl substituted with NR⁸R⁹, C₁-C₄-alkyl substituted with OR¹⁰, and cyclopropyl;
R³ is independently selected from C₁-C₄-alkyl, C₁-C₄-haloalkyl, C₀-C₃-alkylene-R^{3a}, and C₂-C₄-alkylene-R^{3b}; wherein R^{3a} is independently at each occurrence selected from cyclopropyl and azetidinyl, said cyclopropyl or azetidinyl groups being optionally substituted with from 1 to 4 R¹ groups; wherein R^{3b} is independently at each occurrence selected from NR⁸R⁹, OR¹⁰ and SR⁸;
R⁴ and R¹² are each independently at each occurrence selected from halo, nitro, cyano, NR⁸R⁹, OR¹⁰, SR⁸, SO₂R⁸, SO₂NR⁸R⁸, CO₂R⁸, C(O)R⁸, CONR⁸R⁸, CR⁸R⁸NR⁸R⁸, CR⁸R⁸OR⁸, C₁-C₄- alkyl, C₂-C₄-alkenyl, C₂-C₄-alkynyl, C₁-C₄-haloalkyl and cyclopropyl;
R⁵ is independently selected from CONR⁸R⁸, unsubstituted phenyl, phenyl substituted with from 1 to 4 R¹¹ groups, and 4- to 10-membered heterocyclyl, wherein said heterocyclyl group may be monocyclic or bicyclic and wherein any given ring of said heterocyclyl group may be saturated, unsaturated or partially unsaturated; wherein any saturated ring or partially unsaturated ring is optionally substituted with a single R¹³ group and/or from 1 to 4 R¹¹ groups and wherein any unsaturated ring is optionally substituted with a single R¹³ group and/or from 1 to 3 R¹² groups;
R⁶ is independently at each occurrence selected from H, halo, C₁-C₄-alkyl, C₁-C₄-alkyl substituted with NR⁸R⁹, C₁-C₄-alkyl substituted with OR¹⁰, and cyclopropyl, or the two R⁶ groups and the carbon atom to which they are attached may together form a C₃-C₆ cycloalkyl ring;
R⁷ and R⁸ are each independently selected from H, C₁-C₄-alkyl and C₁-C₄-haloalkyl;
R⁹ is independently at each occurrence selected from H, C₁-C₄-alkyl, C(O)—C₁-C₄-alkyl and S(O)₂—C₁-C₄-alkyl; or R⁸ and R⁹, together with the nitrogen atom to which they are attached together form a C₅-C₈-heterocycloalkyl group optionally substituted with from 0 to 4 R¹¹ groups;
R¹⁰ is independently at each occurrence selected from H, C₁-C₄-alkyl, C(O)—C₁-C₄-alkyl and C₁-C₄-haloalkyl;
R¹¹ is independently at each occurrence selected from ═O, halo, nitro, cyano, NR⁸R⁹, OR¹⁴, SR⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl and cyclopropyl;
R¹³ is independently selected from C₃-C₆-cycloalkyl, 3- to 6-membered-heterocycloalkyl, CR⁸R⁸NR⁸R⁹ and CR⁸R⁸OR⁸; wherein where R¹³ is cycloalkyl or heterocycloalkyl, R¹³ is optionally substituted with from 1 to 4 R¹¹ groups;
R¹⁴ is independently at each occurrence selected from H, C₁-C₄-alkyl; C₁-C₄-haloalkyl and C(O)—C₁-C₄-alkyl; and
m is an integer selected from 0, 1, 2, 3 and 4;
wherein any of the aforementioned alkyl or cycloalkyl (e.g. cyclopropyl) groups is optionally substituted, where chemically possible, by 1 to 5 substituents which are each independently at each occurrence selected from the group consisting of: halo, oxo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

2. A compound of claim 1, wherein X³ is CR¹.

3. A compound of claim 2, wherein R¹ is H.

4. A compound of any one of claims 1 to 3, wherein R² is independently selected from H, fluoro, C₁-C₃-alkyl, C₁-C₃-fluoroalkyl and cyclopropyl.

5. A compound of any one of claims 1 to 3, wherein R² is H.

6. A compound of any one of claims 1 to 5, wherein R⁷ is H.

7. A compound of any one of claims 1 to 6, wherein R³ is independently selected from C₁-C₄-alkyl and cyclopropyl.

8. A compound of any one of claims 1 to 7, wherein m is 1.

9. A compound of claim 8, wherein R⁴ is selected from C₁-C₃-alkyl and cyclopropyl.

10. A compound of any one of claims 1 to 9, wherein R⁵ is imidazole, optionally substituted with a single R¹³ group and/or from 1 to 3 R¹² groups.

11. A compound of any one of claims 1 to 9, wherein R⁵ is pyridine, optionally substituted with a single R¹³ group and/or from 1 to 3 R¹² groups.

12. A compound of claim 11, wherein R⁵ has the structure: 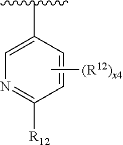
wherein x4 is an integer independently selected from 0, 1 and 2.

13. A compound of claim 11, wherein R⁵ has the structure 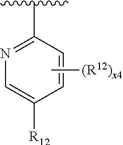
wherein x4 is an integer independently selected from 0, 1 and 2.

14. A compound of any one of claims 1 to 13, wherein R⁶ is independently at each occurrence selected from H and C₁-C₄-alkyl.

15. A compound of any one of claims 1 to 13, wherein the two R⁶ groups and the carbon atom to which they are attached together form a C₃-C₆ cycloalkyl ring.

16. A compound of formula (I) may be selected from:

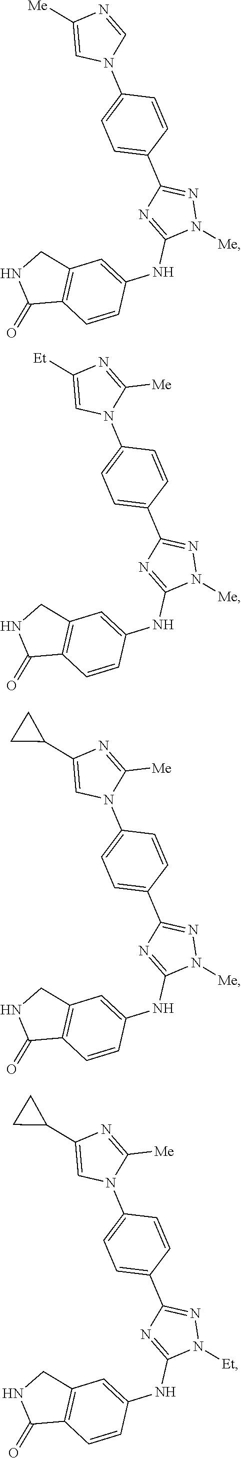

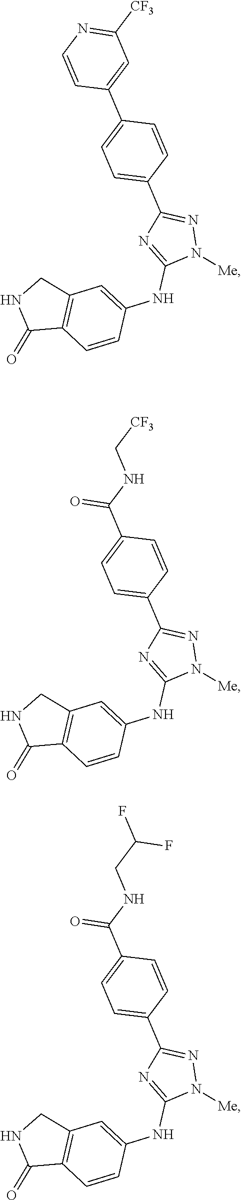 [SMILES: Cn1nc(-c2ccc(-c3ccnc(C(F)(F)F)c3)cc2)nc1Nc1ccc2c(c1)CNC2=O ; Cn1nc(-c2ccc(C(=O)NCC(F)(F)F)cc2)nc1Nc1ccc2c(c1)CNC2=O ; Cn1nc(-c2ccc(C(=O)NCC(F)F)cc2)nc1Nc1ccc2c(c1)CNC2=O]

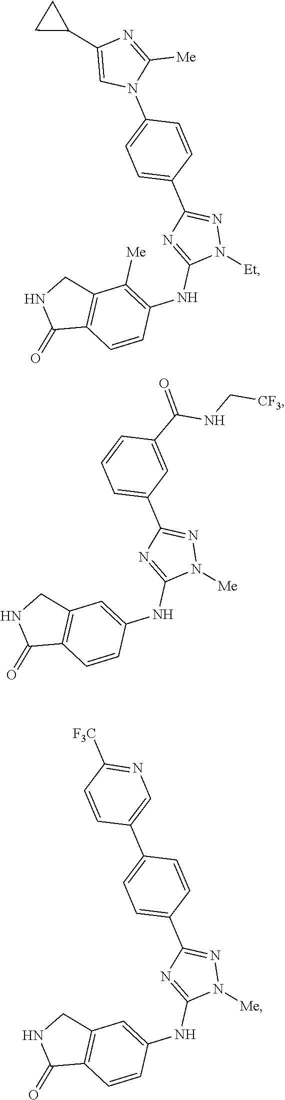 [SMILES: CCn1nc(-c2ccc(-n3cc(C4CC4)nc3C)cc2)nc1Nc1ccc2c(c1C)CNC2=O ; Cn1nc(-c2cccc(C(=O)NCC(F)(F)F)c2)nc1Nc1ccc2c(c1)CNC2=O ; Cn1nc(-c2ccc(-c3ccc(C(F)(F)F)nc3)cc2)nc1Nc1ccc2c(c1)CNC2=O]

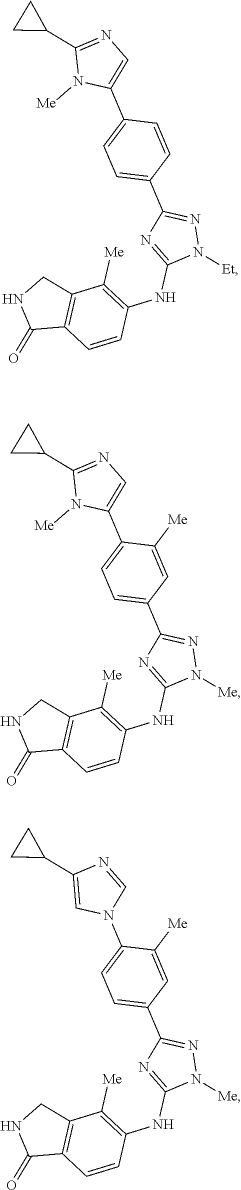 [SMILES: CCn1nc(-c2ccc(-c3cnc(C4CC4)n3C)cc2)nc1Nc1ccc2c(c1C)CNC2=O ; Cc1cc(-c2nc(Nc3ccc4c(c3C)CNC4=O)n(C)n2)ccc1-c1cnc(C2CC2)n1C ; Cc1cc(-c2nc(Nc3ccc4c(c3C)CNC4=O)n(C)n2)ccc1-n1cnc(C2CC2)c1]

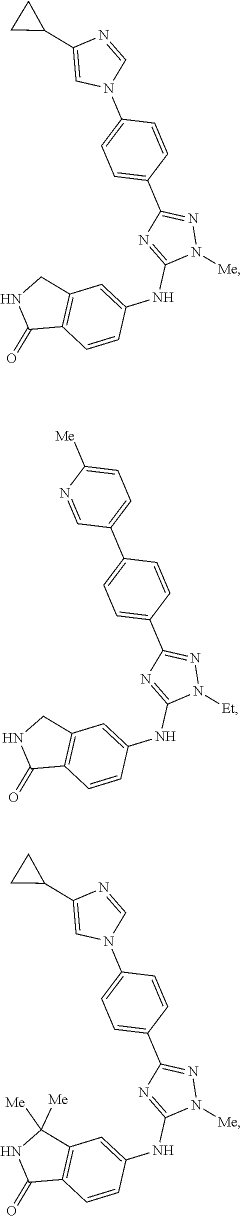 [SMILES: Cn1nc(-c2ccc(-n3cnc(C4CC4)c3)cc2)nc1Nc1ccc2c(c1)CNC2=O ; CCn1nc(-c2ccc(-c3ccc(C)nc3)cc2)nc1Nc1ccc2c(c1)CNC2=O ; Cn1nc(-c2ccc(-n3cnc(C4CC4)c3)cc2)nc1Nc1ccc2c(c1)C(C)(C)NC2=O]

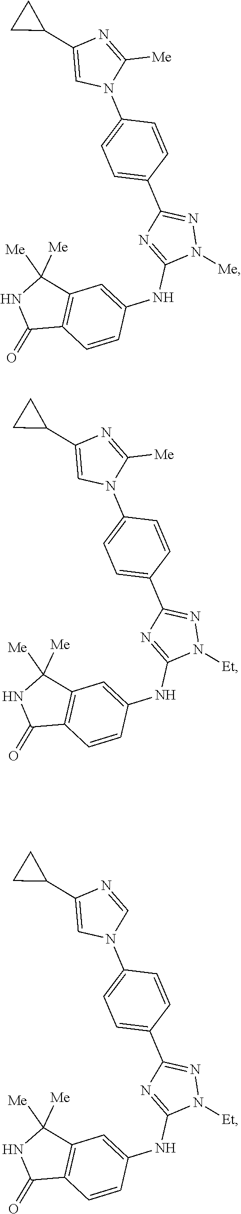

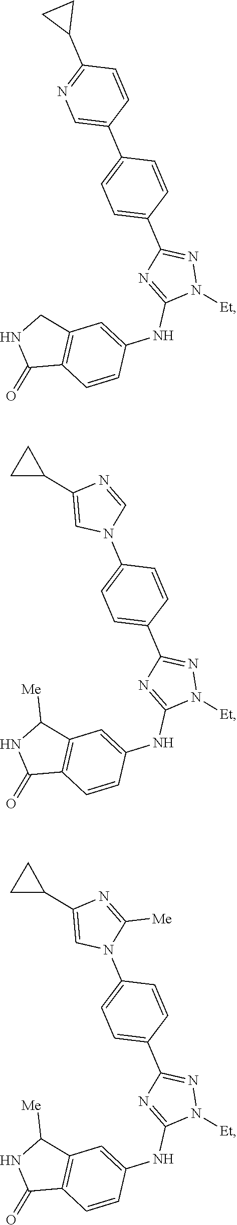 [SMILES: CCn1nc(-c2ccc(-c3ccc(C4CC4)nc3)cc2)nc1Nc1ccc2c(c1)CNC2=O ; CCn1nc(-c2ccc(-n3cnc(C4CC4)c3)cc2)nc1Nc1ccc2c(c1)C(C)NC2=O ; CCn1nc(-c2ccc(-n3cc(C4CC4)nc3C)cc2)nc1Nc1ccc2c(c1)C(C)NC2=O]

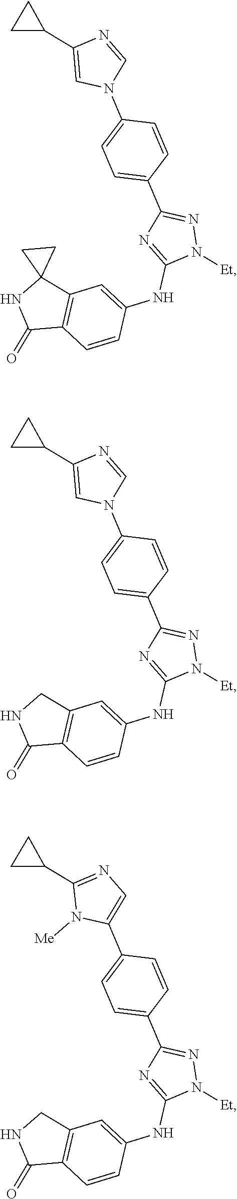 [SMILES: CCn1nc(-c2ccc(-n3cnc(C4CC4)c3)cc2)nc1Nc1ccc2c(c1)C1(CC1)NC2=O ; CCn1nc(-c2ccc(-n3cnc(C4CC4)c3)cc2)nc1Nc1ccc2c(c1)CNC2=O ; CCn1nc(-c2ccc(-c3cnc(C4CC4)n3C)cc2)nc1Nc1ccc2c(c1)CNC2=O]

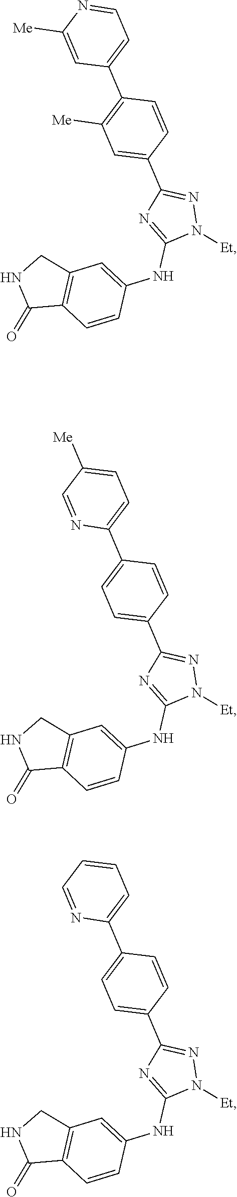 [SMILES: CCn1nc(-c2ccc(-c3ccnc(C)c3)c(C)c2)nc1Nc1ccc2c(c1)CNC2=O ; CCn1nc(-c2ccc(-c3ccc(C)cn3)cc2)nc1Nc1ccc2c(c1)CNC2=O ; CCn1nc(-c2ccc(-c3ccccn3)cc2)nc1Nc1ccc2c(c1)CNC2=O]

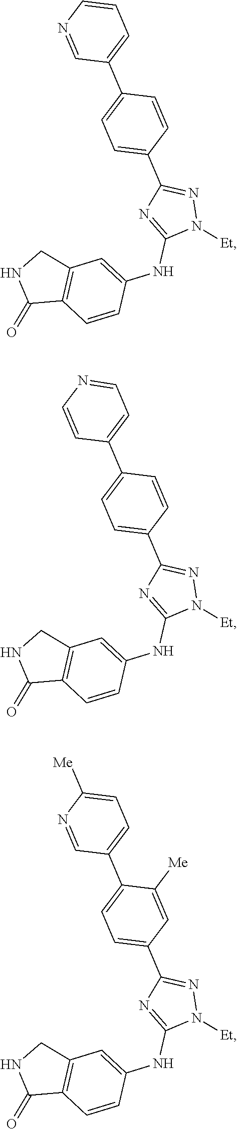 [SMILES: CCn1nc(-c2ccc(-c3cccnc3)cc2)nc1Nc1ccc2c(c1)CNC2=O ; CCn1nc(-c2ccc(-c3ccncc3)cc2)nc1Nc1ccc2c(c1)CNC2=O ; CCn1nc(-c2ccc(-c3ccc(C)nc3)c(C)c2)nc1Nc1ccc2c(c1)CNC2=O]

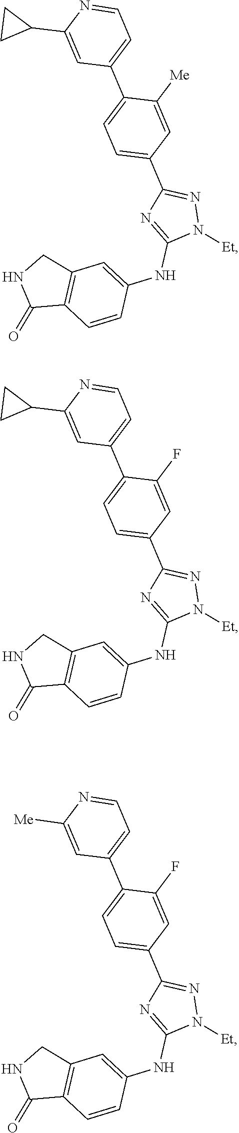 [SMILES: CCn1nc(-c2ccc(-c3ccnc(C4CC4)c3)c(C)c2)nc1Nc1ccc2c(c1)CNC2=O ; CCn1nc(-c2ccc(-c3ccnc(C4CC4)c3)c(F)c2)nc1Nc1ccc2c(c1)CNC2=O ; CCn1nc(-c2ccc(-c3ccnc(C)c3)c(F)c2)nc1Nc1ccc2c(c1)CNC2=O]

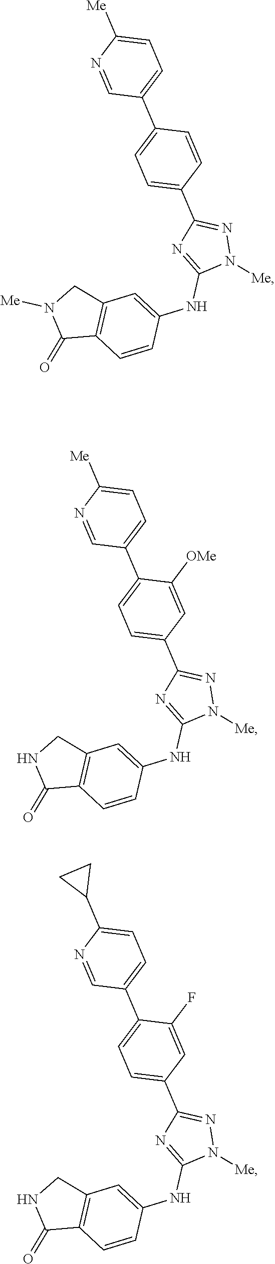 [SMILES: Cc1ccc(-c2ccc(-c3nc(Nc4ccc5c(c4)CN(C)C5=O)n(C)n3)cc2)cn1 ; COc1cc(-c2nc(Nc3ccc4c(c3)CNC4=O)n(C)n2)ccc1-c1ccc(C)nc1 ; Cn1nc(-c2ccc(-c3ccc(C4CC4)nc3)c(F)c2)nc1Nc1ccc2c(c1)CNC2=O]

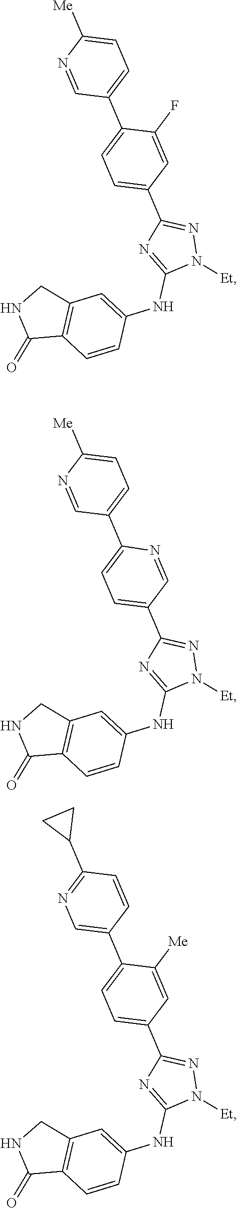

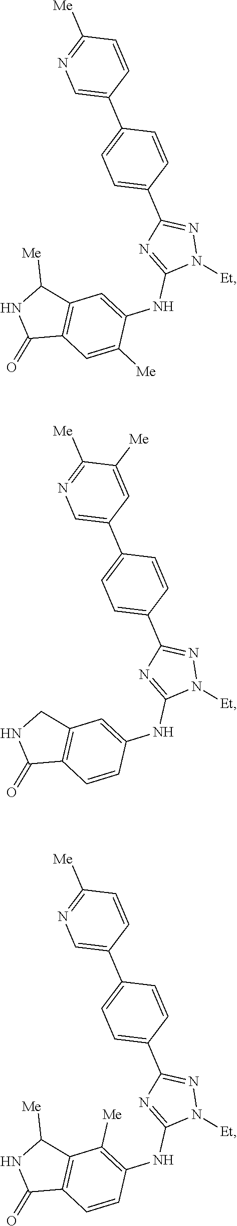 [SMILES: CCn1nc(-c2ccc(-c3ccc(C)nc3)cc2)nc1Nc1cc2c(cc1C)C(=O)NC2C ; CCn1nc(-c2ccc(-c3cnc(C)c(C)c3)cc2)nc1Nc1ccc2c(c1)CNC2=O ; CCn1nc(-c2ccc(-c3ccc(C)nc3)cc2)nc1Nc1ccc2c(c1C)C(C)NC2=O]

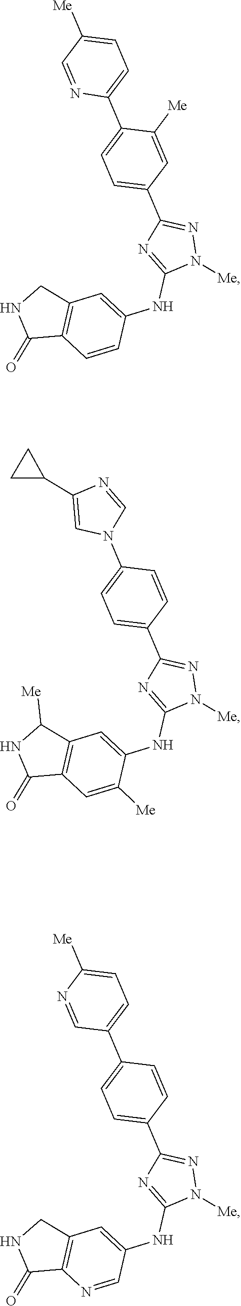 [SMILES: Cc1ccc(-c2ccc(-c3nc(Nc4ccc5c(c4)CNC5=O)n(C)n3)cc2C)nc1 ; Cc1cc2c(cc1Nc1nc(-c3ccc(-n4cnc(C5CC5)c4)cc3)nn1C)C(C)NC2=O ; Cc1ccc(-c2ccc(-c3nc(Nc4cnc5c(c4)CNC5=O)n(C)n3)cc2)cn1]

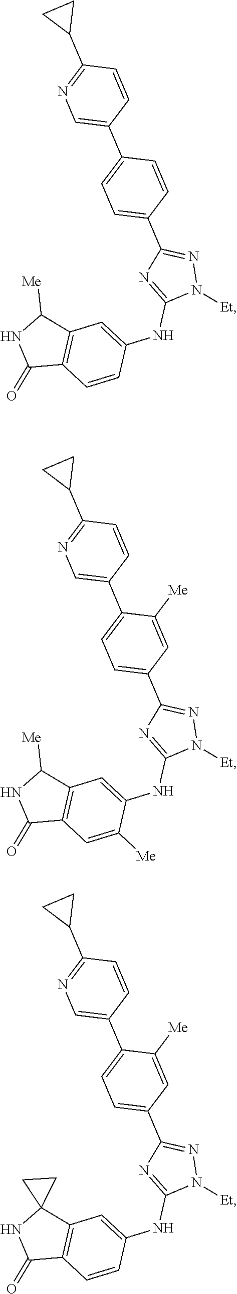 [SMILES: CCn1nc(-c2ccc(-c3ccc(C4CC4)nc3)cc2)nc1Nc1ccc2c(c1)C(C)NC2=O ; CCn1nc(-c2ccc(-c3ccc(C4CC4)nc3)c(C)c2)nc1Nc1cc2c(cc1C)C(=O)NC2C ; CCn1nc(-c2ccc(-c3ccc(C4CC4)nc3)c(C)c2)nc1Nc1ccc2c(c1)C1(CC1)NC2=O]

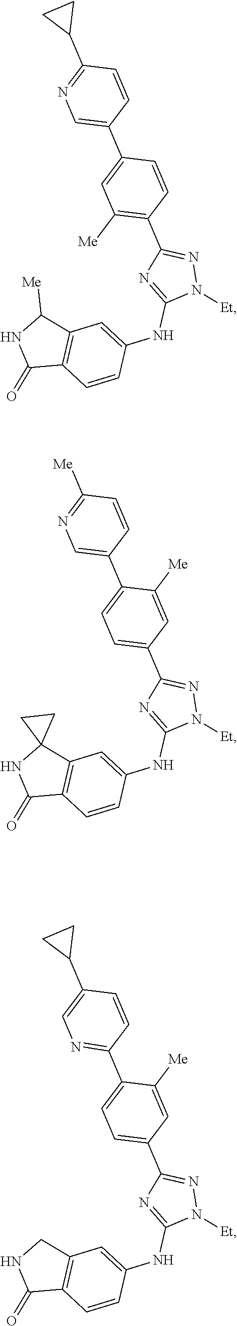 [SMILES: CCn1nc(-c2ccc(-c3ccc(C4CC4)nc3)cc2C)nc1Nc1ccc2c(c1)C(C)NC2=O ; CCn1nc(-c2ccc(-c3ccc(C)nc3)c(C)c2)nc1Nc1ccc2c(c1)C1(CC1)NC2=O ; CCn1nc(-c2ccc(-c3ccc(C4CC4)cn3)c(C)c2)nc1Nc1ccc2c(c1)CNC2=O]

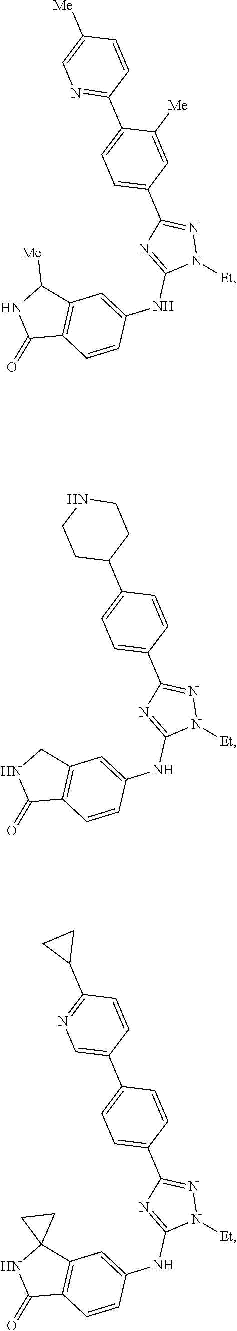 [SMILES: CCn1nc(-c2ccc(-c3ccc(C)cn3)c(C)c2)nc1Nc1ccc2c(c1)C(C)NC2=O ; CCn1nc(-c2ccc(C3CCNCC3)cc2)nc1Nc1ccc2c(c1)CNC2=O ; CCn1nc(-c2ccc(-c3ccc(C4CC4)nc3)cc2)nc1Nc1ccc2c(c1)C1(CC1)NC2=O]

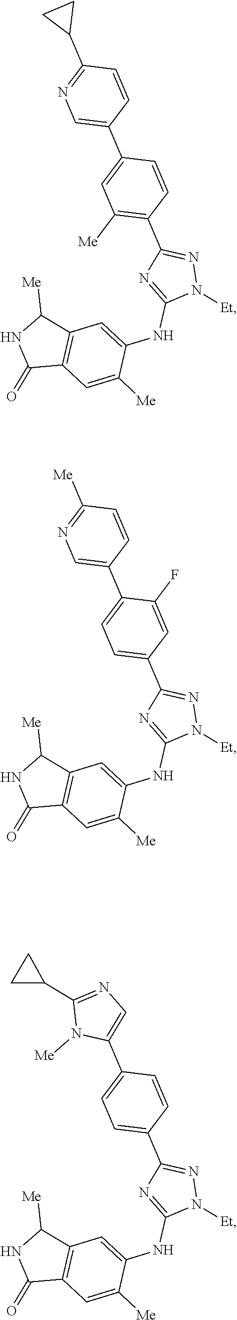 [SMILES: CCn1nc(-c2ccc(-c3ccc(C4CC4)nc3)cc2C)nc1Nc1cc2c(cc1C)C(=O)NC2C ; CCn1nc(-c2ccc(-c3ccc(C)nc3)c(F)c2)nc1Nc1cc2c(cc1C)C(=O)NC2C ; CCn1nc(-c2ccc(-c3cnc(C4CC4)n3C)cc2)nc1Nc1cc2c(cc1C)C(=O)NC2C]

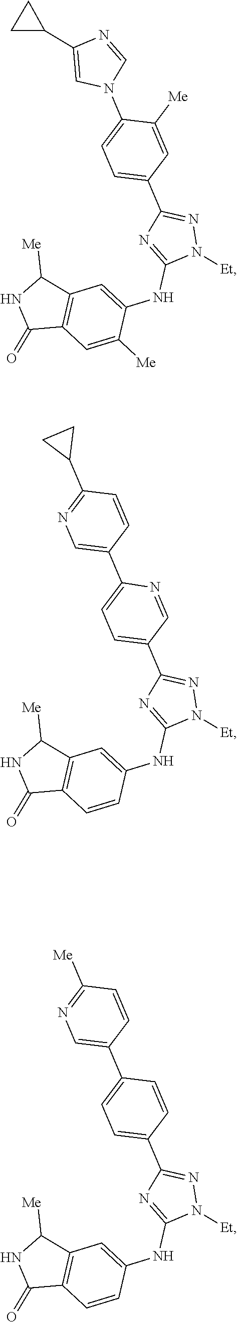 [SMILES: CCn1nc(-c2ccc(-n3cnc(C4CC4)c3)c(C)c2)nc1Nc1cc2c(cc1C)C(=O)NC2C ; CCn1nc(-c2ccc(-c3ccc(C4CC4)nc3)nc2)nc1Nc1ccc2c(c1)C(C)NC2=O ; CCn1nc(-c2ccc(-c3ccc(C)nc3)cc2)nc1Nc1ccc2c(c1)C(C)NC2=O]

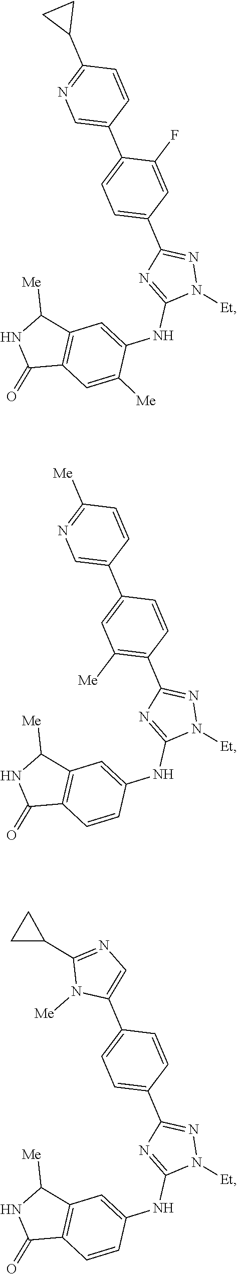 [SMILES: CCn1nc(-c2ccc(-c3ccc(C4CC4)nc3)c(F)c2)nc1Nc1cc2c(cc1C)C(=O)NC2C ; CCn1nc(-c2ccc(-c3ccc(C)nc3)cc2C)nc1Nc1ccc2c(c1)C(C)NC2=O ; CCn1nc(-c2ccc(-c3cnc(C4CC4)n3C)cc2)nc1Nc1ccc2c(c1)C(C)NC2=O]

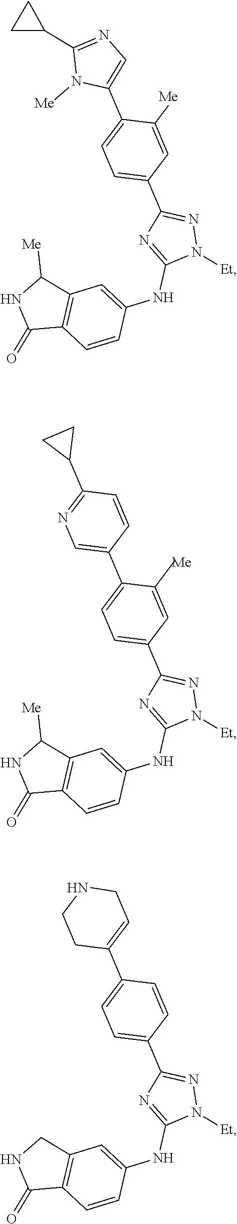 [SMILES: CCn1nc(-c2ccc(-c3cnc(C4CC4)n3C)c(C)c2)nc1Nc1ccc2c(c1)C(C)NC2=O ; CCn1nc(-c2ccc(-c3ccc(C4CC4)nc3)c(C)c2)nc1Nc1ccc2c(c1)C(C)NC2=O ; CCn1nc(-c2ccc(C3=CCNCC3)cc2)nc1Nc1ccc2c(c1)CNC2=O]

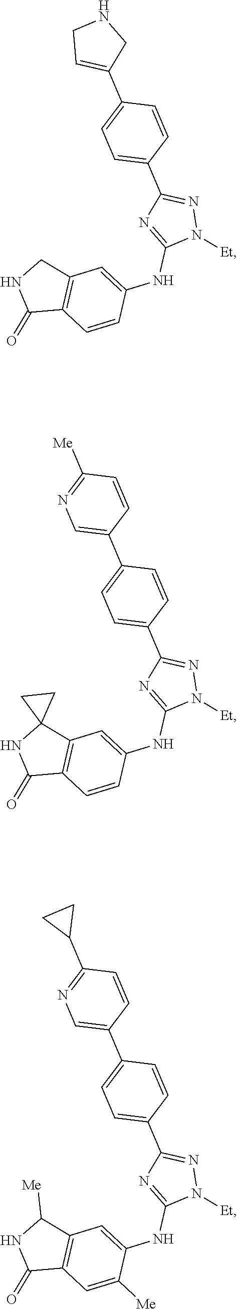 [SMILES: CCn1nc(-c2ccc(C3=CCNC3)cc2)nc1Nc1ccc2c(c1)CNC2=O ; CCn1nc(-c2ccc(-c3ccc(C)nc3)cc2)nc1Nc1ccc2c(c1)C1(CC1)NC2=O ; CCn1nc(-c2ccc(-c3ccc(C4CC4)nc3)cc2)nc1Nc1cc2c(cc1C)C(=O)NC2C]

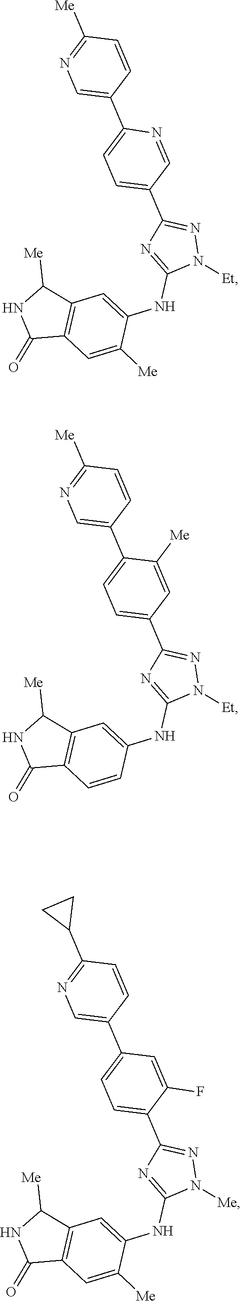 [SMILES: CCn1nc(-c2ccc(-c3ccc(C)nc3)nc2)nc1Nc1cc2c(cc1C)C(=O)NC2C ; CCn1nc(-c2ccc(-c3ccc(C)nc3)c(C)c2)nc1Nc1ccc2c(c1)C(C)NC2=O ; Cc1cc2c(cc1Nc1nc(-c3ccc(-c4ccc(C5CC5)nc4)cc3F)nn1C)C(C)NC2=O]

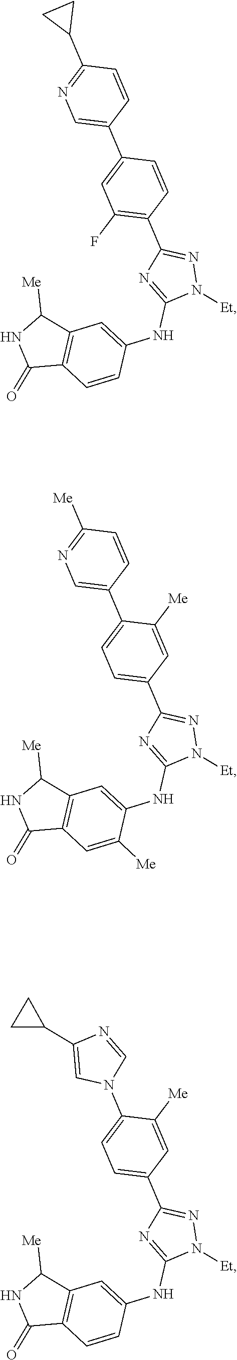 [SMILES: CCn1nc(-c2ccc(-c3ccc(C4CC4)nc3)cc2F)nc1Nc1ccc2c(c1)C(C)NC2=O ; CCn1nc(-c2ccc(-c3ccc(C)nc3)c(C)c2)nc1Nc1cc2c(cc1C)C(=O)NC2C ; CCn1nc(-c2ccc(-n3cnc(C4CC4)c3)c(C)c2)nc1Nc1ccc2c(c1)C(C)NC2=O]

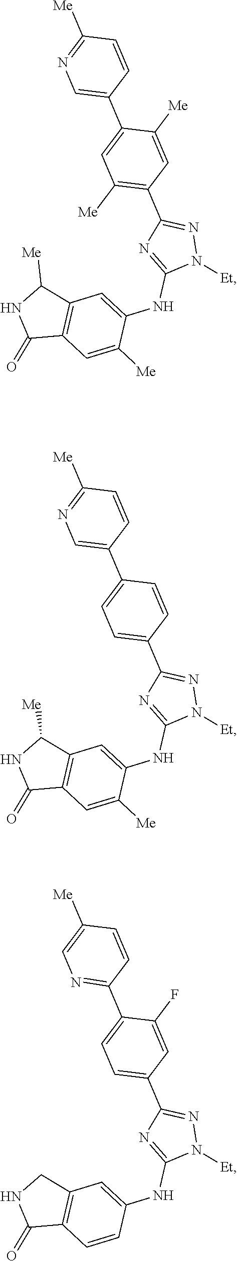 [SMILES: CCn1nc(-c2cc(C)c(-c3ccc(C)nc3)cc2C)nc1Nc1cc2c(cc1C)C(=O)NC2C ; CCn1nc(-c2ccc(-c3ccc(C)nc3)cc2)nc1Nc1cc2c(cc1C)C(=O)N[C@@H]2C ; CCn1nc(-c2ccc(-c3ccc(C)cn3)c(F)c2)nc1Nc1ccc2c(c1)CNC2=O]

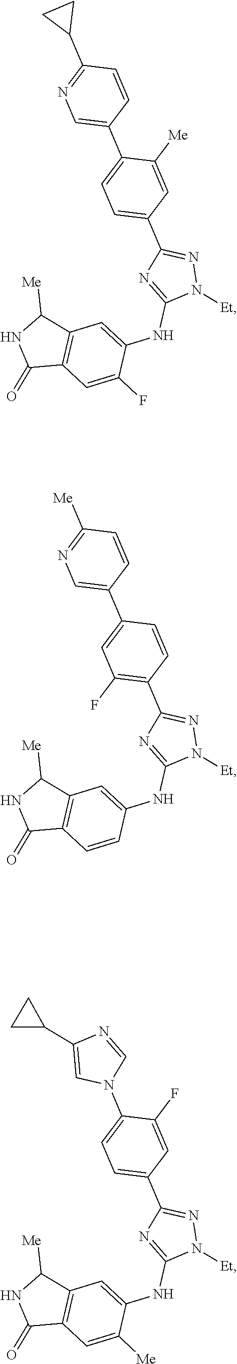 [SMILES: CCn1nc(-c2ccc(-c3ccc(C4CC4)nc3)c(C)c2)nc1Nc1cc2c(cc1F)C(=O)NC2C ; CCn1nc(-c2ccc(-c3ccc(C)nc3)cc2F)nc1Nc1ccc2c(c1)C(C)NC2=O ; CCn1nc(-c2ccc(-n3cnc(C4CC4)c3)c(F)c2)nc1Nc1cc2c(cc1C)C(=O)NC2C]

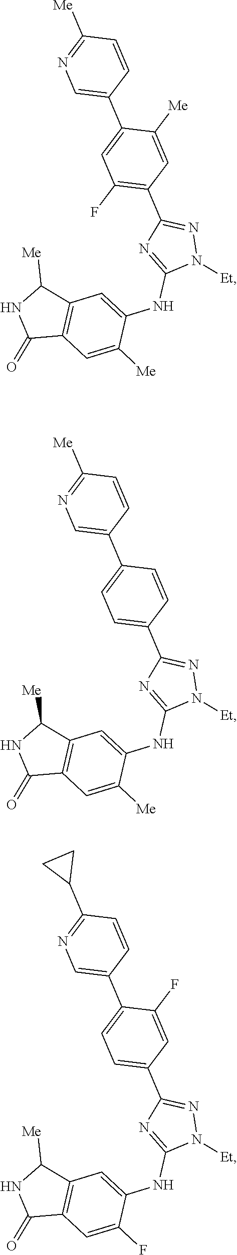 [SMILES: CCn1nc(-c2cc(C)c(-c3ccc(C)nc3)cc2F)nc1Nc1cc2c(cc1C)C(=O)NC2C ; CCn1nc(-c2ccc(-c3ccc(C)nc3)cc2)nc1Nc1cc2c(cc1C)C(=O)N[C@H]2C ; CCn1nc(-c2ccc(-c3ccc(C4CC4)nc3)c(F)c2)nc1Nc1cc2c(cc1F)C(=O)NC2C]

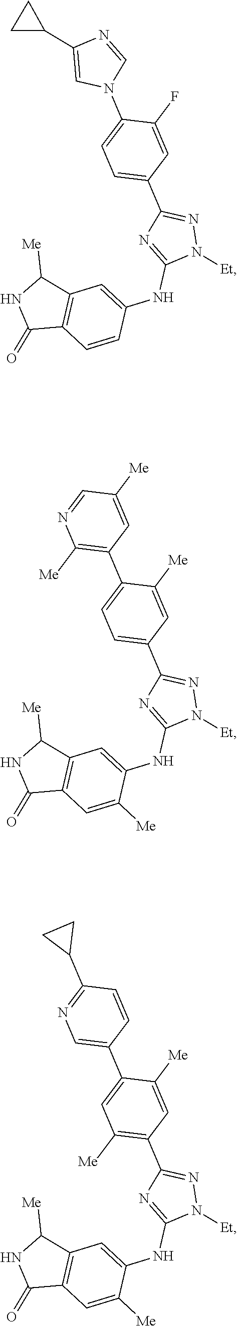 [SMILES: CCn1nc(-c2ccc(-n3cnc(C4CC4)c3)c(F)c2)nc1Nc1ccc2c(c1)C(C)NC2=O ; CCn1nc(-c2ccc(-c3cc(C)cnc3C)c(C)c2)nc1Nc1cc2c(cc1C)C(=O)NC2C ; CCn1nc(-c2cc(C)c(-c3ccc(C4CC4)nc3)cc2C)nc1Nc1cc2c(cc1C)C(=O)NC2C]

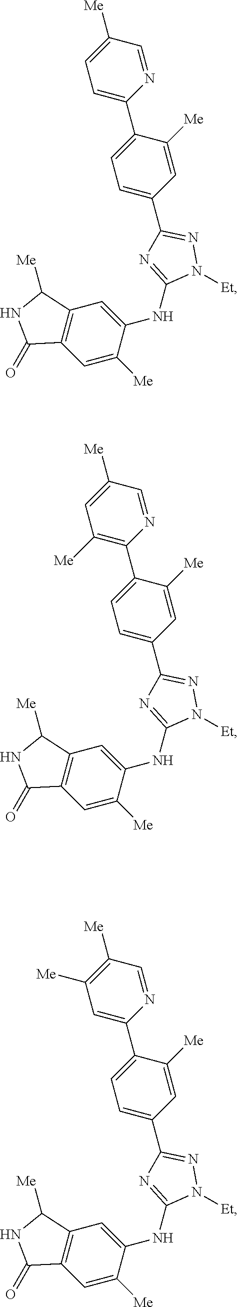 [SMILES: CCn1nc(-c2ccc(-c3ccc(C)cn3)c(C)c2)nc1Nc1cc2c(cc1C)C(=O)NC2C ; CCn1nc(-c2ccc(-c3ncc(C)cc3C)c(C)c2)nc1Nc1cc2c(cc1C)C(=O)NC2C ; CCn1nc(-c2ccc(-c3cc(C)c(C)cn3)c(C)c2)nc1Nc1cc2c(cc1C)C(=O)NC2C]

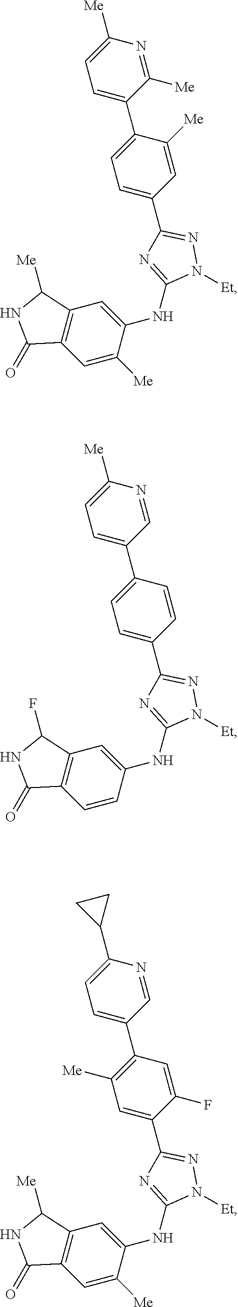 [SMILES: CCn1nc(-c2ccc(-c3ccc(C)nc3C)c(C)c2)nc1Nc1cc2c(cc1C)C(=O)NC2C ; CCn1nc(-c2ccc(-c3ccc(C)nc3)cc2)nc1Nc1ccc2c(c1)C(F)NC2=O ; CCn1nc(-c2cc(C)c(-c3ccc(C4CC4)nc3)cc2F)nc1Nc1cc2c(cc1C)C(=O)NC2C]

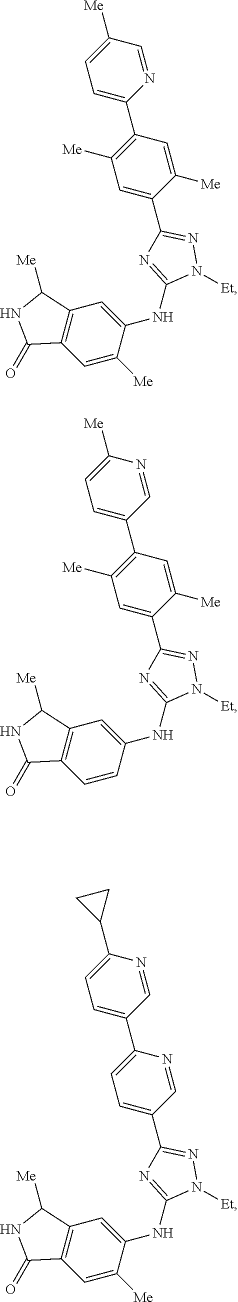 [SMILES: CCn1nc(-c2cc(C)c(-c3ccc(C)cn3)cc2C)nc1Nc1cc2c(cc1C)C(=O)NC2C ; CCn1nc(-c2cc(C)c(-c3ccc(C)nc3)cc2C)nc1Nc1ccc2c(c1)C(C)NC2=O ; CCn1nc(-c2ccc(-c3ccc(C4CC4)nc3)nc2)nc1Nc1cc2c(cc1C)C(=O)NC2C]

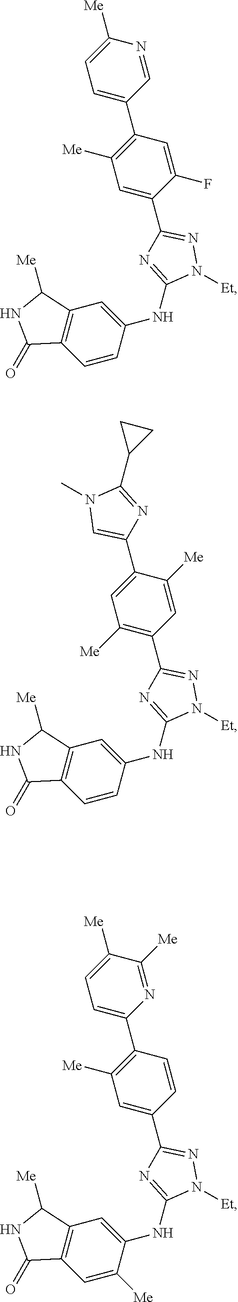 [SMILES: CCn1nc(-c2cc(C)c(-c3ccc(C)nc3)cc2F)nc1Nc1ccc2c(c1)C(C)NC2=O ; CCn1nc(-c2cc(C)c(-c3cn(C)c(C4CC4)n3)cc2C)nc1Nc1ccc2c(c1)C(C)NC2=O ; CCn1nc(-c2ccc(-c3ccc(C)c(C)n3)c(C)c2)nc1Nc1cc2c(cc1C)C(=O)NC2C]

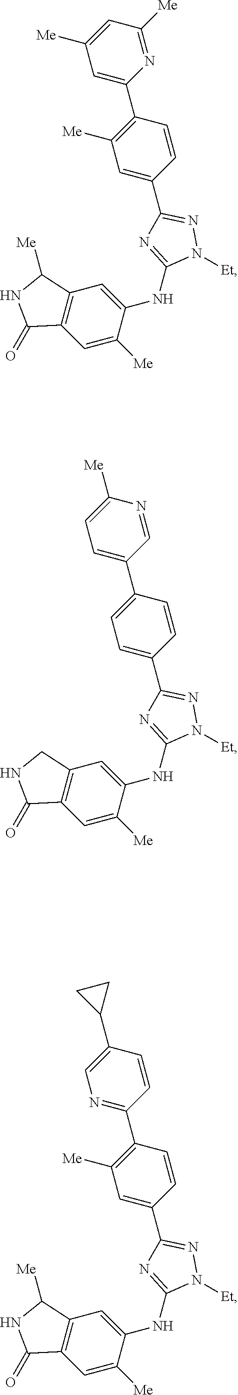 [SMILES: CCn1nc(-c2ccc(-c3cc(C)cc(C)n3)c(C)c2)nc1Nc1cc2c(cc1C)C(=O)NC2C ; CCn1nc(-c2ccc(-c3ccc(C)nc3)cc2)nc1Nc1cc2c(cc1C)C(=O)NC2 ; CCn1nc(-c2ccc(-c3ccc(C4CC4)cn3)c(C)c2)nc1Nc1cc2c(cc1C)C(=O)NC2C]

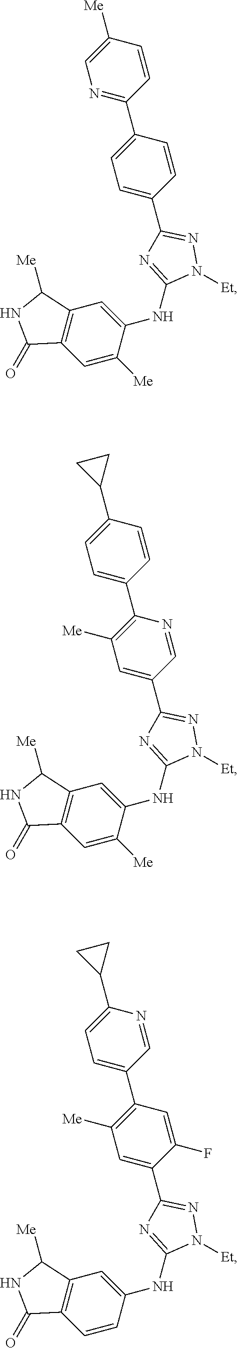 [SMILES: CCn1nc(-c2ccc(-c3ccc(C)cn3)cc2)nc1Nc1cc2c(cc1C)C(=O)NC2C ; CCn1nc(-c2cnc(-c3ccc(C4CC4)cc3)c(C)c2)nc1Nc1cc2c(cc1C)C(=O)NC2C ; CCn1nc(-c2cc(C)c(-c3ccc(C4CC4)nc3)cc2F)nc1Nc1ccc2c(c1)C(C)NC2=O]

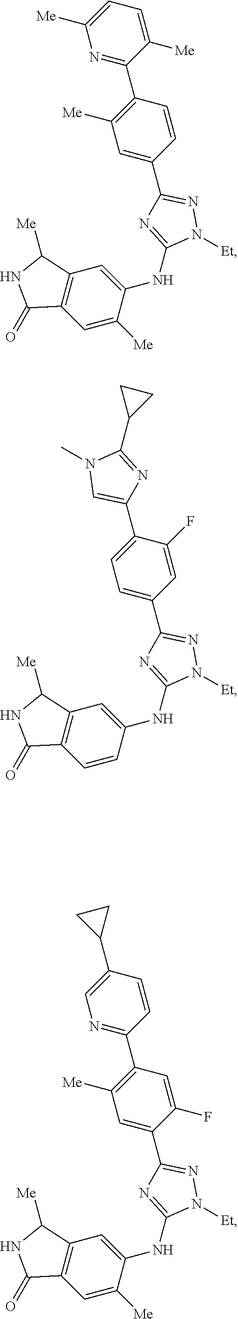

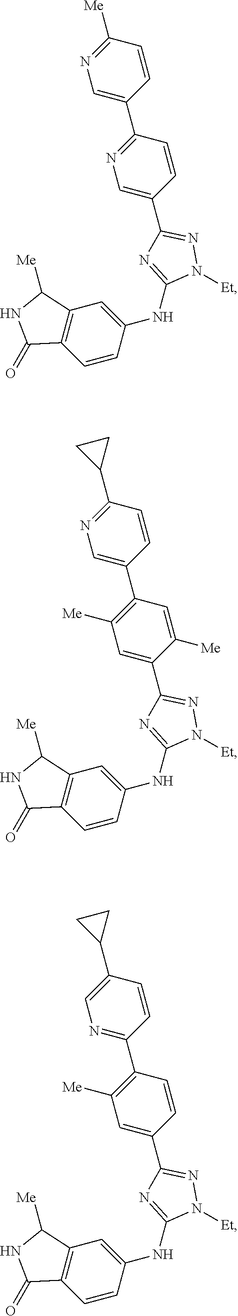 [SMILES: CCn1nc(-c2ccc(-c3ccc(C)nc3)nc2)nc1Nc1ccc2c(c1)C(C)NC2=O ; CCn1nc(-c2cc(C)c(-c3ccc(C4CC4)nc3)cc2C)nc1Nc1ccc2c(c1)C(C)NC2=O ; CCn1nc(-c2ccc(-c3ccc(C4CC4)cn3)c(C)c2)nc1Nc1ccc2c(c1)C(C)NC2=O]

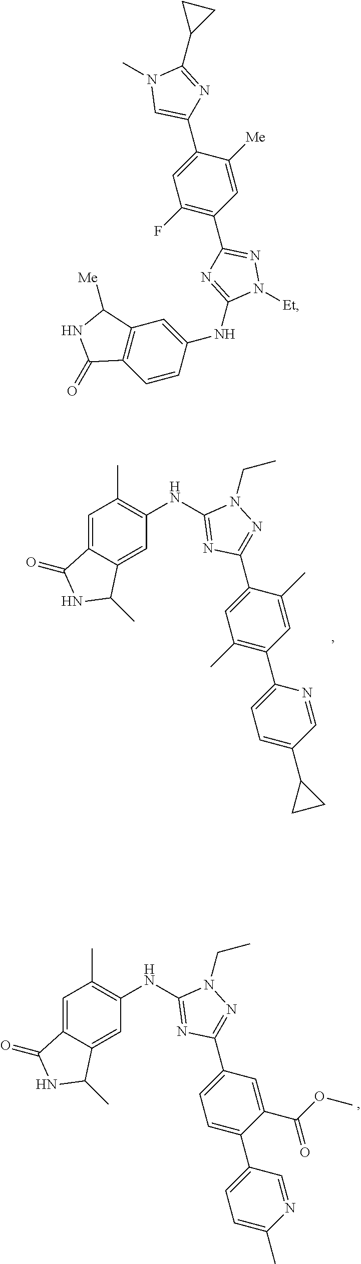 [SMILES: CCn1nc(-c2cc(C)c(-c3cn(C)c(C4CC4)n3)cc2F)nc1Nc1ccc2c(c1)C(C)NC2=O ; CCn1nc(-c2cc(C)c(-c3ccc(C4CC4)cn3)cc2C)nc1Nc1cc2c(cc1C)C(=O)NC2C ; CCn1nc(-c2ccc(-c3ccc(C)nc3)c(C(=O)OC)c2)nc1Nc1cc2c(cc1C)C(=O)NC2C]

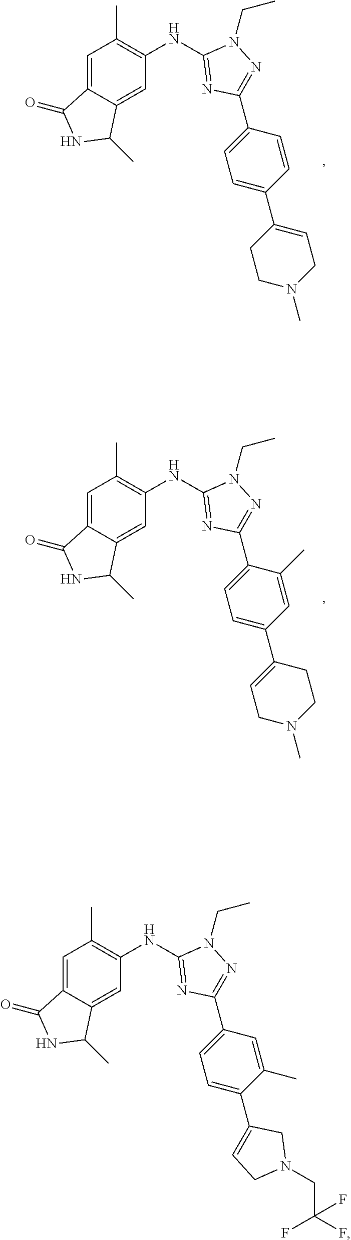 [SMILES: CCn1nc(-c2ccc(C3=CCN(C)CC3)cc2)nc1Nc1cc2c(cc1C)C(=O)NC2C ; CCn1nc(-c2ccc(C3=CCN(C)CC3)cc2C)nc1Nc1cc2c(cc1C)C(=O)NC2C ; CCn1nc(-c2ccc(C3=CCN(CC(F)(F)F)C3)c(C)c2)nc1Nc1cc2c(cc1C)C(=O)NC2C]

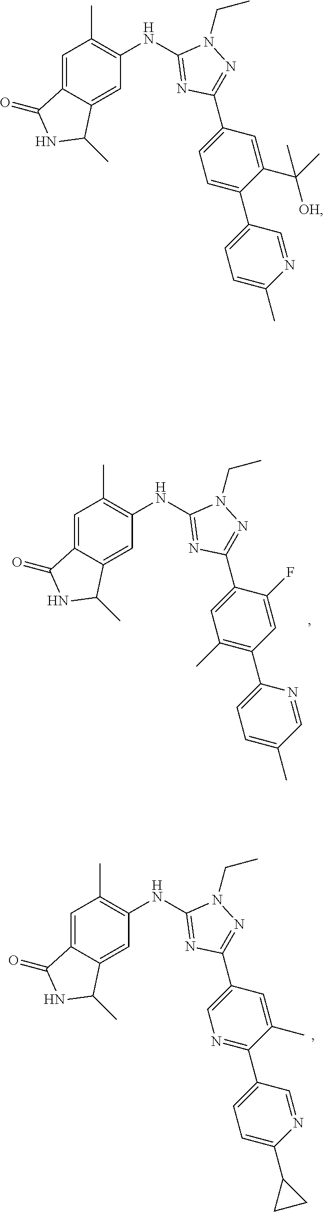 [SMILES: CCn1nc(-c2ccc(-c3ccc(C)nc3)c(C(C)(C)O)c2)nc1Nc1cc2c(cc1C)C(=O)NC2C ; CCn1nc(-c2cc(C)c(-c3ccc(C)cn3)cc2F)nc1Nc1cc2c(cc1C)C(=O)NC2C ; CCn1nc(-c2cnc(-c3ccc(C4CC4)nc3)c(C)c2)nc1Nc1cc2c(cc1C)C(=O)NC2C]

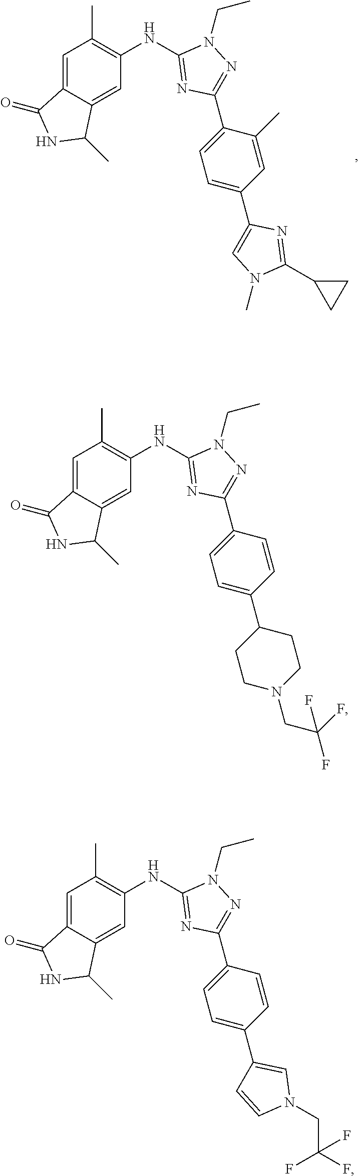 [SMILES: CCn1nc(-c2ccc(-c3cn(C)c(C4CC4)n3)cc2C)nc1Nc1cc2c(cc1C)C(=O)NC2C ; CCn1nc(-c2ccc(C3CCN(CC(F)(F)F)CC3)cc2)nc1Nc1cc2c(cc1C)C(=O)NC2C ; CCn1nc(-c2ccc(-c3ccn(CC(F)(F)F)c3)cc2)nc1Nc1cc2c(cc1C)C(=O)NC2C]

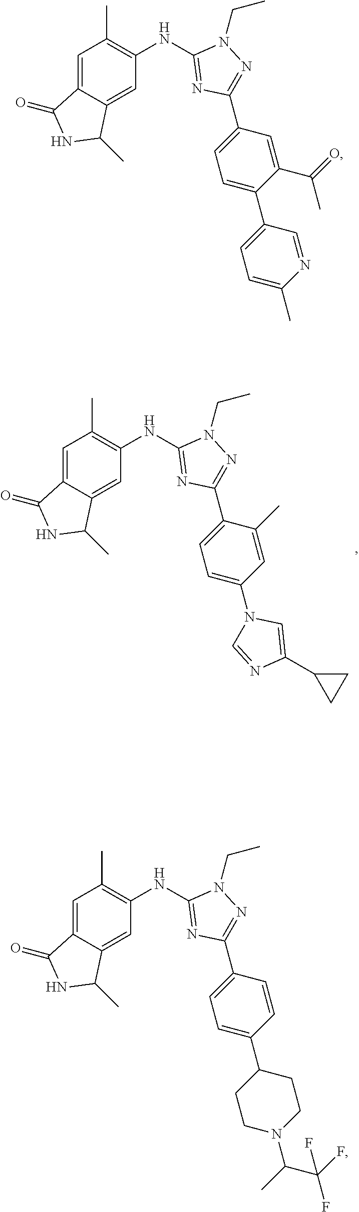 [SMILES: CCn1nc(-c2ccc(-c3ccc(C)nc3)c(C(C)=O)c2)nc1Nc1cc2c(cc1C)C(=O)NC2C ; CCn1nc(-c2ccc(-n3cnc(C4CC4)c3)cc2C)nc1Nc1cc2c(cc1C)C(=O)NC2C ; CCn1nc(-c2ccc(C3CCN(C(C)C(F)(F)F)CC3)cc2)nc1Nc1cc2c(cc1C)C(=O)NC2C]

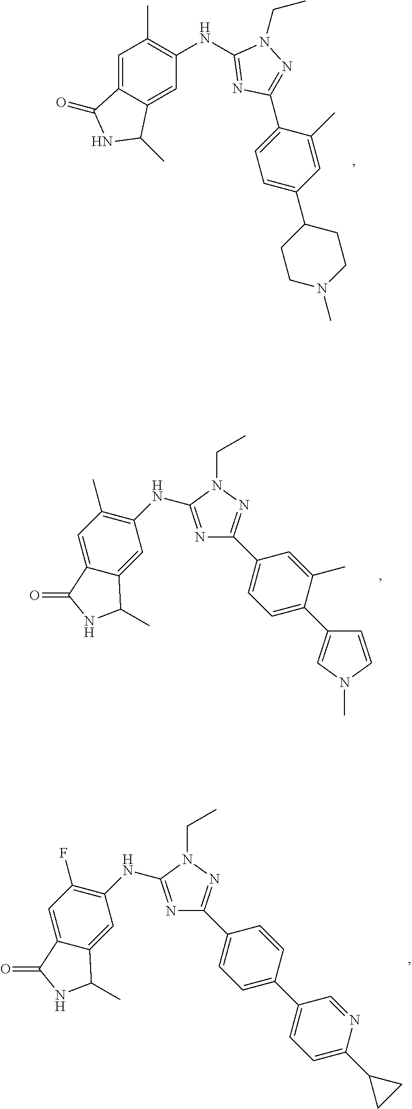 [SMILES: CCn1nc(-c2ccc(C3CCN(C)CC3)cc2C)nc1Nc1cc2c(cc1C)C(=O)NC2C ; CCn1nc(-c2ccc(-c3ccn(C)c3)c(C)c2)nc1Nc1cc2c(cc1C)C(=O)NC2C ; CCn1nc(-c2ccc(-c3ccc(C4CC4)nc3)cc2)nc1Nc1cc2c(cc1F)C(=O)NC2C]

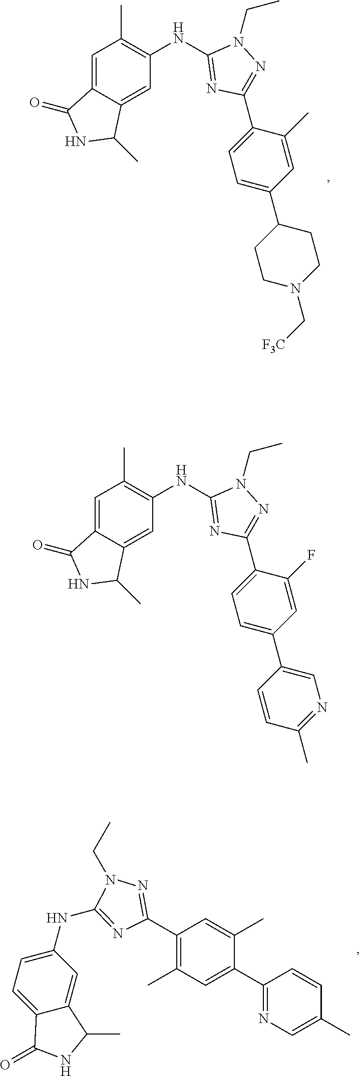 [SMILES: CCn1nc(-c2ccc(C3CCN(CC(F)(F)F)CC3)cc2C)nc1Nc1cc2c(cc1C)C(=O)NC2C ; CCn1nc(-c2ccc(-c3ccc(C)nc3)cc2F)nc1Nc1cc2c(cc1C)C(=O)NC2C ; CCn1nc(-c2cc(C)c(-c3ccc(C)cn3)cc2C)nc1Nc1ccc2c(c1)C(C)NC2=O]

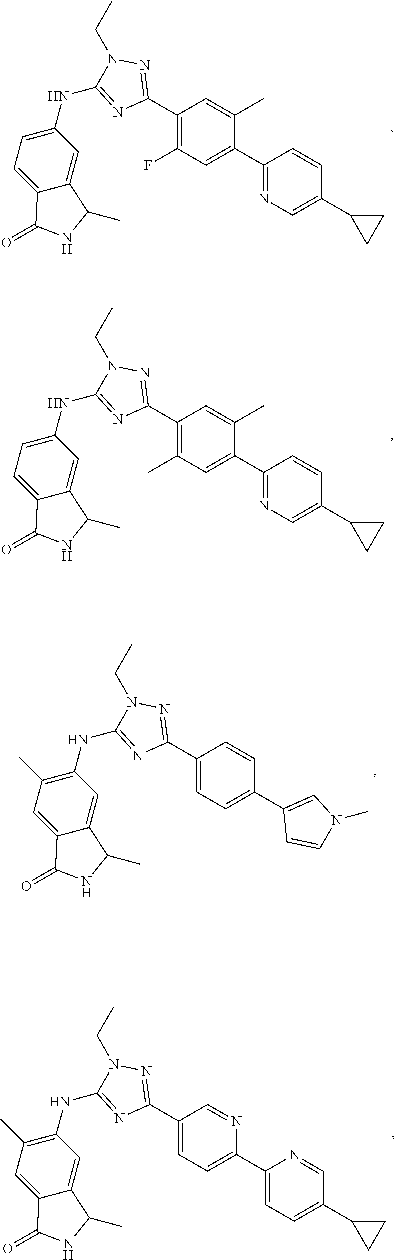

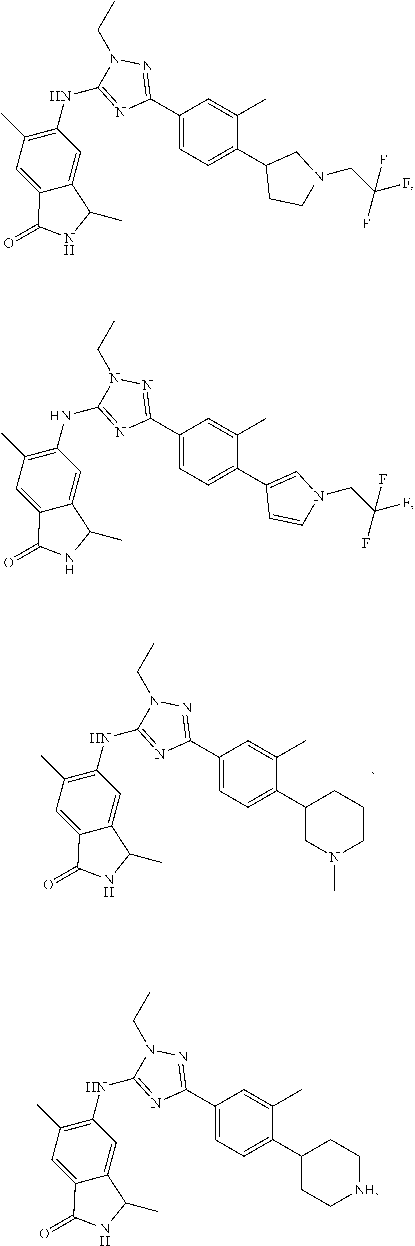 [SMILES: CCn1nc(-c2ccc(C3CCN(CC(F)(F)F)C3)c(C)c2)nc1Nc1cc2c(cc1C)C(=O)NC2C ; CCn1nc(-c2ccc(-c3ccn(CC(F)(F)F)c3)c(C)c2)nc1Nc1cc2c(cc1C)C(=O)NC2C ; CCn1nc(-c2ccc(C3CCCN(C)C3)c(C)c2)nc1Nc1cc2c(cc1C)C(=O)NC2C ; CCn1nc(-c2ccc(C3CCNCC3)c(C)c2)nc1Nc1cc2c(cc1C)C(=O)NC2C]

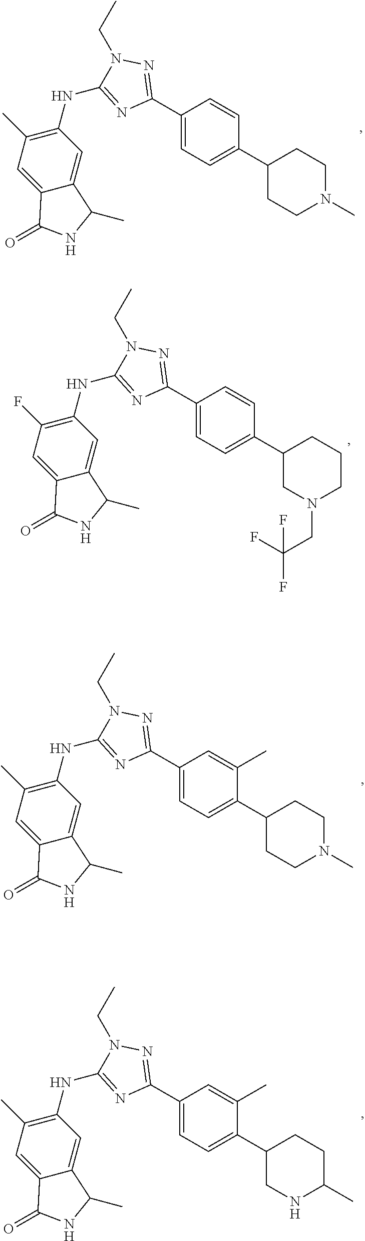

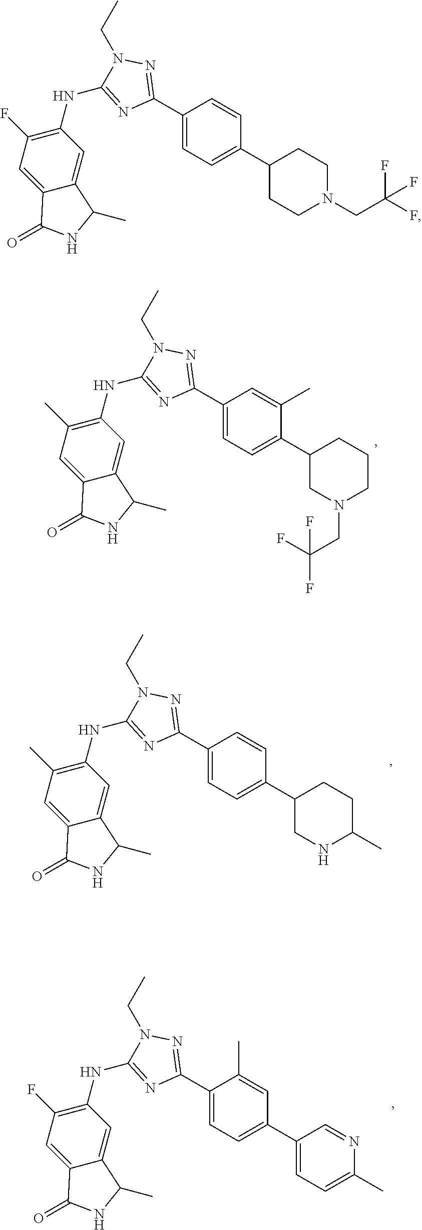

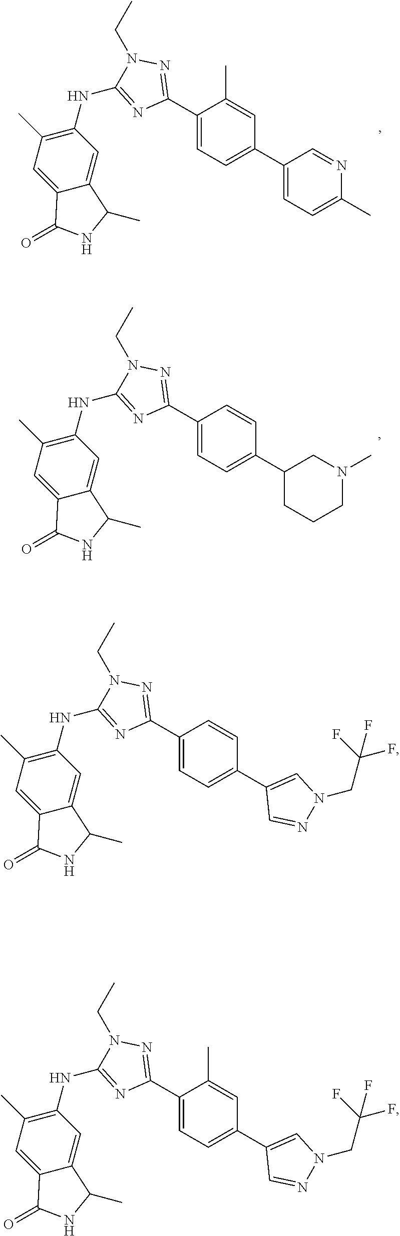

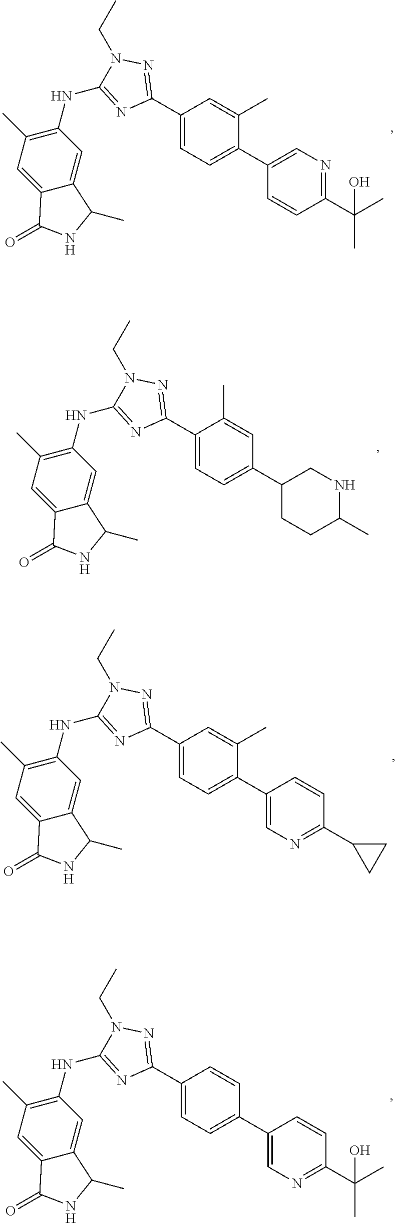

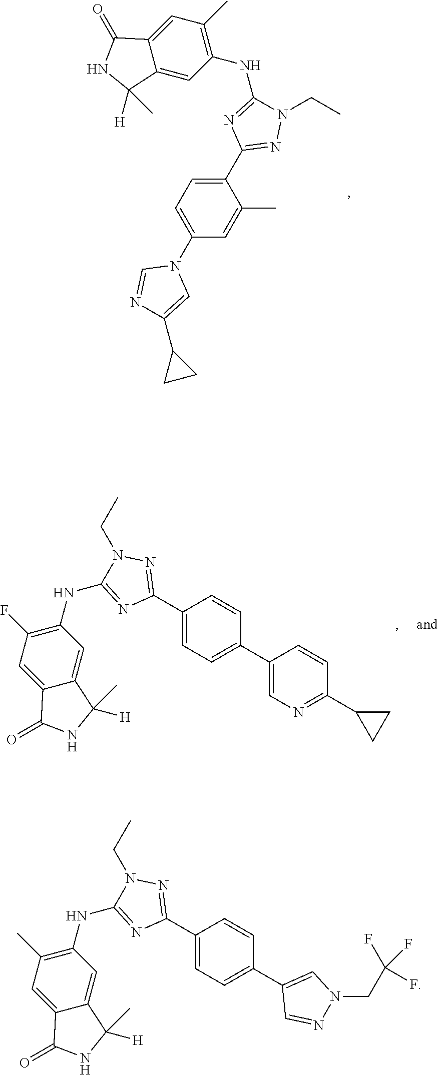 [SMILES: CCn1nc(-c2ccc(-n3cnc(C4CC4)c3)cc2C)nc1Nc1cc2c(cc1C)C(=O)NC2C ; CCn1nc(-c2ccc(-c3ccc(C4CC4)nc3)cc2)nc1Nc1cc2c(cc1F)C(=O)NC2C ; CCn1nc(-c2ccc(-c3cnn(CC(F)(F)F)c3)cc2)nc1Nc1cc2c(cc1C)C(=O)NC2C]

17. A pharmaceutical formulation comprising a compound of any one of claims 1 to 16 and a pharmaceutically acceptable excipient.

18. A compound of any one of claims 1 to 16 for use as a medicament.

19. A compound of any one of claims 1 to 16 for use in treating a disease or disorder selected from: fibrotic diseases, auto-immune, inflammatory-fibrotic conditions, inflammatory conditions, central nervous system disorders, or cancer.
